(19) United States
(12) Patent Application Publication
(10) Pub. No.: US 2016/0068583 A1
(43) Pub. Date: Mar. 10, 2016

(54) Interleukin-10 Compositions and Uses Thereof

(71) Applicant: ARMO BIOSCIENCES, INC., Redwood City, CA (US)

(72) Inventors: Peter Van Vlasselaer, Woodside, CA (US); Scott McCauley, San Francisco, CA (US)

(21) Appl. No.: 14/779,928

(22) PCT Filed: Apr. 23, 2014
(86) PCT No.: PCT/US14/35201

§ 371 Date: Sep. 24, 2015

Related U.S. Application Data
(60) Provisional application No. 61/815,657, filed on Apr. 24, 2013.

(51) Int. Cl. C07K 14/54; A61K 38/20; G01N 33/68; A61K 39/395; A61K 47/48; A61K 9/00; C07K 16/24

(52) U.S. Cl. CPC C07K 14/5428; A61K 9/0019; A61K 38/2066; C07K 16/244; A61K 39/3955; A61K 47/48215; G01N 33/6869; C07K 2317/92; C07K 2317/21; C07K 2317/24; C07K 2317/622

(57) ABSTRACT

Interleukin-10 muteins and other interleukin-10-related molecules are described, as well as methods of identifying interleukin-10 muteins and other interleukin-10-related molecules. Also described herein are modifications of the foregoing, which modifications may enhance a property (e.g., half-life) of the muteins or other molecules compared to human interleukin-10. Particular interleukin-10 muteins and related molecules have comparable immunogenicity to human interleukin-10 and/or bioactivity at least comparable to human interleukin-10. Pharmaceutical compositions and methods of use are also described herein.

### CROSS-REFERENCE TO RELATED APPLICATION

This application claims priority benefit of U.S. provisional application Ser. No. 61/815,657, filed Apr. 24, 2013, which application is incorporated herein in its entirety.

### FIELD OF THE INVENTION

The present invention relates to, among other things, interleukin-10 muteins and other interleukin-10-related molecules, modifications of the foregoing, and associated uses thereof.

### INTRODUCTION

The cytokine interleukin-10 (IL-10) is a pleiotropic cytokine that regulates multiple immune responses through actions on T cells, B cells, macrophages, and antigen presenting cells (APC). IL-10 may suppress immune responses by inhibiting expression of IL-1α, IL-1β, IL-6, IL-8, TNF-α, GM-CSF and G-CSF in activated monocytes and activated macrophages, and it also suppresses IFN-γ production by NK cells. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of IL-2- and IL-4-treated thymocytes, enhancing the viability of B cells, and stimulating the expression of MHC class II.

As a result of its pleiotropic activity, IL-10 has been linked to a broad range of diseases, disorders and conditions, including inflammatory conditions, immune-related disorders, fibrotic disorders and cancer. Clinical and pre-clinical evaluations with IL-10 for a number of such diseases, disorders and conditions have solidified its therapeutic potential. Moreover, pegylated IL-10 has been shown to be more efficacious than non-pegylated IL-10 in certain therapeutic settings.

In view of the prevalence and severity of IL-10-associated diseases, disorders and conditions, novel IL-10 agents and modifications thereof would be of tremendous value in the treatment and prevention of IL-10-associated diseases, disorders and conditions.

### SUMMARY

The present disclosure relates to IL-10 compositions and uses thereof. The terms “IL-10”, “IL-10 polypeptide(s),” “IL-10-agent(s)”, “IL-10 molecule(s)” and the like are intended to be construed broadly and include, for example, human and non-human IL-10-related polypeptides, including homologs, variants (including muteins), and fragments thereof, as well as IL-10 polypeptides having, for example, a leader sequence (e.g., a signal peptide). Particular embodiments relate to modifications of the foregoing. In particular embodiments, the modification(s) improves at least one property or other characteristic (e.g., efficacy) of the peptides compared to unmodified versions of the peptides thereof. Further embodiments of the present disclosure pertain to methods and other technologies for identifying specific amino acid residues or domains of IL-10 that may be modified according to the methods described herein. Methods of using (e.g., in the treatment or prevention of a disorder or a symptom thereof), identifying and/or generating the peptides described herein are also aspects of the present disclosure. Other aspects include, for example, pharmaceutical compositions comprising the peptides.

Human IL-10 (and IL-10 from other species) exists as a homodimer. Each monomer of wild-type human IL-10 comprises 178 amino acids, the first 18 of which comprise a signal peptide. As set forth in detail hereafter, each 160 amino acid monomer of mature human IL-10 (hIL-10) comprises six helices (A-F) linked by short loops, which are also referred to herein as inter-helix junctions. For the sake of clarity, inter-helix junctions can comprise one or more amino acid residues (generally fewer than 10 residues).

Amino acid residues and regions of the IL-10 helices, inter-helices junctions and kinks (described hereafter) that can or cannot be mutated and/or modified are discussed hereafter. By way of example, amino acid residues and regions that are buried within the three-dimensional core of IL-10 or that are involved with receptor binding are generally not candidates for modification.

The present disclosure contemplates peptides comprising a substitution that would facilitate the attachment of a PEG or other moiety to at least one amino acid residue. Examples of such peptides are described in detail hereafter.

In particular embodiments of the present disclosure, a mutant IL-10 or a modified IL-10 peptide is less immunogenic (i.e., stimulates less of an immune response) than the corresponding unmodified IL-10 peptide. In other embodiments, a modified IL-10 peptide is immunogenic-neutral (i.e., immunogenicity is not altered in a therapeutically relevant way) than the corresponding unmodified IL-10 peptide. Methods are described herein for evaluating the immunogenicity of the IL-10 peptides described herein. In still further embodiments, a modified peptide has and improvement in at least one property (e.g., a physical property, including solubility, bioavailability, serum half-life, and circulation time). Such properties are described further hereafter.

The present disclosure contemplates peptides comprising the amino acid sequence of SEQ ID NO:2, wherein the peptides comprise at least one amino acid substitution, deletion or addition, and wherein the substitution(s), deletion(s) or addition(s) does not, for example, adversely affect immunogenicity. The present disclosure also contemplates peptides having at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:2, wherein the peptides a) are not more immunogenic than the peptide of SEQ ID NO:2, and/or b) have a bioactivity at least equal to the bioactivity of the peptide of SEQ ID NO:2, and/or c) have at least one property (e.g., a physical property, including solubility, bioavailability, serum half-life, and circulation time) that is improved compared to the peptide of SEQ ID NO:2. It will be apparent to the skilled artisan that utilization of different methodologies (e.g., different methods of quantifying the exact concentration of IL-10 and/or different methods of producing IL-10) may result in IL-10 that is more or less active—either in apparent activity due to differences in calculating protein concentration or in actual activity—than this reference standard. By leveraging their skill and experience, the skilled artisan will be able to factor in these differences in determining the relative bioactivities of an IL-10 molecule versus hIL-10. In some embodiments, each monomer of such peptides has at least 100, at least 110, at least 125, at least 140, at least 145, at least 150, at least 151, at least 152, at least 153, at least 154, at least 155, at least 156, at least 157, at least 158, or at least 159 amino acid residues.

In some embodiments, the amino acid residue addition(s), deletion(s), or substitution(s) of the aforementioned peptides does not disrupt the intramolecular disulfide bonds of the peptides or the non-covalent interactions between the two monomer subunits of the peptides. However, it should be noted that such an addition(s), deletion(s), or substitution(s) might possibly disrupt one or more of the intra-monomeric non-covalent bonds (e.g., hydrogen bonds), but that such disruption should not have a therapeutically relevant effect on protein function. According to the teachings of the present disclosure, an amino acid substitution may be a conservative substitution, and/or an amino acid substitution is not a substitution at one or more of amino acid residues 12, 62, 108 and 114.

In particular embodiments, the present disclosure contemplates peptides having a bioactivity at least equal to the bioactivity of SEQ ID NO:2. Bioactivity may be determined by any method known in the art, including a chemokine release assay, a TNFα inhibition assay or an MC/9 cell proliferation assay. Exemplary protocols for such assays are described herein. Likewise, the immunogenicity of the peptides may be predicted or determined by any method known to the skilled artisan, including prediction by screening for at least one of T-cell epitopes or B-cell epitopes. In one aspect, immunogenicity is predicted by an in silico system and/or in an ex vivo assay system.

The instant disclosure also contemplates peptides comprising the amino acid sequence of SEQ ID NO:2, wherein the peptides comprise at least one amino acid substitution of a surface-exposed amino acid residue, and wherein the substitution does not adversely affect immunogenicity and/or another property or characteristic. In certain embodiments, these peptides also do not comprise substitution of any amino acid residues involved with receptor binding. However, it is to be understood that substitution, deletion, and/or addition of one or more amino acid residues within the IL-10 receptor binding region, or in close proximity thereto, that may be tolerated are contemplated by the present disclosure.

In further embodiments, the peptides described in the preceding paragraph comprise a) a Pre-helix A; b) a Helix A; c) an A/B Inter-helix Junction; d) a Helix B; e) a B/C Inter-helix Junction; f) a Helix C; g) a C/D Inter-helix Junction; h) a Helix D; i) a D/E Inter-helix Junction; j) a Helix E; k) an E/F Inter-helix Junction; l) a Helix F; and m) a Post-helix F; wherein such peptides further comprise at least one of: i) substitution of at least one amino acid residue of Pre-helix A other than amino acid residues 12 (C), 15 (F) or 16 (P); or ii) substitution of at least one amino acid residue of Helix A other than amino acid residues 19-24 (LPNMLR (SEQ ID NO:33)), 26-30 (LRDAF (SEQ ID NO:34)), 33-39 17 (VKTFFQM (SEQ ID NO:35)), or 41 (D); or iii) substitution of at least one amino acid residue of Helix B other than amino acid residues 52 (L), 53 (L), or 56 (F); or iv) substitution of the amino acid residue of the B/C Inter-helix Junction; or v) substitution of at least one amino acid residue of Helix C other than amino acid residues 62 (C), 64 (A), 65 (L), 68 (M), 69 (I), 71-73 (FYL), 76 (V), 77 (M), or 80 (A); or vi) substitution of at least one amino acid residue of the C/D Inter-helix Junction; or vii) substitution of at least one amino acid residue of Helix D other than amino acid residues 87 (I), 91 (V), 94 (L), 98 (L), 101 (L), 105 (L), or 108 (C); or viii) substitution of at least one amino acid residue of the D/E Inter-helix Junction other than amino acid residues 111 (F), 112 (L), or 114 (C); or ix) substitution of at least one amino acid residue of Helix E other than amino acid residues 120 (A), 121 (V), 124 (V), 127 (A), 128 (F) or 131 (L); or x) substitution of the amino acid residue of the E/F Inter-helix Junction; or xi) substitution of at least one amino acid residue of Helix F other than amino acid residues 136-156 (IYKAMSEFDIFINYIEAYMTM (SEQ ID NO:36)), 158 (I) or 159 (R); or xii) substitution of the amino acid residue of Post-helix F. The boundaries of these regions are set forth in FIG. 3C. The tyrosine at amino acid residue 59 is a candidate for modification (e.g., pegylation).

In some embodiments the amino acid residue addition(s), deletion(s), or substitution(s) of the peptides described in the preceding paragraph does not disrupt the intramolecular disulfide bonds of the peptides or the non-covalent interactions between the two monomer subunits of the peptides. It should be noted, however, that such an addition(s), deletion(s), or substitution(s) might possibly disrupt one or more of the intra-monomeric non-covalent bonds (e.g., hydrogen bonds), but that such disruption should not have a therapeutically relevant effect on protein function. In other embodiments the amino acid substitution may be a conservative substitution, and/or the amino acid substitution is not a substitution at one or more of amino acid residues 12, 62, 108 and 114. The bioactivity and immunogenicity of these peptides may be assessed according to the teachings set forth herein.

Particular embodiments of the present disclosure contemplate modification(s) of the peptides described herein, wherein the modification(s) does not alter the amino acid sequence of the peptides (i.e., no amino acid substitutions, additions or deletions are introduced into the IL-10 primary amino acid sequence), and wherein the modification(s) improves or otherwise enhances at least one property or other characteristic (e.g., a pharmacokinetic parameter or efficacy) of the peptides compared to unmodified versions of the peptides.

In some embodiments, modification of the IL-10 peptides does not cause a detrimental effect on immunogenicity of a level that is therapeutically relevant, and in still further embodiments the modified IL-10 is less immunogenic than unmodified IL-10.

The present disclosure contemplates the introduction of any modification that may be advantageous. Thus, in particular embodiments, the modification improves at least one physical property of the peptide (e.g., solubility, bioavailability, serum half-life, and circulation time). Other modifications include introducing means for blocking receptor cleavage and increasing affinity for the IL-10 receptor(s) (or modifying the off-rate so that the IL-10 molecule will be docked with the receptor(s) for a longer duration).

In some embodiments, the modification is pegylation and the modified peptide is PEG-IL-10. The pegylated peptides may comprise at least one PEG molecule covalently attached to at least one amino acid residue of at least one monomer of IL-10. The PEG molecule may be conjugated to IL-10 through a linker; linkers are described in detail hereafter. Such pegylated peptides may comprise a mixture of mono-pegylated and di-pegylated IL-10. References herein to “mono-pegylated” or “di-pegylated”, or equivalents thereof, are meant to be construed more broadly than to just mono-pegylated and di-pegylated IL-10. To illustrate, two or more different sites on each IL10 monomer might be modified by introducing more than one mutation and then modifying each of them; tyrosine 59 might be pegylated in combination with one or more modified mutant; or tyrosine 59 might be pegylated in combination with pegylation of the N-terminus. Exemplary pegylation conditions are described herein. The PEG component may be any PEG tolerated by the peptides. By way of example, the PEG component of the modified peptide has a molecular mass from 5 kDa to 20 kD in some embodiments, a molecular mass greater than 20 kDa in other embodiments, or a molecular mass of at least 30 kD in still other embodiments. PEGs having other molecular mass values are described herein.

The present disclosure contemplates any modification to the peptides that imparts a desired property, including improvement (e.g., masking) of a property of the unmodified peptides. In some embodiments the modified peptides comprise an Fc fusion molecule; a serum albumin (e.g., HSA or BSA), which may be in the form of an HSA fusion molecule or an albumin conjugate; or an albumin binding domain. The modified peptides may be glycosylated or hesylated. Detailed descriptions of the foregoing are described elsewhere within the present disclosure.

In particular embodiments, the modification is site-specific. In further embodiments, the modification comprises a linker. Some modified IL-10 molecules may comprise more than one type of modification. The types of modifications and the methods of introducing such modifications to the IL-10 peptides described herein are not limiting, and the skilled artisan can envisage other such modifications and methods.

The peptides described herein may be produced recombinantly. The present disclosure contemplates nucleic acid molecules encoding the peptides, wherein the nucleic acid molecules may be operably linked to an expression control element that confers expression of the nucleic acid molecule encoding the peptide in vitro, in a cell or in vivo. Vectors (e.g., a viral vector) may comprise such nucleic acid molecules. Further embodiments entail transformed or host cells that express the peptides described herein.

The present disclosure also contemplates the use of gene therapy in conjunction with the teachings herein. For gene therapy uses and methods, a cell in a subject can be transformed with a nucleic acid that encodes an IL-10-related polypeptide as set forth herein in vivo. Alternatively, a cell can be transformed in vitro with a transgene or polynucleotide, and then transplanted into a tissue of subject in order to effect treatment. In addition, a primary cell isolate or an established cell line can be transformed with a transgene or polynucleotide that encodes an IL-10-related polypeptide, and then optionally transplanted into a tissue of a subject.

The peptides of the present disclosure may comprise an epitope(s) that binds (specifically or non-specifically) to an antibody. Particular embodiments comprise an activating antibody, for example, an anti-IL-10R1/R2-complex antibody that mimics IL-10 activation through these receptors.

The antibody may be monoclonal or polyclonal, and may be, for example, human or humanized. Embodiments include an antibody that comprises a light chain variable region and a heavy chain variable region present in separate polypeptides or in a single polypeptide, or an antibody that comprises a heavy chain constant region that is, e.g., an IgG1, IgG2, IgG3, or IgG4 isotope. The antibody may be, for example, a Fv, scFv, Fab, F(ab′)₂, or Fab′ antibody, or it may be a single chain Fv (scFv) antibody (which may be multimerized).

In further embodiments, an antibody of the present disclosure binds the peptides with an affinity of from about 10⁷ M⁻¹ to about 10¹² M⁻¹. An antibody may comprise a covalently linked moiety selected from a lipid moiety, a fatty acid moiety, a polysaccharide moiety, and a carbohydrate moiety. Embodiments are also contemplated wherein an antibody comprises an affinity domain, may be immobilized on a solid support, comprises a covalently linked non-peptide polymer (e.g., a poly(ethylene)glycol polymer) or is detectably labeled.

The present disclosure includes pharmaceutical compositions comprising the peptides or antibodies described herein, and a pharmaceutically acceptable diluent, carrier or excipient. In some embodiments, the excipient is an isotonic injection solution. The pharmaceutical compositions may be suitable for administration to a subject (e.g., a human), and may comprise one or more additional prophylactic or therapeutic agents. In certain embodiments, the pharmaceutical compositions are contained in a sterile container (e.g., a single- or multi-use vial or a syringe). A kit may contain the sterile container(s), and the kit may also contain one or more additional sterile containers comprising at least one additional prophylactic or therapeutic agent or any other agent that may be used in pharmacological therapy. Examples of such aspects are set forth herein.

Additional embodiments of the present disclosure comprise a method of treating or preventing a disease, disorder or condition in a subject (e.g., a human), comprising administering a therapeutically effective amount of a peptide described herein. Further embodiments comprise a method of treating or preventing a disease, disorder or condition in a subject, comprising administering a therapeutically effective amount of an antibody described herein. In various embodiments of the present disclosure, the disease, disorder or condition is a proliferative disorder, including a cancer or a cancer-related disorder (e.g., a solid tumor or a hematological disorder) or a fibrotic disorder, such as cirrhosis, NASH and NAFLD; an immune or inflammatory disorder, including inflammatory bowel disease, psoriasis, rheumatoid arthritis, multiple sclerosis, and Alzheimer's disease; thrombosis or a thrombotic condition or disorder, including a state of hypercoagulation; a fibrotic disorder; a viral disorder, including, but not limited to, human immunodeficiency virus, hepatitis B virus, hepatitis C virus and cytomegalovirus; a cardiovascular disorder, including atherosclerosis or other cardiovascular-related disorders wherein the subject may have elevated cholesterol and/or other abnormal metabolic-related parameters (e.g., abnormal blood glucose levels, insulin levels, or lipid levels).

In the methods of treating or preventing a disease, disorder or condition, administration of the therapeutically effective amount of a peptide (or an antibody) described herein may be by any route appropriate for the peptide (or antibody), including parenteral injection (e.g., subcutaneously). One or more additional prophylactic or therapeutic agents may be administered with (e.g., prior to, simultaneously with, or subsequent to) the peptide (or antibody), and/or it may be administered separate from or combined with the peptide (or antibody).

### BRIEF DESCRIPTION OF THE DRAWINGS

FIG. 1A is a protein crystal structure ribbon representation (top view) of the human IL-10 monomer. The six helices are labeled A-F.

FIG. 1B is a protein crystal structure ribbon representation (side view) of the human IL-10 monomer. The six helices are labeled A-F.

FIG. 2A is a protein crystal structure ribbon representation (top view) of the human IL-10 homodimer. One monomer is gray and the other monomer is black. The six helices are labeled A-F.

FIG. 2B is a protein crystal structure ribbon representation (side view) of the human IL-10 homodimer. One monomer is gray and the other monomer is black.

FIG. 3A depicts the complete 178 amino acid human IL-10 sequence (SEQ ID NO:1). The 18 amino acid signal peptide is underlined.

FIG. 3B depicts the 160 amino acid mature human IL-10 sequence. (SEQ ID NO:2)

FIG. 3C depicts the mature human IL-10 amino acid sequence indicating the regions corresponding to Helices A-F, the regions corresponding to each of the Loops, and the regions/locations of the Kinks.

FIG. 4A is a protein crystal structure ribbon representation (top view) of the human IL-10 homodimer (gray) bound to two human IL10R1/α receptors (black).

FIG. 4B is a protein crystal structure ribbon representation (side view) of the human IL-10 homodimer (gray) bound to two human IL10R1/α receptors (black).

FIG. 5 illustrates which amino acid residues of the mature human IL-10 amino acid sequence are candidates for pegylation.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

The present disclosure contemplates mutant IL-10 molecules (e.g., muteins) and other IL-10-related molecules, as well as methods of their identification and their use. As described herein, the IL-10 molecules may be modified to, for example, enhance a property of native human IL-10, including half-life extension. Particular IL-10 molecules have comparable immunogenicity to human IL-10, and/or bioactivity at least comparable to human IL-10, and/or an improvement in at least one property (e.g., a physical property, including solubility, bioavailability, serum half-life, and circulation time).

Thus, for example, IL-10 molecules that have comparable immunogenicity to hIL-10 but have substantially less bioactivity than hIL-10 are encompassed herein. The skilled artisan will recognize that such molecules may be viable therapeutics due to, e.g., a very long half-life. The IL-10 molecules described herein, and compositions (e.g., pharmaceutical compositions) thereof, may be used to treat and/or prevent various diseases, disorders and conditions, and/or the symptoms thereof, including, for example, inflammatory- and immune-related disorders, fibrotic disorders, cancer and cancer-related disorders, and cardiovascular disorders (e.g., atherosclerosis).

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it may correspond to a sequence of a naturally occurring human polypeptide or nucleic acid molecule. In addition to the human polypeptides and the nucleic acid molecules which encode them, the present disclosure contemplates IL-10-related polypeptides and corresponding nucleic acid molecules from other species.

### DEFINITIONS

Unless otherwise indicated, the following terms are intended to have the meaning set forth below. Other terms are defined elsewhere throughout the specification.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, IL-10 or PEG-IL-10), a nucleic acid (e.g., a nucleic acid encoding native human IL-10), a pharmaceutical composition comprising the foregoing, or a diagnostic agent; to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering IL-10 or a pharmaceutical composition comprising IL-10) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, or condition afflicting a subject. Thus, treatment includes inhibiting (e.g., arresting the development or further development of the disease, disorder or condition or clinical symptoms association therewith) an active disease. The terms may also be used in other contexts, such as situations where IL-10 or PEG-IL-10 contacts an IL-10 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering IL-10 or a pharmaceutical composition comprising IL-10) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration may be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-10) or subjective parameter (e.g., a subject's feeling of well-being).

The term “small molecules” refers to chemical compounds having a molecular weight that is less than about 10 kDa, less than about 2 kDa, or less than about 1 kDa. Small molecules include, but are not limited to, inorganic molecules, organic molecules, organic molecules containing an inorganic component, molecules comprising a radioactive atom, and synthetic molecules. Therapeutically, a small molecule may be more permeable to cells, less susceptible to degradation, and less likely to elicit an immune response than large molecules.

The term “ligand” refers to, for example, a peptide, a polypeptide, a membrane-associated or membrane-bound molecule, or a complex thereof, that can act as an agonist or antagonist of a receptor. “Ligand” encompasses natural and synthetic ligands, e.g., cytokines, cytokine variants, analogs, muteins, and binding compositions derived from antibodies, as well as, e.g., peptide mimetics of cytokines and peptide mimetics of antibodies. The term also encompasses an agent that is neither an agonist nor antagonist, but that can bind to a receptor without significantly influencing its biological properties, e.g., signaling or adhesion. Moreover, the term includes a membrane-bound ligand that has been changed, e.g., by chemical or recombinant methods, to a soluble version of the membrane-bound ligand. A ligand or receptor may be entirely intracellular, that is, it may reside in the cytosol, nucleus, or some other intracellular compartment. The complex of a ligand and receptor is termed a “ligand-receptor complex.”

The terms “inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of an IL-10 molecule (or the nucleic acid molecules encoding them), either directly or indirectly; or to enhance the ability of a molecule to produce an effect comparable to that of an IL-10 molecule. The term “modulator” is meant to refer broadly to molecules that can effect the activities described above. By way of example, a modulator of, e.g., a gene, a receptor, a ligand, or a cell, is a molecule that alters an activity of the gene, receptor, ligand, or cell, where activity can be activated, inhibited, or altered in its regulatory properties. A modulator may act alone, or it may use a cofactor, e.g., a protein, metal ion, or small molecule. The term “modulator” includes agents that operate through the same mechanism of action as IL-10 (i.e., agents that modulate the same signaling pathway as IL-10 in a manner analogous thereto) and are capable of eliciting a biological response comparable to (or greater than) that of IL-10.

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term may also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently (though not always) means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity. As previously indicated, the skilled artisan recognizes that use of different methodologies may result in IL-10 that is more or less active—either in apparent activity due to differences in calculating protein concentration or in actual activity—than a hIL-10 reference standard. The skilled artisan will be able to factor in these differences in determining the relative bioactivities of an IL-10 molecule versus hIL-10.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like.

As used herein, the terms “variants” and “homologs” are used interchangeably to refer to amino acid or DNA sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring DNA sequences and proteins encoded thereby and their isoforms, as well as splice variants of a protein or gene. The terms also encompass nucleic acid sequences that vary in one or more bases from a naturally-occurring DNA sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes. Unless otherwise indicated, use of terms such as “mutant of IL-10” refer to IL-10 muteins.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein in reference to native human IL-10 or an IL-10 mutein, the terms “modified”, “modification” and the like refer to one or more changes that enhance a desired property of human IL-10 or an IL-10 mutein. Such desired properties include, for example, prolonging the circulation half-life, increasing the stability, reducing the clearance, altering the immunogenicity or allergenicity, and enabling the raising of particular antibodies (e.g., by introduction of unique epitopes) for use in detection assays. As discussed in detail hereafter, modifications to human IL-10 or an IL-10 mutein that may be carried out include, but are not limited to, pegylation (covalent attachment of one or more molecules of polyethylene glycol (PEG), or derivatives thereof); glycosylation (e.g., N-glycosylation), polysialylation and hesylation; albumin fusion; albumin binding through, for example, a conjugated fatty acid chain (acylation); Fc-fusion; and fusion with a PEG mimetic. In some embodiments, linkers are used in such modifications and are described hereafter.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it may naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least ten times greater, at least 20-times greater, or at least 100-times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239).

### IL-10 and PEG-IL-10

The anti-inflammatory cytokine IL-10, also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, -β, -γ, -δ, -ε, -κ, -Ω, and -τ) and interferon-like molecules (limitin, IL-28A, IL-28B, and IL-29).

IL-10 is a cytokine with pleiotropic effects in immunoregulation and inflammation. It is produced by mast cells, counteracting the inflammatory effect that these cells have at the site of an allergic reaction. While it is capable of inhibiting the synthesis of pro-inflammatory cytokines such as IFN-γ, IL-2, IL-3, TNFα and GM-CSF, IL-10, it is also stimulatory towards certain T cells and mast cells and stimulates B-cell maturation, proliferation and antibody production. IL-10 can block NF-κB activity and is involved in the regulation of the JAK-STAT signaling pathway. It also induces the cytotoxic activity of CD8+ T-cells and the antibody production of B-cells, and it suppresses macrophage activity and tumor-promoting inflammation. The regulation of CD8+ T-cells is dose-dependent, wherein higher doses induce stronger cytotoxic responses.

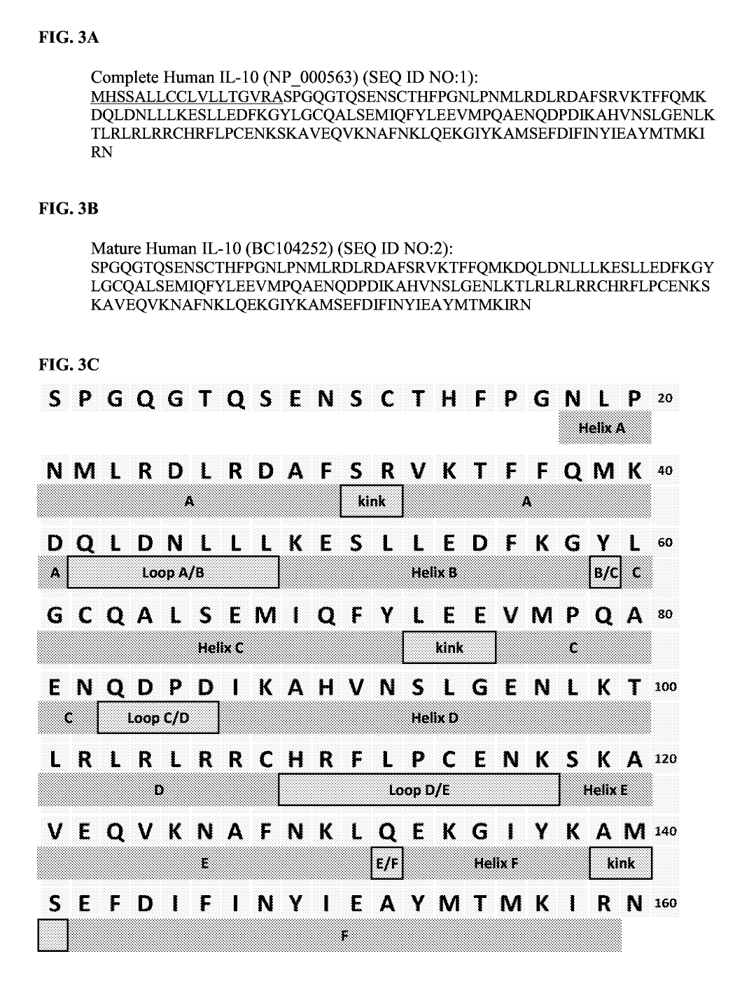

Human IL-10 is a homodimer with a molecular mass of 37 kDa, wherein each 18.5 kDa monomer comprises 178 amino acids, the first 18 of which comprise a signal peptide, and two pairs of cysteine residues that form two intramolecular disulfide bonds. Each monomer of mature hIL-10 comprises 160 amino acid residues. The IL-10 dimer becomes biologically inactive upon disruption of the non-covalent interactions between the two monomer subunits. FIG. 3A depicts the complete 178 amino acid human IL-10 sequence (the 18 amino acid signal peptide is underlined), and FIG. 3B depicts the 160 amino acid mature human IL-10 sequence.

The present disclosure contemplates human IL-10 and murine IL-10, which exhibit 80% homology, and use thereof. In addition, the scope of the present disclosure includes IL-10 orthologs, and modified forms thereof, from other mammalian species, including rat (accession NP_{—}036986.2; GI 148747382); cow (accession NP_{—}776513.1; GI 41386772); sheep (accession NP_{—}001009327.1; GI 57164347); dog (accession ABY86619.1; GI 166244598); and rabbit (accession AAC23839.1; GI 3242896).

The IL-10 receptor, a type II cytokine receptor, consists of alpha and beta subunits, which are also referred to as R1 and R2, respectively. Receptor activation requires binding to both alpha and beta. One homodimer of an IL-10 polypeptide binds to alpha and the other homodimer of the same IL-10 polypeptide binds to beta.

The utility of recombinant human IL-10 is frequently limited by its relatively short serum half-life, which may be due to, for example, renal clearance, proteolytic degradation, receptor mediated uptake and monomerization in the blood stream. As a result, various approaches have been explored to improve the pharmacokinetic profile of IL-10 without disrupting its dimeric structure and thus adversely affecting its activity. Pegylation of IL-10 results in improvement of certain pharmacokinetic parameters (e.g., serum half-life) and/or enhancement of activity. For example, particular embodiments of the present disclosure involve methods of optimizing the treatment of proliferative disorders (e.g., cancer) with pegylated IL-10 muteins.

As previously indicated, the present disclosure also contemplates the use of gene therapy in conjunction with the teachings herein. Gene therapy is effected by delivering genetic material, usually packaged in a vector, to endogenous cells within a subject in order to introduce novel genes, to introduce additional copies of pre-existing genes, to impair the functioning of existing genes, or to repair existing but non-functioning genes. Once inside cells, the nucleic acid is expressed by the cell machinery, resulting in the production of the protein of interest. In the context of the present disclosure, gene therapy is used as a therapeutic to deliver nucleic acid that encodes an IL-10 agent for use in the treatment or prevention of a disease, disorder or condition described herein.

As alluded to above, for gene therapy uses and methods, a cell in a subject can be transformed with a nucleic acid that encodes an IL-10-related polypeptide as set forth herein in vivo. Alternatively, a cell can be transformed in vitro with a transgene or polynucleotide, and then transplanted into a tissue of a subject in order to effect treatment. In addition, a primary cell isolate or an established cell line can be transformed with a transgene or polynucleotide that encodes an IL-10-related polypeptide, and then optionally transplanted into a tissue of a subject.

As used herein, the terms “pegylated IL-10” and PEG-IL-10″ refer to an IL-10 molecule having one or more polyethylene glycol molecules covalently attached to at least one amino acid residue of the IL-10 protein, generally via a linker, such that the attachment is stable. The terms “monopegylated IL-10” and “mono-PEG-IL-10” indicate that one polyethylene glycol molecule is covalently attached to a single amino acid residue on one subunit of the IL-10 dimer, generally via a linker. In certain embodiments, the PEG-IL-10 used in the present disclosure is a mono-PEG-IL-10 in which one to nine PEG molecules are covalently attached via a linker to the alpha amino group of the amino acid residue at the N-terminus of one subunit of the IL-10 dimer. Linkers are described further hereafter.

Monopegylation on one IL-10 subunit generally results in a non-homogeneous mixture of non-pegylated, monopegylated and dipegylated IL-10 due to subunit shuffling. Moreover, allowing a pegylation reaction to proceed to completion will generally result in non-specific and multi-pegylated IL-10, thus reducing its bioactivity. Thus, particular embodiments of the present disclosure comprise the administration of a mixture of mono- and di-pegylated IL-10 produced by the methods described herein. As previously indicated, references herein to “mono-pegylated” or “di-pegylated”, or equivalents thereof, are meant to be construed more broadly than to just mono-pegylated and di-pegylated IL-10. Thus, two or more different sites on each IL-10 monomer might be modified by introducing more than one mutation and then modifying each of them. By way of further example, tyrosine 59 might be pegylated in combination with one or more modified mutant; or tyrosine 59 might be pegylated in combination with pegylation of the N-terminus. Exemplary pegylation conditions are described in, e.g., the Experimental section.

In particular embodiments, the average molecular weight of the PEG moiety is between about 5 kDa and about 50 kDa. For example, the PEG moiety may have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Although the present disclosure does not require use of a specific method or site of PEG attachment to IL-10, it is frequently advantageous that pegylation does not alter, or only minimally alters, the activity of the IL-10 molecule. In certain embodiments, the impact of any increase in half-life is greater than the impact of any decrease in biological activity. The biological activity of PEG-IL-10 is typically measured by assessing the levels of inflammatory cytokines (e.g., TNF-α or IFN-γ) in the serum of subjects challenged with a bacterial antigen (lipopolysaccharide (LPS)) and treated with PEG-IL-10, as described in U.S. Pat. No. 7,052,686.

IL-10 variants can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are usually predetermined variants not found in nature, although some may be post-translational variants, e.g., glycosylated variants. Any variant of IL-10 can be used provided it retains a suitable level of IL-10 activity. In the tumor context, suitable IL-10 activity includes, for example, CD8+ T cell infiltration into tumor sites, expression of inflammatory cytokines such as IFN-γ, IL-4, IL-6, IL-10, and RANK-L, from these infiltrating cells, and increased levels of TNF-α or IFN-γ in biological samples.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2)R, K; 3) F, Y, H, W, R; 4) G, A, T, S; 5) Q, N; and 6) D, E. Guidance for substitutions, insertions, or deletions may be based on alignments of amino acid sequences of different variant proteins or proteins from different species. Thus, in addition to any naturally-occurring IL-10 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution. If should be noted that one or more unnatural amino acids may be introduced into IL-10 as a means of fostering site-specific conjugation.

The present disclosure also contemplates active fragments (e.g., subsequences) of mature IL-10 containing contiguous amino acid residues derived from the mature IL-10. The length of contiguous amino acid residues of a peptide or a polypeptide subsequence varies depending on the specific naturally-occurring amino acid sequence from which the subsequence is derived. In general, peptides and polypeptides may be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-10 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

As discussed further below, the IL-10 polypeptides may be isolated from a natural source (e.g., an environment other than its naturally-occurring environment) and may also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides may also be synthetically produced (e.g., by cell-free chemical synthesis).

Nucleic acid molecules encoding the IL-10 molecules are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

Identification of Modified IL-10 Molecules with Desirable Characteristics

The present disclosure is drawn, in part, to the manipulation of protein function through mutagenesis of, and other modifications to, IL-10. In some embodiments, the present disclosure contemplates modified IL-10 molecules wherein one or more advantageous characteristics have been added to IL-10 (in cases where the characteristic(s) is not present in the unmodified IL-10), and/or enhanced (in cases where the characteristic(s) is present in the unmodified IL-10, albeit in a less-than-optimal amount). As discussed further hereafter, such molecules may be identified and synthesized through rational drug design approaches comprising, for example, generation of a series of point mutations in human IL-10. This series of point mutations may be evaluated to determine the nature and extent of the properties (e.g., efficacy) of the members in the series.

In some embodiments, the point mutations are used to facilitate the synthesis of, for example, modified IL-10 peptides, wherein the peptides comprise covalent or non-covalent modifications (e.g., pegylation, Fc-fusions, and HSA fusions). In turn, systematic assessment of the modified peptides can be performed to define the locations of the IL-10 primary amino acid sequence where modifications can be effected while a) retaining protein bioactivity; b) enhancing certain protein functions (e.g., increasing duration of the IL-10-IL-10 receptor docking interaction; c) deemphasizing certain IL-10 functions while maintaining others; or d) some combination of a)-c).

One goal of the rational drug design approaches contemplated herein is identification of those amino acid residues and regions of IL-10 that can be modified without having deleterious effects on bioactivity, while allowing other attributes to be added or enhanced. Another goal of these rational drug design approaches is to define amino acid residues and regions of IL-10 where modifications can be used to selectivity deemphasize certain IL-10 functions while maintaining or enhancing the others. Thus, in certain embodiments, the IL-10 molecules (e.g., muteins) or modified IL-10 molecules accentuate one or more roles of IL-10 while deemphasizing one or more different roles; accentuate one or more roles of IL-10 while not affecting the others (e.g., retaining normal levels of IL-10 activity); or deemphasize one or more roles of IL-10 while not affecting the others.

In particular embodiments, the modification(s) described herein improves at least one property or other characteristic (e.g., efficacy) of the peptides compared to unmodified versions of the peptides thereof. Further embodiments of the present disclosure pertain to methods and other technologies for identifying specific amino acid residues or domains of IL-10 that may be modified according to the methods described herein. Methods of using (e.g., in the treatment or prevention of a disorder or a symptom thereof), identifying and/or generating the peptides described herein are also aspects of the present disclosure. Other aspects include, for example, pharmaceutical compositions comprising the peptides.

Although identification of certain IL-10 functional domains and generation of particular types of IL-10 conjugates have been described, the literature is devoid of any description of the types of Il-10 molecules described herein and the methods for identifying them. Thus, while Gesser, B., et al. ((1997) Proc. Natl. Acad. Sci. 94:14620-25) describe the identification of two nonapeptides found to possess certain IL-10-like activities, one located at the C-terminal portion of IL-10 and the other close to the N-terminal part, Gesser et al. do not describe any IL-10 mutants or modified IL-10 mutants. Furthermore, IL-10 polypeptides wherein an amino acid residue having an attachment group for a non-polypeptide moiety is introduced or removed in order to adapt the polypeptides to make them more susceptible to conjugation with a non-polypeptide moiety (U.S. Patent Publn. No. 2003/0186386) are also vastly different from the IL-10 molecules and methodologies described herein.

In particular embodiments, the present disclosure contemplates generation of a series of point mutations in human IL-10 and expression of those mutated IL-10 proteins (e.g., muteins) in, for example, a mammalian or bacterial system. The present disclosure contemplates the use of any expression system compatible with the mutant IL-10 molecules described herein. Mammalian protein expression systems are contemplated in particular embodiments, while in other embodiments candidate protein expression systems include those derived from bacteria (e.g., E. coli, Corynebacterium, P. fluorescens, and B. subtilis), yeast (e.g, S. cerevisiae), and baculovirus-infected insect cells. Cell-based or cell-free expression systems may be used. Most recombinant cytokines are produced in bacterial inclusion bodies, then purified and refolded.

Bacterial cells are frequently employed to express cytokines, a method which typically involves protein refolding. However, it can be advantageous to initially use a mammalian expression system in order to determine whether a mutated protein will be expressed. If the mammalian cell can express the mutated protein, then protein folding likely was not disrupted by the mutation. There is frequently a close correlation between the ability of a mammalian cell line to fold and secrete a mutant molecule and the viability of that molecule as a candidate for further evaluation. Conversely, if initial expression is carried out in bacteria and a mutated protein is not properly refolded, then it would not be clear whether the mutation was disruptive or the protein refolding protocol was sub-optimal.

Mutant IL-10 molecules that do not significantly disrupt protein folding and secretion in an expression system (e.g., a mammalian cell line-based expression system) may be candidates for further evaluation. For example, such mutant IL-10 molecules may be sufficiently purified to enable bioactivity analysis in one or more in vivo or in vitro/ex vivo assays, including the TNFα inhibition assay and the MC/9 cell proliferation assays described herein. By way of further example, such mutant IL-10 molecules may be evaluated in an in vitro assay that provides an IL-10/IL10R1 or IL-10/IL10R1/IL10R2 affinity measurement. In addition, in vivo models (e.g., an in vivo murine endotoxemia model) have been described and may be used in assessment of the IL-10 molecules described herein (see, e.g., Howard, M. et al., (1993) J. Exp. Med. 177:1205-08).

In particular embodiments, the mutant IL-10 polypeptide molecules (e.g., muteins) are modified by, for example, pegylation. These modified IL-10 molecules may then be evaluated to determine their impact on protein function. Modified IL-10 molecules exhibiting favorable characteristics (e.g., nominal or no impact on protein function) may be candidates for further modification (e.g., larger or branched PEGs) and evaluation (e.g., solubility).

In addition, the present disclosure contemplates evaluation of the mutant IL-10 peptides and modified IL-10 peptides using one or more assays for determining immunogenicity, such as those in vitro, ex vivo, or in silico immunogenicity assays described herein. Modified IL-10 molecules exhibiting particular favorable characteristics (e.g., enhanced efficacy without an increase in immunogenicity as determined in silico) may be candidates for further evaluation, including in vivo immunogenicity analysis and/or additional analyses in an in vivo setting. In particular embodiments, these modified IL-10 molecules are not more immunogenic than the corresponding unmodified IL-10 molecules.

Also encompassed herein are other IL-10 molecules, including IL-10 fragments; polypeptides based on IL-10 monomers; molecules that comprise an IL-10 monomer complexed with a heterologous protein; and IL-10 fusion proteins that comprise IL-10 fused, at the nucleic acid level, to one or more therapeutic agents (e.g., an anti-inflammatory biologic). Such molecules may be modified using the approaches described herein or any other approach known to the skilled artisan.

The rational drug design approaches of the present disclosure may utilize crystallographic data from a number of sources, including data obtained from the published crystal structure of IL-10 (Zdanov, A. et al, (1995) Structure (Lond) 3:591-601 and Walter, M. and Nagabhushan, T., (1995) Biochemistry (38):12118-25); a model of the crystal structure of hIL-10 with its soluble receptor (Zdanov, A. et al., (1996) Protein Sci. (10):1955-62); and the crystal structure of the IL-10/IL-10R1 complex (Josephson, K. et al., (2001) Immunity (1):35-46). Though insufficient and incomplete in and of themselves, the information and data described in such sources may represent a component used in the identification of IL-10 amino acid residues and domains that may be modified. As a result of leveraging such information and data, mutant IL-10 molecules (e.g., muteins) and modified mutant IL-10 molecules (and, in some embodiments, modified native hIL-10) were identified having the advantageous and/or desirable characteristics described herein.

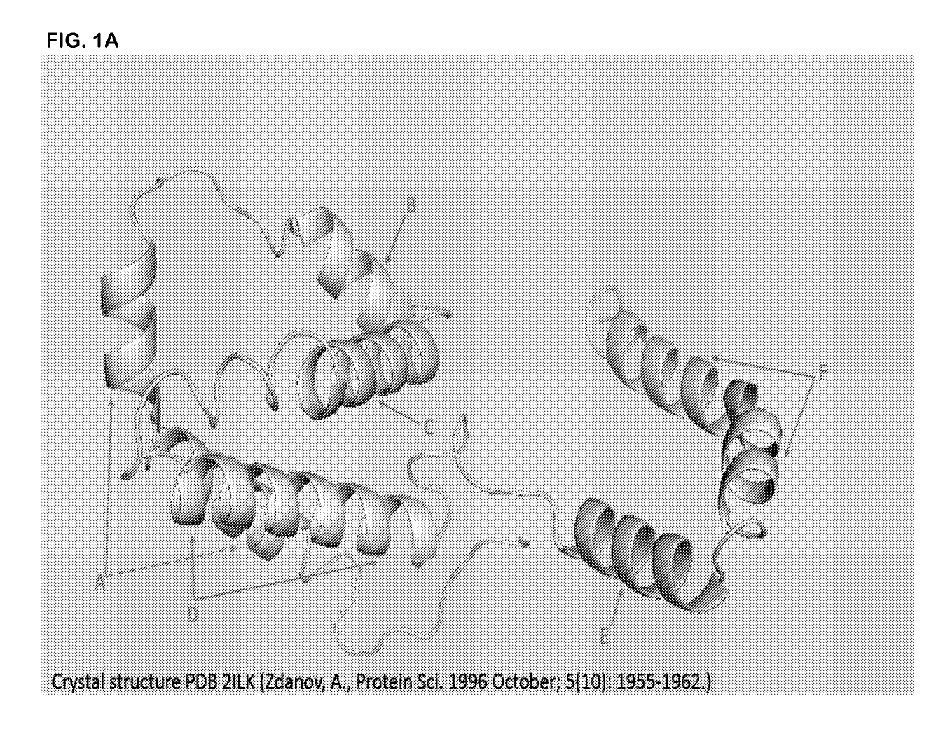

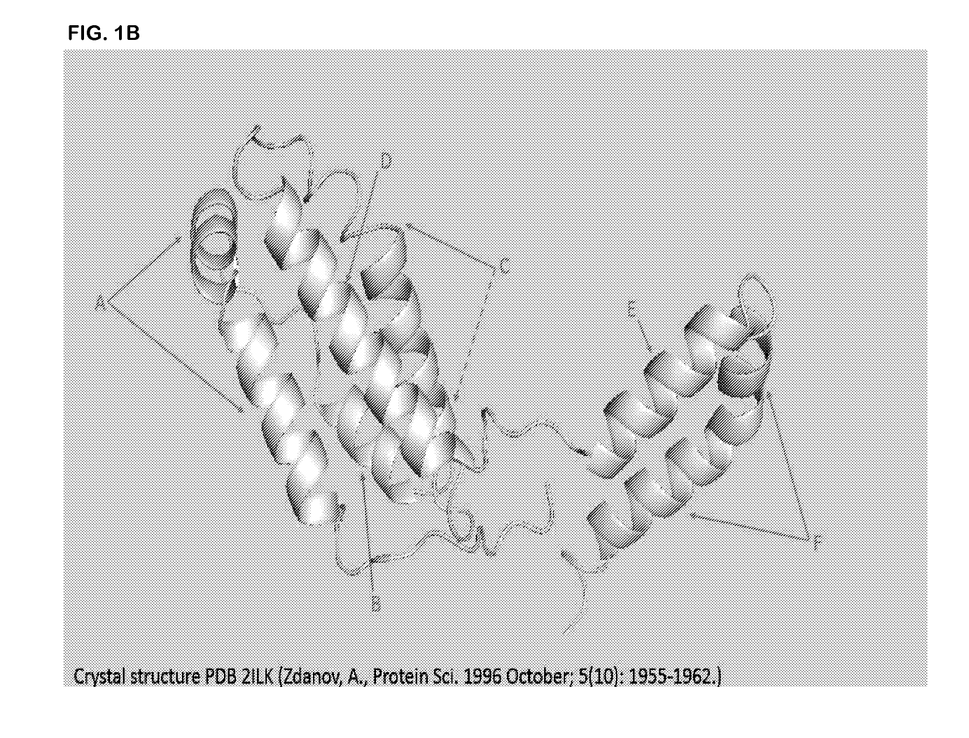

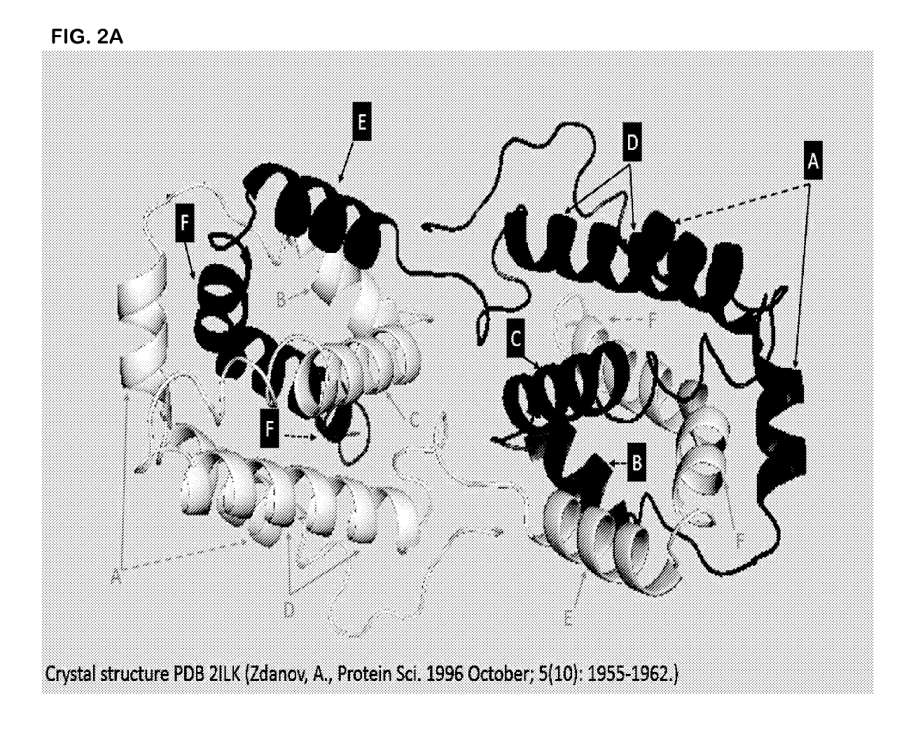

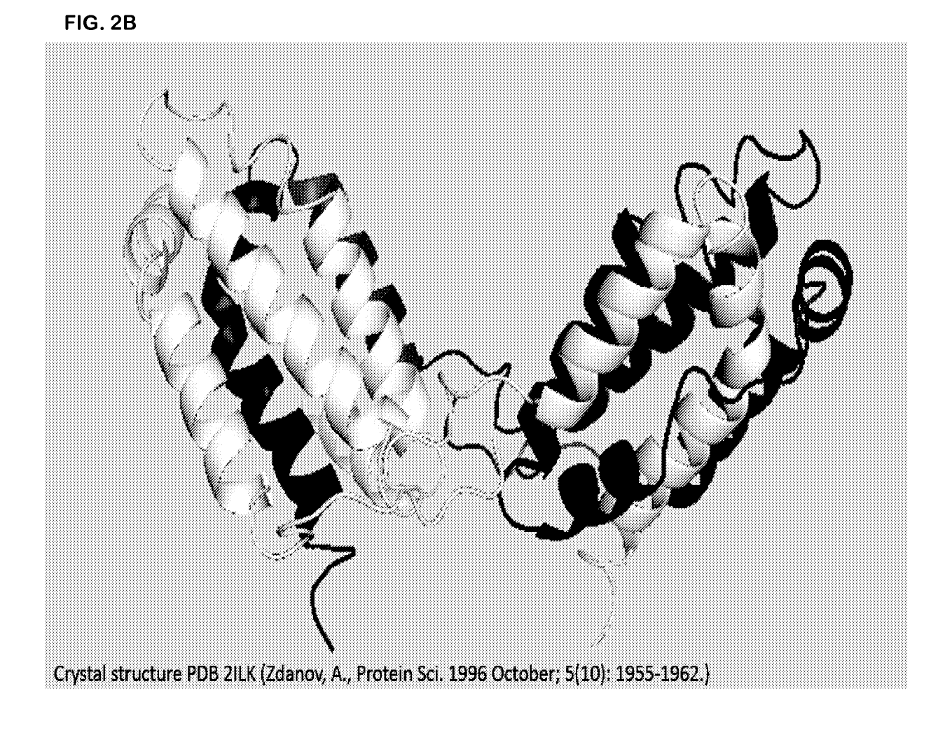

Each 160 amino acid monomer of mature human IL-10 (hIL-10) comprises six helices linked by short loops, also referred to herein as inter-helix junctions. FIGS. 1A and 1B depict protein crystal structure ribbon representations (top view and side view, respectively) of the hIL-10 monomer, wherein the six helices are labeled A-F. FIGS. 2A and 2B depict protein crystal structure ribbon representations (top view and side view, respectively) of the hIL-10 homodimer; the six helices of each monomer are labeled A-F in FIG. 2A. FIG. 3C depicts the mature hIL-10 amino acid sequence indicating the regions corresponding to Helices A-F and the regions corresponding to each of the inter-helix junctions (loops). FIG. 3C also indicates that Helices A, C and F have kinks (regions within the hIL-10 three-dimensional structure wherein the sequence has, e.g., a severe bend) comprising stretches of several amino acids. Although the amino acid residues defining each helix, inter-helix junction and kink are accepted in the literature, it will be appreciated that skilled artisans may differ regarding which residues form the precise boundaries of each domain and inter-helix junction, and that any such differences do not impact the teachings set forth herein.

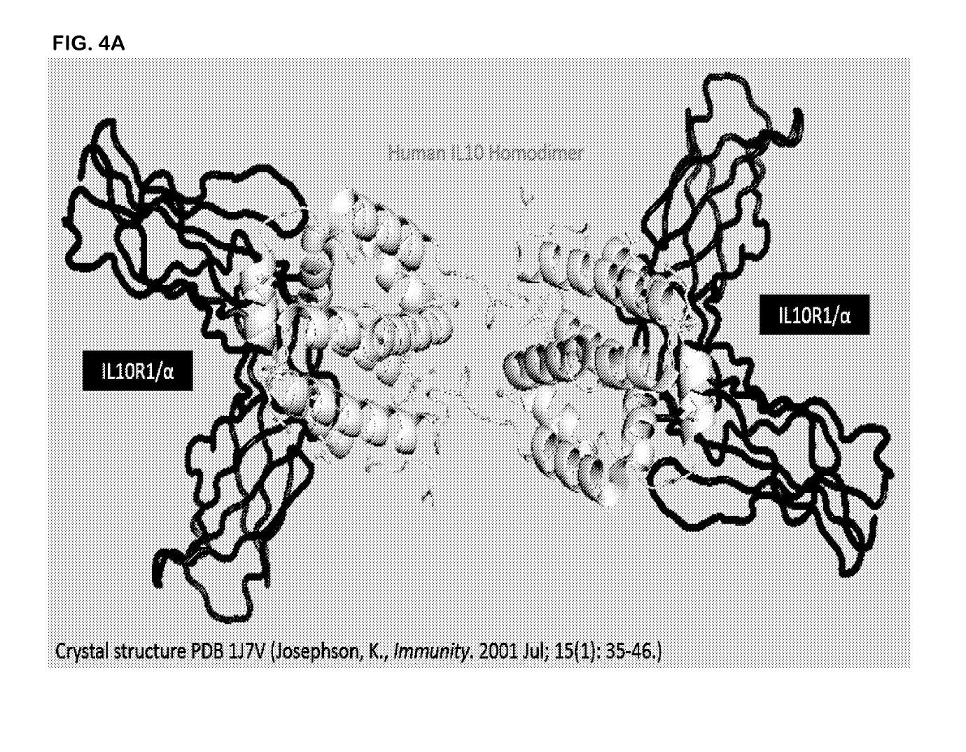

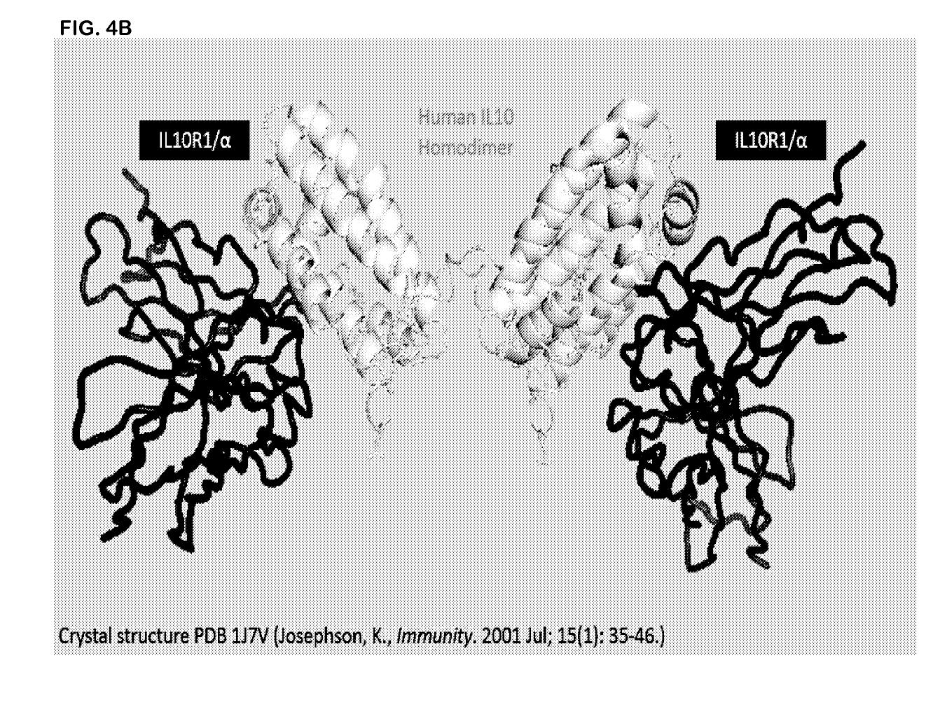

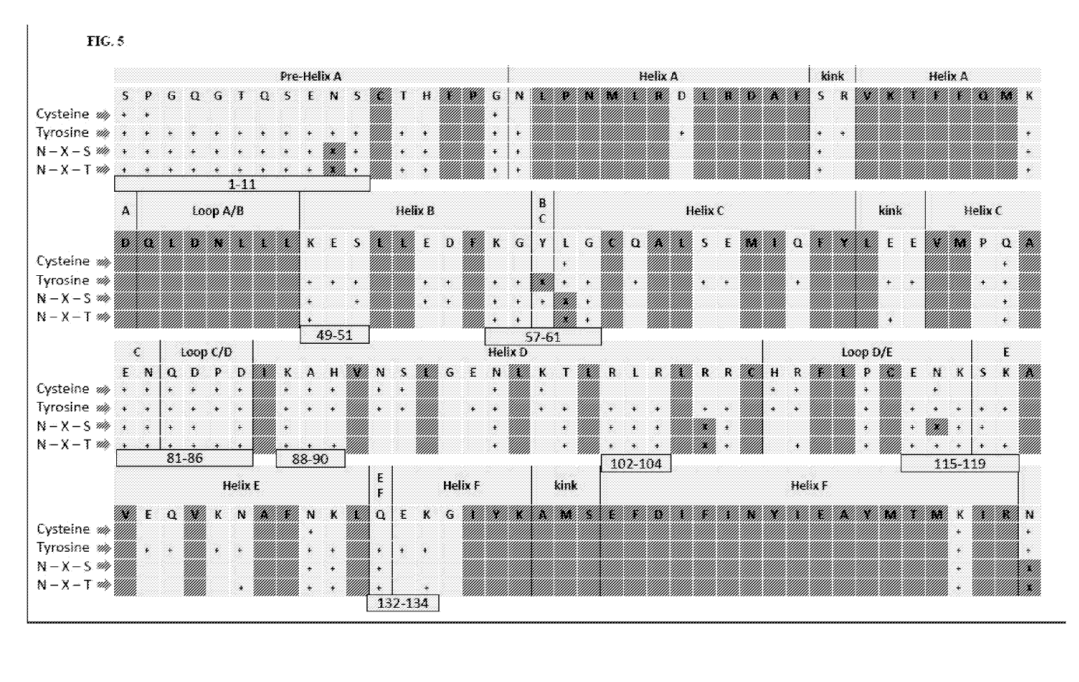

As previously noted, the IL-10 receptor comprises alpha and beta subunits, which are also referred to as R1 and R2, respectively. While the mechanics of IL-10 receptor binding have not been thoroughly elucidated, it has been shown that IL-10 signalling requires contributions from both IL-10R1 and IL-10R2. This may occur through one IL-10 homodimer independently binding both IL-10R1 and IL-10R2 combined with some type of clustering event, or by one IL-10 homodimer forming a single complex with both IL-10R1 and IL-10R2. FIGS. 4A and 4B depict protein crystal structure ribbon representations (top view and side view, respectively) of the human IL10 homodimer (gray) bound to two human IL10R1/α receptors (black).

Amino acid residues likely to be poor candidates for modification (e.g., pegylation) include: residues in a hydrophobic core, which are likely to be inaccessible to modification; residues contacting IL10R1/2 receptors; residues in close proximity to the IL10R1/2-IL-10-binding interface; and cysteine residues involved in disulfide bonds, which are generally non-reactive with cysteine-based pegylation chemistries (though cysteine pegylation of disulfide bonds has been accomplished using defined pegylation conditions). In contrast, amino acid residues likely to be good candidates for potential modification (e.g., pegylation) include: surface-exposed residues not involved in protein-protein interactions; residues that form the inter-helices junctions; or the residues prior to Helix A (“Pre-helix A”, as defined hereafter) or the residue subsequent to Helix F (“Post-helix F”, as defined hereafter). The tyrosine at amino acid residue 59 is one candidate for modification (e.g., pegylation). Modification of the amino acid residues that form a kink may have a more limited set of substitutions that will be tolerated.

As set forth elsewhere herein, chemistries currently exist for pegylation of a polypeptide's N-terminus, lysine residues, cysteine residues, histidine residues, arginine residues, aspartic acid residues, glutamic acid residues, serine residues, threonine residues, tyrosine residues, and C-terminus. As indicated above, the present disclosure contemplates the introduction of unnatural amino acid residues which may, in turn, be pegylated. However, only some of these amino acid residues (e.g., tyrosine residues (and the N-terminus)) can routinely be pegylated in a site-specific manner. Pegylation of other amino acids can only be effected in a site-specific manner under complex conditions, while pegylation of other amino acids (e.g., glutamic acid and serine residues) results in too many positional isomers to be useful.

Based on the teachings set forth herein, modification of amino acid residues via a combination of mutagenesis and site-specific chemistries is not predicted to be feasible for 58 residues likely to be buried within a hydrophobic core; 4 residues likely to be involved in disulfide bonds; and 27 residues likely to be in contact with IL-10R1/α (7 of which are also predicted to be buried within a hydrophobic core region). In addition, 10 residues are in close proximity to the putative IL-10 and IL-10R1/α binding interface but may not directly interact with IL-10R1/α; although it is predicted that modification of these residues will also not be feasible, the present disclosure recognizes that one or more of these residues might tolerate modification and, if so, such modifications are encompassed herein. Conversely, based on the teachings set forth herein, modification of amino acid residues via a combination of mutagenesis and site-specific chemistries is predicted to be feasible for 78 residues likely to be surface-exposed and not integrally involved in IL-10R1/α binding or disulfide bonding.

The amino acid residues corresponding to each helix and inter-helix junction are set forth hereafter, as are the residues that occur before Helix A (“Pre-helix A”) and after Helix F (“Post-helix F”): Pre-helix A=1-17; Helix A=18-41; A/B Inter-helix Junction=42-48; Helix B=49-58; B/C Inter-helix Junction=59; Helix C=60-82; C/D Inter-helix Junction=83-86; Helix D=87-108; D/E Inter-helix Junction=109-117; Helix E=118-131; E/F Inter-helix Junction=132; Helix F=133-159; and Post-helix F=160. Based on the teachings of the present disclosure, in particular embodiments the peptides comprise at least one substitution in the 160 amino acid IL-10 monomer at amino acid residues and regions identified herein as being able to accommodate such substitutions. These peptides may be modified as described herein.

Thus, the peptides of the present disclosure may comprise a) a Pre-helix A; b) a Helix A; c) an A/B Inter-helix Junction; d) a Helix B; e) a B/C Inter-helix Junction; f) a Helix C; g) a C/D Inter-helix Junction; h) a Helix D; i) a D/E Inter-helix Junction; j) a Helix E; k) an E/F Inter-helix Junction; l) a Helix F; and m) a Post-helix F; wherein such peptides further comprise at least one of: i) substitution of at least one amino acid residue of Pre-helix A other than amino acid residues 12 (C), 15 (F) or 16 (P); or ii) substitution of at least one amino acid residue of Helix A other than amino acid residues 19-24 (LPNMLR (SEQ ID NO:33)), 26-30 (LRDAF (SEQ ID NO:34)), 33-39 (VKTFFQM (SEQ ID NO:35)), or 41 (D); or iii) substitution of at least one amino acid residue of Helix B other than amino acid residues 52 (L), 53 (L), or 56 (F); or iv) substitution of the amino acid residue of the B/C Inter-helix Junction; or v) substitution of at least one amino acid residue of Helix C other than amino acid residues 62 (C), 64 (A), 65 (L), 68 (M), 69 (I), 71-73 (FYL), 76 (V), 77 (M), or 80 (A); or vi) substitution of at least one amino acid residue of the C/D Inter-helix Junction; or vii) substitution of at least one amino acid residue of Helix D other than amino acid residues 87 (I), 91 (V), 94 (L), 98 (L), 101 (L), 105 (L), or 108 (C); or viii) substitution of at least one amino acid residue of the D/E Inter-helix Junction other than amino acid residues 111 (F), 112 (L), or 114 (C); or ix) substitution of at least one amino acid residue of Helix E other than amino acid residues 120 (A), 121 (V), 124 (V), 127 (A), 128 (F) or 131 (L); or x) substitution of the amino acid residue of the E/F Inter-helix Junction; or xi) substitution of at least one amino acid residue of Helix F other than amino acid residues 136-156 (IYKAMSEFDIFINYIEAYMTM ((SEQ ID NO:36)), 158 (I) or 159 (R); or xii) substitution of the amino acid residue of Post-helix F. These peptides may be modified as described herein.

As described in detail in the Experimental section and as indicated in FIG. 5, 78 residues of the mature human IL-10 polypeptide are more likely surface exposed in the homodimer and are less likely to be involved in receptor binding, and these 78 residues represent sites that might possibly tolerate mutations by substitution of an amino acid that will serve as an anchor for a PEG. Of these 78 possible locations, some mutants are eliminated at specific locations for various reasons: residue 59 (Y) cannot be mutated to a tyrosine because human IL-10 already contains a tyrosine at that position; for residues at 10 (N), and 60 (L), 106 (R), introducing an N-glycosylation site would interfere with cysteine bonding and probably destroy the protein's bioactivity; residue 116 (N) already contains an N-X-S N-glycosylation motif so only an N-X-T motif can be introduced; for residue 160 (N), because the N-glycosylation motif is three amino acids long (N-X-S or N-X-T), an N-glycosylation site cannot be introduced at the last residue of a protein. Due to the motif for an N-glycosylation site spanning three amino acids (N-X-S or N-X-T, where X≠P), it was frequently necessary to introduce a mutation outside of the 78 residues described, but it should be noted that these mutations were designed so that the N-glycosylation would occur at these 78 locations on human IL-10, and hence the N-glycosylation mutation still serves as a means of testing these 78 locations.

The mutants (e.g., cysteine, tyrosine, N-X-S and N-X-T; see FIG. 5) were generated using the methods described herein and were evaluated in an MC/9 assay to determine biological activity. Of those mutants possessing biological activity, 76 mutants were identified as being potential candidates for serving as an anchor site for a PEG moiety.

Some embodiments of the present disclosure contemplate peptides comprising at least one amino acid substitution in at least one of the following regions: 1-11, 49-51, 57-61, 81-86, 88-90, 102-104, 115-119, or 132-134. In other embodiments, the peptides comprise at least one amino acid substitution at least at one of the following positions: 1-11, 13, 14, 17, 18, 25, 31, 32, 40, 49-51, 54, 55, 57-61, 63, 66, 67, 70, 74, 75, 78, 79, 81-86, 88-90, 92, 93, 96, 97, 99, 100, 102-104, 106, 107, 109, 110, 113, 115-119, 122, 123, 125, 126, 129, 130, 132-134, 157 or 160.

### Immunogenicity Considerations of Modified Forms of IL-10

Immunogenicity, the ability of an antigen to elicit humoral (B-cell) and/or cell-mediated (T-cell) immune responses in a subject, can be categorized as ‘desirable’ or ‘undesirable’. Desirable immunogenicity typically refers to the subject's immune response mounted against a pathogen (e.g., a virus or bacterium) that is provoked by vaccine injection. In this context, the immune response is advantageous. Conversely, undesirable immunogenicity typically refers to the subject's immune response mounted against an antigen like a therapeutic protein (e.g., IL-10); the immune response can, for example, result in anti-drug-antibodies (ADAs) that adversely impact the therapeutic protein's effectiveness or its pharmacokinetic parameters, and/or contribute to other adverse effects. In this context, the immune response is disadvantageous.

There are a number of subject-specific and product-specific factors that affect a subject's immune reaction to a protein therapeutic. Subject-specific factors include the immunologic status and competence of the subject; prior sensitization/history of allergy; route of administration; dose and frequency of administration; genetic status of the subject; and the subject's status of immune tolerance to endogenous protein. Product-specific factors affecting immunogenicity include product origin (foreign or endogenous); product's primary molecular structure/post-translational modifications, tertiary and quaternary structure, etc.; presence of product aggregates; conjugation/modification (e.g., glycosylation and pegylation); impurities with adjuvant activity; product's immunomodulatory properties; and formulation.

Autologous or human-like polypeptide therapeutics have proven to be surprisingly immunogenic in some applications, and surprisingly non-immunogenic in others. Particular IL-10 muteins and other modified versions of IL-10 (e.g., pegylated IL-10 and IL and IL-10 domains) are likely to provoke a range of humoral and cell-mediated immune responses.

As discussed further herein, the removal or modification of T-cell epitopes and/or B-cell epitopes can reduce immunogenicity. Indeed, in certain contexts, conjugation of one or more amino acid residues with a ‘masking agent’ (e.g., a PEG) and/or changes to the amino acids residues themselves (by, e.g., substitutions) may dramatically reduce the immunogenicity of an otherwise highly immunogenic protein.

T-Cell Epitopes.

As discussed further below, in contrast to the complex three-dimensional B-cell epitopes that often depend on secondary and tertiary protein structure, CD4+ T-cell epitopes are linear peptide sequences typically ranging from about 11 to about 20 amino acid residues in length. Comparative analysis of a range of proteins for which clinical immunogenicity data exists shows a strong relationship between the presence and potency of T-cell epitopes with the immunogenicity of the corresponding protein.

In silico screening tools are frequently used as an initial step in a comprehensive T-cell epitope assessment. The induction of helper CD4+ T-cell responses to a peptide requires peptide binding to MHC class II. Analysis of such peptide binding data can be exploited in the development process of therapeutic proteins. By way of example, Antitope Ltd (Cambridge, UK) has a proprietary in silico molecular modeling technology (iTope™) that models the binding of peptides to 34 MHC class II alleles. The contribution of individual amino acid residues to peptide binding can be determined for each allele, and these data can then be used in the design of ‘de-immunized’ sequence variants in which T-cell epitopes are mutated to disrupt binding.

In addition, ‘immunoinformatics’ algorithms and other technologies for identifying T-cell epitopes can be used to triage protein therapeutics into higher-risk and lower-risk categories. To illustrate, protein sequences can be parsed into overlapping 9-mer peptide frames which are then evaluated for binding potential to each of eight common class II HLA alleles that “cover” the genetic backgrounds of most humans. By calculating the density of high-scoring frames within a protein, it is possible to estimate a protein's overall “immunogenicity score”. In addition, sub-regions of densely-packed, high scoring frames or “clusters” of potential immunogenicity can be identified, and cluster scores can be calculated and compiled. A protein's “immunogenicity score”, along with other determinants of immunogenicity, can then be used to determine the likelihood that a protein will illicit an immune response.

Additional means of reducing a therapeutic protein's immunogenicity may be employed. Technologies (e.g., Antitope's proprietary EpiScreen™ human ex vivo T cell assay system) can be used to determine helper CD4+ T-cell responses to proteins, peptides, formulations, etc. Data generated from the use of such technologies can be used to map helper CD4+ T-cell epitopes within the sequence of the starting protein, and the T-cell epitopes can then be removed from the protein by one or more of the following: designing mutations in order to reduce/eliminate binding to human MHC class II; targeting T-cell receptor contact residues to disrupt recognition of peptide/MHC class II complexes; conducting structural and homology analysis to guide the targeting and substitution of key amino acid residues in order to maintain desired protein activity; and prioritizing T-cell epitopes for removal based on potency.

B-Cell Epitopes.

While accurate predictors for T-cell epitopes exist, currently the prediction of B-cell epitopes is inherently more difficult.

B-cell epitopes can be placed in one of two categories. In the first category, epitopes are defined by the primary amino acid sequence of a particular region of a protein, and the components of the epitope are situated sequentially on the protein. These linear B-cell epitopes generally range from about 5 to about 20 amino acid residues in length. In the second category, epitopes are defined by the conformational structure of a protein, and the components of the epitope are situated on separate parts of the protein that are brought into proximity of each other in the folded secondary or tertiary structure of the native protein. Because most B-cell epitopes are based on the conformational structure of a protein, B-cell epitopes are more difficult to identify than T-cell epitopes (which are determined by their primary amino acid sequence).

Examples of previously used sequence-based B-cell epitope predictors include technologies described by Saha S, and Raghava G P (“ABCPred technology”) (Proteins (2006) 65:40-48); Chen et al. (Amino Acids (2007) 33:423-28); El-Manzalawy Y, et al. (“BCPred” technology) (J Mol Recognit (2008) 21:243-55); Sweredoski M J, and Baldi P (“COBEpro” technology) (Protein Eng Des Sel (2009) 22:113-20); Wee U, et al. (“BayesB” technology) (BMC Genomics (2010) 11:S21); and Ansari H R, and Raghava G P (“CBTOPE” technology) (Immunome Res (2010) 6:6).

B-cell Epitope prediction using Support vector machine Tool (“BEST”) is a promising new B-cell epitope technology (Gao J, et al. (2012) PLoS ONE 7(6): e40104. doi:10.1371/journal.pone.0040104). The BEST method predicts epitopes from antigen sequences, in contrast to many previous methods that predict only from short sequence fragments, using a new architecture based on averaging selected scores generated from sliding 20-mers by a Support Vector Machine (SVM). The SVM predictor utilizes a comprehensive and custom-designed set of inputs generated by combining information derived from the chain, sequence conservation, similarity to known (training) epitopes, and predicted secondary structure and relative solvent accessibility. In addition, several commercial entities utilize proprietary technologies to assess B-cell epitopes (e.g., ProImmune's B-cell ELISpot technology; ProImmune Ltd.; Oxford, UK).

For purposes of assessing immunogenicity, it is useful to focus on potential T-cell epitopes, which generally, though not always, drive antigen-specific B-cell responses.

### Methods of Production of IL-10

A polypeptide of the present disclosure can be produced by any suitable method, including non-recombinant (e.g., chemical synthesis) and recombinant methods.

Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis may be performed as described hereafter. The alpha functions (Nα) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α,α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, 1-(4,4-dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Nα-protected amino acid (e.g., the Fmoc amino acid) to the PAL, Wang or Rink anchor can, for example, be carried out with the aid of coupling reagents such as N,N′-dicyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Nα-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Nα-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at 25° C. The previously mentioned reagents for coupling the first Nα-Fmoc amino acid to the PAL, Wang or Rink anchor are suitable as coupling reagents. Active esters of the protected amino acid, or chlorides or fluorides or symmetric anhydrides thereof, can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition, the peptide can be cleaved from the support by re-esterification, e.g., with methanol.

The acidic solution that is obtained may be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

### Recombinant Production

Methods describing the preparation of human and mouse IL-10 can be found in, for example, U.S. Pat. No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Site-specific mutagenesis (also referred to as site-directed mutagenesis and oligonucleotide-directed mutagenesis) can be used to generate specific mutations in DNA to produce rationally-designed proteins of the present disclosure (e.g., particular IL-10 muteins and other modified versions of IL-10, including domains thereof) having improved or desirable properties. Techniques for site-specific mutagenesis are well known in the art. Early site-specific mutagenesis methods (e.g., Kunkel's method; cassette mutagenesis; PCR site-directed mutagenesis; and whole plasmid mutagenesis, including SPRINP) have been replaced by more precise and efficient methods, such as various in vivo methods that include Delitto perfetto (see Storici F. and Resnick M A, (2006) Methods in Enzymology 409:329-45); transplacement “pop-in pop-out”; direct gene deletion and site-specific mutagenesis with PCR and one recyclable marker; direct gene deletion and site-specific mutagenesis with PCR and one recyclable marker using long homologous regions; and in vivo site-directed mutagenesis with synthetic oligonucleotides (and see, e.g., In Vitro Mutagenesis Protocols (Methods in Molecular Biology), 2nd Ed. ISBN 978-0896039100). In addition, tools for effecting site-specific mutagenesis are commercially available (e.g., Stratagene Corp., La Jolla, Calif.).

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that may be used as host cells include insect cells, mammalian cells, and/or plant cells. Where mammalian host cells are used, they may include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host may be present to facilitate selection of cells containing the vector. Moreover, the expression construct may include additional elements. For example, the expression vector may have one or two replication systems, thus allowing it to be maintained in organisms, for example, in mammalian or insect cells for expression and in a prokaryotic host for cloning and amplification. In addition, the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein may be isolated using metal chelate chromatography methods. Proteins may contain modifications to facilitate isolation.

The polypeptides may be prepared in substantially pure or isolated form (e.g., free from other polypeptides). The polypeptides can be present in a composition that is enriched for the polypeptide relative to other components that may be present (e.g., other polypeptides or other host cell components). For example, purified polypeptide may be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-10 polypeptide may be generated using recombinant techniques to manipulate different IL-10-related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-10 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-10 can be substituted.

In another example, one or more amide linkages (—CO—NH—) in IL-10 can be replaced with a linkage which is an isostere of an amide linkage, such as —CH₂NH—, —CH₂S—, —CH₂CH₂—, —CH═CH-(cis and trans), —COCH₂—, —CH(OH)CH₂— or —CH₂SO—. One or more amide linkages in IL-10 can also be replaced by, for example, a reduced isostere pseudopeptide bond. See Couder et al. (1993) Int. J. Peptide Protein Res. 41:181-184. Such replacements and how to effect them are known to those of ordinary skill in the art.

### Amino Acid Substitutions

One or more amino acid substitutions can be made in an IL-10 polypeptide. The following are non-limiting examples:

a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma,gamma′-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-10 comprises one or more naturally occurring non-genetically encoded L-amino acids, synthetic L-amino acids, or D-enantiomers of an amino acid. In some embodiments, IL-10 comprises only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

A cysteine residue or a cysteine analog can be introduced into an IL-10 polypeptide to provide for linkage to another peptide via a disulfide linkage or to provide for cyclization of the IL-10 polypeptide. Methods of introducing a cysteine or cysteine analog are known in the art (see, e.g., U.S. Pat. No. 8,067,532).

An IL-10 polypeptide can be cyclized. One or more cysteines or cysteine analogs can be introduced into an IL-10 polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ-CO— or —(CH2)ₙ-C₆H₄—CO—) with functional groups which allow for the introduction of a bridge. Some examples are disulfides, disulfide mimetics such as the —(CH2)ₙ- carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-10 polypeptide is replaced with one or more D-amino acids.

In some cases, an IL-10 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. [See, e.g., Jameson et al. (1994) Nature 368:744; and Brady et al. (1994) Nature 368:692].

An IL-10 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:3); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:4); Transportan GWTLNSAGYLLGKINLKALAALAKKIL (SEQ ID NO:5); KALAWEAKLAKALAKALAKHLAKALAKALKCEA (SEQ ID NO:6); and RQIKIWFQNRRMKWKK (SEQ ID NO:7). Exemplary PTDs include, but are not limited to, YGRKKRRQRRR (SEQ ID NO:8), RKKRRQRRR (SEQ ID NO:9); an arginine homopolymer of from 3 arginine residues to 50 arginine residues; exemplary PTD domain amino acid sequences include, but are not limited to, any of the following: YGRKKRRQRRR (SEQ ID NO:10); RKKRRQRR (SEQ ID NO:11); YARAAARQARA (SEQ ID NO:12); THRLPRRRRRR (SEQ ID NO:13); and GGRRARRRRRR (SEQ ID NO:14).

The carboxyl group COR₃ of the amino acid at the C-terminal end of an IL-10 polypeptide can be present in a free form (R₃═OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR₁R₂ at the N-terminus of an IL-10 polypeptide can be present in a free form (R₁═H and R₂═H) or in the form of a physiologically-tolerated salt such as, e.g., a chloride or acetate. The amino group can also be acetylated with acids such that R₁═H and R₂=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂═C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

Particular Modifications to Enhance and/or Mimic IL-10 Function

It is frequently beneficial, and sometimes imperative, to improve one of more physical properties of the treatment modalities disclosed herein (e.g., an IL-10 mutein) and/or the manner in which they are administered. Improvements of physical properties include, for example, modulating immunogenicity; methods of increasing water solubility, bioavailability, serum half-life, and/or therapeutic half-life; and/or modulating biological activity. Certain modifications may also be useful to, for example, raise antibodies for use in detection assays (e.g., epitope tags) and to provide for ease of protein purification. Such improvements must generally be imparted without adversely impacting the bioactivity of the treatment modality and/or increasing its immunogenicity.

Pegylation of IL-10 is one particular modification contemplated by the present disclosure, while other modifications include, but are not limited to, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins. In addition, PEG mimetics represent other modifications contemplated herein.

Pegylation:

The clinical effectiveness of protein therapeutics is often limited by short plasma half-life and susceptibility to protease degradation. Studies of various therapeutic proteins have shown that such difficulties may be overcome by various modifications, including conjugating or linking the polypeptide sequence to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

In addition to the beneficial effects of pegylation on pharmacokinetic parameters, pegylation itself may enhance activity. For example, PEG-IL-10 has been shown to be more efficacious against certain cancers than unpegylated IL-10 (see, e.g., EP 206636A2).

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight (molecular mass) of the PEG used in the present disclosure is not restricted to any particular range. Certain embodiments have molecular weights between 5 kDa and 20 kDa, while other embodiments have molecular weights between 4 kDa and 10 kDa. Further embodiments describing PEGs having additional molecular weights are described elsewhere herein.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography may be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein. PEG may be bound to a polypeptide of the present disclosure via a terminal reactive group (a “spacer”) which mediates a bond between the free amino or carboxyl groups of one or more of the polypeptide sequences and polyethylene glycol. The PEG having the spacer which may be bound to the free amino group includes N-hydroxysuccinylimide polyethylene glycol, which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH≧7), and longer reaction time tend to increase the number of PEGs attached. Various means known in the art may be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

As indicated above, pegylation most frequently occurs at the N-terminus, the side chain of lysine residues, and the imidazole group on the side chain of histidine residues. The usefulness of such pegylation has been enhanced by refinement by, for example, optimization of reaction conditions and improvement of purification processes. More recent residue-specific chemistries have enabled pegylation of arginine, aspartic acid, cysteine, glutamic acid, serine, threonine, and tyrosine, as well as the carboxy-terminus. Some of these amino acid residues can be specifically pegylated, while others are more promiscuous or only result in site-specific pegylation under certain conditions.

Current approaches allowing pegylation of additional amino acid residues include bridging pegylation (disulfide bridges), enzymatic pegylation (glutamines and C-terminus) and glycopegylation (sites of 0- and N-glycosylation or the glycans of a glycoprotein), and heterobifunctional pegylation. Further approaches are drawn to pegylation of proteins containing unnatural amino acids, intein fusion proteins for C-terminal pegylation, transglutaminase-mediated pegylation, sortase A-mediated pegylation, and releasable and non-covalent pegylation. In addition, combination of specific pegylation approaches with genetic engineering techniques has enabled the polyethylene glycan polymer to essentially couple at any position on the protein surface due to, for example, substitution of specific amino acid residues in a polypeptide with a natural or unnatural amino acid bearing an orthogonal reactive group. See generally, e.g., Pasut, G. and Veronese, F. M., (2012) J. Controlled Release 161:461-72; Roberts, M. J. et al., (2012) Advanced Drug Delivery Rev. 64:116-27; Jevsevar, S. et al., (2010) Biotechnol. J. 5:113-28; and Yoshioka, Y. (2011) Chem. Central J. 5:25.

The therapeutic value of pegylation molecules is well validated. Previous and/or current pharmaceutical products include: OMONTYS (Affymax/Takeda); PEGLOTICASE (Savient); CIMZIA (Nektar/UCB Pharma); MACUGEN (Prizer); NEULASTA (Amgen); SOMAVERT (Prizer); PEGASYS (Roche); DOXIL (Ortho Biotech) and PEGINTRON (Schering-Plough).

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Glycosylation:

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present.

Glycosylation can dramatically affect the physical properties (e.g., solubility) of polypeptides such as IL-10 and can also be important in protein stability, secretion, and subcellular localization. Glycosylated polypeptides may also exhibit enhanced stability or may improve one or more pharmacokinetic properties, such as half-life. In addition, solubility improvements can, for example, enable the generation of formulations more suitable for pharmaceutical administration than formulations comprising the non-glycosylated polypeptide.

Proper glycosylation can be essential for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the nucleic acid level, particularly by mutating the nucleic acid encoding the polypeptide at preselected bases such that codons are generated that will translate into the desired amino acids. Another means of increasing the number of carbohydrate moieties on the polypeptide is by chemical or enzymatic coupling of glycosides to the polypeptide. Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Dihydrofolate reductase (DHFR)—deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

Polysialylation:

The present disclosure also contemplates the use of polysialylation, the conjugation of polypeptides to the naturally occurring, biodegradable α-(2→8)-linked polysialic acid (“PSA”) in order to improve the polypeptides' stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of in vivo activity, and reduction in immunogenicity and antigenicity (see, e.g., G. Gregoriadis et al., Int. J. Pharmaceutics (2005) 300(1-2):125-30). As with modifications with other conjugates (e.g., PEG), various techniques for site-specific polysialylation are available (see, e.g., T. Lindhout et al., (2011) PNAS 108(18)7397-7402).

Albumin Fusion:

Additional suitable components and molecules for conjugation include albumins such as human serum albumin (HSA), cyno serum albumin, and bovine serum albumin (BSA).

Mature HSA, a 585 amino acid polypeptide (˜67 kDa) having a serum half-life of ˜20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulphide bridges. The three primary drug binding regions of albumin are located on each of the three domains within sub-domains IB, IIA and IIIA.

Albumin synthesis takes place in the liver, which produces the short-lived, primary product preproalbumin. Thus, the full-length HSA has a signal peptide of 18 amino acids (MKWVTFISLLFLFSSAYS; SEQ ID NO:15) followed by a pro-domain of 6 amino acids (RGVFRR; SEQ ID NO:16); this 24 amino acid residue peptide may be referred to as the pre-pro domain. HSA can be expressed and secreted using its endogenous signal peptide as a pre-pro-domain. Alternatively, HSA can be expressed and secreted using a IgK signal peptide fused to a mature construct. Preproalbumin is rapidly co-translationally cleaved in the endoplasmic reticulum lumen at its amino terminus to produce the stable, 609-amino acid precursor polypeptide, proalbumin. Proalbumin then passes to the Golgi apparatus, where it is converted to the 585 amino acid mature albumin by a furin-dependent amino-terminal cleavage.

The primary amino acid sequences, structure, and function of albumins are highly conserved across species, as are the processes of albumin synthesis and secretion. Albumin serum proteins comparable to HSA are found in, for example, cynomolgus monkeys, cows, dogs, rabbits and rats. Of the non-human species, bovine serum albumin (BSA) is the most structurally similar to HSA (see, e.g., Kosa et al., November 2007 J Pharm Sci. 96(11):3117-24). The present disclosure contemplates the use of albumin from non-human species, including, but not limited to, those set forth above, in, for example, the drug development process.

According to the present disclosure, albumin may be conjugated to a drug molecule (e.g., a polypeptide described herein) at the carboxyl terminus, the amino terminus, both the carboxyl and amino termini, and internally (see, e.g., U.S. Pat. No. 5,876,969 and U.S. Pat. No. 7,056,701).

In the HSA-drug molecule conjugates contemplated by the present disclosure, various forms of albumin may be used, such as albumin secretion pre-sequences and variants thereof, fragments and variants thereof, and HSA variants. Such forms generally possess one or more desired albumin activities. In additional embodiments, the present disclosure involves fusion proteins comprising a polypeptide drug molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or a modified version thereof.

Intracellular cleavage may be carried out enzymatically by, for example, furin or caspase. Cells express a low level of these endogenous enzymes, which are capable of cleaving a portion of the fusion molecules intracellularly. Thus, some of the polypeptides are secreted from the cell without being conjugated to HSA, while others are secreted in the form of fusion molecules that comprise HSA. Embodiments of the present disclosure contemplate the use of various furin fusion constructs. For example, constructs may be designed that comprise the sequence RGRR (SEQ ID NO:17), RKRKKR (SEQ ID NO:18), RKKR (SEQ ID NO:19), or RRRKKR (SEQ ID NO:20).

The present disclosure also contemplates extra-cellular cleavage (ex-vivo cleavage) whereby the fusion molecules are secreted from the cell, subjected to purification, and then cleaved. It is understood that the excision may dissociate the entire HSA-linker complex from the mature IL-10, or less that the entire HSA-linker complex.

As alluded to above, fusion of albumin to one or more polypeptides of the present disclosure can, for example, be achieved by genetic manipulation, such that the nucleic acid coding for HSA, or a fragment thereof, is joined to the nucleic acid coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake through the cell membrane, incorporation and expression of exogenous genetic material (exogenous nucleic acid). Transformation occurs naturally in some bacteria, but it can also be effected by artificial means in other cells.

Furthermore, albumin itself may be modified to extend its circulating half-life. Fusion of the modified albumin to IL-10 can be attained by the genetic manipulation techniques described above or by chemical conjugation; the resulting fusion molecule has a half-life that exceeds that of fusions with non-modified albumin (see WO2011/051489).

Alternative Albumin Binding Strategies:

Several albumin-binding strategies have been developed as alternatives to direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

The present disclosure contemplates fusion proteins which comprise an albumin binding domain (ABD) polypeptide sequence and the sequence of one or more of the polypeptides described herein. Any ABD polypeptide sequence described in the literature can be a component of the fusion proteins. The components of the fusion proteins can be optionally covalently bonded through a linker, such as those linkers described herein. In some embodiments of the present disclosure, the fusion proteins comprise the ABD polypeptide sequence as an N-terminal moiety and the polypeptides described herein as a C-terminal moiety.

The present disclosure also contemplates fusion proteins comprising a fragment of an albumin binding polypeptide, which fragment substantially retains albumin binding; or a multimer of albumin binding polypeptides or fragments thereof comprising at least two albumin binding polypeptides or fragments thereof as monomer units. For a general discussion of ABD and related technologies, see WO 2012/050923, WO 2012/050930, WO 2012/004384 and WO 2009/016043.

Conjugation with Other Molecules:

Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Thus, the present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule.

A conjugate modification may result in a polypeptide sequence that retains activity with an additional or complementary function or activity derived from the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disease, disorder or condition as set forth herein (e.g., cancer).

An IL-10 polypeptide may also be conjugated to large, slowly metabolized macromolecules such as proteins; polysaccharides, such as sepharose, agarose, cellulose, or cellulose beads; polymeric amino acids, such as polyglutamic acid or polylysine; amino acid copolymers; inactivated virus particles; inactivated bacterial toxins, such as toxoid from diphtheria, tetanus, cholera, or leukotoxin molecules; inactivated bacteria; and dendritic cells. Such conjugated forms, if desired, can be used to produce antibodies against a polypeptide of the present disclosure.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates, magnetic beads, test strips, and membranes.

Purification methods such as cation exchange chromatography may be used to separate conjugates by charge difference, which effectively separates conjugates into their various molecular weights. For example, the cation exchange column can be loaded and then washed with ˜20 mM sodium acetate, pH ˜4, and then eluted with a linear (0 M to 0.5 M) NaCl gradient buffered at a pH of from about 3 to 5.5, e.g., at pH ˜4.5. The content of the fractions obtained by cation exchange chromatography may be identified by molecular weight using conventional methods, for example, mass spectroscopy, SDS-PAGE, or other known methods for separating molecular entities by molecular weight.

Fc-Fusion Molecules:

In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Other Modifications:

The present disclosure contemplates the use of other modifications, currently known or developed in the future, of IL-10 to improve one or more properties. One such method involves modification of the polypeptide sequences by hesylation, which utilizes hydroxyethyl starch derivatives linked to other molecules in order to modify the polypeptide sequences' characteristics. Various aspects of hesylation are described in, for example, U.S. Patent Appln. Nos. 2007/0134197 and 2006/0258607.

The present disclosure also contemplates fusion molecules comprising Small Ubiquitin-like Modifier (SUMO) as a fusion tag (LifeSensors, Inc.; Malvern, Pa.). Fusion of a polypeptide described herein to SUMO may convey several beneficial effects, including enhancement of expression, improvement in solubility, and/or assistance in the development of purification methods. SUMO proteases recognize the tertiary structure of SUMO and cleave the fusion protein at the C-terminus of SUMO, thus releasing a polypeptide described herein with the desired N-terminal amino acid.

The present disclosure also contemplates the use of PASylation™ (XL-Protein GmbH (Freising, Germany)). This technology expands the apparent molecular size of a protein of interest, without having a negative impact on the therapeutic bioactivity of the protein, beyond the pore size of the renal glomeruli, thereby decreasing renal clearance of the protein.

Linkers:

Linkers and their use have been described above. Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Exemplary flexible linkers include glycine polymers (G)ₙ, glycine-serine polymers (for example, (GS)ₙ, GSGGSₙ (SEQ ID NO:21), GGGSₙ (SEQ ID NO:22), (GₘSₒ)ₙ, (GₘSₒGₘ)ₙ, (GₘSₒGₘSₒGₘ)ₙ (SEQ ID NO:23), (GSGGSₘ)ₙ (SEQ ID NO:24), (GSGSₘG)ₙ (SEQ ID NO:25) and (GGGSₘ)ₙ (SEQ ID NO:26), and combinations thereof, where m, and o are each independently selected from an integer of at least one), glycine-alanine polymers, alanine-serine polymers, and other flexible linkers. Glycine and glycine-serine polymers are relatively unstructured, and therefore may serve as a neutral tether between components. Exemplary flexible linkers include, but are not limited to GGSG (SEQ ID NO:27), GGSGG (SEQ ID NO:28), GSGSG (SEQ ID NO:29), GSGGG (SEQ ID NO:30), GGGSG (SEQ ID NO:31), and GSSSG (SEQ ID NO:32).

In certain embodiments of the present disclosure, PEG is conjugated to IL-10 through an activated linker that is covalently attached to one or more PEG molecules. A linker is “activated” if it is chemically reactive and ready for covalent attachment to a reactive group on a peptide. The present disclosure contemplates the use of any activated linker provided that it can accommodate one or more PEG molecules and form a covalent bond with an amino acid residue under suitable reaction conditions. In particular aspects, the activated linker attaches to an alpha amino group in a highly selective manner over other attachment sites (e.g., the epsilon amino group of lysine or the imino group of histidine).

In some embodiments, activated PEG can be represented by the formula: (PEG)_{b}-L′, where PEG covalently attaches to a carbon atom of the linker to form an ether bond, b is 1 to 9 (i.e., 1 to 9 PEG molecules can be attached to the linker), and L′ contains a reactive group (an activated moiety) which can react with, for example, an amino or imino group on an amino acid residue to provide a covalent attachment of the PEG to IL-10. In other embodiments, an activated linker (L′) contains an aldehyde of the formula RCHO, where R is a linear or branched C₁₋₁₁ alkyl; after covalent attachment of an activated linker to IL-10, the linker contains 2 to 12 carbon atoms. The present disclosure contemplates embodiments wherein propionaldehyde is an exemplary activated linker. PEG-propionaldehyde (CH₂CH₂CHO) is described in U.S. Pat. No. 5,252,714 and is commercially available (e.g., Shearwater Polymers (Huntsville, Ala.). Other activated PEG-linkers can be obtained commercially from, e.g., Shearwater Polymers and Enzon, Inc. (Piscataway, N.J.).

In some embodiments, it is desirable to covalently attach more than one PEG molecule to IL-10, and a suitable activated branched (i.e., “multi-armed”) linker can be used. Any suitable branched PEG linker that covalently attaches two or more PEG molecules to an amino group on an amino acid residue of IL-10 (e.g., to an alpha amino group at the N-terminus) can be used. In particular embodiments, a branched linker used in this invention contains two or three PEG molecules. By way of example, a branched PEG linker can be a linear or branched aliphatic group that is hydrolytically stable and contains an activated moiety (e.g., an aldehyde group), which reacts with an amino group of an amino acid residue, as described above; the aliphatic group of a branched linker can contain 2 to 12 carbons. In some embodiments, an aliphatic group can be a t-butyl which contains as many as three PEG molecules on each of three carbon atoms (i.e., a total of 9 PEG molecules) and a reactive aldehyde moiety on the fourth carbon of the t-butyl.

Further exemplary branched PEG linkers are described in U.S. Pat. Nos. 5,643,575, 5,919,455, 7,052,868, and 5,932,462. The skilled artisan can prepare modifications to branched PEG linkers by, e.g., addition of a reactive aldehyde moiety. Methods for preparing linkers for use are also well known in the art, and are described in, e.g., the US patents listed above.

Exemplary linkers used in HSA conjugates are known in the art and include heterobifunctional linkers, such as [succinimidyl 4-[N-maleimidomethyl]cyclohexane-1-carboxylate (SMCC), 6-maleimidohexanoic acid N-hydroxysuccinimide ester (MHS), and N-[γ-maleimidobutyryloxy]sulfosuccinimide ester (GMBS)]. See Ehrilich, G K et al., Bioconjug. Chem. (2013 Dec. 18); 24(12):2015-24. Further examples of HSA linkers and conjugates thereof are described in, e.g., US20120003221.

### Therapeutic and Prophylactic Uses

The present disclosure contemplates the use of the IL-10 polypeptides described herein (e.g., PEG-IL-10) in the treatment or prevention of a broad range of diseases, disorders and/or conditions, and/or the symptoms thereof. While particular uses are described in detail hereafter, it is to be understood that the present disclosure is not so limited. Furthermore, although general categories of particular diseases, disorders and conditions are set forth hereafter, some of the diseases, disorders and conditions may be a member of more than one category (e.g., cancer- and fibrotic-related disorders), and others may not be a member of any of the disclosed categories.

Fibrotic Disorders and Cancer.

In accordance with the present disclosure, an IL-10 molecule can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. The disclosure contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09). In particular embodiments, the tumor or cancer is colon cancer, ovarian cancer, breast cancer, melanoma, lung cancer, glioblastoma, or leukemia. The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the present disclosure provides methods for treating a proliferative condition, cancer, tumor, or precancerous condition with an IL-10 molecule and at least one additional therapeutic or diagnostic agent, examples of which are set forth elsewhere herein.

The present disclosure also provides methods of treating or preventing fibrotic diseases, disorders and conditions. As used herein, the phrase “fibrotic diseases, disorders and conditions”, and similar terms (e.g., “fibrotic disorders”) and phrases, is to be construed broadly such that it includes any condition which may result in the formation of fibrotic tissue or scar tissue (e.g., fibrosis in one or more tissues). By way of example, injuries (e.g., wounds) that may give rise to scar tissue include wounds to the skin, eye, lung, kidney, liver, central nervous system, and cardiovascular system. The phrase also encompasses scar tissue formation resulting from stroke, and tissue adhesion, for example, as a result of injury or surgery.

As used herein the term “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue.

Fibrotic disorders include, but are not limited to, fibrosis arising from wound healing, systemic and local scleroderma, atherosclerosis, restenosis, pulmonary inflammation and fibrosis, idiopathic pulmonary fibrosis, interstitial lung disease, liver cirrhosis, fibrosis as a result of chronic hepatitis B or C infection, kidney disease (e.g., glomerulonephritis), heart disease resulting from scar tissue, keloids and hypertrophic scars, and eye diseases such as macular degeneration, and retinal and vitreal retinopathy. Additional fibrotic diseases include chemotherapeutic drug-induced fibrosis, radiation-induced fibrosis, and injuries and burns.

Fibrotic disorders are often hepatic-related, and there is frequently a nexus between such disorders and the inappropriate accumulation of liver cholesterol and triglycerides within the hepatocytes. This accumulation appears to result in a pro-inflammatory response that leads to liver fibrosis and cirrhosis. Hepatic disorders having a fibrotic component include non-alcoholic fatty liver disease (NAFLD) and non-alcoholic steatohepatitis (NASH). NAFLD occurs when steatosis (fat deposition in the liver) is present that is not due to excessive alcohol use. It is related to insulin resistance and the metabolic syndrome. NASH is the most extreme form of NAFLD, and is regarded as a major cause of cirrhosis of the liver of unknown cause.

Cardiovascular Diseases.

The present disclosure also contemplates the use of the IL-10 molecules described herein to treat and/or prevent certain cardiovascular- and/or associated metabolic-related diseases, disorders and conditions, as well as disorders associated therewith.

As used herein, the terms “cardiovascular disease”, “heart disease” and the like refer to any disease that affects the cardiovascular system, primarily cardiac disease, vascular diseases of the brain and kidney, and peripheral arterial diseases. Cardiovascular disease is a constellation of diseases that includes coronary heart disease (i.e., ischemic heart disease or coronary artery disease), atherosclerosis, cardiomyopathy, hypertension, hypertensive heart disease, cor pulmonale, cardiac dysrhythmias, endocarditis, cerebrovascular disease, and peripheral arterial disease. Cardiovascular disease is the leading cause of deaths worldwide, and while it usually affects older adults, the antecedents of cardiovascular disease, notably atherosclerosis, begin in early life.

Particular embodiments of the present disclosure are directed to the use of IL-10 polypeptides to treat and/or prevent atherosclerosis, a chronic condition in which an artery wall thickens to form plaques as a result of the accumulation of fatty materials such as cholesterol and triglycerides. Atherosclerosis frequently involves a chronic inflammatory response in the walls of arteries, caused largely by the accumulation of macrophages and promoted by low-density lipoproteins (LDL) without adequate removal of fats and cholesterol from the macrophages by functional high-density lipoproteins. Chronically expanding atherosclerotic lesions can cause complete closure of the lumen, which may only manifest when the lumen stenosis is so severe that blood supply to downstream tissue(s) is insufficient, resulting in ischemia.

The IL-10 polypeptides may be particularly advantageous in the treatment and/or prevention of cholesterol-related disorders, which may be associated with, for example, cardiovascular disease (e.g. atherosclerosis), cerebrovascular disease (e.g., stroke), and peripheral vascular disease. By way of example, but not limitation, the IL-10 polypeptides may be used for lowering a subject's blood cholesterol level. In determining whether a subject has hypercholesterolemia, there is no firm demarcation between normal and abnormal cholesterol levels, and interpretation of values needs to be made in relation to other health conditions and risk factors. Nonetheless, the following guidelines are generally used in the United States: total cholesterol <200 mg/dL is desirable, 200-239 mg/dL is borderline high, and ≧240 mg/dL is high. Higher levels of total cholesterol increase the risk of cardiovascular disease, and levels of LDL or non-HDL cholesterol are both predictive of future coronary heart disease. When assessing hypercholesterolemia, it is frequently useful to measure all lipoprotein subfractions (VLDL, IDL, LDL and HDL). A particular therapeutic goal is to decrease LDL while maintaining or increasing HDL.

Thrombosis and Thrombotic Conditions.

Thrombosis, the formation of a thrombus (blood clot) inside a blood vessel resulting in obstruction of the flow of blood through the circulatory system, may be caused by abnormalities in one or more of the following (Virchow's triad): hypercoagulability, endothelial cell injury, or disturbed blood flow (stasis, turbulence).

Thrombosis is generally categorized as venous or arterial, each of which can be presented by several subtypes. Venous thrombosis includes deep vein thrombosis (DVT), portal vein thrombosis, renal vein thrombosis, jugular vein thrombosis, Budd-Chiari syndrome, Paget-Schroetter disease, and cerebral venous sinus thrombosis. Arterial thrombosis includes stroke and myocardial infarction.

Other diseases, disorders and conditions are contemplated by the present disclosure, including atrial thrombosis and Polycythemia vera (also known as erythema, primary polycythemia and polycythemia rubra vera), a myeloproliferative blood disorder in which the bone marrow makes too many RBCs, WBCs and/or platelets.

Immune and Inflammatory Conditions.

As used herein, terms such as “immune disease”, “immune condition”, “immune disorder”, “inflammatory disease”, “inflammatory condition”, “inflammatory disorder” and the like are meant to broadly encompass any immune- or inflammatory-related condition (e.g., pathological inflammation and autoimmune diseases). Such conditions frequently are inextricably intertwined with other diseases, disorders and conditions. By way of example, an “immune condition” may refer to proliferative conditions, such as cancer, tumors, and angiogenesis; including infections (acute and chronic), tumors, and cancers that resist eradication by the immune system.

A non-limiting list of immune- and inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (e.g., Crohn's disease and ulcerative colitis), allergic contact dermatitis and other eczemas, systemic sclerosis, transplantation and multiple sclerosis.

Some of the aforementioned diseases, disorders and conditions for which an IL-10 molecule may be particularly efficacious (due to, for example, limitations of current therapies) are described in more detail hereafter.

The IL-10 polypeptides of the present disclosure may be particularly effective in the treatment and prevention of inflammatory bowel diseases (IBD). IBD comprises Crohn's disease (CD) and ulcerative colitis (UC), both of which are idiopathic chronic diseases that can affect any part of the gastrointestinal tract, and are associated with many untoward effects, and patients with prolonged UC are at an increased risk of developing colon cancer. Current IBD treatments are aimed at controlling inflammatory symptoms, and while certain agents (e.g., corticosteroids, aminosalicylates and standard immunosuppressive agents (e.g., cyclosporine, azathioprine, and methotrexate)) have met with limited success, long-term therapy may cause liver damage (e.g., fibrosis or cirrhosis) and bone marrow suppression, and patients often become refractory to such treatments.

Psoriasis, a constellation of common immune-mediated chronic skin diseases, affects more than 4.5 million people in the U.S., of which 1.5 million are considered to have a moderate-to severe form of the disease. Moreover, over 10% of patients with psoriasis develop psoriatic arthritis, which damages the bone and connective tissue around the joints. An improved understanding of the underlying physiology of psoriasis has resulted in the introduction of agents that, for example, target the activity of T lymphocytes and cytokines responsible for the inflammatory nature of the disease. Such agents include the TNF-α inhibitors (also used in the treatment of rheumatoid arthritis (RA)), including ENBREL (etanercept), REMICADE (infliximab) and HUMIRA (adalimumab)), and T-cell inhibitors such as AMEVIVE (alefacept) and RAPTIVA (efalizumab). Though several of these agents are effective to some extent in certain patient populations, none have been shown to effectively treat all patients.

Rheumatoid Arthritis (RA), which is generally characterized by chronic inflammation in the membrane lining (the synovium) of the joints, affects approximately 1% of the U.S. population (˜2.1 million people). Further understanding of the role of cytokines, including TNF-α and IL-1, in the inflammatory process has enabled the development and introduction of a new class of disease-modifying antirheumatic drugs (DMARDs). Agents (some of which overlap with treatment modalities for RA) include ENBREL (etanercept), REMICADE (infliximab), HUMIRA (adalimumab) and KINERET (anakinra) Though some of these agents relieve symptoms, inhibit progression of structural damage, and improve physical function in particular patient populations, there is still a need for alternative agents with improved efficacy, complementary mechanisms of action, and fewer/less severe adverse effects.

Subjects suffering from multiple sclerosis (MS), a seriously debilitating autoimmune disease comprising multiple areas of inflammation and scarring of the myelin in the brain and spinal cord, may be particularly helped by the IL-10 polypeptides described herein, as current treatments only alleviate symptoms or delay the progression of disability.

Similarly, the IL-10 polypeptides may be particularly advantageous for subjects afflicted with neurodegenerative disorders, such as Alzheimer's disease (AD), a brain disorder that seriously impairs patients' thought, memory, and language processes; and Parkinson's disease (PD), a progressive disorder of the CNS characterized by, for example, abnormal movement, rigidity and tremor. These disorders are progressive and debilitating, and no curative agents are available.

Viral Diseases.

There has been increased interest in the role of IL-10 in viral diseases. IL-10 has been postulated to produce both stimulatory and inhibitory effects depending on its receptor binding activity.

The effect of inhibiting IL-10 function in order to increase antiviral immunity and vaccine efficacy has been considered (see Wilson, E., (2011) Curr. Top. Microbiol. Immunol. 350:39-65). Moreover, the role of IL-10 in human immunodeficiency virus (HIV) function has been studied. In addition to the inhibition of human immunodeficiency virus type 1 (HIV-1) replication, IL-10 may also promote viral persistence by inactivation of effector immune mechanisms (Naicker, D., et al., (2009) J. Infect. Dis. 200 (3):448-452). Another study has identified an IL-10-producing subset of B-cells able to regulate T-cell immunity in chronic hepatitis B virus (HBV) infection. A close temporal correlation was observed between IL-10 levels and fluctuations in viral load, and in vitro blockade of IL-10 was found to rescue polyfunctional, virus-specific CD8+ T-cell responses (Das, A., et al., J. Immunol., Sep. 12, 2012 1103139 (on-line)).

Although the aforementioned studies indicate that IL-10 inhibition may be beneficial, particular viral infections that comprise a CD8+ T-cell component may be candidates for treatment and/or prevention through the administration of IL-10. This is supported by the positive role that IL-10 plays in certain cancers by modulation of regulatory T cells and/or CD8+ T cells. The use of IL-10 therapy in viral contexts has also been discussed elsewhere (see, e.g., J. Virol. July 2011 vol. 85 no. 14 6822-683; and Loebbermann J, et al. (2012) PLoS ONE 7(2): e32371. doi:10.1371/journal.pone.0032371).

The present disclosure contemplates the use of the IL-10 polypeptides in the treatment and/or prevention of any viral disease, disorder or condition for which treatment with IL-10 may be beneficial. Examples of viral diseases, disorders and conditions that are contemplated include hepatitis B, hepatitis C, HIV, herpes virus and cytomegalovirus (CMV).

### Pharmaceutical Compositions

The IL-10 polypeptides of the present disclosure may be in the form of compositions suitable for administration to a subject. In general, such compositions are “pharmaceutical compositions” comprising IL-10 and one or more pharmaceutically acceptable or physiologically acceptable diluents, carriers or excipients. In certain embodiments, the IL-10 polypeptides are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present disclosure.

The pharmaceutical compositions typically comprise a therapeutically effective amount of an IL-10 polypeptide contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver IL-10, including implants (e.g., implantable pumps) and catheter systems, slow injection pumps and devices, all of which are well known to the skilled artisan. Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or

oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates the administration of the IL-10 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The IL-10 polypeptides contemplated by the present disclosure may be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Routes of Administration

The present disclosure contemplates the administration of IL-10 molecules, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, may also be utilized to release the IL-10 molecules disclosed herein over a defined period of time.

Particular embodiments of the present disclosure contemplate parenteral administration, and in further particular embodiments the parenteral administration is subcutaneous.

### Combination Therapy

The present disclosure contemplates the use of IL-10 molecules in combination with one or more active therapeutic agents (e.g., cytokines) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the IL-10 polypeptides and the one or more active therapeutic agents or other prophylactic or therapeutic modalities are administered or applied sequentially, e.g., where one agent is administered prior to one or more other agents. In other embodiments, the IL-10 polypeptides and the one or more active therapeutic agents or other prophylactic or therapeutic modalities are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The IL-10 polypeptides of the present disclosure may be used in combination with at least one other (active) agent in any manner appropriate under the circumstances. In one embodiment, treatment with the at least one active agent and at least one IL-10 polypeptide of the present disclosure is maintained over a period of time. In another embodiment, treatment with the at least one active agent is reduced or discontinued (e.g., when the subject is stable), while treatment with the IL-10 polypeptide of the present disclosure is maintained at a constant dosing regimen. In a further embodiment, treatment with the at least one active agent is reduced or discontinued (e.g., when the subject is stable), while treatment with the IL-10 polypeptide of the present disclosure is reduced (e.g., lower dose, less frequent dosing or shorter treatment regimen). In yet another embodiment, treatment with the at least one active agent is reduced or discontinued (e.g., when the subject is stable), and treatment with the IL-10 polypeptide of the present disclosure is increased (e.g., higher dose, more frequent dosing or longer treatment regimen). In yet another embodiment, treatment with the at least one active agent is maintained and treatment with the IL-10 polypeptide of the present disclosure is reduced or discontinued (e.g., lower dose, less frequent dosing or shorter treatment regimen). In yet another embodiment, treatment with the at least one active agent and treatment with the IL-10 polypeptide of the present disclosure are reduced or discontinued (e.g., lower dose, less frequent dosing or shorter treatment regimen).

Fibrotic Disorders and Cancer.

The present disclosure provides methods for treating and/or preventing a proliferative condition; a fibrotic disease, disorder, or condition; cancer, tumor, or precancerous disease, disorder or condition with an IL-10 molecule and at least one additional therapeutic or diagnostic agent.

Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In certain embodiments, combination therapy comprises administration of a hormone or related hormonal agent.

Additional treatment modalities that may be used in combination with the IL-10 polypeptides include a cytokine or cytokine antagonist, such as IL-12, INFα, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

Therapeutic agents useful in combination therapy for the treatment of fibrotic disorders are well known to the skilled artisan. By way of example, agents such as those described herein for the treatment of insulin resistant-states (e.g., diabetes mellitus type 2) and the metabolic syndrome (e.g., metformin, thiazolidinediones, and statins) may help control NAFLD and NASH, particularly manifestations thereof. Vitamin E has also been shown to help control NAFLD and NASH in some patients.

The present disclosure encompasses pharmaceutically acceptable salts, acids or derivatives of any of the above.

Cardiovascular Diseases.

The present disclosure provides methods for treating and/or preventing certain cardiovascular- and/or metabolic-related diseases, disorders and conditions, as well as disorders associated therewith, with an IL-10 molecule and at least one additional therapeutic or diagnostic agent.

Examples of therapeutic agents useful in combination therapy for the treatment of hypercholesterolemia (and atherosclerosis as well) include statins (e.g., CRESTOR, LESCOL, LIPITOR, MEVACOR, PRAVACOL, and ZOCOR), which inhibit the enzymatic synthesis of cholesterol; bile acid resins (e.g., COLESTID, LO-CHOLEST, PREVALITE, QUESTRAN, and WELCHOL), which sequester cholesterol and prevent its absorption; ezetimibe (ZETIA), which blocks cholesterol absorption; fibric acid (e.g., TRICOR), which reduces triglycerides and may modestly increase HDL; niacin (e.g., NIACOR), which modestly lowers LDL cholesterol and triglycerides; and/or a combination of the aforementioned (e.g., VYTORIN (ezetimibe with simvastatin). Alternative cholesterol treatments that may be candidates for use in combination with the IL-10 polypeptides described herein include various supplements and herbs (e.g., garlic, policosanol, and guggul). The present disclosure encompasses pharmaceutically acceptable salts, acids or derivatives of any of the above.

Immune and Inflammatory Conditions.

The present disclosure provides methods for treating and/or preventing immune- and/or inflammatory-related diseases, disorders and conditions, as well as disorders associated therewith, with an IL-10 molecule and at least one additional therapeutic or diagnostic agent.

Examples of therapeutic agents useful in combination therapy include, but are not limited to, the following: non-steroidal anti-inflammatory drug (NSAID) such as aspirin, ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone). Other combinations include cyclooxygenase-2 (COX-2) inhibitors.

Other active agents for combination include steroids such as prednisolone, prednisone, methylprednisolone, betamethasone, dexamethasone, or hydrocortisone. Such a combination may be especially advantageous since one or more adverse affects of the steroid can be reduced or even eliminated by tapering the steroid dose required.

Additional examples of active agents that may be used in combinations for treating, for example, rheumatoid arthritis, include cytokine suppressive anti-inflammatory drug(s) (CSAIDs); antibodies to, or antagonists of, other human cytokines or growth factors, for example, TNF, LT, IL-1β, IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists such as chimeric, humanized or human TNF antibodies, REMICADE, anti-TNF antibody fragments (e.g., CDP870), and soluble p55 or p75 TNF receptors, derivatives thereof, p75TNFRIgG (ENBREL.) or p55TNFR1gG (LENERCEPT), soluble IL-13 receptor (sIL-13), and also TNFα-converting enzyme (TACE) inhibitors; similarly, IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the IL-10 polypeptides described herein include interferon-β1a (AVONEX); interferon-β1b (BETASERON); copaxone; hyperbaric oxygen; intravenous immunoglobulin; clabribine; and antibodies to, or antagonists of, other human cytokines or growth factors (e.g., antibodies to CD40 ligand and CD80).

The present disclosure encompasses pharmaceutically acceptable salts, acids or derivatives of any of the above.

Viral Diseases.

The present disclosure provides methods for treating and/or preventing viral diseases, disorders and conditions, as well as disorders associated therewith, with an IL-10 molecule and at least one additional therapeutic or diagnostic agent (e.g., one or more other antiviral agents and/or one or more agents not associated with viral therapy).

Such combination therapy includes anti-viral agents targeting various viral life-cycle stages and having different mechanisms of action, including, but not limiting to, the following: inhibitors of viral uncoating (e.g., amantadine and rimantidine); reverse transcriptase inhibitors (e.g., acyclovir, zidovudine, and lamivudine); agents that target integrase; agents that block attachment of transcription factors to viral DNA; agents (e.g., antisense molecules) that impact translation (e.g., fomivirsen); agents that modulate translation/ribozyme function; protease inhibitors; viral assembly modulators (e.g., rifampicin); and agents that prevent release of viral particles (e.g., zanamivir and oseltamivir). Treatment and/or prevention of certain viral infections (e.g., HIV) frequently entail a group (“cocktail”) of antiviral agents.

Other antiviral agents contemplated for use in combination with IL-10 polypeptides include, but are not limited to, the following: abacavir, adefovir, amantadine, amprenavir, ampligen, arbidol, atazanavir, atripla, boceprevirertet, cidofovir, combivir, darunavir, delavirdine, didanosine, docosanol, edoxudine, efavirenz, emtricitabine, enfuvirtide, entecavir, famciclovir, fosamprenavir, foscarnet, fosfonet, ganciclovir, ibacitabine, imunovir, idoxuridine, imiquimod, indinavir, inosine, various interferons (e.g., peginterferon alfa-2a), lopinavir, loviride, maraviroc, moroxydine, methisazone, nelfinavir, nevirapine, nexavir, penciclovir, peramivir, pleconaril, podophyllotoxin, raltegravir, ribavirin, ritonavir, pyramidine, saquinavir, stavudine, telaprevir, tenofovir, tipranavir, trifluridine, trizivir, tromantadine, truvada, valaciclovir, valganciclovir, vicriviroc, vidarabine, viramidine, and zalcitabine.

The present disclosure encompasses pharmaceutically acceptable salts, acids or derivatives of any of the above.

### Dosing

The IL-10 polypeptides of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the IL-10 molecules of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

The amount of an IL-10 molecule necessary to treat a disease, disorder or condition described herein is based on the IL-10 activity of the conjugated protein, which can be determined by IL-10 activity assays known in the art. By way of example, in the tumor context suitable IL-10 activity includes, for example, CD8+ T-cell infiltration into tumor sites, expression of inflammatory cytokines, such as IFN-γ, IL-4, IL-6, IL-10, and RANK-L, from these infiltrating cells, and increased levels of TNF-α or IFN-γ in biological samples.

The therapeutically effective amount of an IL-10 molecule can range from about 0.01 to about 100 μg protein/kg of body weight/day, from about 0.1 to 20 μg protein/kg of body weight/day, from about 0.5 to 10 μg protein/kg of body weight/day, or from about 1 to 4 μg protein/kg of body weight/day. In some embodiments, the therapeutically effective amount of an IL-10 molecule can range from about 1 to 16 μg protein/kg of body weight/day. The present disclosure contemplates the administration of an IL-10 molecule by continuous infusion to delivery, e.g., about 50 to 800 μg protein/kg of body weight/day. The infusion rate may be varied based on evaluation of, for example, adverse effects and blood cell counts.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or 1000.0 milligrams of the active ingredient.

In certain embodiments, the dosage of the disclosed IL-10 polypeptide is contained in a “unit dosage form”. The phrase “unit dosage form” refers to physically discrete units, each unit containing a predetermined amount of a IL-10 polypeptide of the present disclosure, either alone or in combination with one or more additional agents, sufficient to produce the desired effect. It will be appreciated that the parameters of a unit dosage form will depend on the particular agent and the effect to be achieved.

### Kits

The present disclosure also contemplates kits comprising IL-10, and pharmaceutical compositions thereof. The kits are generally in the form of a physical structure housing various components, as described below, and may be utilized, for example, in practicing the methods described herein (e.g., administration of an IL-10 molecule to a subject in need of restoring cholesterol homeostasis).

A kit can include one or more of the IL-10 polypeptides disclosed herein (provided in, e.g., a sterile container), which may be in the form of a pharmaceutical composition suitable for administration to a subject. The IL-10 polypeptides can be provided in a form that is ready for use or in a form requiring, for example, reconstitution or dilution prior to administration. When the IL-10 polypeptides are in a form that needs to be reconstituted by a user, the kit may also include buffers, pharmaceutically acceptable excipients, and the like, packaged with or separately from the IL-10 polypeptides. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); ng=nanogram; μg=microgram; mg=milligram; g=gram; kg=kilogram; dl or dL=deciliter; μl or μL=microliter; ml or mL=milliliter; l or L=liter; nM=nanomolar; μM=micromolar; mM=millimolar; M=molar; kDa=kilodalton; i.m.=intramuscular(ly); i.p.=intraperitoneal(ly); s.c.=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; DMEM=Dulbeco's Modification of Eagle's Medium; GC=genome copy; ELISA=enzyme-linked immuno sorbent assay; EDTA=ethylenediaminetetraacetic acid; PMA=phorbol myristate acetate; rhIL-10=recombinant human IL-10; LPS=lipopolysaccarhide.

### Materials and Methods

The following general materials and methods may be used in the Examples below:

Standard methods in molecular biology are described (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Louis et al. (2002) Basic Histology: Text and Atlas, McGraw-Hill, New York, N.Y.).

Depletion of immune cells (CD4⁺ and CD8⁺ T-cells) may be effected by antibody-mediated elimination. For example, 250 μg of CD4- or CD8-specific antibodies may be injected weekly, and cell depletions verified using FACS and IHC analysis.

Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypher™ (TimeLogic Corp., Crystal Bay, Nev.).

Immunocompetent Balb/C or B-cell-deficient Balb/C mice were obtained from The Jackson Lab., Bar Harbor, Me. and used in accordance with standard procedures (see, e.g., Martin et al (2001) Infect. Immun., 69(11):7067-73 and Compton et al. (2004) Comp. Med. 54(6):681-89). Other mice strains suitable for the experimental work contemplated by the present disclosure are known to the skilled artisan and are generally available from The Jackson Lab.

Unless otherwise indicated, PDV6 squamous cell carcinoma of the skin was used in the experiments described herein (see, e.g., Langowski et al. (2006) Nature 442:461-465). Other oncology-related models and cell lines, such as Ep2 mammary carcinoma, CT26 colon carcinoma, and 4T1 breast carcinoma models, may be used (see, e.g., Langowski et al. (2006) Nature 442:461-465) and are known to the skilled artisan. Non-oncology-related models and cell lines (e.g., models of inflammation) may also be used and are known to the skilled artisan.

Serum IL-10 concentration levels and exposure levels may be determined by standard methods used in the art. For example, a serum exposure level assay can be performed by collecting whole blood (˜50 μL/mouse) from mouse tail snips into plain capillary tubes, separating serum and blood cells by centrifugation, and determining IL-10 exposure levels by standard ELISA kits (e.g., R&D Systems) and techniques. Alternatively, or in addition, the ELISA protocol described below (or a similar protocol) can be adapted to measure serum levels of human IL-10 as a means of determining in vivo half-life of a mutein or modified mutein.

### Generation and Assessment of Muteins

Assembly of the Human IL-10 Expression Vector, pSecTag2hygro-huIL10.

A human IL-10 mammalian expression vector was assembled by amplifying the complete human IL-10 open reading frame via PCR using Platinum Pfx DNA Polymerase (Life Technologies #11708-039, following manufacturer's protocol) using pCMV6-XL5-human-IL10 (Origene #SC300099, Genbank accession #NM 00057.2) as a DNA template and primers 5′-tataGCTAGCCACCATGCACAGCTCAGCACTGC-3′ (SEQ ID NO:34) and 5′-tataGGGCCCTCAGTTTCGTATCTTCATTG-3′ (SEQ ID NO:35), and the resultant PCR reaction was purified using a QIAquick PCR Purification Kit (Qiagen #28106). The purified human IL-10 PCR fragment and the mammalian expression vector pSecTag2hygro (B) (Life Technologies #V910-20) were digested with ApaI and NheI (New England Biolabs, Ipswich, Mass.) for one hour at 37° C. with Calf Intestinal Phosphotase (New England Biolabs, Ipswich, Mass.) added to the pSecTag2hygro (B) digestion. The digested DNA fragments were run on a 1% agarose gel (Lonza #54803) for one hour at 100V, and then excised and purified using a QIAquick Gel Extraction Kit (Qiagen #28706). The human IL-10 PCR fragment was ligated into the pSecTag2hygro (B) vector using the Rapid DNA Ligation Kit (Roche #11635379001), transformed into One Shot TOP10 Chemically Competent E. coli (Life Technologies #C404006), plated to agar plates containing 100 μg/mL ampicillin and grown overnight at 37° C. The following day, bacterial colonies were picked individually and placed into 3 mL cultures containing LB+100 μg/mL ampicillin and grown for 8-20 hours at 37° C. in a shaking incubator at 200 RPM. Two (2) ml of each culture was then aliquoted to 2 mL tubes, the cells pelleted at 6000 RPM in a table-top centrifuge for 10 minutes, the media aspirated, and the DNA purified away from the bacteria using a QIAprep Spin Miniprep Kit (Qiagen #27106). Correct expression vectors were identified via DNA sequencing (MC Lab, South San Francisco, Calif.).

Generation of Mutein Expression Vectors.

Human IL10 mutein expression vectors were assembled by mutating the previously described human IL-10 mammalian expression vector pSecTag2hygro-huIL10 using a Quikchange II Site-Directed Mutagenesis Kit (Agilent Technologies #200524) following the manufacturer's protocol with the following clarifications: primers did not always meet the recommended Tm; the PCR reaction was cycled for 16-18 rounds with an extension time of 6-7 minutes; 4 μL of the DpnI-treated reaction was transformed into One Shot TOP10 Chemically Competent Cells (Life Technologies #C404006) as previously described. Three (3) mL miniprep cultures were grown, purified, and sequence-verified as previously described. For muteins in which a Cysteine was inserted, a 400 mL culture was grown and purified. Briefly, one bacterial colony was picked into 400 mL LB+100 μg/mL ampicillin, and grown for 12-20 hours at 37° C. in a shaking incubator at 200 RPM in a 2 L baffled Erlenmeyer flask. The culture was then pelleted in a centrifuge (6000 RPM in a Beckman Avanti J-25T in a JA-10 rotor for 20 minutes), the media aspirated, and the DNA extracted using an EndoFree Plasmid Mega Kit (Qiagen, #12381), following the manufacturer's protocol (with very minor changes, of a type familiar to the skilled artisan, made to the DNA precipitation methodology to increase the final DNA concentration).

Muteins which required multiple amino acid changes were assembled by inserting one mutation at a time. The introduction of the N-glycosylation motifs, N-X-S and N-X-T, sometimes required the introduction of three mutations since X≠P (Proline). Table 1 details the DNA template and primer sets used for the generation of the pSecTag2hygro-huIL10 expression vector, as well as all mutein expression vectors. The numbering convention used for the muteins assigns the start codon as the first position, hence the first 18 residues (MHSSALLCCLVLLTGVRA (SEQ ID NO:37)) comprise the signal peptide and the first residue of the mature protein would be Serine 19.

TABLE 1

| Expression | Template | | |
|---|---|---|---|
| Vector | DNA Used | | |
| Generated | for PCR | Primer Set Used for PCR | SEQ ID NO: |
| pSecTag2hygro- | pCMV- | tataGCTAGCCACCATGCACAGCTCAGCACTGC | SEQ NO ID: 38 |
| huIL10 | XL6-human | tataGGGCCCTCAGTTTCGTATCTTCATTG | SEQ NO ID: 39 |
| | IL-10 | | |
| | (Accession # | | |
| | NM00572.2, | | |
| | Origene | | |
| | #SC300099) | | |
| pSecTag2hygro- | pSecTag2hygro- | CTGACTGGGGTGAGGGCCtGCCCAGGCCAGGGCAC | SEQ NO ID: 40 |
| huIL10 S19C | huIL10 | GTGCCCTGGCCTGGGCaGGCCCTCACCCCAGTCAG | SEQ NO ID: 41 |
| pSecTag2hygro- | pSecTag2hygro- | GGGGTGAGGGCCAGCtgcGGCCAGGGCACCCAG | SEQ NO ID: 42 |
| huIL10 P20C | huIL10 | CTGGGTGCCCTGGCCgcaGCTGGCCCTCACCCC | SEQ NO ID: 43 |
| pSecTag2hygro- | pSecTag2hygro- | GGGTGAGGGCCAGCCCAtGCCAGGGCACCCAGTC | SEQ NO ID: 44 |
| huIL10 G21C | huIL10 | GACTGGGTGCCCTGGCaTGGGCTGGCCCTCACCC | SEQ NO ID: 45 |
| pSecTag2hygro- | pSecTag2hygro- | GAGGGCCAGCCCAGGCtgcGGCACCCAGTCTGAG | SEQ NO ID: 46 |
| huIL10 Q22C | huIL10 | CTCAGACTGGGTGCCgcaGCCTGGGCTGGCCCTC | SEQ NO ID: 47 |
| pSecTag2hygro- | pSecTag2hygro- | GGGCCAGCCCAGGCCAGtGCACCCAGTCTGAGAAC | SEQ NO ID: 48 |
| huIL10 G23C | huIL10 | GTTCTCAGACTGGGTGCaCTGGCCTGGGCTGGCCC | SEQ NO ID: 49 |
| pSecTag2hygro- | pSecTag2hygro- | CCAGCCCAGGCCAGGGCtgCCAGTCTGAGAACAGC | SEQ NO ID: 50 |
| huIL10 T24C | huIL10 | GCTGTTCTCAGACTGGcaGCCCTGGCCTGGGCTGG | SEQ NO ID: 51 |
| pSecTag2hygro- | pSecTag2hygro- | CCAGGCCAGGGCACCtgcTCTGAGAACAGCTGCAC | SEQ NO ID: 52 |
| huIL10 Q25C | huIL10 | GTGCAGCTGTTCTCAGAgcaGGTGCCCTGGCCTGG | SEQ NO ID: 53 |
| pSecTag2hygro- | pSecTag2hygro- | GGCCAGGGCACCCAGTgTGAGAACAGCTGCACCC | SEQ NO ID: 54 |
| huIL10 S26C | huIL10 | GGGTGCAGCTGTTCTCAcACTGGGTGCCCTGGCC | SEQ NO ID: 55 |
| pSecTag2hygro- | pSecTag2hygro- | GCCAGGGCACCCAGTCTtgcAACAGCTGCACCCAC | SEQ NO ID: 56 |
| huIL10 E27C | huIL10 | GTGGGTGCAGCTGTTgcaAGACTGGGTGCCCTGGC | SEQ NO ID: 57 |
| pSecTag2hygro- | pSecTag2hygro- | GGGCACCCAGTCTGAGtgCAGCTGCACCCACTTCC | SEQ NO ID: 58 |
| huIL10 N28C | huIL10 | GGAAGTGGGTGCAGCTGcaCTCAGACTGGGTGCCC | SEQ NO ID: 59 |
| pSecTag2hygro- | pSecTag2hygro- | GCACCCAGTCTGAGAACtGCTGCACCCACTTCCC | SEQ NO ID: 60 |
| huIL10 S29C | huIL10 | GGGAAGTGGGTGCAGCaGTTCTCAGACTGGGTGC | SEQ NO ID: 61 |
| pSecTag2hygro- | pSecTag2hygro- | GTCTGAGAACAGCTGCtgCCACTTCCCAGGCAACC | SEQ NO ID: 62 |
| huIL10 T31C | huIL10 | GGTTGCCTGGGAAGTGGcaGCAGCTGTTCTCAGAC | SEQ NO ID: 63 |
| pSecTag2hygro- | pSecTag2hygro- | GAGAACAGCTGCACCtgCTTCCCAGGCAACCTGCC | SEQ NO ID: 64 |
| huIL10 H32C | huIL10 | GGCAGGTTGCCTGGGAAGcaGGTGCAGCTGTTCTC | SEQ NO ID: 65 |
| pSecTag2hygro- | pSecTag2hygro- | GCTGCACCCACTTCCCAtGCAACCTGCCTAACATG | SEQ NO ID: 66 |
| huIL10 G35C | huIL10 | CATGTTAGGCAGGTTGCaTGGGAAGTGGGTGCAGC | SEQ NO ID: 67 |
| pSecTag2hygro- | pSecTag2hygro- | CACCCACTTCCCAGGCtgCCTGCCTAACATGCTTC | SEQ NO ID: 68 |
| huIL10 N36C | huIL10 | GAAGCATGTTAGGCAGGcaGCCTGGGAAGTGGGTG | SEQ NO ID: 69 |
| pSecTag2hygro- | pSecTag2hygro- | GCCTAACATGCTTCGAtgTCTCCGAGATGCCTTC | SEQ NO ID: 70 |
| huIL10 D43C | huIL10 | GAAGGCATCTCGGAGAcaTCGAAGCATGTTAGGC | SEQ NO ID: 71 |
| pSecTag2hygro- | pSecTag2hygro- | ATCTCCGAGATGCCTTCtGCAGAGTGAAGACTTTC | SEQ NO ID: 72 |
| huIL10 S49C | huIL10 | GAAAGTCTTCACTCTGCaGAAGGCATCTCGGAGAT | SEQ NO ID: 73 |
| pSecTag2hygro- | pSecTag2hygro- | CCGAGATGCCTTCAGCtGcGTGAAGACTTTCTTTC | SEQ NO ID: 74 |
| huIL10 R50C | huIL10 | GAAAGAAAGTCTTCACgCaGCTGAAGGCATCTCGG | SEQ NO ID: 75 |
| pSecTag2hygro- | pSecTag2hygro- | CTTTCTTTCAAATGtgcGATCAGCTGGACAACTTG | SEQ NO ID: 76 |
| huIL10 K58C | huIL10 | CAAGTTGTCCAGCTGATCgcaCATTTGAAAGAAAG | SEQ NO ID: 77 |
| pSecTag2hygro- | pSecTag2hygro- | GGACAACTTGTTGTTAtgcGAGTCCTTGCTGGAGG | SEQ NO ID: 78 |
| huIL10 K67C | huIL10 | CCTCCAGCAAGGACTCgcaTAACAACAAGTTGTCC | SEQ NO ID: 79 |
| pSecTag2hygro- | pSecTag2hygro- | CAACTTGTTGTTAAAGtgcTCCTTGCTGGAGGAC | SEQ NO ID: 80 |
| huIL10 E68C | huIL10 | GTCCTCCAGCAAGGAgcaCTTTAACAACAAGTTG | SEQ NO ID: 81 |
| pSecTag2hygro- | pSecTag2hygro- | CTTGTTGTTAAAGGAGTgCTTGCTGGAGGACTTTA | SEQ NO ID: 82 |
| huIL10 S69C | huIL10 | TAAAGTCCTCCAGCAAGcACTCCTTTAACAACAAG | SEQ NO ID: 83 |
| pSecTag2hygro- | pSecTag2hygro- | GGAGTCCTTGCTGtgcGACTTTAAGGGTTACCTGG | SEQ NO ID: 84 |
| huIL10 E72C | huIL10 | CCAGGTAACCCTTAAAGTCgcaCAGCAAGGACTCC | SEQ NO ID: 85 |
| pSecTag2hygro- | pSecTag2hygro- | GGAGTCCTTGCTGGAGtgCTTTAAGGGTTACCTGG | SEQ NO ID: 86 |
| huIL10 D73C | huIL10 | CCAGGTAACCCTTAAAGcaCTCCAGCAAGGACTCC | SEQ NO ID: 87 |
| pSecTag2hygro- | pSecTag2hygro- | CTTGCTGGAGGACTTTtgcGGTTACCTGGGTTGCC | SEQ NO ID: 88 |
| huIL10 K75C | huIL10 | GGCAACCCAGGTAACCgcaAAAGTCCTCCAGCAAG | SEQ NO ID: 89 |
| pSecTag2hygro- | pSecTag2hygro- | GCTGGAGGACTTTAAGtGTTACCTGGGTTGCCAAG | SEQ NO ID: 90 |
| huIL10 G76C | huIL10 | CTTGGCAACCCAGGTAACaCTTAAAGTCCTCCAGC | SEQ NO ID: 91 |
| pSecTag2hygro- | pSecTag2hygro- | GGAGGACTTTAAGGGTTgCCTGGGTTGCCAAGCC | SEQ NO ID: 92 |
| huIL10 Y77C | huIL10 | GGCTTGGCAACCCAGGcAACCCTTAAAGTCCTCC | SEQ NO ID: 93 |
| pSecTag2hygro- | pSecTag2hygro- | CTTTAAGGGTTACtgcGGTTGCCAAGCC | SEQ NO ID: 94 |
| huIL10 L78C | huIL10 | GGCTTGGCAACCgcaGTAACCCTTAAAG | SEQ NO ID: 95 |
| pSecTag2hygro- | pSecTag2hygro- | CTTTAAGGGTTACCTGtGTTGCCAAGCCTTGTCTG | SEQ NO ID: 96 |
| huIL10 G79C | huIL10 | CAGACAAGGCTTGGCAACaCAGGTAACCCTTAAAG | SEQ NO ID: 97 |
| pSecTag2hygro- | pSecTag2hygro- | GGGTTACCTGGGTTGCtgcGCCTTGTCTGAGATG | SEQ NO ID: 98 |
| huIL10 Q81C | huIL10 | CATCTCAGACAAGGCgcaGCAACCCAGGTAACCC | SEQ NO ID: 99 |
| pSecTag2hygro- | pSecTag2hygro- | GTTGCCAAGCCTTGTgTGAGATGATCCAGTTTTAC | SEQ NO ID: 100 |
| huIL10 S84C | huIL10 | GTAAAACTGGATCATCTCAcACAAGGCTTGGCAAC | SEQ NO ID: 101 |
| pSecTag2hygro- | pSecTag2hygro- | GTTGCCAAGCCTTGTCTtgcATGATCCAGTTTTAC | SEQ NO ID: 102 |
| huIL10 E85C | huIL10 | GTAAAACTGGATCATgcaAGACAAGGCTTGGCAAC | SEQ NO ID: 103 |
| pSecTag2hygro- | pSecTag2hygro- | CTTGTCTGAGATGATCtgcTTTTACCTGGAGGAGG | SEQ NO ID: 104 |
| huIL10 Q88C | huIL10 | CCTCCTCCAGGTAAAAgcaGATCATCTCAGACAAG | SEQ NO ID: 105 |
| pSecTag2hygro- | pSecTag2hygro- | GATCCAGTTTTACCTGtgcGAGGTGATGCCCCAAG | SEQ NO ID: 106 |
| huIL10 E92C | huIL10 | CTTGGGGCATCACCTCgcaCAGGTAAAACTGGATC | SEQ NO ID: 107 |
| pSecTag2hygro- | pSecTag2hygro- | GTTTTACCTGGAGtgcGTGATGCCCCAAGC | SEQ NO ID: 108 |
| huIL10 E93C | huIL10 | GCTTGGGGCATCACgcaCTCCAGGTAAAAC | SEQ NO ID: 109 |
| pSecTag2hygro- | pSecTag2hygro- | CCTGGAGGAGGTGATGtgCCAAGCTGAGAACCAAG | SEQ NO ID: 110 |
| huIL10 P96C | huIL10 | CTTGGTTCTCAGCTTGGcaCATCACCTCCTCCAGG | SEQ NO ID: 111 |
| pSecTag2hygro- | pSecTag2hygro- | GGAGGAGGTGATGCCCtgcGCTGAGAACCAAGACC | SEQ NO ID: 112 |
| huIL10 Q97C | huIL10 | GGTCTTGGTTCTCAGCgcaGGGCATCACCTCCTCC | SEQ NO ID: 113 |
| pSecTag2hygro- | pSecTag2hygro- | GGTGATGCCCCAAGCTtgcAACCAAGACCCAGAC | SEQ NO ID: 114 |
| huIL10 E99C | huIL10 | GTCTGGGTCTTGGTTgcaAGCTTGGGGCATCACC | SEQ NO ID: 115 |
| pSecTag2hygro- | pSecTag2hygro- | GATGCCCCAAGCTGAGtgCCAAGACCCAGACATC | SEQ NO ID: 116 |
| huIL10 N100C | huIL10 | GATGTCTGGGTCTTGGcaCTCAGCTTGGGGCATC | SEQ NO ID: 117 |
| pSecTag2hygro- | pSecTag2hygro- | CCAAGCTGAGAACtgcGACCCAGACATCAAGGCGC | SEQ NO ID: 118 |
| huIL10 Q101C | huIL10 | GCGCCTTGATGTCTGGGTCgcaGTTCTCAGCTTGG | SEQ NO ID: 119 |
| pSecTag2hygro- | pSecTag2hygro- | CAAGCTGAGAACCAAtgCCCAGACATCAAGGCGC | SEQ NO ID: 120 |
| huIL10 D102C | huIL10 | GCGCCTTGATGTCTGGGcaTTGGTTCTCAGCTTG | SEQ NO ID: 121 |
| pSecTag2hygro- | pSecTag2hygro- | GCTGAGAACCAAGACtgcGACATCAAGGCGCATG | SEQ NO ID: 122 |
| huIL10 P103C | huIL10 | CATGCGCCTTGATGTCgcaGTCTTGGTTCTCAGC | SEQ NO ID: 123 |
| pSecTag2hygro- | pSecTag2hygro- | CTGAGAACCAAGACCCAtgCATCAAGGCGCATGTG | SEQ NO ID: 124 |
| huIL10 D104C | huIL10 | CACATGCGCCTTGATGcaTGGGTCTTGGTTCTCAG | SEQ NO ID: 125 |
| pSecTag2hygro- | pSecTag2hygro- | CCAAGACCCAGACATCtgcGCGCATGTGAACTCCC | SEQ NO ID: 126 |
| huIL10 K106C | huIL10 | GGGAGTTCACATGCGCgcaGATGTCTGGGTCTTGG | SEQ NO ID: 127 |
| pSecTag2hygro- | pSecTag2hygro- | GACCCAGACATCAAGtgcCATGTGAACTCCCTGGG | SEQ NO ID: 128 |
| huIL10 A107C | huIL10 | CCCAGGGAGTTCACATGgcaCTTGATGTCTGGGTC | SEQ NO ID: 129 |
| pSecTag2hygro- | pSecTag2hygro- | CCCAGACATCAAGGCGtgcGTGAACTCCCTGGGGG | SEQ NO ID: 130 |
| huIL10 H108C | huIL10 | CCCCCAGGGAGTTCACgcaCGCCTTGATGTCTGGG | SEQ NO ID: 131 |
| pSecTag2hygro- | pSecTag2hygro- | CATCAAGGCGCATGTGtgCTCCCTGGGGGAGAACC | SEQ NO ID: 132 |
| huIL10 N110C | huIL10 | GGTTCTCCCCCAGGGAGcaCACATGCGCCTTGATG | SEQ NO ID: 133 |
| pSecTag2hygro- | pSecTag2hygro- | CAAGGCGCATGTGAACTgCCTGGGGGAGAACCTG | SEQ NO ID: 134 |
| huIL10 S111C | huIL10 | CAGGTTCTCCCCCAGGcAGTTCACATGCGCCTTG | SEQ NO ID: 135 |
| pSecTag2hygro- | pSecTag2hygro- | GCATGTGAACTCCCTGtGcGAGAACCTGAAGACCC | SEQ NO ID: 136 |
| huIL10 G113C | huIL10 | GGGTCTTCAGGTTCTCgCaCAGGGAGTTCACATGC | SEQ NO ID: 137 |
| pSecTag2hygro- | pSecTag2hygro- | GTGAACTCCCTGGGGtgcAACCTGAAGACCCTCAG | SEQ NO ID: 138 |
| huIL10 E114C | huIL10 | CTGAGGGTCTTCAGGTTgcaCCCCAGGGAGTTCAC | SEQ NO ID: 139 |
| pSecTag2hygro- | pSecTag2hygro- | GAACTCCCTGGGGGAGtgCCTGAAGACCCTCAGGC | SEQ NO ID: 140 |
| huIL10 N115C | huIL10 | GCCTGAGGGTCTTCAGGcaCTCCCCCAGGGAGTTC | SEQ NO ID: 141 |
| pSecTag2hygro- | pSecTag2hygro- | CCTGGGGGAGAACCTGtgcACCCTCAGGCTGAGGC | SEQ NO ID: 142 |
| huIL10 K117C | huIL10 | GCCTCAGCCTGAGGGTgcaCAGGTTCTCCCCCAGG | SEQ NO ID: 143 |
| pSecTag2hygro- | pSecTag2hygro- | GGGGGAGAACCTGAAGtgCCTCAGGCTGAGGCTAC | SEQ NO ID: 144 |
| huIL10 T118C | huIL10 | GTAGCCTCAGCCTGAGGcaCTTCAGGTTCTCCCCC | SEQ NO ID: 145 |
| pSecTag2hygro- | pSecTag2hygro- | GAACCTGAAGACCCTCtGcCTGAGGCTACGGCGC | SEQ NO ID: 146 |
| huIL10 R120C | huIL10 | GCGCCGTAGCCTCAGgCaGAGGGTCTTCAGGTTC | SEQ NO ID: 147 |
| pSecTag2hygro- | pSecTag2hygro- | CCTGAAGACCCTCAGGtgcAGGCTACGGCGCTGTC | SEQ NO ID: 148 |
| huIL10 L121C | huIL10 | GACAGCGCCGTAGCCTgcaCCTGAGGGTCTTCAGG | SEQ NO ID: 149 |
| pSecTag2hygro- | pSecTag2hygro- | GAAGACCCTCAGGCTGtgcCTACGGCGCTGTCATC | SEQ NO ID: 150 |
| huIL10 R122C | huIL10 | GATGACAGCGCCGTAGgcaCAGCCTGAGGGTCTTC | SEQ NO ID: 151 |
| pSecTag2hygro- | pSecTag2hygro- | CCTCAGGCTGAGGCTAtGcCGCTGTCATCGATTTC | SEQ NO ID: 152 |
| huIL10 R124C | huIL10 | GAAATCGATGACAGCGgCaTAGCCTCAGCCTGAGG | SEQ NO ID: 153 |
| pSecTag2hygro- | pSecTag2hygro- | CAGGCTGAGGCTACGGtGCTGTCATCGATTTCTTC | SEQ NO ID: 154 |
| huIL10 R125C | huIL10 | GAAGAAATCGATGACAGCaCCGTAGCCTCAGCCTG | SEQ NO ID: 155 |
| pSecTag2hygro- | pSecTag2hygro- | GAGGCTACGGCGCTGTtgTCGATTTCTTCCCTGTG | SEQ NO ID: 156 |
| huIL10 H127C | huIL10 | CACAGGGAAGAAATCGAcaACAGCGCCGTAGCCTC | SEQ NO ID: 157 |
| pSecTag2hygro- | pSecTag2hygro- | GCTACGGCGCTGTCATtGcTTTCTTCCCTGTG | SEQ NO ID: 158 |
| huIL10 R128C | huIL10 | CACAGGGAAGAAAgCaATGACAGCGCCGTAGC | SEQ NO ID: 159 |
| pSecTag2hygro- | pSecTag2hygro- | GCTGTCATCGATTTCTTtgCTGTGAAAACAAGAGC | SEQ NO ID: 160 |
| huIL10 P131C | huIL10 | GCTCTTGTTTTCACAGcaAAGAAATCGATGACAGC | SEQ NO ID: 161 |
| pSecTag2hygro- | pSecTag2hygro- | CGATTTCTTCCCTGTtgcAACAAGAGCAAGGCCG | SEQ NO ID: 162 |
| huIL10 E133C | huIL10 | CGGCCTTGCTCTTGTTgcaACAGGGAAGAAATCG | SEQ NO ID: 163 |
| pSecTag2hygro- | pSecTag2hygro- | GATTTCTTCCCTGTGAAtgCAAGAGCAAGGCCGTG | SEQ NO ID: 164 |
| huIL10 N134C | huIL10 | CACGGCCTTGCTCTTGcaTTCACAGGGAAGAAATC | SEQ NO ID: 165 |
| pSecTag2hygro- | pSecTag2hygro- | CTTCCCTGTGAAAACtgcAGCAAGGCCGTGGAGC | SEQ NO ID: 166 |
| huIL10 K135C | huIL10 | GCTCCACGGCCTTGCTgcaGTTTTCACAGGGAAG | SEQ NO ID: 167 |
| pSecTag2hygro- | pSecTag2hygro- | CTTCCCTGTGAAAACAAGtGCAAGGCCGTGGAGC | SEQ NO ID: 168 |
| huIL10 S136C | huIL10 | GCTCCACGGCCTTGCaCTTGTTTTCACAGGGAAG | SEQ NO ID: 169 |
| pSecTag2hygro- | pSecTag2hygro- | CCTGTGAAAACAAGAGCtgcGCCGTGGAGCAGGTG | SEQ NO ID: 170 |
| huIL10 K137C | huIL10 | CACCTGCTCCACGGCgcaGCTCTTGTTTTCACAGG | SEQ NO ID: 171 |
| pSecTag2hygro- | pSecTag2hygro- | GAGCAAGGCCGTGtgcCAGGTGAAGAATGCCTTTA | SEQ NO ID: 172 |
| huIL10 E140C | huIL10 | TAAAGGCATTCTTCACCTGgcaCACGGCCTTGCTC | SEQ NO ID: 173 |
| pSecTag2hygro- | pSecTag2hygro- | GAGCAAGGCCGTGGAGtgcGTGAAGAATGCCTTTA | SEQ NO ID: 174 |
| huIL10 Q141C | huIL10 | TAAAGGCATTCTTCACgcaCTCCACGGCCTTGCTC | SEQ NO ID: 175 |
| pSecTag2hygro- | pSecTag2hygro- | GAGCAAGGCCGTGGAGCAGGTGtgcAATGCCTTTA | SEQ NO ID: 176 |
| huIL10 K143C | huIL10 | ATAAGCTCCAAG | |
| | | CTTGGAGCTTATTAAAGGCATTgcaCACCTGCTCCA | SEQ NO ID: 177 |
| | | CGGCCTTGCTC | |
| pSecTag2hygro- | pSecTag2hygro- | CGTGGAGCAGGTGAAGtgcGCCTTTAATAAGCTCC | SEQ NO ID: 178 |
| huIL10 N144C | huIL10 | GGAGCTTATTAAAGGCgcaCTTCACCTGCTCCACG | SEQ NO ID: 179 |
| pSecTag2hygro- | pSecTag2hygro- | GGTGAAGAATGCCTTTtgTAAGCTCCAAGAGAAAG | SEQ NO ID: 180 |
| huIL10 N147C | huIL10 | CTTTCTCTTGGAGCTTAcaAAAGGCATTCTTCACC | SEQ NO ID: 181 |
| pSecTag2hygro- | pSecTag2hygro- | GAAGAATGCCTTTAATtgcCTCCAAGAGAAAGGC | SEQ NO ID: 182 |
| huIL10 K148C | huIL10 | GCCTTTCTCTTGGAGgcaATTAAAGGCATTCTTC | SEQ NO ID: 183 |
| pSecTag2hygro- | pSecTag2hygro- | GCCTTTAATAAGCTCtgcGAGAAAGGCATCTAC | SEQ NO ID: 184 |
| huIL10 Q150C | huIL10 | GTAGATGCCTTTCTCgcaGAGCTTATTAAAGGC | SEQ NO ID: 185 |
| pSecTag2hygro- | pSecTag2hygro- | CTTTAATAAGCTCCAAtgcAAAGGCATCTACAAAG | SEQ NO ID: 186 |
| huIL10 E151C | huIL10 | CTTTGTAGATGCCTTTgcaTTGGAGCTTATTAAAG | SEQ NO ID: 187 |
| pSecTag2hygro- | pSecTag2hygro- | CTTTAATAAGCTCCAAGAGtgcGGCATCTACAAAG | SEQ NO ID: 188 |
| huIL10 K152C | huIL10 | CTTTGTAGATGCCgcaCTCTTGGAGCTTATTAAAG | SEQ NO ID: 189 |
| pSecTag2hygro- | pSecTag2hygro- | GCTCCAAGAGAAACCATCTACAAAGCCATGAGTG | SEQ NO ID: 190 |
| huIL10 G153C | huIL10 | CACTCATGGCTTTGTAGATGCaTTTCTCTTGGAGC | SEQ NO ID: 191 |
| pSecTag2hygro- | pSecTag2hygro- | GCCTACATGACAATGtgcATACGAAACTGAGGGCC | SEQ NO ID: 192 |
| huIL10 K175C | huIL10 | GGCCCTCAGTTTCGTATgcaCATTGTCATGTAGGC | SEQ NO ID: 193 |
| pSecTag2hygro- | pSecTag2hygro- | CATGACAATGAAGATACGAtgCTGAGGGCCCGAAC | SEQ NO ID: 194 |
| huIL10 N178C | huIL10 | GTTCGGGCCCTCAGcaTCGTATCTTCATTGTCATG | SEQ NO ID: 195 |
| pSecTag2hygro- | pSecTag2hygro- | GACTGGGGTGAGGGCCtaCCCAGGCCAGGGCACCC | SEQ NO ID: 196 |
| huIL10 519Y | huIL10 | GGGTGCCCTGGCCTGGGtaGGCCCTCACCCCAGTC | SEQ NO ID: 197 |
| pSecTag2hygro- | pSecTag2hygro- | CTGGGGTGAGGGCCAGCtacGGCCAGGGCACCCAG | SEQ NO ID: 198 |
| huIL10 P20Y | huIL10 | CTGGGTGCCCTGGCCgtaGCTGGCCCTCACCCCAG | SEQ NO ID: 199 |
| pSecTag2hygro- | pSecTag2hygro- | GGTGAGGGCCAGCCCAtaCCAGGGCACCCAGTCTG | SEQ NO ID: 200 |
| huIL10 G21Y | huIL10 | CAGACTGGGTGCCCTGGtaTGGGCTGGCCCTCACC | SEQ NO ID: 201 |
| pSecTag2hygro- | pSecTag2hygro- | GAGGGCCAGCCCAGGCtAcGGCACCCAGTCTGAG | SEQ NO ID: 202 |
| huIL10 Q22Y | huIL10 | CTCAGACTGGGTGCCgTaGCCTGGGCTGGCCCTC | SEQ NO ID: 203 |
| pSecTag2hygro- | pSecTag2hygro- | GGGCCAGCCCAGGCCAGtaCACCCAGTCTGAGAAC | SEQ NO ID: 204 |
| huIL10 G23Y | huIL10 | GTTCTCAGACTGGGTGtaCTGGCCTGGGCTGGCCC | SEQ NO ID: 205 |
| pSecTag2hygro- | pSecTag2hygro- | CCAGCCCAGGCCAGGGCtaCCAGTCTGAGAACAGC | SEQ NO ID: 206 |
| huIL10 T24Y | huIL10 | GCTGTTCTCAGACTGGtaGCCCTGGCCTGGGCTGG | SEQ NO ID: 207 |
| pSecTag2hygro- | pSecTag2hygro- | GCCCAGGCCAGGGCACCtAcTCTGAGAACAGCTGC | SEQ NO ID: 208 |
| huIL10 Q25Y | huIL10 | GCAGCTGTTCTCAGAgTaGGTGCCCTGGCCTGGGC | SEQ NO ID: 209 |
| pSecTag2hygro- | pSecTag2hygro- | GGCCAGGGCACCCAGTacGAGAACAGCTGCACCC | SEQ NO ID: 210 |
| huIL10 S26Y | huIL10 | GGGTGCAGCTGTTCTCgtACTGGGTGCCCTGGCC | SEQ NO ID: 211 |
| pSecTag2hygro- | pSecTag2hygro- | GCCAGGGCACCCAGTCTtAcAACAGCTGCACCCAC | SEQ NO ID: 212 |
| huIL10 E27Y | huIL10 | GTGGGTGCAGCTGTTgTaAGACTGGGTGCCCTGGC | SEQ NO ID: 213 |
| pSecTag2hygro- | pSecTag2hygro- | GGGCACCCAGTCTGAGtACAGCTGCACCCACTTCC | SEQ NO ID: 214 |
| huIL10 N28Y | huIL10 | GGAAGTGGGTGCAGCTGTaCTCAGACTGGGTGCCC | SEQ NO ID: 215 |
| pSecTag2hygro- | pSecTag2hygro- | GGCACCCAGTCTGAGAACtaCTGCACCCACTTCCC | SEQ NO ID: 216 |
| huIL10 S29Y | huIL10 | GGGAAGTGGGTGCAGtaGTTCTCAGACTGGGTGCC | SEQ NO ID: 217 |
| pSecTag2hygro- | pSecTag2hygro- | GTCTGAGAACAGCTGCtaCCACTTCCCAGGCAACC | SEQ NO ID: 218 |
| huIL10 T31Y | huIL10 | GGTTGCCTGGGAAGTGGtaGCAGCTGTTCTCAGAC | SEQ NO ID: 219 |
| pSecTag2hygro- | pSecTag2hygro- | CTGAGAACAGCTGCACCtACTTCCCAGGCAACCTG | SEQ NO ID: 220 |
| huIL10 H32Y | huIL10 | CAGGTTGCCTGGGAAGTaGGTGCAGCTGTTCTCAG | SEQ NO ID: 221 |
| pSecTag2hygro- | pSecTag2hygro- | CTGCACCCACTTCCCAtaCAACCTGCCTAACATGC | SEQ NO ID: 222 |
| huIL10 G35Y | huIL10 | GCATGTTAGGCAGGTTGtaTGGGAAGTGGGTGCAG | SEQ NO ID: 223 |
| pSecTag2hygro- | pSecTag2hygro- | CACCCACTTCCCAGGCtACCTGCCTAACATGCTTC | SEQ NO ID: 224 |
| huIL10 N36Y | huIL10 | GAAGCATGTTAGGCAGGTaGCCTGGGAAGTGGGTG | SEQ NO ID: 225 |
| pSecTag2hygro- | pSecTag2hygro- | GCCTAACATGCTTCGAtAcCTCCGAGATGCCTTC | SEQ NO ID: 226 |
| huIL10 D43Y | huIL10 | GAAGGCATCTCGGAGgTaTCGAAGCATGTTAGGC | SEQ NO ID: 227 |
| pSecTag2hygro- | pSecTag2hygro- | GATCTCCGAGATGCCTTCtaCAGAGTGAAGACTTTC | SEQ NO ID: 228 |
| huIL10 S49Y | huIL10 | GAAAGTCTTCACTCTGtaGAAGGCATCTCGGAGATC | SEQ NO ID: 229 |
| pSecTag2hygro- | pSecTag2hygro- | CCGAGATGCCTTCAGCtacGTGAAGACTTTCTTTC | SEQ NO ID: 230 |
| huIL10 R50Y | huIL10 | GAAAGAAAGTCTTCACgtaGCTGAAGGCATCTCGG | SEQ NO ID: 231 |
| pSecTag2hygro- | pSecTag2hygro- | GACTTTCTTTCAAATGtAcGATCAGCTGGACAAC | SEQ NO ID: 232 |
| huIL10 K58Y | huIL10 | GTTGTCCAGCTGATCgTaCATTTGAAAGAAAGTC | SEQ NO ID: 233 |
| pSecTag2hygro- | pSecTag2hygro- | GGACAACTTGTTGTTAtAcGAGTCCTTGCTGGAGG | SEQ NO ID: 234 |
| huIL10 K67Y | huIL10 | CCTCCAGCAAGGACTCgTaTAACAACAAGTTGTCC | SEQ NO ID: 235 |
| pSecTag2hygro- | pSecTag2hygro- | CAACTTGTTGTTAAAGtAcTCCTTGCTGGAGGAC | SEQ NO ID: 236 |
| huIL10 E68Y | huIL10 | GTCCTCCAGCAAGGAgTaCTTTAACAACAAGTTG | SEQ NO ID: 237 |
| pSecTag2hygro- | pSecTag2hygro- | CTTGTTGTTAAAGGAGTaCTTGCTGGAGGACTTTA | SEQ NO ID: 238 |
| huIL10 S69Y | huIL10 | AGG | |
| | | CCTTAAAGTCCTCCAGCAAGtACTCCTTTAACAAC | SEQ NO ID: 239 |
| | | AAG | |
| pSecTag2hygro- | pSecTag2hygro- | AAAGGAGTCCTTGCTGtAcGACTTTAAGGGTTACC | SEQ NO ID: 240 |
| huIL10 E72Y | huIL10 | GGTAACCCTTAAAGTCgTaCAGCAAGGACTCCTTT | SEQ NO ID: 241 |
| pSecTag2hygro- | pSecTag2hygro- | GGAGTCCTTGCTGGAGtACTTTAAGGGTTACCTGG | SEQ NO ID: 242 |
| huIL10 D73Y | huIL10 | CCAGGTAACCCTTAAAGTaCTCCAGCAAGGACTCC | SEQ NO ID: 243 |
| pSecTag2hygro- | pSecTag2hygro- | CTTGCTGGAGGACTTTtAcGGTTACCTGGGTTGCC | SEQ NO ID: 244 |
| huIL10 K75Y | huIL10 | GGCAACCCAGGTAACCgTaAAAGTCCTCCAGCAAG | SEQ NO ID: 245 |
| pSecTag2hygro- | pSecTag2hygro- | GCTGGAGGACTTTAAGtacTACCTGGGTTGCCAAG | SEQ NO ID: 246 |
| huIL10 G76Y | huIL10 | CTTGGCAACCCAGGTAgtaCTTAAAGTCCTCCAGC | SEQ NO ID: 247 |
| pSecTag2hygro- | pSecTag2hygro- | GGACTTTAAGGGTTACtacGGTTGCCAAGCCTTG | SEQ NO ID: 248 |
| huIL10 L78Y | huIL10 | CAAGGCTTGGCAACCgtaGTAACCCTTAAAGTCC | SEQ NO ID: 249 |
| pSecTag2hygro- | pSecTag2hygro- | CTTTAAGGGTTACCTGtacTGCCAAGCCTTGTCTG | SEQ NO ID: 250 |
| huIL10 G79Y | huIL10 | CAGACAAGGCTTGGCAgtaCAGGTAACCCTTAAAG | SEQ NO ID: 251 |
| pSecTag2hygro- | pSecTag2hygro- | GGGTTACCTGGGTTGCtAcGCCTTGTCTGAGATG | SEQ NO ID: 252 |
| huIL10 Q81Y | huIL10 | CATCTCAGACAAGGCgTaGCAACCCAGGTAACCC | SEQ NO ID: 253 |
| pSecTag2hygro- | pSecTag2hygro- | GTTGCCAAGCCTTGTacGAGATGATCCAGTTTTAC | SEQ NO ID: 254 |
| huIL10 S84Y | huIL10 | GTAAAACTGGATCATCTCgtACAAGGCTTGGCAAC | SEQ NO ID: 255 |
| pSecTag2hygro- | pSecTag2hygro- | GTTGCCAAGCCTTGTCTtAcATGATCCAGTTTTAC | SEQ NO ID: 256 |
| huIL10 E85Y | huIL10 | GTAAAACTGGATCATgTaAGACAAGGCTTGGCAAC | SEQ NO ID: 257 |
| pSecTag2hygro- | pSecTag2hygro- | CTTGTCTGAGATGATCtAcTTTTACCTGGAGGAGG | SEQ NO ID: 258 |
| huIL10 Q88Y | huIL10 | CCTCCTCCAGGTAAAAgTaGATCATCTCAGACAAG | SEQ NO ID: 259 |
| pSecTag2hygro- | pSecTag2hygro- | GATCCAGTTTTACCTGtAcGAGGTGATGCCCCAAG | SEQ NO ID: 260 |
| huIL10 E92Y | huIL10 | CTTGGGGCATCACCTCgTaCAGGTAAAACTGGATC | SEQ NO ID: 261 |
| pSecTag2hygro- | pSecTag2hygro- | CCAGTTTTACCTGGAGtAcGTGATGCCCCAAGCTG | SEQ NO ID: 262 |
| huIL10 E93Y | huIL10 | CAGCTTGGGGCATCACgTaCTCCAGGTAAAACTGG | SEQ NO ID: 263 |
| pSecTag2hygro- | pSecTag2hygro- | CCTGGAGGAGGTGATGtaCCAAGCTGAGAACCAAG | SEQ NO ID: 264 |
| huIL10 P96Y | huIL10 | CTTGGTTCTCAGCTTGGtaCATCACCTCCTCCAGG | SEQ NO ID: 265 |
| pSecTag2hygro- | pSecTag2hygro- | GGAGGAGGTGATGCCCtAcGCTGAGAACCAAGACC | SEQ NO ID: 266 |
| huIL10 Q97Y | huIL10 | GGTCTTGGTTCTCAGCgTaGGGCATCACCTCCTCC | SEQ NO ID: 267 |
| pSecTag2hygro- | pSecTag2hygro- | GGTGATGCCCCAAGCTtAcAACCAAGACCCAGAC | SEQ NO ID: 268 |
| huIL10 E99Y | huIL10 | GTCTGGGTCTTGGTTgTaAGCTTGGGGCATCACC | SEQ NO ID: 269 |
| pSecTag2hygro- | pSecTag2hygro- | GATGCCCCAAGCTGAGtACCAAGACCCAGACATC | SEQ NO ID: 270 |
| huIL10 N100Y | huIL10 | GATGTCTGGGTCTTGGTaCTCAGCTTGGGGCATC | SEQ NO ID: 271 |
| pSecTag2hygro- | pSecTag2hygro- | GCCCCAAGCTGAGAACtAcGACCCAGACATCAAGG | SEQ NO ID: 272 |
| huIL10 Q101Y | huIL10 | CCTTGATGTCTGGGTCgTaGTTCTCAGCTTGGGGC | SEQ NO ID: 273 |
| pSecTag2hygro- | pSecTag2hygro- | CCAAGCTGAGAACCAAtACCCAGACATCAAGGCGC | SEQ NO ID: 274 |
| huIL10 D102Y | huIL10 | GCGCCTTGATGTCTGGGTaTTGGTTCTCAGCTTGG | SEQ NO ID: 275 |
| pSecTag2hygro- | pSecTag2hygro- | GCTGAGAACCAAGACtacGACATCAAGGCGCATG | SEQ NO ID: 276 |
| huIL10 P103Y | huIL10 | CATGCGCCTTGATGTCgtaGTCTTGGTTCTCAGC | SEQ NO ID: 277 |
| pSecTag2hygro- | pSecTag2hygro- | CTGAGAACCAAGACCCAtACATCAAGGCGCATGTG | SEQ NO ID: 278 |
| huIL10 D104Y | huIL10 | CACATGCGCCTTGATGTaTGGGTCTTGGTTCTCAG | SEQ NO ID: 279 |
| pSecTag2hygro- | pSecTag2hygro- | CCAAGACCCAGACATCtAcGCGCATGTGAACTCCC | SEQ NO ID: 280 |
| huIL10 K106Y | huIL10 | GGGAGTTCACATGCGCgTaGATGTCTGGGTCTTGG | SEQ NO ID: 281 |
| pSecTag2hygro- | pSecTag2hygro- | GACCCAGACATCAAGtacCATGTGAACTCCCTGGG | SEQ NO ID: 282 |
| huIL10 A107Y | huIL10 | CCCAGGGAGTTCACATGgtaCTTGATGTCTGGGTC | SEQ NO ID: 283 |
| pSecTag2hygro- | pSecTag2hygro- | CCCAGACATCAAGGCGtAcGTGAACTCCCTGGGGG | SEQ NO ID: 284 |
| huIL10 H108Y | huIL10 | CCCCCAGGGAGTTCACgTaCGCCTTGATGTCTGGG | SEQ NO ID: 285 |
| pSecTag2hygro- | pSecTag2hygro- | GGCGCATGTGtACTCCCTGGGGG | SEQ NO ID: 286 |
| huIL10 N110Y | huIL10 | CCCCCAGGGAGTaCACATGCGCC | SEQ NO ID: 287 |
| pSecTag2hygro- | pSecTag2hygro- | GGCGCATGTGAACTaCCTGGGGGAGAAC | SEQ NO ID: 288 |
| huIL10 S111Y | huIL10 | GTTCTCCCCCAGGtAGTTCACATGCGCC | SEQ NO ID: 289 |
| pSecTag2hygro- | pSecTag2hygro- | GCATGTGAACTCCCTGtacGAGAACCTGAAGACCC | SEQ NO ID: 290 |
| huIL10 G113Y | huIL10 | GGGTCTTCAGGTTCTCgtaCAGGGAGTTCACATGC | SEQ NO ID: 291 |
| pSecTag2hygro- | pSecTag2hygro- | GTGAACTCCCTGGGGtAcAACCTGAAGACCCTCAG | SEQ NO ID: 292 |
| huIL10 E114Y | huIL10 | CTGAGGGTCTTCAGGTTgTaCCCCAGGGAGTTCAC | SEQ NO ID: 293 |
| pSecTag2hygro- | pSecTag2hygro- | GAACTCCCTGGGGGAGtACCTGAAGACCCTCAGGC | SEQ NO ID: 294 |
| huIL10 N115Y | huIL10 | GCCTGAGGGTCTTCAGGTaCTCCCCCAGGGAGTTC | SEQ NO ID: 295 |
| pSecTag2hygro- | pSecTag2hygro- | CCTGGGGGAGAACCTGtAcACCCTCAGGCTGAGGC | SEQ NO ID: 296 |
| huIL10 K117Y | huIL10 | GCCTCAGCCTGAGGGTgTaCAGGTTCTCCCCCAGG | SEQ NO ID: 297 |
| pSecTag2hygro- | pSecTag2hygro- | GGAGAACCTGAAGtaCCTCAGGCTGAGG | SEQ NO ID: 298 |
| huIL10 T118Y | huIL10 | CCTCAGCCTGAGGtaCTTCAGGTTCTCC | SEQ NO ID: 299 |
| pSecTag2hygro- | pSecTag2hygro- | GAACCTGAAGACCCTCtacCTGAGGCTACGGCGC | SEQ NO ID: 300 |
| huIL10 R120Y | huIL10 | GCGCCGTAGCCTCAGgtaGAGGGTCTTCAGGTTC | SEQ NO ID: 301 |
| pSecTag2hygro- | pSecTag2hygro- | CCTGAAGACCCTCAGGtacAGGCTACGGCGCTGTC | SEQ NO ID: 302 |
| huIL10 L121Y | huIL10 | GACAGCGCCGTAGCCTgtaCCTGAGGGTCTTCAGG | SEQ NO ID: 303 |
| pSecTag2hygro- | pSecTag2hygro- | GAAGACCCTCAGGCTGtacCTACGGCGCTGTCATC | SEQ NO ID: 304 |
| huIL10 R122Y | huIL10 | GATGACAGCGCCGTAGgtaCAGCCTGAGGGTCTTC | SEQ NO ID: 305 |
| pSecTag2hygro- | pSecTag2hygro- | CCTCAGGCTGAGGCTAtacCGCTGTCATCGATTTC | SEQ NO ID: 306 |
| huIL10 R124Y | huIL10 | GAAATCGATGACAGCGgtaTAGCCTCAGCCTGAGG | SEQ NO ID: 307 |
| pSecTag2hygro- | pSecTag2hygro- | CAGGCTGAGGCTACGGtaCTGTCATCGATTTCTTC | SEQ NO ID: 308 |
| huIL10 R125Y | huIL10 | GAAGAAATCGATGACAGtaCCGTAGCCTCAGCCTG | SEQ NO ID: 309 |
| pSecTag2hygro- | pSecTag2hygro- | GAGGCTACGGCGCTGTtAcCGATTTCTTCCCTGTG | SEQ NO ID: 310 |
| huIL10 H127Y | huIL10 | CACAGGGAAGAAATCGgTaACAGCGCCGTAGCCTC | SEQ NO ID: 311 |
| pSecTag2hygro- | pSecTag2hygro- | GCTACGGCGCTGTCATtacTTTCTTCCCTGTGAAAAC | SEQ NO ID: 312 |
| huIL10 R128Y | huIL10 | GTTTTCACAGGGAAGAAAgtaATGACAGCGCCGTAGC | SEQ NO ID: 313 |
| pSecTag2hygro- | pSecTag2hygro- | GCTGTCATCGATTTCTTtaCTGTGAAAACAAGAGC | SEQ NO ID: 314 |
| huIL10 P131Y | huIL10 | GCTCTTGTTTTCACAGtaAAGAAATCGATGACAGC | SEQ NO ID: 315 |
| pSecTag2hygro- | pSecTag2hygro- | CGATTTCTTCCCTGTtAcAACAAGAGCAAGGCCG | SEQ NO ID: 316 |
| huIL10 E133Y | huIL10 | CGGCCTTGCTCTTGTTgTaACAGGGAAGAAATCG | SEQ NO ID: 317 |
| pSecTag2hygro- | pSecTag2hygro- | GATTTCTTCCCTGTGAAtACAAGAGCAAGGCCGTG | SEQ NO ID: 318 |
| huIL10 N134Y | huIL10 | CACGGCCTTGCTCTTGTaTTCACAGGGAAGAAATC | SEQ NO ID: 319 |
| pSecTag2hygro- | pSecTag2hygro- | CTTCCCTGTGAAAACtAcAGCAAGGCCGTGGAGC | SEQ NO ID: 320 |
| huIL10 K135Y | huIL10 | GCTCCACGGCCTTGCTgTaGTTTTCACAGGGAAG | SEQ NO ID: 321 |
| pSecTag2hygro- | pSecTag2hygro- | CCCTGTGAAAACAAGtaCAAGGCCGTGGAGCAGG | SEQ NO ID: 322 |
| huIL10 S136Y | huIL10 | CCTGCTCCACGGCCTTGtaCTTGTTTTCACAGGG | SEQ NO ID: 323 |
| pSecTag2hygro- | pSecTag2hygro- | CTGTGAAAACAAGAGCtAcGCCGTGGAGCAGGTG | SEQ NO ID: 324 |
| huIL10 K137Y | huIL10 | CACCTGCTCCACGGCgTaGCTCTTGTTTTCACAG | SEQ NO ID: 325 |
| pSecTag2hygro- | pSecTag2hygro- | CAAGAGCAAGGCCGTGtAcCAGGTGAAGAATGCC | SEQ NO ID: 326 |
| huIL10 E140Y | huIL10 | GGCATTCTTCACCTGgTaCACGGCCTTGCTCTTG | SEQ NO ID: 327 |
| pSecTag2hygro- | pSecTag2hygro- | GAGCAAGGCCGTGGAGtAcGTGAAGAATGCCTTTA | SEQ NO ID: 328 |
| huIL10 Q141Y | huIL10 | TAAAGGCATTCTTCACgTaCTCCACGGCCTTGCTC | SEQ NO ID: 329 |
| pSecTag2hygro- | pSecTag2hygro- | CAAGGCCGTGGAGCAGGTGtAcAATGCCTTTAATA | SEQ NO ID: 330 |
| huIL10 K143Y | huIL10 | AGCTCC | |
| | | GGAGCTTATTAAAGGCATTgTaCACCTGCTCCACG | SEQ NO ID: 331 |
| | | GCCTTG | |
| pSecTag2hygro- | pSecTag2hygro- | CGTGGAGCAGGTGAAGtAcGCCTTTAATAAGCTCC | SEQ NO ID: 332 |
| huIL10 N144Y | huIL10 | GGAGCTTATTAAAGGCgTaCTTCACCTGCTCCACG | SEQ NO ID: 333 |
| pSecTag2hygro- | pSecTag2hygro- | GAAGAATGCCTTTtAcAAGCTCCAAGAG | SEQ NO ID: 334 |
| huIL10 N147Y | huIL10 | CTCTTGGAGCTTgTaAAAGGCATTCTTC | SEQ NO ID: 335 |
| pSecTag2hygro- | pSecTag2hygro- | GAAGAATGCCTTTAATtAcCTCCAAGAGAAAGGC | SEQ NO ID: 336 |
| huIL10 K148Y | huIL10 | GCCTTTCTCTTGGAGgTaATTAAAGGCATTCTTC | SEQ NO ID: 337 |
| pSecTag2hygro- | pSecTag2hygro- | GCCTTTAATAAGCTCtAcGAGAAAGGCATCTAC | SEQ NO ID: 338 |
| huIL10 Q150Y | huIL10 | GTAGATGCCTTTCTCgTaGAGCTTATTAAAGGC | SEQ NO ID: 339 |
| pSecTag2hygro- | pSecTag2hygro- | CTTTAATAAGCTCCAAtAcAAAGGCATCTACAAAG | SEQ NO ID: 340 |
| huIL10 E151Y | huIL10 | CTTTGTAGATGCCTTTgTaTTGGAGCTTATTAAAG | SEQ NO ID: 341 |
| pSecTag2hygro- | pSecTag2hygro- | CTTTAATAAGCTCCAAGAGtAcGGCATCTACAAAG | SEQ NO ID: 342 |
| huIL10 K152Y | huIL10 | CC | |
| | | GGCTTTGTAGATGCCgTaCTCTTGGAGCTTATTAA | SEQ NO ID: 343 |
| | | AG | |
| pSecTag2hygro- | pSecTag2hygro- | GCTCCAAGAGAAAtaCATCTACAAAGCCATGAGTG | SEQ NO ID: 344 |
| huIL10 G153Y | huIL10 | CACTCATGGCTTTGTAGATGtaTTTCTCTTGGAGC | SEQ NO ID: 345 |
| pSecTag2hygro- | pSecTag2hygro- | GCCTACATGACAATGtAcATACGAAACTGAGGGCC | SEQ NO ID: 346 |
| huIL10 K175Y | huIL10 | GGCCCTCAGTTTCGTATgTaCATTGTCATGTAGGC | SEQ NO ID: 347 |
| pSecTag2hygro- | pSecTag2hygro- | CATGACAATGAAGATACGAtACTGAGGGCCCGAAC | SEQ NO ID: 348 |
| huIL10 N178Y | huIL10 | GTTCGGGCCCTCAGTaTCGTATCTTCATTGTCATG | SEQ NO ID: 349 |
| pSecTag2hygro- | pSecTag2hygro- | GACTGGGGTGAGGGCCAaCCCAGGCCAGGGCACCC | SEQ NO ID: 350 |
| huIL10 S19N | huIL10 | GGGTGCCCTGGCCTGGGtTGGCCCTCACCCCAGTC | SEQ NO ID: 351 |
| pSecTag2hygro- | pSecTag2hygro- | GGGTGAGGGCCAaCCCAaGCCAGGGCACCCAGTC | SEQ NO ID: 352 |
| huIL10 S19N, | huIL10 S19N | GACTGGGTGCCCTGGCtTGGGtTGGCCCTCACCC | SEQ NO ID: 353 |
| G21S | | | |
| pSecTag2hygro- | pSecTag2hygro- | GGGGTGAGGGCCAaCggtaGCCAGGGCACCCAG | SEQ NO ID: 354 |
| huIL10 S19N, | huIL10 Sl9N, | CTGGGTGCCCTGGCtaccGtTGGCCCTCACCCC | SEQ NO ID: 355 |
| P20G, G21S | G21S | | |
| pSecTag2hygro- | pSecTag2hygro- | GGTGAGGGCCAaCCCAacCCAGGGCACCCAGTCTG | SEQ NO ID: 356 |
| huIL10 S19N, | huIL10 S19N | CAGACTGGGTGCCCTGGgtTGGGtTGGCCCTCACC | SEQ NO ID: 357 |
| G21T | | | |
| pSecTag2hygro- | pSecTag2hygro- | GGTGAGGGCCAaCggtacCCAGGGCACCC | SEQ NO ID: 358 |
| huIL10 S19N, | huIL10 S19N, | GGGTGCCCTGGgtaccGtTGGCCCTCACC | SEQ NO ID: 359 |
| P20A, G21T | G21T | | |
| pSecTag2hygro- | pSecTag2hygro- | GGGGTGAGGGCCAGCaacGGCCAGGGCACCCAGTC | SEQ NO ID: 360 |
| huIL10 P20N | huIL10 | GACTGGGTGCCCTGGCCgttGCTGGCCCTCACCCC | SEQ NO ID: 361 |
| pSecTag2hygro- | pSecTag2hygro- | GGGCCAGCaacGGCagcGGCACCCAGTCTGAGAAC | SEQ NO ID: 362 |
| huIL10 P20N, | huIL10 P20N | GTTCTCAGACTGGGTGCCgctGCCgttGCTGGCCC | SEQ NO ID: 363 |
| Q22S | | | |
| pSecTag2hygro- | pSecTag2hygro- | GAGGGCCAGCaacGGCaccGGCACCCAGTCTGAG | SEQ NO ID: 364 |
| huIL10 P20N, | huIL10 P20N | CTCAGACTGGGTGCCggtGCCgttGCTGGCCCTC | SEQ NO ID: 365 |
| Q22T | | | |
| pSecTag2hygro- | pSecTag2hygro- | GGTGAGGGCCAGCCCAaaCCAGGGCACCCAGTCTG | SEQ NO ID: 366 |
| huIL10 G21N | huIL10 | CAGACTGGGTGCCCTGGttTGGGCTGGCCCTCACC | SEQ NO ID: 367 |
| pSecTag2hygro- | pSecTag2hygro- | GGGCCAGCCCAaaCCAGaGCACCCAGTCTGAGAAC | SEQ NO ID: 368 |
| huIL10 G21N, | huIL10 G21N | GTTCTCAGACTGGGTGCtCTGGttTGGGCTGGCCC | SEQ NO ID: 369 |
| G23S | | | |
| pSecTag2hygro- | pSecTag2hygro- | GGGCCAGCCCAaaCCAGacCACCCAGTCTGAGAAC | SEQ NO ID: 370 |
| huIL10 G21N, | huIL10 G21N | GTTCTCAGACTGGGTGgtCTGGttTGGGCTGGCCC | SEQ NO ID: 371 |
| G23T | | | |
| pSecTag2hygro- | pSecTag2hygro- | GAGGGCCAGCCCAGGCaAcGGCACCCAGTCTGAG | SEQ NO ID: 372 |
| huIL10 Q22N | huIL10 | CTCAGACTGGGTGCCgTtGCCTGGGCTGGCCCTC | SEQ NO ID: 373 |
| pSecTag2hygro- | pSecTag2hygro- | CAGCCCAGGCaAcGGCAgCCAGTCTGAGAACAGC | SEQ NO ID: 374 |
| huIL10 Q22N, | huIL10 Q22N | GCTGTTCTCAGACTGGcTGCCgTtGCCTGGGCTG | SEQ NO ID: 375 |
| T24S | | | |
| pSecTag2hygro- | pSecTag2hygro- | GGGCCAGCCCAGGCCAGaaCACCCAGTCTGAGAAC | SEQ NO ID: 376 |
| huIL10 G23N | huIL10 | GTTCTCAGACTGGGTGttCTGGCCTGGGCTGGCCC | SEQ NO ID: 377 |
| pSecTag2hygro- | pSecTag2hygro- | GCCCAGGCCAGaaCACCagcTCTGAGAACAGCTGC | SEQ NO ID: 378 |
| huIL10 G23N, | huIL10 G23N | GCAGCTGTTCTCAGAgctGGTGttCTGGCCTGGGC | SEQ NO ID: 379 |
| Q25S | | | |
| pSecTag2hygro- | pSecTag2hygro- | CCCAGGCCAGaaCACCaccTCTGAGAACAGCTGCAC | SEQ NO ID: 380 |
| huIL10 G23N, | huIL10 G23N | GTGCAGCTGTTCTCAGAggtGGTGttCTGGCCTGGG | SEQ NO ID: 381 |
| Q25T | | | |
| pSecTag2hygro- | pSecTag2hygro- | CCAGCCCAGGCCAGGGCAaCCAGTCTGAGAACAGC | SEQ NO ID: 382 |
| huIL10 T24N | huIL10 | GCTGTTCTCAGACTGGtTGCCCTGGCCTGGGCTGG | SEQ NO ID: 383 |
| pSecTag2hygro- | pSecTag2hygro- | CAGGCCAGGGCAaCCAGaCcGAGAACAGCTGCACC | SEQ NO ID: 384 |
| huIL10 T24N, | huIL10 T24N | GGTGCAGCTGTTCTCgGtCTGGtTGCCCTGGCCTG | SEQ NO ID: 385 |
| S26T | | | |
| pSecTag2hygro- | pSecTag2hygro- | CCAGGCCAGGGCACCaAcTCTGAGAACAGCTGCAC | SEQ NO ID: 386 |
| huIL10 Q25N | huIL10 | GTGCAGCTGTTCTCAGAgTtGGTGCCCTGGCCTGG | SEQ NO ID: 387 |
| pSecTag2hygro- | pSecTag2hygro- | GCCAGGGCACCaAcTCTagcAACAGCTGCACCCAC | SEQ NO ID: 388 |
| huIL10 Q25N, | huIL10 Q25N | GTGGGTGCAGCTGTTgctAGAgTtGGTGCCCTGGC | SEQ NO ID: 389 |
| E27S | | | |
| pSecTag2hygro- | pSecTag2hygro- | GCCAGGGCACCaAcTCTaccAACAGCTGCACCCAC | SEQ NO ID: 390 |
| huIL10 Q25N, | huIL10 Q25N | GTGGGTGCAGCTGTTggtAGAgTtGGTGCCCTGGC | SEQ NO ID: 391 |
| E27T | | | |
| pSecTag2hygro- | pSecTag2hygro- | GGCCAGGGCACCCAGaacGAGAACAGCTGCACCC | SEQ NO ID: 392 |
| huIL10 S26N | huIL10 | GGGTGCAGCTGTTCTCgttCTGGGTGCCCTGGCC | SEQ NO ID: 393 |
| pSecTag2hygro- | pSecTag2hygro- | GGGCACCCAGaacGAGAgCAGCTGCACCCACTTCC | SEQ NO ID: 394 |
| huIL10 S26N, | huIL10 S26N | GGAAGTGGGTGCAGCTGcTCTCgttCTGGGTGCCC | SEQ NO ID: 395 |
| N28S | | | |
| pSecTag2hygro- | pSecTag2hygro- | GGGCACCCAGaacGAGAcCAGCTGCACCCACTTCC | SEQ NO ID: 396 |
| huIL10 S26N, | huIL10 S26N | GGAAGTGGGTGCAGCTGgTCTCgttCTGGGTGCCC | SEQ NO ID: 397 |
| N28T | | | |
| pSecTag2hygro- | pSecTag2hy | CCAGGGCACCCAGTCTaAcAACAGCTGCACCCAC | SEQ NO ID: 398 |
| huIL10 E27N | gro-huIL10 | GTGGGTGCAGCTGTTgTtAGACTGGGTGCCCTGG | SEQ NO ID: 399 |
| pSecTag2hygro- | pSecTag2hygro- | CACCCAGTCTaAcAACAcCTGCACCCACTTCCCAG | SEQ NO ID: 400 |
| huIL10 E27N, | huIL10 E27N | CTGGGAAGTGGGTGCAGgTGTTgTtAGACTGGGTG | SEQ NO ID: 401 |
| S29T | | | |
| pSecTag2hygro- | pSecTag2hygro- | CACCCAGTCTGAGAACAaCTGCACCCACTTCCCAG | SEQ NO ID: 402 |
| huIL10 S29N | huIL10 | CTGGGAAGTGGGTGCAGtTGTTCTCAGACTGGGTG | SEQ NO ID: 403 |
| pSecTag2hygro- | pSecTag2hygro- | GTCTGAGAACAaCTGCAgCCACTTCCCAGGCAACC | SEQ NO ID: 404 |
| huIL10 S29N, | huIL10 S29N | GGTTGCCTGGGAAGTGGcTGCAGtTGTTCTCAGAC | SEQ NO ID: 405 |
| T31S | | | |
| pSecTag2hygro- | pSecTag2hygro- | GTCTGAGAACAGCTGCAaCCACTTCCCAGGCAACC | SEQ NO ID: 406 |
| huIL10 T31N | huIL10 | GGTTGCCTGGGAAGTGGtTGCAGCTGTTCTCAGAC | SEQ NO ID: 407 |
| pSecTag2hygro- | pSecTag2hygro- | GAACAGCTGCAaCCACagCCCAGGCAACCTGCC | SEQ NO ID: 408 |
| huIL10 T31N, | huIL10 T31N | GGCAGGTTGCCTGGGctGTGGtTGCAGCTGTTC | SEQ NO ID: 409 |
| F33S | | | |
| pSecTag2hygro- | pSecTag2hygro- | GAGAACAGCTGCAaCCACacCCCAGGCAACCTGCC | SEQ NO ID: 410 |
| huIL10 T31N, | huIL10 T31N | GGCAGGTTGCCTGGGgtGTGGtTGCAGCTGTTCTC | SEQ NO ID: 411 |
| F33T | | | |
| pSecTag2hygro- | pSecTag2hygro- | CTGAGAACAGCTGCACCaACTTCCCAGGCAACCTG | SEQ NO ID: 412 |
| huIL10 H32N | huIL10 | CAGGTTGCCTGGGAAGTtGGTGCAGCTGTTCTCAG | SEQ NO ID: 413 |
| pSecTag2hygro- | pSecTag2hygro- | GCTGCACCaACTTCagcGGCAACCTGCCTAACATG | SEQ NO ID: 414 |
| huIL10 H32N, | huIL10 H32N | CATGTTAGGCAGGTTGCCgctGAAGTtGGTGCAGC | SEQ NO ID: 415 |
| P34S | | | |
| pSecTag2hygro- | pSecTag2hygro- | CAGCTGCACCaACTTCaCcGGCAACCTGCCTAAC | SEQ NO ID: 416 |
| huIL10 H32N, | huIL10 H32N | GTTAGGCAGGTTGCCgGtGAAGTtGGTGCAGCTG | SEQ NO ID: 417 |
| P34T | | | |
| pSecTag2hygro- | pSecTag2hygro- | CTGCACCCACTTCCCAaaCAACCTGCCTAACATGC | SEQ NO ID: 418 |
| huIL10 G35N | huIL10 | GCATGTTAGGCAGGTTGttTGGGAAGTGGGTGCAG | SEQ NO ID: 419 |
| pSecTag2hygro- | pSecTag2hygro- | CCACTTCCCAaaCAACagcCCTAACATGCTTCGAG | SEQ NO ID: 420 |
| huIL10 G35N, | huIL10 G35N | CTCGAAGCATGTTAGGgctGTTGttTGGGAAGTGG | SEQ NO ID: 421 |
| L37S | | | |
| pSecTag2hygro- | pSecTag2hygro- | CCACTTCCCAaaCAACaccCCTAACATGCTTCGAG | SEQ NO ID: 422 |
| huIL10 G35N, | huIL10 G35N | CTCGAAGCATGTTAGGggtGTTGttTGGGAAGTGG | SEQ NO ID: 423 |
| L37T | | | |
| pSecTag2hygro- | pSecTag2hygro- | CTTCCCAGGCAACCTGagcAACATGCTTCGAGATC | SEQ NO ID: 424 |
| huIL10 P38S | huIL10 | GATCTCGAAGCATGTTgctCAGGTTGCCTGGGAAG | SEQ NO ID: 425 |
| pSecTag2hygro- | pSecTag2hygro- | CTTCCCAGGCAACCTGaCcAACATGCTTCGAGATC | SEQ NO ID: 426 |
| huIL10 P38T | huIL10 | GATCTCGAAGCATGTTgGtCAGGTTGCCTGGGAAG | SEQ NO ID: 427 |
| pSecTag2hygro- | pSecTag2hygro- | CCTAACATGCTTCGAaAcCTCCGAGATGCCTTCAG | SEQ NO ID: 428 |
| huIL10 D43N | huIL10 | CTGAAGGCATCTCGGAGgTtTCGAAGCATGTTAGG | SEQ NO ID: 429 |
| pSecTag2hygro- | pSecTag2hygro- | CATGCTTCGAaAcCTCagcGATGCCTTCAGCAGAG | SEQ NO ID: 430 |
| huIL10 D43N, | huIL10 D43N | CTCTGCTGAAGGCATCgctGAGgTtTCGAAGCATG | SEQ NO ID: 431 |
| R45S | | | |
| pSecTag2hygro- | pSecTag2hygro- | CATGCTTCGAaAcCTCaccGATGCCTTCAGCAGAG | SEQ NO ID: 432 |
| huIL10 D43N, | huIL10 D43N | CTCTGCTGAAGGCATCggtGAGgTtTCGAAGCATG | SEQ NO ID: 433 |
| R45T | | | |
| pSecTag2hygro- | pSecTag2hygro- | CTCCGAGATGCCTTCAaCAGAGTGAAGACTTTC | SEQ NO ID: 434 |
| huIL10 S49N | huIL10 | GAAAGTCTTCACTCTGtTGAAGGCATCTCGGAG | SEQ NO ID: 435 |
| pSecTag2hygro- | pSecTag2hygro- | GATGCCTTCAaCAGAagcAAGACTTTCTTTCAAAT | SEQ NO ID: 436 |
| huIL10 S49N, | huIL10 S49N | ATTTGAAAGAAAGTCTTgctTCTGtTGAAGGCATC | SEQ NO ID: 437 |
| V51S | | | |
| pSecTag2hygro- | pSecTag2hygro- | GATGCCTTCAaCAGAaccAAGACTTTCTTTCAAATG | SEQ NO ID: 438 |
| huIL10 S49N, | huIL10 S49N | CATTTGAAAGAAAGTCTTggtTCTGtTGAAGGCATC | SEQ NO ID: 439 |
| V51T | | | |
| pSecTag2hygro- | pSecTag2hygro- | CCGAGATGCCTTCAGCAacGTGAAGACTTTCTTTC | SEQ NO ID: 440 |
| huIL10 R50N | huIL10 | GAAAGAAAGTCTTCACgtTGCTGAAGGCATCTCGG | SEQ NO ID: 441 |
| pSecTag2hygro- | pSecTag2hygro- | CCTTCAGCAacGTGAgcACTTTCTTTCAAATGAAG | SEQ NO ID: 442 |
| huIL10 R50N, | huIL10 R50N | CTTCATTTGAAAGAAAGTgcTCACgtTGCTGAAGG | SEQ NO ID: 443 |
| K52S | | | |
| pSecTag2hygro- | pSecTag2hygro- | GCCTTCAGCAacGTGAccACTTTCTTTCAAATGAAG | SEQ NO ID: 444 |
| huIL10 R50N, | huIL10 R50N | CTTCATTTGAAAGAAAGTggTCACgtTGCTGAAGGC | SEQ NO ID: 445 |
| K52T | | | |
| pSecTag2hygro- | pSecTag2hygro- | CTTTCTTTCAAATGAAcGATCAGCTGGACAACTTG | SEQ NO ID: 446 |
| huIL10 K58N | huIL10 | CAAGTTGTCCAGCTGATCgTTCATTTGAAAGAAAG | SEQ NO ID: 447 |
| pSecTag2hygro- | pSecTag2hygro- | CTTTCAAATGAAcGATagcCTGGACAACTTGTTG | SEQ NO ID: 448 |
| huIL10 K58N, | huIL10 K58N | CAACAAGTTGTCCAGgctATCgTTCATTTGAAAG | SEQ NO ID: 449 |
| Q60S | | | |
| pSecTag2hygro- | pSecTag2hygro- | CTTTCAAATGAAcGATaccCTGGACAACTTGTTG | SEQ NO ID: 450 |
| huIL10 K58N, | huIL10 K58N | CAACAAGTTGTCCAGggtATCgTTCATTTGAAAG | SEQ NO ID: 451 |
| Q60T | | | |
| pSecTag2hygro- | pSecTag2hygro- | GTTGTTAAATGAGTCCTTGCTGGAGG | SEQ NO ID: 452 |
| huIL10 K67N | huIL10 | CCTCCAGCAAGGACTCATTTAACAAC | SEQ NO ID: 453 |
| pSecTag2hygro- | pSecTag2hygro- | TGTTGTTAAAtGAGagCTTGCTGGAGGACTTTAAG | SEQ NO ID: 454 |
| huIL10 K67N, | huIL10 K67N | CTTAAAGTCCTCCAGCAAGctCTCaTTTAACAACA | SEQ NO ID: 455 |
| S69T | | | |
| pSecTag2hygro- | pSecTag2hygro- | CAACTTGTTGTTAAAGaAcTCCTTGCTGGAGGAC | SEQ NO ID: 456 |
| huIL10 E68N | huIL10 | GTCCTCCAGCAAGGAgTtCTTTAACAACAAGTTG | SEQ NO ID: 457 |
| pSecTag2hygro- | pSecTag2hygro- | GTTGTTAAAGaAcTCCagcCTGGAGGACTTTAAGG | SEQ NO ID: 458 |
| huIL10 E68N, | huIL10 E68N | CCTTAAAGTCCTCCAGgctGGAgTtCTTTAACAAC | SEQ NO ID: 459 |
| L70S | | | |
| pSecTag2hygro- | pSecTag2hygro- | GTTGTTAAAGaAcTCCaccCTGGAGGACTTTAAGG | SEQ NO ID: 460 |
| huIL10 E68N, | huIL10 E68N | CCTTAAAGTCCTCCAGggtGGAgTtCTTTAACAAC | SEQ NO ID: 461 |
| L70T | | | |
| pSecTag2hygro- | pSecTag2hygro- | TGTTGTTAAAGGAGaaCTTGCTGGAGGACTTTAAG | SEQ NO ID: 462 |
| huIL10 S69N | huIL10 | CTTAAAGTCCTCCAGCAAGttCTCCTTTAACAACA | SEQ NO ID: 463 |
| pSecTag2hygro- | pSecTag2hygro- | GTTAAAGGAGaaCTTGagcGAGGACTTTAAGGGTT | SEQ NO ID: 464 |
| huIL10 S69N, | huIL10 S69N | AACCCTTAAAGTCCTCgctCAAGttCTCCTTTAAC | SEQ NO ID: 465 |
| L71S | | | |
| pSecTag2hygro- | pSecTag2hygro- | GTTAAAGGAGaaCTTGaccGAGGACTTTAAGGGTTAC | SEQ NO ID: 466 |
| huIL10 S69N, | huIL10 S69N | GTAACCCTTAAAGTCCTCggtCAAGttCTCCTTTAAC | SEQ NO ID: 467 |
| L71T | | | |
| pSecTag2hygro- | pSecTag2hygro- | GGAGTCCTTGCTGaAcGACTTTAAGGGTTACCTGG | SEQ NO ID: 468 |
| huIL10 E72N | huIL10 | CCAGGTAACCCTTAAAGTCgTtCAGCAAGGACTCC | SEQ NO ID: 469 |
| pSecTag2hygro- | pSecTag2hygro- | GTCCTTGCTGaAcGACagcAAGGGTTACCTGGG | SEQ NO ID: 470 |
| huIL10 E72N, | huIL10 E72N | CCCAGGTAACCCTTgctGTCgTtCAGCAAGGAC | SEQ NO ID: 471 |
| F74S | | | |
| pSecTag2hygro- | pSecTag2hygro- | GTCCTTGCTGaAcGACaccAAGGGTTACCTGGGTTG | SEQ NO ID: 472 |
| huIL10 E72N, | huIL10 E72N | CAACCCAGGTAACCCTTggtGTCgTtCAGCAAGGAC | SEQ NO ID: 473 |
| F74T | | | |
| pSecTag2hygro- | pSecTag2hygro- | GGAGTCCTTGCTGGAGaACTTTAAGGGTTACCTGG | SEQ NO ID: 474 |
| huIL10 D73N | huIL10 | CCAGGTAACCCTTAAAGTtCTCCAGCAAGGACTCC | SEQ NO ID: 475 |
| pSecTag2hygro- | pSecTag2hygro- | CTTGCTGGAGaACTTTAgcGGTTACCTGGGTTGCC | SEQ NO ID: 476 |
| huIL10 D73N, | huIL10 D73N | GGCAACCCAGGTAACCgcTAAAGTtCTCCAGCAAG | SEQ NO ID: 477 |
| K75S | | | |
| pSecTag2hygro- | pSecTag2hygro- | CTTGCTGGAGaACTTTAccGGTTACCTGGGTTGCC | SEQ NO ID: 478 |
| huIL10 D73N, | huIL10 D73N | GGCAACCCAGGTAACCggTAAAGTtCTCCAGCAAG | SEQ NO ID: 479 |
| K75T | | | |
| pSecTag2hygro- | pSecTag2hygro- | CTTGCTGGAGGACTTTAAcGGTTACCTGGGTTGCC | SEQ NO ID: 480 |
| huIL10 K75N | huIL10 | GGCAACCCAGGTAACCgTTAAAGTCCTCCAGCAAG | SEQ NO ID: 481 |
| pSecTag2hygro- | pSecTag2hygro- | GGAGGACTTTAAcGGTagCCTGGGTTGCCAAGCC | SEQ NO ID: 482 |
| huIL10 K75N, | huIL10 K75N | GGCTTGGCAACCCAGGctACCgTTAAAGTCCTCC | SEQ NO ID: 483 |
| Y77S | | | |
| pSecTag2hygro- | pSecTag2hygro- | GGAGGACTTTAAcGGTacCCTGGGTTGCCAAGCC | SEQ NO ID: 484 |
| huIL10 K75N, | huIL10 K75N | GGCTTGGCAACCCAGGgtACCgTTAAAGTCCTCC | SEQ NO ID: 485 |
| Y77T | | | |
| pSecTag2hygro- | pSecTag2hygro- | GCTGGAGGACTTTAAGaacTACCTGGGTTGCCAAG | SEQ NO ID: 486 |
| huIL10 G76N | huIL10 | CTTGGCAACCCAGGTAgttCTTAAAGTCCTCCAGC | SEQ NO ID: 487 |
| pSecTag2hygro- | pSecTag2hygro- | GGACTTTAAGaacTACagcGGTTGCCAAGCCTTG | SEQ NO ID: 488 |
| huIL10 G76N, | huIL10 G76N | CAAGGCTTGGCAACCgctGTAgttCTTAAAGTCC | SEQ NO ID: 489 |
| L78S | | | |
| pSecTag2hygro- | pSecTag2hygro- | GGACTTTAAGaacTACaccGGTTGCCAAGCCTTG | SEQ NO ID: 490 |
| huIL10 G76N, | huIL10 G76N | CAAGGCTTGGCAACCggtGTAgttCTTAAAGTCC | SEQ NO ID: 491 |
| L78T | | | |
| pSecTag2hygro- | pSecTag2hygro- | CTGGAGGACTTTAAGGGTaACCTGGGTTGCCAAGC | SEQ NO ID: 492 |
| huIL10 Y77N | huIL10 | GCTTGGCAACCCAGGTtACCCTTAAAGTCCTCCAG | SEQ NO ID: 493 |
| pSecTag2hygro- | pSecTag2hygro- | TTAAGGGTaACCTGaGcTGCCAAGCCTTGTC | SEQ NO ID: 494 |
| huIL10 Y77N, | huIL10 Y77N | GACAAGGCTTGGCAgCtCAGGTtACCCTTAA | SEQ NO ID: 495 |
| G79S | | | |
| pSecTag2hygro- | pSecTag2hygro- | CTTTAAGGGTTACCTGaacTGCCAAGCCTTGTCTG | SEQ NO ID: 496 |
| huIL10 G79N | huIL10 | CAGACAAGGCTTGGCAgttCAGGTAACCCTTAAAG | SEQ NO ID: 497 |
| pSecTag2hygro- | pSecTag2hygro- | GGGTTACCTGaacTGCagcGCCTTGTCTGAGATG | SEQ NO ID: 498 |
| huIL10 G79N, | huIL10 G79N | CATCTCAGACAAGGCgctGCAgttCAGGTAACCC | SEQ NO ID: 499 |
| Q81S | | | |
| pSecTag2hygro- | pSecTag2hygro- | GGGTTACCTGaacTGCaccGCCTTGTCTGAGATG | SEQ NO ID: 500 |
| huIL10 G79N, | huIL10 G79N | CATCTCAGACAAGGCggtGCAgttCAGGTAACCC | SEQ NO ID: 501 |
| Q81T | | | |
| pSecTag2hygro- | pSecTag2hygro- | GGGTTACCTGGGTTGCaAcGCCTTGTCTGAGATG | SEQ NO ID: 502 |
| huIL10 Q81N | huIL10 | CATCTCAGACAAGGCgTtGCAACCCAGGTAACCC | SEQ NO ID: 503 |
| pSecTag2hygro- | pSecTag2hygro- | CCTGGGTTGCaAcGCCagcTCTGAGATGATCCAG | SEQ NO ID: 504 |
| huIL10 Q81N, | huIL10 Q81N | CTGGATCATCTCAGAgctGGCgTtGCAACCCAGG | SEQ NO ID: 505 |
| L83S | | | |
| pSecTag2hygro- | pSecTag2hygro- | CCTGGGTTGCaAcGCCaccTCTGAGATGATCCAG | SEQ NO ID: 506 |
| huIL10 Q81N, | huIL10 Q81N | CTGGATCATCTCAGAggtGGCgTtGCAACCCAGG | SEQ NO ID: 507 |
| L83T | | | |
| pSecTag2hygro- | pSecTag2hygro- | GTTGCCAAGCCTTGaacGAGATGATCCAGTTTTAC | SEQ NO ID: 508 |
| huIL10 S84N | huIL10 | GTAAAACTGGATCATCTCgttCAAGGCTTGGCAAC | SEQ NO ID: 509 |
| pSecTag2hygro- | pSecTag2hygro- | CCAAGCCTTGaacGAGAgcATCCAGTTTTACCTGG | SEQ NO ID: 510 |
| huIL10 S84N, | huIL10 S84N | CCAGGTAAAACTGGATgcTCTCgttCAAGGCTTGG | SEQ NO ID: 511 |
| M86S | | | |
| pSecTag2hygro- | pSecTag2hygro- | CCAAGCCTTGaacGAGAccATCCAGTTTTACCTGG | SEQ NO ID: 512 |
| huIL10 S84N, | huIL10 S84N | CCAGGTAAAACTGGATggTCTCgttCAAGGCTTGG | SEQ NO ID: 513 |
| M86T | | | |
| pSecTag2hygro- | pSecTag2hygro- | GTTGCCAAGCCTTGTCTaAcATGATCCAGTTTTAC | SEQ NO ID: 514 |
| huIL10 E85N | huIL10 | GTAAAACTGGATCATgTtAGACAAGGCTTGGCAAC | SEQ NO ID: 515 |
| pSecTag2hygro- | pSecTag2hygro- | GCCTTGTCTaAcATGAgCCAGTTTTACCTGGAGG | SEQ NO ID: 516 |
| huIL10 E85N, | huIL10 E85N | CCTCCAGGTAAAACTGGcTCATgTtAGACAAGGC | SEQ NO ID: 517 |
| I87S | | | |
| pSecTag2hygro- | pSecTag2hygro- | GCCTTGTCTaAcATGAcCCAGTTTTACCTGGAGG | SEQ NO ID: 518 |
| huIL10 E85N, | huIL10 E85N | CCTCCAGGTAAAACTGGgTCATgTtAGACAAGGC | SEQ NO ID: 519 |
| I87T | | | |
| pSecTag2hygro- | pSecTag2hygro- | CTTGTCTGAGATGATCaAcTTTTACCTGGAGGAGG | SEQ NO ID: 520 |
| huIL10 Q88N | huIL10 | CCTCCTCCAGGTAAAAgTtGATCATCTCAGACAAG | SEQ NO ID: 521 |
| pSecTag2hygro- | pSecTag2hygro- | CTGAGATGATCaAcTTTagCCTGGAGGAGGTGATG | SEQ NO ID: 522 |
| huIL10 Q88N, | huIL10 Q88N | CATCACCTCCTCCAGGctAAAgTtGATCATCTCAG | SEQ NO ID: 523 |
| Y90S | | | |
| pSecTag2hygro- | pSecTag2hygro- | CTGAGATGATCaAcTTTacCCTGGAGGAGGTGATG | SEQ NO ID: 524 |
| huIL10 Q88N, | huIL10 Q88N | CATCACCTCCTCCAGGgtAAAgTtGATCATCTCAG | SEQ NO ID: 525 |
| Y90T | | | |
| pSecTag2hygro- | pSecTag2hygro- | GATCCAGTTTTACCTGaAcGAGGTGATGCCCCAAG | SEQ NO ID: 526 |
| huIL10 E92N | huIL10 | CTTGGGGCATCACCTCgTtCAGGTAAAACTGGATC | SEQ NO ID: 527 |
| pSecTag2hygro- | pSecTag2hygro- | GTTTTACCTGaAcGAGagcATGCCCCAAGCTGAG | SEQ NO ID: 528 |
| huIL10 E92N, | huIL10 E92N | CTCAGCTTGGGGCATgctCTCgTtCAGGTAAAAC | SEQ NO ID: 529 |
| V94S | | | |
| pSecTag2hygro- | pSecTag2hygro- | GTTTTACCTGaAcGAGaccATGCCCCAAGCTGAG | SEQ NO ID: 530 |
| huIL10 E92N, | huIL10 E92N | CTCAGCTTGGGGCATggtCTCgTtCAGGTAAAAC | SEQ NO ID: 531 |
| V94T | | | |
| pSecTag2hygro- | pSecTag2hygro- | CCAGTTTTACCTGGAGaAcGTGATGCCCCAAGCTG | SEQ NO ID: 532 |
| huIL10 E93N | huIL10 | CAGCTTGGGGCATCACgTtCTCCAGGTAAAACTGG | SEQ NO ID: 533 |
| pSecTag2hygro- | pSecTag2hygro- | CCTGGAGaAcGTGAgcCCCCAAGCTGAGAACCAAG | SEQ NO ID: 534 |
| huIL10 E93N, | huIL10 E93N | CTTGGTTCTCAGCTTGGGGgcTCACgTtCTCCAGG | SEQ NO ID: 535 |
| M95S | | | |
| pSecTag2hygro- | pSecTag2hygro- | CCTGGAGaAcGTGAccCCCCAAGCTGAGAACCAAG | SEQ NO ID: 536 |
| huIL10 E93N, | huIL10 E93N | CTTGGTTCTCAGCTTGGGGggTCACgTtCTCCAGG | SEQ NO ID: 537 |
| M95T | | | |
| pSecTag2hygro- | pSecTag2hygro- | CCTGGAGGAGGTGATGaaCCAAGCTGAGAACCAAG | SEQ NO ID: 538 |
| huIL10 P96N | huIL10 | CTTGGTTCTCAGCTTGGttCATCACCTCCTCCAGG | SEQ NO ID: 539 |
| pSecTag2hygro- | pSecTag2hygro- | GGAGGTGATGaaCCAAagcGAGAACCAAGACCCAG | SEQ NO ID: 540 |
| huIL10 P96N, | huIL10 P96N | CTGGGTCTTGGTTCTCgctTTGGttCATCACCTCC | SEQ NO ID: 541 |
| A98S | | | |
| pSecTag2hygro- | pSecTag2hygro- | GGAGGTGATGaaCCAAaCcGAGAACCAAGACCCAG | SEQ NO ID: 542 |
| huIL10 P96N, | huIL10 P96N | CTGGGTCTTGGTTCTCgGtTTGGttCATCACCTCC | SEQ NO ID: 543 |
| A98T | | | |
| pSecTag2hygro- | pSecTag2hygro- | GGAGGAGGTGATGCCCaAcGCTGAGAACCAAGACC | SEQ NO ID: 544 |
| huIL10 Q97N | huIL10 | GGTCTTGGTTCTCAGCgTtGGGCATCACCTCCTCC | SEQ NO ID: 545 |
| pSecTag2hygro- | pSecTag2hygro- | GGTGATGCCCaAcGCTagcAACCAAGACCCAGAC | SEQ NO ID: 546 |
| huIL10 Q97N, | huIL10 Q97N | GTCTGGGTCTTGGTTgctAGCgTtGGGCATCACC | SEQ NO ID: 547 |
| E99S | | | |
| pSecTag2hygro- | pSecTag2hygro- | GGTGATGCCCaAcGCTaccAACCAAGACCCAGAC | SEQ NO ID: 548 |
| huIL10 Q97N, | huIL10 Q97N | GTCTGGGTCTTGGTTggtAGCgTtGGGCATCACC | SEQ NO ID: 549 |
| E99T | | | |
| pSecTag2hygro- | pSecTag2hygro- | GGTGATGCCCCAAGCTaAcAACCAAGACCCAGAC | SEQ NO ID: 550 |
| huIL10 E99N | huIL10 | GTCTGGGTCTTGGTTgTtAGCTTGGGGCATCACC | SEQ NO ID: 551 |
| pSecTag2hygro- | pSecTag2hygro- | GCCCCAAGCTaAcAACagcGACCCAGACATCAAGG | SEQ NO ID: 552 |
| huIL10 E99N, | huIL10 E99N | CCTTGATGTCTGGGTCgctGTTgTtAGCTTGGGGC | SEQ NO ID: 553 |
| Q101S | | | |
| pSecTag2hygro- | pSecTag2hygro- | GCCCCAAGCTaAcAACaccGACCCAGACATCAAGG | SEQ NO ID: 554 |
| huIL10 E99N, | huIL10 E99N | CCTTGATGTCTGGGTCggtGTTgTtAGCTTGGGGC | SEQ NO ID: 555 |
| Q101T | | | |
| pSecTag2hygro- | pSecTag2hygro- | GCCCCAAGCTGAGAACaAcGACCCAGACATCAAGG | SEQ NO ID: 556 |
| huIL10 Q101N | huIL10 | CCTTGATGTCTGGGTCgTtGTTCTCAGCTTGGGGC | SEQ NO ID: 557 |
| pSecTag2hygro- | pSecTag2hygro- | GCTGAGAACaAcGACagcGACATCAAGGCGCATG | SEQ NO ID: 558 |
| huIL10 Q101N, | huIL10 Q101N | CATGCGCCTTGATGTCgctGTCgTtGTTCTCAGC | SEQ NO ID: 559 |
| P103S | | | |
| pSecTag2hygro- | pSecTag2hygro- | GCTGAGAACaAcGACaCcGACATCAAGGCGCATG | SEQ NO ID: 560 |
| huIL10 Q101N, | huIL10 Q101N | CATGCGCCTTGATGTCgGtGTCgTtGTTCTCAGC | SEQ NO ID: 561 |
| P103T | | | |
| pSecTag2hygro- | pSecTag2hygro- | GCCCCAAGCTGAGAACCAAAGCCCAGACATCAAGG | SEQ NO ID: 562 |
| huIL10 D102S | huIL10 | CG | |
| | | CGCCTTGATGTCTGGGCTTTGGTTCTCAGCTTGGG | SEQ NO ID: 563 |
| | | GC | |
| pSecTag2hygro- | pSecTag2hygro- | CCAAGCTGAGAACCAAacCCCAGACATCAAGGCGC | SEQ NO ID: 564 |
| huIL10 D102T | huIL10 | GCGCCTTGATGTCTGGGgtTTGGTTCTCAGCTTGG | SEQ NO ID: 565 |
| pSecTag2hygro- | pSecTag2hygro- | CCAAGCTGAGAACCAAaACCCAGACATCAAGGCGC | SEQ NO ID: 566 |
| huIL10 D102N | huIL10 | GCGCCTTGATGTCTGGGTtTTGGTTCTCAGCTTGG | SEQ NO ID: 567 |
| pSecTag2hygro- | pSecTag2hygro- | CTGAGAACCAAaACCCAagCATCAAGGCGCATGTG | SEQ NO ID: 568 |
| huIL10 D102N, | huIL10 D102N | CACATGCGCCTTGATGctTGGGTtTTGGTTCTCAG | SEQ NO ID: 569 |
| D104S | | | |
| pSecTag2hygro- | pSecTag2hygro- | CTGAGAACCAAaACCCAacCATCAAGGCGCATGTG | SEQ NO ID: 570 |
| huIL10 D102N, | huIL10 D102N | CACATGCGCCTTGATGgtTGGGTtTTGGTTCTCAG | SEQ NO ID: 571 |
| D104T | | | |
| pSecTag2hygro- | pSecTag2hygro- | GCTGAGAACCAAGACaacGACATCAAGGCGCATG | SEQ NO ID: 572 |
| huIL10 P103N | huIL10 | CATGCGCCTTGATGTCgttGTCTTGGTTCTCAGC | SEQ NO ID: 573 |
| pSecTag2hygro- | pSecTag2hygro- | GAACCAAGACaacGACAgCAAGGCGCATGTGAAC | SEQ NO ID: 574 |
| huIL10 P103N, | huIL10 P103N | GTTCACATGCGCCTTGcTGTCgttGTCTTGGTTC | SEQ NO ID: 575 |
| I105S | | | |
| pSecTag2hygro- | pSecTag2hygro- | GAACCAAGACaacGACAcCAAGGCGCATGTGAAC | SEQ NO ID: 576 |
| huIL10 P103N, | huIL10 P103N | GTTCACATGCGCCTTGgTGTCgttGTCTTGGTTC | SEQ NO ID: 577 |
| I105T | | | |
| pSecTag2hygro- | pSecTag2hygro- | CTGAGAACCAAGACCCAaACATCAAGGCGCATGTG | SEQ NO ID: 578 |
| huIL10 D104N | huIL10 | CACATGCGCCTTGATGTtTGGGTCTTGGTTCTCAG | SEQ NO ID: 579 |
| pSecTag2hygro- | pSecTag2hygro- | CCAAGACCCAaACATCAgcGCGCATGTGAACTCCC | SEQ NO ID: 580 |
| huIL10 D104N, | huIL10 D104N | GGGAGTTCACATGCGCgcTGATGTtTGGGTCTTGG | SEQ NO ID: 581 |
| K106S | | | |
| pSecTag2hygro- | pSecTag2hygro- | CCAAGACCCAaACATCAccGCGCATGTGAACTCCC | SEQ NO ID: 582 |
| huIL10 D104N, | huIL10 D104N | GGGAGTTCACATGCGCggTGATGTtTGGGTCTTGG | SEQ NO ID: 583 |
| K106T | | | |
| pSecTag2hygro- | pSecTag2hygro- | CCAAGACCCAGACATCAAcGCGCATGTGAACTCCC | SEQ NO ID: 584 |
| huIL10 K106N | huIL10 | GGGAGTTCACATGCGCgTTGATGTCTGGGTCTTGG | SEQ NO ID: 585 |
| pSecTag2hygro- | pSecTag2hygro- | CCCAGACATCAAcGCGagcGTGAACTCCCTGGGGG | SEQ NO ID: 586 |
| huIL10 K106N, | huIL10 K106N | CCCCCAGGGAGTTCACgctCGCgTTGATGTCTGGG | SEQ NO ID: 587 |
| H108S | | | |
| pSecTag2hygro- | pSecTag2hygro- | CCCAGACATCAAcGCGaccGTGAACTCCCTGGGGG | SEQ NO ID: 588 |
| huIL10 K106N, | huIL10 K106N | CCCCCAGGGAGTTCACggtCGCgTTGATGTCTGGG | SEQ NO ID: 589 |
| H108T | | | |
| pSecTag2hygro- | pSecTag2hygro- | GACCCAGACATCAAGaacCATGTGAACTCCCTGGG | SEQ NO ID: 590 |
| huIL10 A107N | huIL10 | CCCAGGGAGTTCACATGgttCTTGATGTCTGGGTC | SEQ NO ID: 591 |
| pSecTag2hygro- | pSecTag2hygro- | CAGACATCAAGaacCATagcAACTCCCTGGGGGAG | SEQ NO ID: 592 |
| huIL10 A107N, | huIL10 A107N | CTCCCCCAGGGAGTTgctATGgttCTTGATGTCTG | SEQ NO ID: 593 |
| V109S | | | |
| pSecTag2hygro- | pSecTag2hygro- | GACATCAAGaacCATaccAACTCCCTGGGGGAG | SEQ NO ID: 594 |
| huIL10 A107N, | huIL10 A107N | CTCCCCCAGGGAGTTggtATGgttCTTGATGTC | SEQ NO ID: 595 |
| V109T | | | |
| pSecTag2hygro- | pSecTag2hygro- | CCCAGACATCAAGGCGaAcGTGAACTCCCTGGGGG | SEQ NO ID: 596 |
| huIL10 H108N | huIL10 | CCCCCAGGGAGTTCACgTtCGCCTTGATGTCTGGG | SEQ NO ID: 597 |
| pSecTag2hygro- | pSecTag2hygro- | CATCAAGGCGaAcGTGAcCTCCCTGGGGGAGAACC | SEQ NO ID: 598 |
| huIL10 H108N, | huIL10 H108N | GGTTCTCCCCCAGGGAGgTCACgTtCGCCTTGATG | SEQ NO ID: 599 |
| N110T | | | |
| pSecTag2hygro- | pSecTag2hygro- | CAAGGCGCATGTGAACaaCCTGGGGGAGAACCTG | SEQ NO ID: 600 |
| huIL10 S111N | huIL10 | CAGGTTCTCCCCCAGGttGTTCACATGCGCCTTG | SEQ NO ID: 601 |
| pSecTag2hygro- | pSecTag2hygro- | GCATGTGAACaaCCTGaGcGAGAACCTGAAGACCC | SEQ NO ID: 602 |
| huIL10 S111N, | huIL10 S111N | GGGTCTTCAGGTTCTCgCtCAGGttGTTCACATGC | SEQ NO ID: 603 |
| G113S | | | |
| pSecTag2hygro- | pSecTag2hygro- | GCATGTGAACaaCCTGaccGAGAACCTGAAGACCC | SEQ NO ID: 604 |
| huIL10 S111N, | huIL10 S111N | GGGTCTTCAGGTTCTCggtCAGGttGTTCACATGC | SEQ NO ID: 605 |
| G113T | | | |
| pSecTag2hygro- | pSecTag2hygro- | CGCATGTGAACTCCtcGGGGGAGAACCTG | SEQ NO ID: 606 |
| huIL10 L112S | huIL10 | CAGGTTCTCCCCCgaGGAGTTCACATGCG | SEQ NO ID: 607 |
| pSecTag2hygro- | pSecTag2hygro- | GGCGCATGTGAACTCCaccGGGGAGAACCTGAAG | SEQ NO ID: 608 |
| huIL10 L112T | huIL10 | CTTCAGGTTCTCCCCggtGGAGTTCACATGCGCC | SEQ NO ID: 609 |
| pSecTag2hygro- | pSecTag2hygro- | GCATGTGAACTCCCTGaacGAGAACCTGAAGACCC | SEQ NO ID: 610 |
| huIL10 G113N | huIL10 | GGGTCTTCAGGTTCTCgttCAGGGAGTTCACATGC | SEQ NO ID: 611 |
| pSecTag2hygro- | pSecTag2hygro- | GAACTCCCTGaacGAGAgCCTGAAGACCCTCAGGC | SEQ NO ID: 612 |
| huIL10 G113N, | huIL10 G113N | GCCTGAGGGTCTTCAGGcTCTCgttCAGGGAGTTC | SEQ NO ID: 613 |
| N115S | | | |
| pSecTag2hygro- | pSecTag2hygro- | GAACTCCCTGaacGAGAcCCTGAAGACCCTCAGGC | SEQ NO ID: 614 |
| huIL10 G113N, | huIL10 G113N | GCCTGAGGGTCTTCAGGgTCTCgttCAGGGAGTTC | SEQ NO ID: 615 |
| N115T | | | |
| pSecTag2hygro- | pSecTag2hygro- | GTGAACTCCCTGGGGaAcAACCTGAAGACCCTCAG | SEQ NO ID: 616 |
| huIL10 E114N | hulL10 | CTGAGGGTCTTCAGGTTgTtCCCCAGGGAGTTCAC | SEQ NO ID: 617 |
| pSecTag2hygro- | pSecTag2hygro- | CTCCCTGGGGaAcAACagcAAGACCCTCAGGCTG | SEQ NO ID: 618 |
| huIL10 E114N, | huIL10 E114N | CAGCCTGAGGGTCTTgctGTTgTtCCCCAGGGAG | SEQ NO ID: 619 |
| L116S | | | |
| pSecTag2hygro- | pSecTag2hygro- | CTCCCTGGGGaAcAACaccAAGACCCTCAGGCTG | SEQ NO ID: 620 |
| huIL10 E114N, | huIL10 E114N | CAGCCTGAGGGTCTTggtGTTgTtCCCCAGGGAG | SEQ NO ID: 621 |
| L116T | | | |
| pSecTag2hygro- | pSecTag2hygro- | CCTGGGGGAGAACCTGAgcACCCTCAGGCTGAGGC | SEQ NO ID: 622 |
| huIL10 K117S | huIL10 | GCCTCAGCCTGAGGGTgcTCAGGTTCTCCCCCAGG | SEQ NO ID: 623 |
| pSecTag2hygro- | pSecTag2hygro- | CCTGGGGGAGAACCTGAccACCCTCAGGCTGAGGC | SEQ NO ID: 624 |
| huIL10 K117T | huIL10 | GCCTCAGCCTGAGGGTggTCAGGTTCTCCCCCAGG | SEQ NO ID: 625 |
| pSecTag2hygro- | pSecTag2hygro- | CCTGGGGGAGAACCTGAAcACCCTCAGGCTGAGGC | SEQ NO ID: 626 |
| huIL10 K117N | huIL10 | GCCTCAGCCTGAGGGTgTTCAGGTTCTCCCCCAGG | SEQ NO ID: 627 |
| pSecTag2hygro- | pSecTag2hygro- | GGAGAACCTGAAcACCagCAGGCTGAGGCTACGGC | SEQ NO ID: 628 |
| huIL10 K117N, | huIL10 K117N | GCCGTAGCCTCAGCCTGctGGTgTTCAGGTTCTCC | SEQ NO ID: 629 |
| L119S | | | |
| pSecTag2hygro- | pSecTag2hygro- | GGAGAACCTGAAcACCacCAGGCTGAGGCTACGGC | SEQ NO ID: 630 |
| huIL10 K117N, | huIL10 K117N | GCCGTAGCCTCAGCCTGgtGGTgTTCAGGTTCTCC | SEQ NO ID: 631 |
| L119T | | | |
| pSecTag2hygro- | pSecTag2hygro- | GGAGAACCTGAAGAaCCTCAGGCTGAGGC | SEQ NO ID: 632 |
| huIL10 T118N | huIL10 | GCCTCAGCCTGAGGtTCTTCAGGTTCTCC | SEQ NO ID: 633 |
| pSecTag2hygro- | pSecTag2hygro- | CCTGAAGAaCCTCAGcCTGAGGCTACGGCGCTGTC | SEQ NO ID: 634 |
| huIL10 T118N, | huIL10 T118N | GACAGCGCCGTAGCCTCAGgCTGAGGtTCTTCAGG | SEQ NO ID: 635 |
| R120S | | | |
| pSecTag2hygro- | pSecTag2hygro- | GAACCTGAAGAaCCTCAccCTGAGGCTACGGCGC | SEQ NO ID: 636 |
| huIL10 T118N, | huIL10 T118N | GCGCCGTAGCCTCAGggTGAGGtTCTTCAGGTTC | SEQ NO ID: 637 |
| R120T | | | |
| pSecTag2hygro- | pSecTag2hygro- | GAACCTGAAGACCCTCAacCTGAGGCTACGGCGC | SEQ NO ID: 638 |
| huIL10 R120N | huIL10 | GCGCCGTAGCCTCAGgtTGAGGGTCTTCAGGTTC | SEQ NO ID: 639 |
| pSecTag2hygro- | pSecTag2hygro- | GACCCTCAacCTGAGcCTACGGCGCTGTCATCG | SEQ NO ID: 640 |
| huIL10 R120N, | huIL10 R120N | CGATGACAGCGCCGTAGgCTCAGgtTGAGGGTC | SEQ NO ID: 641 |
| R122S | | | |
| pSecTag2hygro- | pSecTag2hygro- | GAAGACCCTCAacCTGAccCTACGGCGCTGTCATCG | SEQ NO ID: 642 |
| huIL10 R120N, | huIL10 R120N | CGATGACAGCGCCGTAGggTCAGgtTGAGGGTCTTC | SEQ NO ID: 643 |
| R122T | | | |
| pSecTag2hygro- | pSecTag2hygro- | CCTGAAGACCCTCAGGaacAGGCTACGGCGCTGTC | SEQ NO ID: 644 |
| huIL10 L121N | huIL10 | GACAGCGCCGTAGCCTgttCCTGAGGGTCTTCAGG | SEQ NO ID: 645 |
| pSecTag2hygro- | pSecTag2hygro- | GACCCTCAGGaacAGGagcCGGCGCTGTCATCGAT | SEQ NO ID: 646 |
| huIL10 L121N, | huIL10 L121N | ATCGATGACAGCGCCGgctCCTgttCCTGAGGGTC | SEQ NO ID: 647 |
| L123S | | | |
| pSecTag2hygro- | pSecTag2hygro- | GACCCTCAGGaacAGGaccCGGCGCTGTCATCG | SEQ NO ID: 648 |
| huIL10 L121N, | huIL10 L121N | CGATGACAGCGCCGggtCCTgttCCTGAGGGTC | SEQ NO ID: 649 |
| L123T | | | |
| pSecTag2hygro- | pSecTag2hygro- | GAAGACCCTCAGGCTGAacCTACGGCGCTGTCATC | SEQ NO ID: 650 |
| huIL10 R122N | huIL10 | GATGACAGCGCCGTAGgtTCAGCCTGAGGGTCTTC | SEQ NO ID: 651 |
| pSecTag2hygro- | pSecTag2hygro- | CCTCAGGCTGAacCTAagcCGCTGTCATCGATTTC | SEQ NO ID: 652 |
| huIL10 R122N, | huIL10 R122N | GAAATCGATGACAGCGgctTAGgtTCAGCCTGAGG | SEQ NO ID: 653 |
| R124S | | | |
| pSecTag2hygro- | pSecTag2hygro- | CCTCAGGCTGAacCTAaccCGCTGTCATCGATTTC | SEQ NO ID: 654 |
| huIL10 R122N, | huIL10 R122N | GAAATCGATGACAGCGggtTAGgtTCAGCCTGAGG | SEQ NO ID: 655 |
| R124T | | | |
| pSecTag2hygro- | pSecTag2hygro- | CAGGCTGAGGCTACGGaaCTGTCATCGATTTCTTC | SEQ NO ID: 656 |
| huIL10 R125N | huIL10 | GAAGAAATCGATGACAGttCCGTAGCCTCAGCCTG | SEQ NO ID: 657 |
| pSecTag2hygro- | pSecTag2hygro- | GAGGCTACGGaaCTGTagcCGATTTCTTCCCTGTG | SEQ NO ID: 658 |
| huIL10 R125N, | huIL10 R125N | CACAGGGAAGAAATCGgctACAGttCCGTAGCCTC | SEQ NO ID: 659 |
| H127S | | | |
| pSecTag2hygro- | pSecTag2hygro- | GAGGCTACGGaaCTGTaccCGATTTCTTCCCTGTG | SEQ NO ID: 660 |
| huIL10 R125N, | huIL10 R125N | CACAGGGAAGAAATCGggtACAGttCCGTAGCCTC | SEQ NO ID: 661 |
| H127T | | | |
| pSecTag2hygro- | pSecTag2hygro- | GAGGCTACGGCGCTGTaAcCGATTTCTTCCCTGTG | SEQ NO ID: 662 |
| huIL10 H127N | huIL10 | CACAGGGAAGAAATCGgTtACAGCGCCGTAGCCTC | SEQ NO ID: 663 |
| pSecTag2hygro- | pSecTag2hygro- | GGCGCTGTaAcCGAagcCTTCCCTGTGAAAACAAG | SEQ NO ID: 664 |
| huIL10 H127N, | huIL10 H127N | CTTGTTTTCACAGGGAAGgctTCGgTtACAGCGCC | SEQ NO ID: 665 |
| F129S | | | |
| pSecTag2hygro- | pSecTag2hygro- | CGGCGCTGTaAcCGAaccCTTCCCTGTGAAAACAAG | SEQ NO ID: 666 |
| huIL10 H127N, | huIL10 H127N | CTTGTTTTCACAGGGAAGggtTCGgTtACAGCGCCG | SEQ NO ID: 667 |
| F129T | | | |
| pSecTag2hygro- | pSecTag2hygro- | GCTACGGCGCTGTCATaacTTTCTTCCCTGTGAA | SEQ NO ID: 668 |
| huIL10 R128N | huIL10 | TTCACAGGGAAGAAAgttATGACAGCGCCGTAGC | SEQ NO ID: 669 |
| pSecTag2hygro- | pSecTag2hygro- | CGCTGTCATaacTTTagcCCCTGTGAAAACAAGAG | SEQ NO ID: 670 |
| huIL10 R128N, | huIL10 R128N | CTCTTGTTTTCACAGGGgctAAAgttATGACAGCG | SEQ NO ID: 671 |
| L130S | | | |
| pSecTag2hygro- | pSecTag2hygro- | GGCGCTGTCATaacTTTaccCCCTGTGAAAACAAG | SEQ NO ID: 672 |
| huIL10 R128N, | huIL10 R128N | CTTGTTTTCACAGGGggtAAAgttATGACAGCGCC | SEQ NO ID: 673 |
| L130T | | | |
| pSecTag2hygro- | pSecTag2hygro- | GCTGTCATCGATTTCTTaaCTGTGAAAACAAGAGC | SEQ NO ID: 674 |
| huIL10 P131N | huIL10 | GCTCTTGTTTTCACAGttAAGAAATCGATGACAGC | SEQ NO ID: 675 |
| pSecTag2hygro- | pSecTag2hygro- | CGATTTCTTaaCTGTagcAACAAGAGCAAGGCCG | SEQ NO ID: 676 |
| huIL10 P131N, | huIL10 P131N | CGGCCTTGCTCTTGTTgctACAGttAAGAAATCG | SEQ NO ID: 677 |
| E133S | | | |
| pSecTag2hygro- | pSecTag2hygro- | CGATTTCTTaaCTGTaccAACAAGAGCAAGGCCG | SEQ NO ID: 678 |
| huIL10 P131N, | huIL10 P131N | CGGCCTTGCTCTTGTTggtACAGttAAGAAATCG | SEQ NO ID: 679 |
| E133T | | | |
| pSecTag2hygro- | pSecTag2hygro- | CGATTTCTTCCCTGTaAcAACAAGAGCAAGGCCG | SEQ NO ID: 680 |
| huIL10 E133N | huIL10 | CGGCCTTGCTCTTGTTgTtACAGGGAAGAAATCG | SEQ NO ID: 681 |
| pSecTag2hygro- | pSecTag2hygro- | CTTCCCTGTaAcAACAgcAGCAAGGCCGTGGAGC | SEQ NO ID: 682 |
| huIL10 E133N, | huIL10 E133N | GCTCCACGGCCTTGCTgcTGTTgTtACAGGGAAG | SEQ NO ID: 683 |
| K135S | | | |
| pSecTag2hygro- | pSecTag2hygro- | CTTCCCTGTaAcAACAccAGCAAGGCCGTGGAGC | SEQ NO ID: 684 |
| huIL10 E133N, | huIL10 E133N | GCTCCACGGCCTTGCTggTGTTgTtACAGGGAAG | SEQ NO ID: 685 |
| K135T | | | |
| pSecTag2hygro- | pSecTag2hygro- | CCCTGTGAAAACAAGAcCAAGGCCGTGGAGCAGG | SEQ NO ID: 686 |
| huIL10 S136T | huIL10 | CCTGCTCCACGGCCTTGgTCTTGTTTTCACAGGG | SEQ NO ID: 687 |
| pSecTag2hygro- | pSecTag2hygro- | CTTCCCTGTGAAAACAAcAGCAAGGCCGTGGAGC | SEQ NO ID: 688 |
| huIL10 K135N | huIL10 | GCTCCACGGCCTTGCTgTTGTTTTCACAGGGAAG | SEQ NO ID: 689 |
| pSecTag2hygro- | pSecTag2hygro- | GTGAAAACAAcAGCAgcGCCGTGGAGCAGGTGAAG | SEQ NO ID: 690 |
| huIL10 K135N, | huIL10 K135N | CTTCACCTGCTCCACGGCgcTGCTgTTGTTTTCAC | SEQ NO ID: 691 |
| K137S | | | |
| pSecTag2hygro- | pSecTag2hygro- | GTGAAAACAAcAGCAccGCCGTGGAGCAGGTGAAG | SEQ NO ID: 692 |
| huIL10 K135N, | huIL10 K135N | CTTCACCTGCTCCACGGCggTGCTgTTGTTTTCAC | SEQ NO ID: 693 |
| K137T | | | |
| pSecTag2hygro- | pSecTag2hygro- | CCCTGTGAAAACAAGAaCAAGGCCGTGGAGCAGG | SEQ NO ID: 694 |
| huIL10 S136N | huIL10 | CCTGCTCCACGGCCTTGtTCTTGTTTTCACAGGG | SEQ NO ID: 695 |
| pSecTag2hygro- | pSecTag2hygro- | GTGAAAACAAGAaCAAGagCGTGGAGCAGGTGAAG | SEQ NO ID: 696 |
| huIL10 S136N, | huIL10 S136N | CTTCACCTGCTCCACGctCTTGtTCTTGTTTTCAC | SEQ NO ID: 697 |
| A138S | | | |
| pSecTag2hygro- | pSecTag2hygro- | GTGAAAACAAGAaCAAGaCCGTGGAGCAGGTGAAG | SEQ NO ID: 698 |
| huIL10 S136N, | huIL10 S136N | CTTCACCTGCTCCACGGtCTTGtTCTTGTTTTCAC | SEQ NO ID: 699 |
| A138T | | | |
| pSecTag2hygro- | pSecTag2hygro- | GTGAAAACAAGAGCAAcGCCGTGGAGCAGGTGAAG | SEQ NO ID: 700 |
| huIL10 K137N | huIL10 | CTTCACCTGCTCCACGGCgTTGCTCTTGTTTTCAC | SEQ NO ID: 701 |
| pSecTag2hygro- | pSecTag2hygro- | AAACAAGAGCAAcGCCagcGAGCAGGTGAAGAATG | SEQ NO ID: 702 |
| huIL10 K137N, | huIL10 K137N | CATTCTTCACCTGCTCgctGGCgTTGCTCTTGTTT | SEQ NO ID: 703 |
| V139S | | | |
| pSecTag2hygro- | pSecTag2hygro- | GAAAACAAGAGCAAcGCCaccGAGCAGGTGAAGAATG | SEQ NO ID: 704 |
| huIL10 K137N, | huIL10 K137N | CATTCTTCACCTGCTCggtGGCgTTGCTCTTGTTTTC | SEQ NO ID: 705 |
| V139T | | | |
| pSecTag2hygro- | pSecTag2hygro- | CAAGAGCAAGGCCGTGaAcCAGGTGAAGAATGCC | SEQ NO ID: 706 |
| huIL10 E140N | huIL10 | GGCATTCTTCACCTGgTtCACGGCCTTGCTCTTG | SEQ NO ID: 707 |
| pSecTag2hygro- | pSecTag2hygro- | GGCCGTGaAcCAGagcAAGAATGCCTTTAATAAGC | SEQ NO ID: 708 |
| huIL10 E140N, | huIL10 E140N | GCTTATTAAAGGCATTCTTgctCTGgTtCACGGCC | SEQ NO ID: 709 |
| V142S | | | |
| pSecTag2hygro- | pSecTag2hygro- | GAGCAAGGCCGTGGAGaAcGTGAAGAATGCCTTTA | SEQ NO ID: 710 |
| huIL10 Q141N | huIL10 | TAAAGGCATTCTTCACgTtCTCCACGGCCTTGCTC | SEQ NO ID: 711 |
| pSecTag2hygro- | pSecTag2hygro- | GGCCGTGGAGaAcGTGAgcAATGCCTTTAATAAGC | SEQ NO ID: 712 |
| huIL10 Q141N, | huIL10 Q141N | GCTTATTAAAGGCATTgcTCACgTtCTCCACGGCC | SEQ NO ID: 713 |
| K143S | | | |
| pSecTag2hygro- | pSecTag2hygro- | GTGGAGCAGGTGAAcAATGCCTTTAATAAG | SEQ NO ID: 714 |
| huIL10 K143N | huIL10 | CTTATTAAAGGCATTgTTCACCTGCTCCAC | SEQ NO ID: 715 |
| pSecTag2hygro- | pSecTag2hygro- | GGAGCAGGTGAAcAATagCTTTAATAAGCTCCAAG | SEQ NO ID: 716 |
| huIL10 K143N, | huIL10 K143N | CTTGGAGCTTATTAAAGctATTgTTCACCTGCTCC | SEQ NO ID: 717 |
| A145S | | | |
| pSecTag2hygro- | pSecTag2hygro- | GGAGCAGGTGAAcAATaCCTTTAATAAGCTCCAAG | SEQ NO ID: 718 |
| huIL10 K143N, | huIL10 K143N | CTTGGAGCTTATTAAAGGtATTgTTCACCTGCTCC | SEQ NO ID: 719 |
| A145T | | | |
| pSecTag2hygro- | pSecTag2hygro- | CAGGTGAAGAATGCCTCTAATAAGCTCCAAGAGAA | SEQ NO ID: 720 |
| huIL10 F146S | huIL10 | AGGC | |
| | | GCCTTTCTCTTGGAGCTTATTAGAGGCATTCTTCA | SEQ NO ID: 721 |
| | | CCTG | |
| pSecTag2hygro- | pSecTag2hygro- | GCAGGTGAAGAATGCCaccAATAAGCTCCAAGAG | SEQ NO ID: 722 |
| huIL10 F146T | huIL10 | CTCTTGGAGCTTATTggtGGCATTCTTCACCTGC | SEQ NO ID: 723 |
| pSecTag2hygro- | pSecTag2hygro- | GAATGCCTTTAATAAcCTCCAAGAGAAAGGCATC | SEQ NO ID: 724 |
| huIL10 K148N | huIL10 | GATGCCTTTCTCTTGGAGgTTATTAAAGGCATTC | SEQ NO ID: 725 |
| pSecTag2hygro- | pSecTag2hygro- | GCCTTTAATAAcCTCagcGAGAAAGGCATCTAC | SEQ NO ID: 726 |
| huIL10 K148N, | huIL10 K148N | GTAGATGCCTTTCTCgctGAGgTTATTAAAGGC | SEQ NO ID: 727 |
| Q1505 | | | |
| pSecTag2hygro- | pSecTag2hygro- | GCCTTTAATAAcCTCaccGAGAAAGGCATCTAC | SEQ NO ID: 728 |
| huIL10 K148N, | huIL10 K148N | GTAGATGCCTTTCTCggtGAGgTTATTAAAGGC | SEQ NO ID: 729 |
| Q150T | | | |
| pSecTag2hygro- | pSecTag2hy | CAGGTGAAGAATGCCTTTAATAAGAGCCAAGAGAA | SEQ NO ID: 730 |
| huIL10 L149S | gro-huIL10 | AGGC | |
| | | GCCTTTCTCTTGGCTCTTATTAAAGGCATTCTTCA | SEQ NO ID: 731 |
| | | CCTG | |
| pSecTag2hygro- | pSecTag2hygro- | GAATGCCTTTAATAAGacCCAAGAGAAAGGCATC | SEQ NO ID: 732 |
| huIL10 L149T | huIL10 | GATGCCTTTCTCTTGGgtCTTATTAAAGGCATTC | SEQ NO ID: 733 |
| pSecTag2hygro- | pSecTag2hygro- | GCCTTTAATAAGCTCaAcGAGAAAGGCATCTACAA | SEQ NO ID: 734 |
| huIL10 Q150N | huIL10 | TTGTAGATGCCTTTCTCgTtGAGCTTATTAAAGGC | SEQ NO ID: 735 |
| pSecTag2hygro- | pSecTag2hygro- | TTAATAAGCTCaAcGAGAgcGGCATCTACAAAGCC | SEQ NO ID: 736 |
| huIL10 Q150N, | huIL10 Q150N | GGCTTTGTAGATGCCgcTCTCgTtGAGCTTATTAA | SEQ NO ID: 737 |
| K152S | | | |
| pSecTag2hygro- | pSecTag2hygro- | TTAATAAGCTCaAcGAGAccGGCATCTACAAAGCC | SEQ NO ID: 738 |
| huIL10 Q150N, | huIL10 Q150N | GGCTTTGTAGATGCCggTCTCgTtGAGCTTATTAA | SEQ NO ID: 739 |
| K152T | | | |
| pSecTag2hygro- | pSecTag2hygro- | CTTTAATAAGCTCCAAaAcAAAGGCATCTACAAAG | SEQ NO ID: 740 |
| huIL10 E151N | huIL10 | CTTTGTAGATGCCTTTgTtTTGGAGCTTATTAAAG | SEQ NO ID: 741 |
| pSecTag2hygro- | pSecTag2hygro- | ATAAGCTCCAAaAcAAAaGCATCTACAAAGCCATG | SEQ NO ID: 742 |
| huIL10 E151N, | huIL10 E151N | CATGGCTTTGTAGATGCtTTTgTtTTGGAGCTTAT | SEQ NO ID: 743 |
| G153S | | | |
| pSecTag2hygro- | pSecTag2hygro- | ATAAGCTCCAAaAcAAAacCATCTACAAAGCCATG | SEQ NO ID: 744 |
| huIL10 E151N, | huIL10 E151N | CATGGCTTTGTAGATGgtTTTgTtTTGGAGCTTAT | SEQ NO ID: 745 |
| G153T | | | |
| pSecTag2hygro- | pSecTag2hygro- | ATAAGCTCCAAGAGAAcGGCATCTACAAAGCCATG | SEQ NO ID: 746 |
| huIL10 K152N | huIL10 | CATGGCTTTGTAGATGCCgTTCTCTTGGAGCTTAT | SEQ NO ID: 747 |
| pSecTag2hygro- | pSecTag2hygro- | GCTCCAAGAGAAcGGCAgCTACAAAGCCATGAGTG | SEQ NO ID: 748 |
| huIL10 K152N, | huIL10 K152N | CACTCATGGCTTTGTAGcTGCCgTTCTCTTGGAGC | SEQ NO ID: 749 |
| I152S | | | |
| pSecTag2hygro- | pSecTag2hygro- | GCTCCAAGAGAAcGGCAcCTACAAAGCCATGAGTG | SEQ NO ID: 750 |
| huIL10 K152N, | huIL10 K152N | CACTCATGGCTTTGTAGgTGCCgTTCTCTTGGAGC | SEQ NO ID: 751 |
| I152T | | | |
| pSecTag2hygro- | pSecTag2hygro- | ATAAGCTCCAAGAGAAAaaCATCTACAAAGCCATG | SEQ NO ID: 752 |
| huIL10 G153N | huIL10 | CATGGCTTTGTAGATGttTTTCTCTTGGAGCTTAT | SEQ NO ID: 753 |
| pSecTag2hygro- | pSecTag2hygro- | CCAAGAGAAAaaCATCagCAAAGCCATGAGTGAG | SEQ NO ID: 754 |
| huIL10 G153N, | huIL10 G153N | CTCACTCATGGCTTTGctGATGttTTTCTCTTGG | SEQ NO ID: 755 |
| Y155S | | | |
| pSecTag2hygro- | pSecTag2hygro- | CCAAGAGAAAaaCATCacCAAAGCCATGAGTGAG | SEQ NO ID: 756 |
| huIL10 G153N, | huIL10 G153N | CTCACTCATGGCTTTGgtGATGifTTTCTCTTGG | SEQ NO ID: 757 |
| Y155T | | | |
| pSecTag2hygro- | pSecTag2hygro- | GCCTACATGACAATGAAcATACGAAACTGAGGGCC | SEQ NO ID: 758 |
| huIL10 K175N | huIL10 | GGCCCTCAGTTTCGTATgTTCATTGTCATGTAGGC | SEQ NO ID: 759 |
| pSecTag2hygro- | pSecTag2hygro- | CATGACAATGAAcATAaGcAACTGAGGGCCCGAAC | SEQ NO ID: 760 |
| huIL10 K175N, | huIL10 K175N | GTTCGGGCCCTCAGTTgCtTATgTTCATTGTCATG | SEQ NO ID: 761 |
| R177S | | | |
| pSecTag2hygro- | pSecTag2hygro- | CATGACAATGAAcATAaccAACTGAGGGCCCGAAC | SEQ NO ID: 762 |
| huIL10 K175N, | huIL10 K175N | GTTCGGGCCCTCAGTTggtTATgTTCATTGTCATG | SEQ NO ID: 763 |
| R177T | | | |

Transfection Protocol.

All human IL-10 expression vectors (wild type and mutein) were transiently expressed in HEK293FT cells (Life Technologies #R700-07). The cells were maintained in 50 mL of DMEM (Life Technologies #11995-073)+10% characterized fetal bovine serum (Hyclon/Thermo Scientific #SH30071.03)+1× Penicillin/Streptomycin (Life Technologies #15140-122) at 37° C. at 5% CO₂ in T175 flasks (Greiner One/CellStar #660175). Upon reaching confluence, the cells were detached with 10 mL of PBS+5 mM EDTA, the cells collected with an additional 10 mL of growth media, pelleted at 1000 RPM in a centrifuge (Beckman Allegra 6R), the media aspirated, the cells resuspended in fresh media, and then split between three T175 flasks each containing 45 mL of growth media.

All non-cysteine mutein expression vectors were transfected into 6-well plates as follows: Hek293 cells were harvested from a confluent T175 flask, the cells collected as previously described and then resuspended in 20 mL of fresh growth media. Seven hundred (700) μL of the cell suspension was added to each well of a 6-well plate (Falcon #353046) containing 2 mL of fresh media and grown overnight as described. The following day, the cells were transfected using Lipofectamine 2000 (Life Technologies #1388795) using the following protocol: 250 μl of OptiMEMI Reduced Serum Media (Life Technologies #31985-088) was aliquoted to two eppindorf tubes, then 10 μl of Lipofectamine 2000 Transfection Reagent (Life Technologies #1388795) was added to one aliquot and 4 μg of DNA to the other. The two solutions were incubated separately for 5 minutes at room temperature and then the transfection complexes were formed by combining the two solutions and incubating at room temperature for an additional 30 minutes. The complete 500 μL mixture was then added drop-wise to one well of the 6-well plate, and returned to the incubator for 4 hours. The transfection media was then aspirated and replaced with DMEM+Penicillin/Streptomycin and grown for approximately 36 hours. The conditioned media was harvested and stored at 4° C.

Cysteine muteins were transfected as described above with the following exceptions: Four T175 flasks with 42-45 mL of growth media were grown to 95% confluence prior to transfection, and the transfection complexes were formed by adding 175 μL of Lipofectamine 2000 to 4.4 mL OptiMEM I and 75-100 μL of DNA to a second 4.4 mL of OptiMEM I. Upon aspiration of the transfection complexes, 50 mL of media was added to each flask.

Mock transfections contained either empty pSecTag2hygro (B) expression vector or no DNA, and were prepared as described for both the cysteine and non-cysteine variants.

Human IL-10 Detection ELISA.

A 96-well plate (Nunc Maxisorp #442404) was coated overnight at 4° C. with 100 μL/well PBS+1 μg/mL anti-human IL-10 antibody 9D7 (Armo Biosciences; Redwood City, Calif.), washed 6×200 μL in DPBS-Tween 20 (Teknova #P0297), blocked in 200 μL/well PBS+5% BSA (Calbiochem #2960) for 2 hours at room temperature on a rocking platform, and washed as previously described. The samples were serially diluted 1:5 down wells A-H in PBS and 100 μL/well was added to the assay plate. Samples were run in duplicate or triplicate. As a positive control purified human IL-10 (Armo Biosciences) was spiked into growth media to a final concentration of 2 μg/mL, while conditioned media from the mock transfections was used a negative control, and both serially diluted as described. The samples were incubated overnight at 4° C. on a rocking platform and then washed as previously described. 100 μL/well of PBS+anti-human-IL-10 antibody 12G8-biotin (Armo Biosciences) was added to each well, incubated for one hour at room temperature on a rocking platform, washed as previously described, and then 100 μL/well of PBS+streptavidin-HRP (Jackson Immuno Research #016-030-084, diluted 1:1000) was added and incubated for an additional 1 hour at room temperature on a rocking platform. The plate was then washed as described and developed with 100 μL/well of 1-Step Ultra TMB-ELISA (Pierce/Thermo #34029) for 1-5 minutes, and then the reaction stopped with 100 μL/well Stop Solution (Life Technologies #SS04). The plate was read on a Molecular Devices M2 plate reader at 450 nm.

MC/9 Bioactivity Assay.

MC/9 cells (ATCC #CRL-8306) were grown in DMEM (Life Technologies)+10% FBS (Hyclon/Thermo)+1× Penicillin/Streptomycin (Life Technologies)+50 μM β-mercaptoethanol (Fisher #O3446I-100)+1× rat T-STIM with ConA (BD #354115) at 37° C. in 5% CO₂. The cells were suspended in growth media at a density of 0.4E6 cells/mL and passaged when the cell density approached 1.5E6 cells/mL (typically after 3-4 days). To passage the cells, an appropriate volume of cell suspension was pelleted at 1000 RPM, the media aspirated, and the cells resuspended in new growth media. A fresh vial was thawed after about four weeks of culturing.

Prior to using the cells in the assay, they were washed three times in growth media without rat T-STIM, by pelleting the cells at 1000 RPM, aspirating the media, and then resuspending them in new media (without T-STIM). For the cysteine muteins, purified proteins were used in the MC/9 assay, while conditioned media from transiently transfected cells was used for the non-cysteine mutants. Samples were run in duplicates or triplicates.

Proteins in conditioned media: A cell suspension of 0.05E6 cells/mL was prepared (without T-STIM), and 50 μL/well (˜5000 cells) was added to each well of an opaque 96-well tissue culture plate (Costar #3917) and returned to the incubator while the test samples are prepared. Conditioned media from the transient transfection was diluted 1:3 in growth media without T-STIM and serially diluted 1:3 down 12 rows, and 100 μL/well was added to the cell suspension. Each plate contained conditioned media from a transient transfection with the wild type IL-10 as well as a mock transfection to use as relative reference for gauging activity. The cells were grown for ˜40 hours at 37° C. and 5% CO₂, allowed to equilibrate to room temperature for 20 minutes, and then 100 μL/well Cell Titer Glo (Promega #G7571) was added to each well. The plates were rocked at 450 rpm for about 30 minutes and read on a Molecular Devices SpectraMax L plate reader at 395 nm with a 1 second integration time.

Purified proteins: The protocol was the same as the conditioned media with the exception that the final concentration of the IL-10 protein in the assay plate was between 200-800 ng/mL.

Numerous assays involving the use of MC/9 cells are described in the literature. IL-10 administration to MC/9 cells (murine cell line with characteristics of mast cells available from Cell Signaling Technology; Danvers, Mass.) causes increased cell proliferation in a dose-dependent manner. An exemplary assay is disclosed by Thompson-Snipes, L. et al. ((1991) J. Exp. Med. 173:507-10), who describe a standard assay protocol in which MC/9 cells are supplemented with IL3+IL10 and IL3+IL4+IL10. Vendors (e.g., R&D Systems, USA; and Cell Signaling Technology, Danvers, Mass.) use the assay as a lot release assay for rhIL10. Those of ordinary skill in the art will be able to modify the standard assay protocol described in Thompson-Snipes, L. et al, such that cells are only supplemented with IL-10.

Purification of Wild Type Human IL-10 and Cysteine Muteins.

Anti-human-IL-10 antibody 9D7 was coupled to CNBr-activated Sepharose 4 Fast Flow (GE Healthcare #71-5000-15 AF, followed manufacturer's protocol) and equilibrated in PBS. 500 μL-1 mL of 9D7-sepaharose was added per 100 mL of conditioned media contained in a glass Econo-Column (Bio-rad, Hercules, Calif.) and incubated for 1-2 hours at room temperature on a rocking platform. The media was allowed to run through the column via gravity flow, washed 1× with 1×PBS (pH 7.4), eluted with 0.1M glycine (pH 2.9) and neutralized with a 10% volume of 1M Tris buffer (pH 8.0). The protein was concentrated and buffer exchanged into PBS (pH 7.4) using an Amicon Ultra Centrifugal Filter Device (Millipore, Billerica, Mass.; 10,000 kD molecular weight cutoff). Protein concentration was determined by spectrophotometer at 280 nm. SEC Analysis of Cysteine Variants. Using a 1100 series HPLC (Agilent Technologies, Santa Clara, Calif.), 20-50 μg of protein was injected on a TSK3000sw column (Tosoh Biosciences, Tokyo, JP), equilibrated with PBS (pH 7.4), and run at a flow rate of 1 mL/min.

Production of Pegylated IL-10

The present disclosure contemplates the synthesis of pegylated IL-10 by any means known to the skilled artisan. The description hereafter of several alternative synthetic schemes for producing mono-PEG-IL-10 and a mix of mono-/di-PEG-IL-10 is meant to be illustrative only. While both mono-PEG-IL-10 and a mix of mono-/di-PEG-IL-10 have many comparable properties, a mix of selectively pegylated mono- and di-PEG-IL-10 improves the yield of the final pegylated product (see, e.g., U.S. Pat. No. 7,052,686 and US Pat. Publn. No. 2011/0250163).

In addition to leveraging her own skills in the production and use of PEGs (and other drug delivery technologies) suitable in the practice of the present disclosure, the skilled artisan is also familiar with many commercial suppliers of PEG-related technologies (and other drug delivery technologies). By way of example, NOF America Corp (Irvine, Calif.) supplies mono-functional Linear PEGs, bi-functional PEGs, multi-arm PESs, branched PEGs, heterofunctional PEGs, forked PEGs, and releasable PEGs; and Parchem (New Rochelle, N.Y.) is a global distributor of PEG products and other specialty raw materials.

Exemplary PEG-IL-10 Synthetic Scheme No. 1

IL-10 may be dialyzed against 10 mM sodium phosphate at pH 7.0, 100 mM NaCl. The dialyzed IL-10 may then be diluted 3.2 times to a concentration of 4 mg/mL using the dialysis buffer. Prior to the addition of the linker, SC-PEG-12K (Delmar Scientific Labs, Maywood, Ill.), 1 volume of 100 mM Na-tetraborate at pH 9.1 can be added into 9 volumes of the diluted IL-10 to raise the pH of the IL-10 solution to 8.6. The SC-PEG-12K linker can be dissolved in the dialysis buffer and the appropriate volume of the linker solution (1.8 to 3.6 mole of linker/mole of IL-10) can be added into the diluted IL-10 solution to start the pegylation reaction. The reaction can be carried out at 5° C. in order to control the rate of the reaction. The reaction solution can be mildly agitated during the pegylation reaction. When the mono-PEG-IL-10 yield, as determined by size exclusion HPLC (SE-HPLC), is close to 40%, the reaction is stopped by adding 1M glycine solution to a final concentration of 30 mM. The pH of the reaction solution is slowly adjusted to 7.0 using an HCl solution, and the reaction solution is then filtered using a 0.2 micron filter and stored at −80.degree ° C.

Exemplary PEG-IL-10 Synthetic Scheme No. 2

Mono-PEG-IL-10 is prepared using methoxy-PEG-aldehyde (PALD-PEG) as a linker (Inhale Therapeutic Systems Inc., Huntsville, Ala.; also available from NOF America Corp (Irvine, Calif.)). PALD-PEG can have molecular weights of 5 KDa, 12 KDa, or 20 KDa. IL-10 is dialyzed and diluted as described above, except the pH of the reaction buffer is between 6.3 and 7.5. Activated PALD-PEG linker is added to reaction buffer at a 1:1 molar ratio. Aqueous cyanoborohydride is added to the reaction mixture to a final concentration of 0.5 to 0.75 mM. The reaction is carried out at room temperature (18-25° C.) for 15-20 hours with mild agitation. The reaction is quenched with 1M glycine. Yields are analyzed by SE-HPLC. Mono-PEG-IL-10 is separated from unreacted IL-10, PEG linker and di-PEG-IL-10 by gel filtration chromatography and characterized by RP-HPLC and bioassay (e.g., stimulation of IL-10-responsive cells or cell lines).

Exemplary PEG-IL-10 Synthetic Scheme No. 3.

IL-10 (e.g., rodent or primate) is dialyzed against 50 mM sodium phosphate, 100 mM sodium chloride pH ranges 5-7.4. A 1:1-1:7 molar ratio of 5K PEG-propylaldehyde is reacted with IL-10 at a concentration of 1-12 mg/mL in the presence of 0.75-30 mM sodium cyanoborohydride. Alternatively the reaction can be activated with picoline borane in a similar manner. The reaction is incubated at 5-30° C. for 3-24 hours.

The pH of the pegylation reaction is adjusted to 6.3, 7.5 mg/mL of hIL-10 is reacted with PEG to make the ratio of IL-10 to PEG linker 1:3.5. The final concentration of cyanoborohydride is ˜25 mM, and the reaction is carried out at 15° C. for 12-15 hours. The mono- and di-PEG IL-10 are the largest products of the reaction, with the concentration of each at ˜45-50% at termination. The reaction may be quenched using an amino acid such as glycine or lysine or, alternatively, Tris buffers. Multiple purification methods can be employed such as gel filtration, anion and cation exchange chromatographies, and size exclusion HPLC (SE-HPLC) to isolate the desired pegylated IL-10 molecules.

Exemplary PEG-IL-10 Synthetic Scheme No. 4.

IL-10 is dialyzed against 10 mM sodium phosphate pH 7.0, 100 mM NaCl. The dialyzed IL-10 is diluted 3.2 times to a concentration of about 0.5 to 12 mg/mL using the dialysis buffer. Prior to the addition of the linker, SC-PEG-12K (Delmar Scientific Laboratories, Maywood, Ill.), one volume of 100 mM Na-tetraborate at pH 9.1 is added into 9 volumes of the diluted IL-10 to raise the pH of the IL-10 solution to 8.6. The SC-PEG-12K linker is dissolved in the dialysis buffer and the appropriate volume of the linker solution (1.8 to 3.6 mole linker per mole of IL-10) is added into the diluted IL-10 solution to initiate the pegylation reaction. The reaction is carried out at 5° C. in order to control the rate of the reaction, and the reaction solution is mildly agitated. When the mono-PEG-IL-10 yield, as determined by size exclusion HPLC (SE-HPLC), is close to 40%, the reaction is stopped by adding 1M glycine solution to a final concentration of 30 mM. The pH of the reaction solution is slowly adjusted to 7.0 using an HCl solution, and the reaction is 0.2 micron filtered and stored at −80° C.

### Assays to Determine the Bioactivity of Modified Forms of IL-10

The present disclosure contemplates the use of any assays and methodologies known in the art for determining the bioactivity of the IL-10 molecules described herein. The assays described hereafter are representative, and not exclusionary.

TNFα Inhibition Assay.

PMA-stimulation of U937 cells (lymphoblast human cell line from lung available from Sigma-Aldrich (#85011440); St. Louis, Mo.) causes the cells to secrete TNFα, and subsequent treatment of these TNFα-secreting cells with human IL-10 causes a decrease in TNFα secretion in a dose-dependent manner.

An exemplary TNFα inhibition assay may be performed using the following protocol. After culturing U937 cells in RMPI containing 10% FBS/FCS and antibiotics, plate 1×10⁵, 90% viable U937 cells in 96-well flat bottom plates (any plasma-treated tissue culture plates (e.g., Nunc; Thermo Scientific, USA) may be used) in triplicate per condition. Plate cells to provide for the following conditions (all in at least triplicate; for ‘media alone’ the number of wells is doubled because one-half will be used for viability after incubation with 10 nM PMA): 5 ng/ml LPS alone; 5 ng/mL LPS+0.1 ng/mL rhIL-10; 5 ng/mL LPS+1 ng/mL rhIL-10; 5 ng/mL LPS+10 ng/mL rhIL-10; 5 ng/mL LPS+100 ng/mL rhIL-10; 5 ng/mL LPS+1000 ng/mL rhIL-10; 5 ng/mL LPS+0.1 ng/mL PEG-rhIL-10; 5 ng/mL LPS+1 ng/mL PEG-rhIL-10; 5 ng/mL LPS+10 ng/mL PEG-rhIL-10; 5 ng/mL LPS+100 ng/mL PEG-rhIL-10; and 5 ng/mL LPS+1000 ng/mL PEG-rhIL-10.

Expose each well to 10 nM PMA in 200 μL for 24 hours, culturing at 37° C. in 5% CO₂ incubator, after which time ˜90% of cells should be adherent. The three extra wells are resuspended, and the cells are counted to assess viability (>90% should be viable). Wash gently but thoroughly 3× with fresh, non-PMA-containing media, ensuring that cells are still in the wells. Add 100 μL per well of media containing the appropriate concentrations (2× as the volume will be diluted by 100%) of rhIL-10 or PEG-rhIL-10, incubate at 37° C. in a 5% CO₂ incubator for 30 minutes. Add 100 μL per well of 10 ng/mL stock LPS to achieve a final concentration of 5 ng/mL LPS in each well, and incubate at 37° C. in a 5% CO₂ incubator for 18-24 hours. Remove supernatant and perform TNFα ELISA according to the manufacturer's instructions. Run each conditioned supernatant in duplicate in ELISA.

CD8+T-Cell IFNγ Secretion Assay.

Activated primary human CD8+ T-cells secrete IFNγ when treated with PEG-IL-10 and then with an anti-CD3 antibody. The following protocol provides an exemplary CD8+ T-cell IFNγ secretion assay. Human primary peripheral blood mononuclear cells (PBMCs) can be isolated according to any standard protocol (see, e.g., Fuss et al. (2009) Current Protocols in Immunology, Unit 7.1, John Wiley, Inc., NY). 2.5 mL of PBMCs (at a cell density of 10 million cells/mL) can be cultured per well with complete RPMI, containing RPMI (Life Technologies; Carlsbad, Calif.), 10 mM HEPES (Life Technologies; Carlsbad, Calif.), 10% Fetal Calf Serum (Hyclone Thermo Fisher Scientific; Waltham, Mass.) and Penicillin/Streptomycin cocktail (Life Technologies; Carlsbad, Calif.), in any standard tissue culture treated 6-well plate (BD; Franklin Lakes, N.J.). Human pegylated-IL-10 can be added to the wells at a final concentration of 100 ng/mL; a final concentration of 10 μg/mL of antibodies blocking the function of inhibitory/checkpoint receptors can also be added in combination with pegylated-IL-10. Cells can be incubated in a humidified 37° C. incubator with 5% CO₂ for 6-7 days. After this incubation, CD8+ T-cells can be isolated using Miltenyi Biotec's MACS cell separation technology according to the manufacture's protocol (Miltenyi Biotec; Auburn, Calif.). The isolated CD8+ T-cells can then be cultured with complete RPMI containing 1 μg/mL anti-CD3 antibody (Affymetrix eBioscience; San Diego, Calif.) in any standard tissue culture plate for 4 hours. After the 4 hour incubation, the media can be collected and assayed for IFNγ using a commercial ELISA kit and following the manufacture's protocol (Affymetrix Bioscience; San Diego, Calif.).

### Tumor Models and Tumor Analysis

Any art-accepted tumor model, assay, and the like can be used to evaluate the effect of the IL-10 molecules described herein on various tumors. The tumor models and tumor analyses described hereafter are representative of those that can be utilized.

Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. PEG-mIL-10 can be administered to the immunocompetent mice, while PEG-hIL-10 treatment can be in the B-cell deficient mice. Tumors are allowed to reach a size of 100-250 mm³ before treatment is started. IL-10, PEG-mIL-10, PEG-hIL-10, or buffer control is administered subcutaneously at a site distant from the tumor implantation. Tumor growth is typically monitored twice weekly using electronic calipers.

Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80° C. Primary tumor growth is typically monitored twice weekly using electronic calipers. Tumor volume may be calculated using the formula (width×length/2) where length is the longer dimension. Tumors are allowed to reach a size of 90-250 mm³ before treatment is started.

### Identifying Mutants Demonstrating Biological Activity

Using the methodologies described herein, an assessment was conducted to determine which of the 160 amino acid residues of the mature human IL-10 protein will tolerate a substitution with a residue conducive to forming an anchor site for a PEG moiety. Residues identified using this assessment were further analyzed to determine whether a substitution will result in a mutant (mutein) exhibiting bioactivity. The skilled artisan will recognize that not all mutants active in an in vitro assay will have activity in an in vivo setting, and vice versa.

The results of the assessment are summarized in FIG. 5. The first two rows of FIG. 5 define the boundaries for each of the regions of IL-10 (i.e., a) Pre-helix A, b) Helix A, c) A/B Inter-helix Junction, d) Helix B, e) B/C Inter-helix Junction, f) Helix C, g) C/D Inter-helix Junction h) Helix D, i) D/E Inter-helix Junction, j) Helix E, k) E/F Inter-helix Junction, l) Helix F, and m) and the amino acid residues of each of the regions, as well as the locations of the intrahelical kinks and the amino acid residues of each kink. The next four rows of FIG. 5 relate to the types of mutations that were introduced at each residue: Cysteine, Tyrosine, N-X-S, and N-X-T; N-X-S, and N-X-T are N-glycosylation motifs.

Referring to the shading in FIG. 5, with the exception of the dark grey boxes with an “x” that are described below, the residues in the dark grey boxes were not mutated or part of the analysis. Based on application of the teachings herein, such residues are not surface-exposed or are involved in receptor binding. The remaining 78 residues in the light grey boxes represent the residues that are more likely to be surface exposed on the homodimer and less likely to interfere with receptor binding. It is to be understood that a skilled artisan may conclude that one or more residues may be categorized differently (i.e., a residue that is in a dark grey box might be placed in a light gray box).

The mutants (e.g., cysteine or tyrosine) were generated using the methods described herein and were evaluated in an MC/9 assay to determine biological activity. If a mutant was expressed and exhibited biological activity, a “+” was placed in the applicable box (e.g., referring to amino acid residue 96, a tyrosine mutant exhibited activity whereas a cysteine mutant did not). For purposes of the assessment, the measurement of any biological activity resulted in the assignment of a “+” sign.

In the columns associated with particular amino acid residues in FIG. 5, some boxes (light grey) contain an “+” while other boxes (dark grey) contain an “x”. In these instances (e.g., 10 (N)), the dark grey “x” boxes could not be mutated for various reasons. Residue 59 (Y) could not be mutated to a tyrosine because human IL-10 already contains a tyrosine at that position. For residues at the 10 (N), and 60 (L), 106 (R), introducing an N-glycosylation site would interfere with cysteine bonding and likely destroyed the bioactivity of the protein. For residue 116 (N), the N-X-S N-glycosylation motif could not be introduced because the protein already contains an N-X-S N-glycosylation motif. For residue 160 (N), because the N-glycosylation motif is three amino acids long (N-X-S or N-X-T), an N-glycosylation site cannot be introduced at the last residue of a protein.

Mutants in light grey boxes without a “+” (e.g., the cysteine row of column 4 (Q)) indicate that the mutants did not express or were not active in the MC/9 assay. However, it should be noted that only cysteine mutants which formed a large degree of heterodimers were tested for activity. Free cysteines allow the protein to form numerous different isoforms or aggregates as it attempts to pair up the free cysteine, and these aggregates might show some activity in a cell-based assay system even though they are not likely to be a therapeutic candidate; thus, these were not evaluated. In comparison, the other mutations were far less likely to introduce aggregates and therefore they were tested for bioactivity.

Based on the foregoing description, 78 amino acid residues may initially be considered as sites for the generation of mutants. Of the 78 potential sites to introduce a mutation, 76 possessed properties that made them viable candidates for anchoring a PEG moiety as two sites did not generate an active protein with any of the tested mutations.

As previously indicated, the present disclosure contemplates peptides comprising a substitution that would facilitate the attachment of a PEG or other moiety to at least one amino acid residue of i) Pre-helix A other than amino acid residues 12 (C), 15 (F) or 16 (P); ii) Helix A other than amino acid residues 19-24 (LPNMLR (SEQ ID NO:33)), 26-30 (LRDAF (SEQ ID NO:34)), 33-39; (VKTFFQM (SEQ ID NO:35)), or 41 (D); iii) Helix B other than amino acid residues 52 (L), 53 (L), or 56 (F); iv) B/C Inter-helix Junction; v) Helix C other than amino acid residues 62 (C), 64 (A), 65 (L), 68 (M), 69 (I), 71-73 (FYL), 76 (V), 77 (M), or 80 (A); vi) C/D Inter-helix Junction; vii) Helix D other than amino acid residues 87 (I), 91 (V), 94 (L), 98 (L), 101 (L), 105 (L), or 108 (C); viii) D/E Inter-helix Junction other than amino acid residues 111 (F), 112 (L), or 114 (C); ix) Helix E other than amino acid residues 120 (A), 121 (V), 124 (V), 127 (A), 128 (F) or 131 (L); x) E/F Inter-helix Junction; xi) Helix F other than amino acid residues 136-156 (IYKAMSEFDIFINYIEAYMTM (SEQ ID NO:36)), 158 (I) or 159 (R); or xii) Post-helix F.

Some embodiments of the present disclosure contemplate peptides comprising at least one amino acid substitution in at least one of the following regions: 1-11, 49-51, 57-61, 81-86, 88-90, 102-104, 115-119, or 132-134. In other embodiments, the peptides comprise at least one amino acid substitution at least at one of the following positions: 1-11, 13, 14, 17, 18, 25, 31, 32, 40, 49-51, 54, 55, 57-61, 63, 66, 67, 70, 74, 75, 78, 79, 81-86, 88-90, 92, 93, 96, 97, 99, 100, 102-104, 106, 107, 109, 110, 113, 115-119, 122, 123, 125, 126, 129, 130, 132-134, 157 or 160.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

1. A peptide comprising:
a) a Pre-helix A, b) a Helix A, c) an A/B Inter-helix Junction, d) a Helix B, e) a B/C Inter-helix Junction, f) a Helix C, g) a C/D Inter-helix Junction h) a Helix D, i) a D/E Inter-helix Junction, j) a Helix E, k) an E/F Inter-helix Junction, l) a Helix F, and m) a Post-helix F; and wherein the peptide further comprises at least one amino acid substitution, addition or deletion to one or more of a)-m).

2. The peptide of claim 1, comprising:
a) a Pre-helix A, b) a Helix A, c) an A/B Inter-helix Junction, d) a Helix B, e) a B/C Inter-helix Junction, f) a Helix C, g) a C/D Inter-helix Junction h) a Helix D, i) a D/E Inter-helix Junction, j) a Helix E, k) an E/F Inter-helix Junction, l) a Helix F, and m) a Post-helix F; and wherein the peptide further comprises at least one amino acid substitution comprising:
substitution of at least one amino acid residue of Pre-helix A other than amino acid residues 12 (C), 15 (F) or 16 (P); or
substitution of at least one amino acid residue of Helix A other than amino acid residues 19-24 (LPNMLR), 26-30 (LRDAF), 33-39; (VKTFFQM), or 41 (D); or
substitution of at least one amino acid residue of Helix B other than amino acid residues 52 (L), 53 (L), or 56 (F); or
substitution of the amino acid residue of the B/C Inter-helix Junction; or
substitution of at least one amino acid residue of Helix C other than amino acid residues 62 (C), 64 (A), 65 (L), 68 (M), 69 (I), 71-73 (FYL), 76 (V), 77 (M), or 80 (A); or
substitution of at least one amino acid residue of the C/D Inter-helix Junction; or
substitution of at least one amino acid residue of Helix D other than amino acid residues 87 (I), 91 (V), 94 (L), 98 (L), 101 (L), 105 (L), or 108 (C); or
substitution of at least one amino acid residue of the D/E Inter-helix Junction other than amino acid residues 111 (F), 112 (L), or 114 (C); or
substitution of at least one amino acid residue of Helix E other than amino acid residues 120 (A), 121 (V), 124 (V), 127 (A), 128 (F) or 131 (L); or
substitution of the amino acid residue of the E/F Inter-helix Junction; or
substitution of at least one amino acid residue of Helix F other than amino acid residues 136-156 (IYKAMSEFDIFINYIEAYMTM), 158 (I) or 159 (R); or
substitution of the amino acid residue of Post-helix F.

3. The peptide of claim 2, wherein the at least one amino acid substitution does not disrupt the non-covalent interactions between the two monomer subunits of the peptide.

4. The peptide of claim 2, wherein the at least one amino acid substitution is a conservative substitution

5. The peptide of claim 2, wherein the peptide has a bioactivity at least equal to the bioactivity of SEQ ID NO:2, wherein the bioactivity is determined in an in vitro assay or an in vivo assay.

6. The peptide of claim 5, wherein the in vitro activity is at least one of a TNFα inhibition assay, an MC/9 cell proliferation assay, or a CD8+T-cell IFNγ secretion assay.

7. The peptide of claim 2, wherein the at least one amino acid substitution does not adversely affect immunogenicity.

8. The peptide of claim 7, wherein the immunogenicity of the peptide is predicted by screening for at least one of T-cell epitopes or B-cell epitopes.

9. The peptide of claim 8, wherein the screening is at least one of an in silico screening system or an ex vivo assay system.

10. The peptide of claim 2, wherein the at least one amino acid substitution is in at least one of the following regions: 1-11, 49-51, 57-61, 81-86, 88-90, 102-104, 115-119, or 132-134.

11. The peptide of claim 2, wherein the at least one amino acid substitution is at least at one of the following positions: 1-11, 13, 14, 17, 18, 25, 31, 32, 40, 49-51, 54, 55, 57-61, 63, 66, 67, 70, 74, 75, 78, 79, 81-86, 88-90, 92, 93, 96, 97, 99, 100, 102-104, 106, 107, 109, 110, 113, 115-119, 122, 123, 125, 126, 129, 130, 132-134, 157 or 160.

12. The peptide of claim 2, wherein the peptide does not comprise substitution of an amino acid residue involved with receptor binding.

13. The peptide of claim 2, wherein the peptide comprises at least one modification to form a modified peptide;
wherein the modification does not alter the amino acid sequence of the peptide, and
wherein the modification improves at least one property of the peptide.

14. The peptide of claim 13, wherein the modified peptide is pegylated.

15. The peptide of claim 14, wherein the modified peptide comprises at least one PEG molecule covalently attached to at least one amino acid residue of at least one monomer of IL-10.

16. The peptide of claim 15, wherein the modified peptide comprises a mixture of mono-pegylated and di-pegylated IL-10.

17. The peptide of claim 15, wherein the PEG component of the modified peptide has a molecular mass from 5 kDa to 20 kDa.

18. The peptide of claim 15, wherein the PEG component of the modified peptide has a molecular mass greater than 20 kDa.

19. The peptide of claim 15, wherein the PEG component of the modified peptide has a molecular mass of at least 30 kD.

20. The peptide of claim 13, wherein the modified peptide is glycosylated.

21. The peptide of claim 13, wherein the modified peptide comprises at least one of an Fc fusion molecule, a serum albumin, or an albumin binding domain (ABD).

22. The peptide of claim 13, wherein the modification is site-specific.

23. The peptide of claim 13, wherein the modification comprises a linker.

24. The peptide of claim 13, wherein the modification improves at least one physical property of the peptide.

25. The peptide of claim 24, wherein the physical property is selected from the group consisting of solubility, bioavailability, serum half-life, and circulation time.

26. The peptide of claim 13, wherein the modified peptide has activity at least comparable to the activity of mature human IL-10.

27. The peptide of claim 2, wherein the peptide is produced recombinantly.

28. A peptide comprising the amino acid sequence of SEQ ID NO:2, wherein the peptide comprises at least one amino acid substitution of a surface-exposed amino acid residue;
and wherein the substitution has at least one of the following effects: (a) improves at least one physical property of the peptide, (b) does not adversely affect the immunogenicity of the peptide, or (c) does not adversely affect the bioactivity of the peptide.

29. The peptide of claim 28, wherein the peptide does not comprise substitution of an amino acid residue involved with receptor binding.

30. The peptide of claim 28, wherein the substitution does not disrupt the intramolecular disulfide bonds of the peptide.

31. The peptide of claim 28, wherein the substitution does not disrupt the non-covalent interactions between the two monomer subunits of the peptide.

32. The peptide of claim 28, wherein the at least one amino acid substitution is a conservative substitution.

33. The peptide of claim 28, wherein the at least one amino acid substitution is not a substitution at one or more of amino acid residues 12, 62, 108 and 114.

34. A peptide comprising at least 90% sequence identity to the amino acid sequence of SEQ ID NO:2, wherein the peptide has a least one of the following characteristics: (a) is not more immunogenic than the peptide of SEQ ID NO:2, (b) has a bioactivity at least equal to the bioactivity of the peptide of SEQ ID NO:2, (c) has an improvement in at least one physical property of the peptide of SEQ ID NO:2.

35. The peptide of claim 34, wherein the peptide has at least 95% amino acid sequence identity.

36. The peptide of claim 34, wherein the peptide has at least 97% amino acid sequence identity.

37. The peptide of claim 34, wherein the peptide has at least 98% amino acid sequence identity.

38. The peptide of claim 34, wherein the peptide has at least 99% amino acid sequence identity.

39. The peptide of claim 34, wherein each monomer of the peptide has at least 125 amino acid residues.

40. The peptide of claim 34, wherein each monomer of the peptide has at least 150 amino acid residues.

41. The peptide of claim 34, wherein each monomer of the peptide has at least 155 amino acid residues.

42. The peptide of claim 34, wherein the peptide comprises at least one amino acid substitution, deletion or addition relative to the amino acid sequence of SEQ ID NO:2.

43. The peptide of claim 42, wherein the peptide does not comprise substitution of an amino acid residue involved with receptor binding.

44. The peptide of claim 42, wherein the peptide comprises at least one amino acid substitution of a surface-exposed amino acid residue.

45. The peptide of claim 42, wherein the at least one addition, deletion, or substitution does not disrupt the intramolecular disulfide bonds of the peptide.

46. The peptide of claim 42, wherein the at least one addition, deletion, or substitution does not disrupt the non-covalent interactions between the two monomer subunits of the peptide.

47. The peptide of claim 42, wherein the at least one amino acid substitution is a conservative substitution.

48. The peptide of claim 42, wherein the at least one amino acid substitution is not a substitution at one or more of amino acid residues 12, 62, 108 and 114.

49. The peptide of claim 28 or 34, wherein the physical property is selected from the group consisting of solubility, bioavailability, serum half-life, and circulation time.

50. The peptide of claim 28 or 34, wherein the peptide has a bioactivity at least equal to the bioactivity of SEQ ID NO:2, wherein the bioactivity is determined in an in vitro assay or an in vivo assay.

51. The peptide of claim 48, wherein the in vitro activity is at least one of a TNFα inhibition assay, an MC/9 cell proliferation assay, or a CD8+T-cell IFNγ secretion assay.

52. The peptide of claim 28 or 34, wherein the immunogenicity of the peptide is predicted by screening for at least one of T-cell epitopes or B-cell epitopes.

53. The peptide of claim 52, wherein the screening is at least one of an in silico screening system or an ex vivo assay system.

54. The peptide of claim 28 or 34, wherein the peptide comprises at least one modification to form a modified peptide;
wherein the modification does not alter the amino acid sequence of the modified peptide, and
wherein the modified peptide has activity at least comparable to the activity of mature human IL-10.

55. The peptide of claim 54, wherein the modified peptide is pegylated.

56. The peptide of claim 55, wherein the modified peptide comprises at least one PEG molecule covalently attached to at least one amino acid residue of at least one monomer of IL-10.

57. The peptide of claim 56, wherein the modified peptide comprises a mixture of mono-pegylated and di-pegylated IL-10.

58. The peptide of claim 56, wherein the PEG component of the modified peptide has a molecular mass from 5 kDa to 20 kDa.

59. The peptide of claim 56, wherein the PEG component of the modified peptide has a molecular mass greater than 20 kDa.

60. The peptide of claim 56, wherein the PEG component of the modified peptide has a molecular mass of at least 30 kD.

61. The peptide of claim 54, wherein the modified peptide is glycosylated.

62. The peptide of claim 54, wherein the modified peptide comprises at least one of an Fc fusion molecule, a serum albumin, or an albumin binding domain (ABD).

63. The peptide of claim 54, wherein the modification is site-specific.

64. The peptide of claim 54, wherein the modification comprises a linker.

65. The peptide of claim 28 or 34, wherein the peptide is produced recombinantly.

66. A nucleic acid molecule encoding a peptide of claim 1, 26 or 32.

67. The nucleic acid molecule of claim 66, wherein the nucleic acid molecule is operably linked to an expression control element that confers expression of the nucleic acid molecule encoding the peptide in vitro, in a cell or in vivo.

68. A vector comprising the nucleic acid molecule of claim 67.

69. The vector of claim 68, wherein the vector comprises a viral vector.

70. A transformed or host cell that expresses a peptide of claim 2, 28 or 34.

71. A pharmaceutical composition, comprising a peptide of claim 2, 28 or 34, and a pharmaceutically acceptable diluent, carrier or excipient.

72. The pharmaceutical composition of claim 71, wherein the excipient is an isotonic injection solution.

73. The pharmaceutical composition of claim 71, wherein the pharmaceutical composition is suitable for human administration.

74. The pharmaceutical composition of claim 71, further comprising at least one additional prophylactic or therapeutic agent.

75. A sterile container comprising the pharmaceutical composition of claim 71.

76. The sterile container of claim 75, wherein the sterile container is a syringe.

77. A kit comprising the sterile container of claim 75.

78. The kit of claim 77, further comprising a second sterile container comprising at least one additional prophylactic or therapeutic agent.

79. An antibody that binds specifically to a peptide of claim 2, 28 or 34.

80. The antibody of claim 79, wherein the antibody is a monoclonal antibody.

81. The antibody of claim 79, wherein the antibody comprises a light chain variable region and a heavy chain variable region present in separate polypeptides.

82. The antibody of claim 79, wherein the antibody comprises a light chain variable region and a heavy chain variable region present in a single polypeptide.

83. The antibody of claim 79, wherein the antibody comprises a heavy chain constant region, and wherein the heavy chain constant region is of the isotype IgG1, IgG2, IgG3, or IgG4.

84. The antibody of claim 79, wherein the antibody is detectably labeled.

85. The antibody of claim 79, wherein the antibody is a Fv, scFv, Fab, F(ab′)₂, or Fab′.

86. The antibody of claim 79, wherein the antibody is a human antibody.

87. The antibody of claim 79, wherein the antibody binds the peptide with an affinity of from about 10⁷ M⁻¹ to about 10¹² M⁻¹.

88. The antibody of claim 79, wherein the antibody comprises a covalently linked moiety selected from a lipid moiety, a fatty acid moiety, a polysaccharide moiety, and a carbohydrate moiety.

89. The antibody of claim 79, wherein the antibody comprises an affinity domain.

90. The antibody of claim 79, wherein the antibody is immobilized on a solid support.

91. The antibody of claim 79, wherein the antibody is a humanized antibody.

92. The antibody of claim 79, wherein the antibody is a single chain Fv (scFv) antibody.

93. The antibody of claim 92, wherein the scFv is multimerized.

94. The antibody of claim 79, wherein the antibody comprises a covalently linked non-peptide polymer.

95. The antibody of claim 94, wherein the polymer is a poly(ethylene glycol) polymer.

96. A pharmaceutical composition comprising an antibody of claim 79, and a pharmaceutically acceptable diluent, carrier, or excipient.

97. The pharmaceutical composition of claim 96, wherein the excipient is an isotonic injection solution.

98. The pharmaceutical composition of claim 96, wherein the pharmaceutical composition is suitable for human administration.

99. The pharmaceutical composition of any one of claim 96, further comprising at least one additional prophylactic or therapeutic agent.

100. A sterile container comprising the pharmaceutical composition of claim 96.

101. The sterile container of claim 100, wherein the sterile container is a syringe.

102. A kit comprising the sterile container of claim 100.

103. The kit of claim 102, further comprising a second sterile container comprising a second therapeutic agent.

104. A method of treating or preventing a disease, disorder or condition in a subject, comprising administering to the subject a therapeutically effective amount of a peptide of claim 2, 28 or 34.

105. The method of claim 104, wherein the disease, disorder or condition is a proliferative disorder.

106. The method of claim 105, wherein the proliferative disorder is a cancer.

107. The method of claim 106, wherein the cancer is a solid tumor or a hematological disorder.

108. The method of claim 104, wherein the disease, disorder or condition is an immune or inflammatory disorder.

109. The method of claim 108, wherein immune or inflammatory disorder is selected from the group consisting of inflammatory bowel disease, psoriasis, rheumatoid arthritis, multiple sclerosis, and Alzheimer's disease.

110. The method of claim 104, wherein the disease, disorder or condition is thrombosis or a thrombotic condition.

111. The method of claim 104, wherein the disease, disorder or condition is a fibrotic disorder.

112. The method of claim 104, wherein the disease, disorder or condition is a viral disorder.

113. The method of claim 112, wherein the viral disorder is selected from the group consisting of human immunodeficiency virus, hepatitis B virus, hepatitis C virus and cytomegalovirus.

114. The method of claim 104, wherein the disease, disorder or condition is a cardiovascular disorder.

115. The method of claim 114, wherein the cardiovascular disorder is atherosclerosis.

116. The method of claim 115, wherein the subject has elevated cholesterol.

117. The method of claim 104, wherein the subject is human.

118. The method of claim 104, wherein the administering is by parenteral injection.

119. The method of claim 118, wherein the parenteral injection is subcutaneous.

120. The method of claim 104, further comprising administering at least one additional prophylactic or therapeutic agent.


SEQUENCE LISTING (763 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 11 aa, Artificial sequence
  Derived from HIV-1 TAT
YGRKKRRQRRR

SEQ ID NO: 4 - protein, 12 aa, Artificial Sequence
  Synthetic polypeptide
RRQRRTSKLMKR

SEQ ID NO: 5 - protein, 27 aa, Artificial Sequence
  Synthetic polypeptide
GWTLNSAGYLLGKINLKALAALAKKIL

SEQ ID NO: 6 - protein, 33 aa, Artificial Sequence
  Synthetic polypeptide
KALAWEAKLAKALAKALAKHLAKALAKALKCEA

SEQ ID NO: 7 - protein, 16 aa, Artificial Sequence
  Synthetic polypeptide
RQIKIWFQNRRMKWKK

SEQ ID NO: 8 - protein, 11 aa, Artificial Sequence
  Synthetic polypeptide
YGRKKRRQRRR

SEQ ID NO: 9 - protein, 9 aa, Artificial Sequence
  Synthetic polypeptide
RKKRRQRRR

SEQ ID NO: 10 - protein, 11 aa, Artificial Sequence
  Synthetic polypeptide
YGRKKRRQRRR

SEQ ID NO: 11 - protein, 8 aa, Artificial Sequence
  Synthetic polypeptide
RKKRRQRR

SEQ ID NO: 12 - protein, 11 aa, Artificial Sequence
  Synthetic polypeptide
YARAAARQARA

SEQ ID NO: 13 - protein, 11 aa, Artificial Sequence
  Synthetic polypeptide
THRLPRRRRRR

SEQ ID NO: 14 - protein, 11 aa, Artificial Sequence
  Synthetic polypeptide
GGRRARRRRRR

SEQ ID NO: 15 - protein, 18 aa, Homo sapiens
MKWVTFISLLFLFSSAYS

SEQ ID NO: 16 - protein, 6 aa, Homo sapiens
RGVFRR

SEQ ID NO: 17 - protein, 4 aa, Artificial Sequence
  Synthetic polypeptide
RGRR

SEQ ID NO: 18 - protein, 6 aa, Artificial Sequence
  Synthetic polypeptide
RKRKKR

SEQ ID NO: 19 - protein, 4 aa, Artificial Sequence
  Synthetic polypeptide
RKKR

SEQ ID NO: 20 - protein, 6 aa, Artificial Sequence
  Synthetic polypeptide
RRRKKR

SEQ ID NO: 21 - protein, 5 aa, Artificial Sequence
  Synthetic polypeptide
GSGGS

SEQ ID NO: 22 - protein, 4 aa, Artificial Sequence
  Synthetic polypeptide
GGGS

SEQ ID NO: 23 - protein, 5 aa, Artificial Sequence
  Synthetic polypeptide
GSGSG

SEQ ID NO: 24 - protein, 5 aa, Artificial Sequence
  Synthetic polypeptide
GSGGS

SEQ ID NO: 25 - protein, 5 aa, Artificial Sequence
  Synthetic polypeptide
GSGSG

SEQ ID NO: 26 - protein, 4 aa, Artificial Sequence
  Synthetic polypeptide
GGGS

SEQ ID NO: 27 - protein, 4 aa, Artificial sequence
  Synthetic oligonucleotide
GGSG

SEQ ID NO: 28 - protein, 5 aa, Artificial Sequence
  Synthetic polypeptide
GGSGG

SEQ ID NO: 29 - protein, 5 aa, Artificial Sequence
  Synthetic polypeptide
GSGSG

SEQ ID NO: 30 - protein, 5 aa, Artificial Sequence
  Synthetic polypeptide
GSGGG

SEQ ID NO: 31 - protein, 5 aa, Artificial Sequence
  Synthetic polypeptide
GGGSG

SEQ ID NO: 32 - protein, 5 aa, Artificial Sequence
  Synthetic polypeptide
GSSSG

SEQ ID NO: 33 - protein, 6 aa, Homo sapiens
LPNMLR

SEQ ID NO: 34 - protein, 5 aa, Homo sapiens
LRDAF

SEQ ID NO: 35 - protein, 7 aa, Homo sapiens
VKTFFQM

SEQ ID NO: 36 - protein, 21 aa, Homo sapiens
IYKAMSEFDIFINYIEAYMTM

SEQ ID NO: 37 - protein, 18 aa, Homo sapiens
MHSSALLCCLVLLTGVRA

SEQ ID NO: 38 - DNA, 33 bp, Artificial Sequence
  Synthetic oligonucleotide
tatagctagccaccatgcacagctcagcactgc

SEQ ID NO: 39 - DNA, 30 bp, Artificial Sequence
  Synthetic oligonucleotide
tatagggccctcagtttcgtatcttcattg

SEQ ID NO: 40 - DNA, 35 bp, Artificial Sequence
  Synthetic oligonucleotide
ctgactggggtgagggcctgcccaggccagggcac

SEQ ID NO: 41 - DNA, 35 bp, Artificial Sequence
  Synthetic oligonucleotide
gtgccctggcctgggcaggccctcaccccagtcag

SEQ ID NO: 42 - DNA, 33 bp, Artificial Sequence
  Synthetic oligonucleotide
ggggtgagggccagctgcggccagggcacccag

SEQ ID NO: 43 - DNA, 33 bp, Artificial Sequence
  Synthetic oligonucleotide
ctgggtgccctggccgcagctggccctcacccc

SEQ ID NO: 44 - DNA, 34 bp, Artificial Sequence
  Synthetic oligonucleotide
gggtgagggccagcccatgccagggcacccagtc

SEQ ID NO: 45 - DNA, 34 bp, Artificial Sequence
  Synthetic oligonucleotide
gactgggtgccctggcatgggctggccctcaccc

SEQ ID NO: 46 - DNA, 34 bp, Artificial Sequence
  Synthetic oligonucleotide
gagggccagcccaggctgcggcacccagtctgag

SEQ ID NO: 47 - DNA, 34 bp, Artificial Sequence
  Synthetic oligonucleotide
ctcagactgggtgccgcagcctgggctggccctc

SEQ ID NO: 48 - DNA, 35 bp, Artificial Sequence
  Synthetic oligonucleotide
gggccagcccaggccagtgcacccagtctgagaac

SEQ ID NO: 49 - DNA, 35 bp, Artificial Sequence
  Synthetic oligonucleotide
gttctcagactgggtgcactggcctgggctggccc

SEQ ID NO: 50 - DNA, 35 bp, Artificial Sequence
  Synthetic oligonucleotide
ccagcccaggccagggctgccagtctgagaacagc

SEQ ID NO: 51 - DNA, 35 bp, Artificial Sequence
  Synthetic oligonucleotide
gctgttctcagactggcagccctggcctgggctgg

SEQ ID NO: 52 - DNA, 35 bp, Artificial Sequence
  Synthetic oligonucleotide
ccaggccagggcacctgctctgagaacagctgcac

SEQ ID NO: 53 - DNA, 35 bp, Artificial Sequence
  Synthetic oligonucleotide
gtgcagctgttctcagagcaggtgccctggcctgg

SEQ ID NO: 54 - DNA, 34 bp, Artificial Sequence
  Synthetic oligonucleotide
ggccagggcacccagtgtgagaacagctgcaccc

SEQ ID NO: 55 - DNA, 34 bp, Artificial Sequence
  Synthetic oligonucleotide
gggtgcagctgttctcacactgggtgccctggcc

SEQ ID NO: 56 - DNA, 35 bp, Artificial Sequence
  Synthetic oligonucleotide
gccagggcacccagtcttgcaacagctgcacccac

SEQ ID NO: 57 - DNA, 35 bp, Artificial Sequence
  Synthetic oligonucleotide
gtgggtgcagctgttgcaagactgggtgccctggc

SEQ ID NO: 58 - DNA, 35 bp, Artificial Sequence
  Synthetic oligonucleotide
gggcacccagtctgagtgcagctgcacccacttcc

SEQ ID NO: 59 - DNA, 35 bp, Artificial Sequence
  Synthetic oligonucleotide
ggaagtgggtgcagctgcactcagactgggtgccc

SEQ ID NO: 60 - DNA, 34 bp, Artificial Sequence
  Synthetic oligonucleotide
gcacccagtctgagaactgctgcacccacttccc

SEQ ID NO: 61 - DNA, 34 bp, Artificial Sequence
  Synthetic oligonucleotide
gggaagtgggtgcagcagttctcagactgggtgc

SEQ ID NO: 62 - DNA, 35 bp, Artificial Sequence
  Synthetic oligonucleotide
gtctgagaacagctgctgccacttcccaggcaacc

SEQ ID NO: 63 - DNA, 35 bp, Artificial Sequence
  Synthetic oligonucleotide
ggttgcctgggaagtggcagcagctgttctcagac

SEQ ID NO: 64 - DNA, 35 bp, Artificial Sequence
  Synthetic oligonucleotide
gagaacagctgcacctgcttcccaggcaacctgcc

SEQ ID NO: 65 - DNA, 35 bp, Artificial Sequence
  Synthetic oligonucleotide
ggcaggttgcctgggaagcaggtgcagctgttctc

SEQ ID NO: 66 - DNA, 35 bp, Artificial Sequence
  Synthetic oligonucleotide
gctgcacccacttcccatgcaacctgcctaacatg

SEQ ID NO: 67 - DNA, 35 bp, Artificial Sequence
  Synthetic oligonucleotide
catgttaggcaggttgcatgggaagtgggtgcagc

SEQ ID NO: 68 - DNA, 35 bp, Artificial Sequence
  Synthetic oligonucleotide
cacccacttcccaggctgcctgcctaacatgcttc

SEQ ID NO: 69 - DNA, 35 bp, Artificial Sequence
  Synthetic oligonucleotide
gaagcatgttaggcaggcagcctgggaagtgggtg

SEQ ID NO: 70 - DNA, 34 bp, Artificial Sequence
  Synthetic oligonucleotide
gcctaacatgcttcgatgtctccgagatgccttc

SEQ ID NO: 71 - DNA, 34 bp, Artificial Sequence
  Synthetic oligonucleotide
gaaggcatctcggagacatcgaagcatgttaggc

SEQ ID NO: 72 - DNA, 35 bp, Artificial Sequence
  Synthetic oligonucleotide
atctccgagatgccttctgcagagtgaagactttc

SEQ ID NO: 73 - DNA, 35 bp, Artificial Sequence
  Synthetic oligonucleotide
gaaagtcttcactctgcagaaggcatctcggagat

SEQ ID NO: 74 - DNA, 35 bp, Artificial Sequence
  Synthetic oligonucleotide
ccgagatgccttcagctgcgtgaagactttctttc

SEQ ID NO: 75 - DNA, 35 bp, Artificial Sequence
  Synthetic oligonucleotide
gaaagaaagtcttcacgcagctgaaggcatctcgg

SEQ ID NO: 76 - DNA, 35 bp, Artificial Sequence
  Synthetic oligonucleotide
ctttctttcaaatgtgcgatcagctggacaacttg

SEQ ID NO: 77 - DNA, 35 bp, Artificial Sequence
  Synthetic oligonucleotide
caagttgtccagctgatcgcacatttgaaagaaag

SEQ ID NO: 78 - DNA, 35 bp, Artificial Sequence
  Synthetic oligonucleotide
ggacaacttgttgttatgcgagtccttgctggagg

SEQ ID NO: 79 - DNA, 35 bp, Artificial Sequence
  Synthetic oligonucleotide
cctccagcaaggactcgcataacaacaagttgtcc

SEQ ID NO: 80 - DNA, 34 bp, Artificial Sequence
  Synthetic oligonucleotide
caacttgttgttaaagtgctccttgctggaggac

SEQ ID NO: 81 - DNA, 34 bp, Artificial Sequence
  Synthetic oligonucleotide
gtcctccagcaaggagcactttaacaacaagttg

SEQ ID NO: 82 - DNA, 35 bp, Artificial Sequence
  Synthetic oligonucleotide
cttgttgttaaaggagtgcttgctggaggacttta

SEQ ID NO: 83 - DNA, 35 bp, Artificial Sequence
  Synthetic oligonucleotide
taaagtcctccagcaagcactcctttaacaacaag

SEQ ID NO: 84 - DNA, 35 bp, Artificial Sequence
  Synthetic oligonucleotide
ggagtccttgctgtgcgactttaagggttacctgg

SEQ ID NO: 85 - DNA, 35 bp, Artificial Sequence
  Synthetic oligonucleotide
ccaggtaacccttaaagtcgcacagcaaggactcc

SEQ ID NO: 86 - DNA, 35 bp, Artificial Sequence
  Synthetic oligonucleotide
ggagtccttgctggagtgctttaagggttacctgg

SEQ ID NO: 87 - DNA, 35 bp, Artificial Sequence
  Synthetic oligonucleotide
ccaggtaacccttaaagcactccagcaaggactcc

SEQ ID NO: 88 - DNA, 35 bp, Artificial Sequence
  Synthetic oligonucleotide
cttgctggaggacttttgcggttacctgggttgcc

SEQ ID NO: 89 - DNA, 35 bp, Artificial Sequence
  Synthetic oligonucleotide
ggcaacccaggtaaccgcaaaagtcctccagcaag

SEQ ID NO: 90 - DNA, 35 bp, Artificial Sequence
  Synthetic oligonucleotide
gctggaggactttaagtgttacctgggttgccaag

SEQ ID NO: 91 - DNA, 35 bp, Artificial Sequence
  Synthetic oligonucleotide
cttggcaacccaggtaacacttaaagtcctccagc

SEQ ID NO: 92 - DNA, 34 bp, Artificial Sequence
  Synthetic oligonucleotide
ggaggactttaagggttgcctgggttgccaagcc

SEQ ID NO: 93 - DNA, 34 bp, Artificial Sequence
  Synthetic oligonucleotide
ggcttggcaacccaggcaacccttaaagtcctcc

SEQ ID NO: 94 - DNA, 28 bp, Artificial Sequence
  Synthetic oligonucleotide
ctttaagggttactgcggttgccaagcc

SEQ ID NO: 95 - DNA, 28 bp, Artificial Sequence
  Synthetic oligonucleotide
ggcttggcaaccgcagtaacccttaaag

SEQ ID NO: 96 - DNA, 35 bp, Artificial Sequence
  Synthetic oligonucleotide
ctttaagggttacctgtgttgccaagccttgtctg

SEQ ID NO: 97 - DNA, 35 bp, Artificial Sequence
  Synthetic oligonucleotide
cagacaaggcttggcaacacaggtaacccttaaag

SEQ ID NO: 98 - DNA, 34 bp, Artificial Sequence
  Synthetic oligonucleotide
gggttacctgggttgctgcgccttgtctgagatg

SEQ ID NO: 99 - DNA, 34 bp, Artificial Sequence
  Synthetic oligonucleotide
catctcagacaaggcgcagcaacccaggtaaccc

SEQ ID NO: 100 - DNA, 35 bp, Artificial Sequence
  Synthetic oligonucleotide
gttgccaagccttgtgtgagatgatccagttttac

SEQ ID NO: 101 - DNA, 35 bp, Artificial Sequence
  Synthetic oligonucleotide
gtaaaactggatcatctcacacaaggcttggcaac

SEQ ID NO: 102 - DNA, 35 bp, Artificial Sequence
  Synthetic oligonucleotide
gttgccaagccttgtcttgcatgatccagttttac

SEQ ID NO: 103 - DNA, 35 bp, Artificial Sequence
  Synthetic oligonucleotide
gtaaaactggatcatgcaagacaaggcttggcaac

SEQ ID NO: 104 - DNA, 35 bp, Artificial Sequence
  Synthetic oligonucleotide
cttgtctgagatgatctgcttttacctggaggagg

SEQ ID NO: 105 - DNA, 35 bp, Artificial Sequence
  Synthetic oligonucleotide
cctcctccaggtaaaagcagatcatctcagacaag

SEQ ID NO: 106 - DNA, 35 bp, Artificial Sequence
  Synthetic oligonucleotide
gatccagttttacctgtgcgaggtgatgccccaag

SEQ ID NO: 107 - DNA, 35 bp, Artificial Sequence
  Synthetic oligonucleotide
cttggggcatcacctcgcacaggtaaaactggatc

SEQ ID NO: 108 - DNA, 30 bp, Artificial Sequence
  Synthetic oligonucleotide
gttttacctggagtgcgtgatgccccaagc

SEQ ID NO: 109 - DNA, 30 bp, Artificial Sequence
  Synthetic oligonucleotide
gcttggggcatcacgcactccaggtaaaac

SEQ ID NO: 110 - DNA, 35 bp, Artificial Sequence
  Synthetic oligonucleotide
cctggaggaggtgatgtgccaagctgagaaccaag

SEQ ID NO: 111 - DNA, 35 bp, Artificial Sequence
  Synthetic oligonucleotide
cttggttctcagcttggcacatcacctcctccagg

SEQ ID NO: 112 - DNA, 35 bp, Artificial Sequence
  Synthetic oligonucleotide
ggaggaggtgatgccctgcgctgagaaccaagacc

SEQ ID NO: 113 - DNA, 35 bp, Artificial Sequence
  Synthetic oligonucleotide
ggtcttggttctcagcgcagggcatcacctcctcc

SEQ ID NO: 114 - DNA, 34 bp, Artificial Sequence
  Synthetic oligonucleotide
ggtgatgccccaagcttgcaaccaagacccagac

SEQ ID NO: 115 - DNA, 34 bp, Artificial Sequence
  Synthetic oligonucleotide
gtctgggtcttggttgcaagcttggggcatcacc

SEQ ID NO: 116 - DNA, 34 bp, Artificial Sequence
  Synthetic oligonucleotide
gatgccccaagctgagtgccaagacccagacatc

SEQ ID NO: 117 - DNA, 34 bp, Artificial Sequence
  Synthetic oligonucleotide
gatgtctgggtcttggcactcagcttggggcatc

SEQ ID NO: 118 - DNA, 35 bp, Artificial Sequence
  Synthetic oligonucleotide
ccaagctgagaactgcgacccagacatcaaggcgc

SEQ ID NO: 119 - DNA, 35 bp, Artificial Sequence
  Synthetic oligonucleotide
gcgccttgatgtctgggtcgcagttctcagcttgg

SEQ ID NO: 120 - DNA, 34 bp, Artificial Sequence
  Synthetic oligonucleotide
caagctgagaaccaatgcccagacatcaaggcgc

SEQ ID NO: 121 - DNA, 34 bp, Artificial Sequence
  Synthetic oligonucleotide
gcgccttgatgtctgggcattggttctcagcttg

SEQ ID NO: 122 - DNA, 34 bp, Artificial Sequence
  Synthetic oligonucleotide
gctgagaaccaagactgcgacatcaaggcgcatg

SEQ ID NO: 123 - DNA, 34 bp, Artificial Sequence
  Synthetic oligonucleotide
catgcgccttgatgtcgcagtcttggttctcagc

SEQ ID NO: 124 - DNA, 35 bp, Artificial Sequence
  Synthetic oligonucleotide
ctgagaaccaagacccatgcatcaaggcgcatgtg

SEQ ID NO: 125 - DNA, 35 bp, Artificial Sequence
  Synthetic oligonucleotide
cacatgcgccttgatgcatgggtcttggttctcag

SEQ ID NO: 126 - DNA, 35 bp, Artificial Sequence
  Synthetic oligonucleotide
ccaagacccagacatctgcgcgcatgtgaactccc

SEQ ID NO: 127 - DNA, 35 bp, Artificial Sequence
  Synthetic oligonucleotide
gggagttcacatgcgcgcagatgtctgggtcttgg

SEQ ID NO: 128 - DNA, 35 bp, Artificial Sequence
  Synthetic oligonucleotide
gacccagacatcaagtgccatgtgaactccctggg

SEQ ID NO: 129 - DNA, 35 bp, Artificial Sequence
  Synthetic oligonucleotide
cccagggagttcacatggcacttgatgtctgggtc

SEQ ID NO: 130 - DNA, 35 bp, Artificial Sequence
  Synthetic oligonucleotide
cccagacatcaaggcgtgcgtgaactccctggggg

SEQ ID NO: 131 - DNA, 35 bp, Artificial Sequence
  Synthetic oligonucleotide
cccccagggagttcacgcacgccttgatgtctggg

SEQ ID NO: 132 - DNA, 35 bp, Artificial Sequence
  Synthetic oligonucleotide
catcaaggcgcatgtgtgctccctgggggagaacc

SEQ ID NO: 133 - DNA, 35 bp, Artificial Sequence
  Synthetic oligonucleotide
ggttctcccccagggagcacacatgcgccttgatg

SEQ ID NO: 134 - DNA, 34 bp, Artificial Sequence
  Synthetic oligonucleotide
caaggcgcatgtgaactgcctgggggagaacctg

SEQ ID NO: 135 - DNA, 34 bp, Artificial Sequence
  Synthetic oligonucleotide
caggttctcccccaggcagttcacatgcgccttg

SEQ ID NO: 136 - DNA, 35 bp, Artificial Sequence
  Synthetic oligonucleotide
gcatgtgaactccctgtgcgagaacctgaagaccc

SEQ ID NO: 137 - DNA, 35 bp, Artificial Sequence
  Synthetic oligonucleotide
gggtcttcaggttctcgcacagggagttcacatgc

SEQ ID NO: 138 - DNA, 35 bp, Artificial Sequence
  Synthetic oligonucleotide
gtgaactccctggggtgcaacctgaagaccctcag

SEQ ID NO: 139 - DNA, 35 bp, Artificial Sequence
  Synthetic oligonucleotide
ctgagggtcttcaggttgcaccccagggagttcac

SEQ ID NO: 140 - DNA, 35 bp, Artificial Sequence
  Synthetic oligonucleotide
gaactccctgggggagtgcctgaagaccctcaggc

SEQ ID NO: 141 - DNA, 35 bp, Artificial Sequence
  Synthetic oligonucleotide
gcctgagggtcttcaggcactcccccagggagttc

SEQ ID NO: 142 - DNA, 35 bp, Artificial Sequence
  Synthetic oligonucleotide
cctgggggagaacctgtgcaccctcaggctgaggc

SEQ ID NO: 143 - DNA, 35 bp, Artificial Sequence
  Synthetic oligonucleotide
gcctcagcctgagggtgcacaggttctcccccagg

SEQ ID NO: 144 - DNA, 35 bp, Artificial Sequence
  Synthetic oligonucleotide
gggggagaacctgaagtgcctcaggctgaggctac

SEQ ID NO: 145 - DNA, 35 bp, Artificial Sequence
  Synthetic oligonucleotide
gtagcctcagcctgaggcacttcaggttctccccc

SEQ ID NO: 146 - DNA, 34 bp, Artificial Sequence
  Synthetic oligonucleotide
gaacctgaagaccctctgcctgaggctacggcgc

SEQ ID NO: 147 - DNA, 34 bp, Artificial Sequence
  Synthetic oligonucleotide
gcgccgtagcctcaggcagagggtcttcaggttc

SEQ ID NO: 148 - DNA, 35 bp, Artificial Sequence
  Synthetic oligonucleotide
cctgaagaccctcaggtgcaggctacggcgctgtc

SEQ ID NO: 149 - DNA, 35 bp, Artificial Sequence
  Synthetic oligonucleotide
gacagcgccgtagcctgcacctgagggtcttcagg

SEQ ID NO: 150 - DNA, 35 bp, Artificial Sequence
  Synthetic oligonucleotide
gaagaccctcaggctgtgcctacggcgctgtcatc

SEQ ID NO: 151 - DNA, 35 bp, Artificial Sequence
  Synthetic oligonucleotide
gatgacagcgccgtaggcacagcctgagggtcttc

SEQ ID NO: 152 - DNA, 35 bp, Artificial Sequence
  Synthetic oligonucleotide
cctcaggctgaggctatgccgctgtcatcgatttc

SEQ ID NO: 153 - DNA, 35 bp, Artificial Sequence
  Synthetic oligonucleotide
gaaatcgatgacagcggcatagcctcagcctgagg

SEQ ID NO: 154 - DNA, 35 bp, Artificial Sequence
  Synthetic oligonucleotide
caggctgaggctacggtgctgtcatcgatttcttc

SEQ ID NO: 155 - DNA, 35 bp, Artificial Sequence
  Synthetic oligonucleotide
gaagaaatcgatgacagcaccgtagcctcagcctg

SEQ ID NO: 156 - DNA, 35 bp, Artificial Sequence
  Synthetic oligonucleotide
gaggctacggcgctgttgtcgatttcttccctgtg

SEQ ID NO: 157 - DNA, 35 bp, Artificial Sequence
  Synthetic oligonucleotide
cacagggaagaaatcgacaacagcgccgtagcctc

SEQ ID NO: 158 - DNA, 32 bp, Artificial Sequence
  Synthetic oligonucleotide
gctacggcgctgtcattgctttcttccctgtg

SEQ ID NO: 159 - DNA, 32 bp, Artificial Sequence
  Synthetic oligonucleotide
cacagggaagaaagcaatgacagcgccgtagc

SEQ ID NO: 160 - DNA, 35 bp, Artificial Sequence
  Synthetic oligonucleotide
gctgtcatcgatttctttgctgtgaaaacaagagc

SEQ ID NO: 161 - DNA, 35 bp, Artificial Sequence
  Synthetic oligonucleotide
gctcttgttttcacagcaaagaaatcgatgacagc

SEQ ID NO: 162 - DNA, 34 bp, Artificial Sequence
  Synthetic oligonucleotide
cgatttcttccctgttgcaacaagagcaaggccg

SEQ ID NO: 163 - DNA, 34 bp, Artificial Sequence
  Synthetic oligonucleotide
cggccttgctcttgttgcaacagggaagaaatcg

SEQ ID NO: 164 - DNA, 35 bp, Artificial Sequence
  Synthetic oligonucleotide
gatttcttccctgtgaatgcaagagcaaggccgtg

SEQ ID NO: 165 - DNA, 35 bp, Artificial Sequence
  Synthetic oligonucleotide
cacggccttgctcttgcattcacagggaagaaatc

SEQ ID NO: 166 - DNA, 34 bp, Artificial Sequence
  Synthetic oligonucleotide
cttccctgtgaaaactgcagcaaggccgtggagc

SEQ ID NO: 167 - DNA, 34 bp, Artificial Sequence
  Synthetic oligonucleotide
gctccacggccttgctgcagttttcacagggaag

SEQ ID NO: 168 - DNA, 34 bp, Artificial Sequence
  Synthetic oligonucleotide
cttccctgtgaaaacaagtgcaaggccgtggagc

SEQ ID NO: 169 - DNA, 34 bp, Artificial Sequence
  Synthetic oligonucleotide
gctccacggccttgcacttgttttcacagggaag

SEQ ID NO: 170 - DNA, 35 bp, Artificial Sequence
  Synthetic oligonucleotide
cctgtgaaaacaagagctgcgccgtggagcaggtg

SEQ ID NO: 171 - DNA, 35 bp, Artificial Sequence
  Synthetic oligonucleotide
cacctgctccacggcgcagctcttgttttcacagg

SEQ ID NO: 172 - DNA, 35 bp, Artificial Sequence
  Synthetic oligonucleotide
gagcaaggccgtgtgccaggtgaagaatgccttta

SEQ ID NO: 173 - DNA, 35 bp, Artificial Sequence
  Synthetic oligonucleotide
taaaggcattcttcacctggcacacggccttgctc

SEQ ID NO: 174 - DNA, 35 bp, Artificial Sequence
  Synthetic oligonucleotide
gagcaaggccgtggagtgcgtgaagaatgccttta

SEQ ID NO: 175 - DNA, 35 bp, Artificial Sequence
  Synthetic oligonucleotide
taaaggcattcttcacgcactccacggccttgctc

SEQ ID NO: 176 - DNA, 47 bp, Artificial Sequence
  Synthetic oligonucleotide
gagcaaggccgtggagcaggtgtgcaatgcctttaataagctccaag

SEQ ID NO: 177 - DNA, 47 bp, Artificial Sequence
  Synthetic oligonucleotide
cttggagcttattaaaggcattgcacacctgctccacggccttgctc

SEQ ID NO: 178 - DNA, 35 bp, Artificial Sequence
  Synthetic oligonucleotide
cgtggagcaggtgaagtgcgcctttaataagctcc

SEQ ID NO: 179 - DNA, 35 bp, Artificial Sequence
  Synthetic oligonucleotide
ggagcttattaaaggcgcacttcacctgctccacg

SEQ ID NO: 180 - DNA, 35 bp, Artificial Sequence
  Synthetic oligonucleotide
ggtgaagaatgccttttgtaagctccaagagaaag

SEQ ID NO: 181 - DNA, 35 bp, Artificial Sequence
  Synthetic oligonucleotide
ctttctcttggagcttacaaaaggcattcttcacc

SEQ ID NO: 182 - DNA, 34 bp, Artificial Sequence
  Synthetic oligonucleotide
gaagaatgcctttaattgcctccaagagaaaggc

SEQ ID NO: 183 - DNA, 34 bp, Artificial Sequence
  Synthetic oligonucleotide
gcctttctcttggaggcaattaaaggcattcttc

SEQ ID NO: 184 - DNA, 33 bp, Artificial Sequence
  Synthetic oligonucleotide
gcctttaataagctctgcgagaaaggcatctac

SEQ ID NO: 185 - DNA, 33 bp, Artificial Sequence
  Synthetic oligonucleotide
gtagatgcctttctcgcagagcttattaaaggc

SEQ ID NO: 186 - DNA, 35 bp, Artificial Sequence
  Synthetic oligonucleotide
ctttaataagctccaatgcaaaggcatctacaaag

SEQ ID NO: 187 - DNA, 35 bp, Artificial Sequence
  Synthetic oligonucleotide
ctttgtagatgcctttgcattggagcttattaaag

SEQ ID NO: 188 - DNA, 35 bp, Artificial Sequence
  Synthetic oligonucleotide
ctttaataagctccaagagtgcggcatctacaaag

SEQ ID NO: 189 - DNA, 35 bp, Artificial Sequence
  Synthetic oligonucleotide
ctttgtagatgccgcactcttggagcttattaaag

SEQ ID NO: 190 - DNA, 35 bp, Artificial Sequence
  Synthetic oligonucleotide
gctccaagagaaatgcatctacaaagccatgagtg

SEQ ID NO: 191 - DNA, 35 bp, Artificial Sequence
  Synthetic oligonucleotide
cactcatggctttgtagatgcatttctcttggagc

SEQ ID NO: 192 - DNA, 35 bp, Artificial Sequence
  Synthetic oligonucleotide
gcctacatgacaatgtgcatacgaaactgagggcc

SEQ ID NO: 193 - DNA, 35 bp, Artificial Sequence
  Synthetic oligonucleotide
ggccctcagtttcgtatgcacattgtcatgtaggc

SEQ ID NO: 194 - DNA, 35 bp, Artificial Sequence
  Synthetic oligonucleotide
catgacaatgaagatacgatgctgagggcccgaac

SEQ ID NO: 195 - DNA, 35 bp, Artificial Sequence
  Synthetic oligonucleotide
gttcgggccctcagcatcgtatcttcattgtcatg

SEQ ID NO: 196 - DNA, 35 bp, Artificial Sequence
  Synthetic oligonucleotide
gactggggtgagggcctacccaggccagggcaccc

SEQ ID NO: 197 - DNA, 35 bp, Artificial Sequence
  Synthetic oligonucleotide
gggtgccctggcctgggtaggccctcaccccagtc

SEQ ID NO: 198 - DNA, 35 bp, Artificial Sequence
  Synthetic oligonucleotide
ctggggtgagggccagctacggccagggcacccag

SEQ ID NO: 199 - DNA, 35 bp, Artificial Sequence
  Synthetic oligonucleotide
ctgggtgccctggccgtagctggccctcaccccag

SEQ ID NO: 200 - DNA, 35 bp, Artificial Sequence
  Synthetic oligonucleotide
ggtgagggccagcccataccagggcacccagtctg

SEQ ID NO: 201 - DNA, 35 bp, Artificial Sequence
  Synthetic oligonucleotide
cagactgggtgccctggtatgggctggccctcacc

SEQ ID NO: 202 - DNA, 34 bp, Artificial Sequence
  Synthetic oligonucleotide
gagggccagcccaggctacggcacccagtctgag

SEQ ID NO: 203 - DNA, 34 bp, Artificial Sequence
  Synthetic oligonucleotide
ctcagactgggtgccgtagcctgggctggccctc

SEQ ID NO: 204 - DNA, 35 bp, Artificial Sequence
  Synthetic oligonucleotide
gggccagcccaggccagtacacccagtctgagaac

SEQ ID NO: 205 - DNA, 35 bp, Artificial Sequence
  Synthetic oligonucleotide
gttctcagactgggtgtactggcctgggctggccc

SEQ ID NO: 206 - DNA, 35 bp, Artificial Sequence
  Synthetic oligonucleotide
ccagcccaggccagggctaccagtctgagaacagc

SEQ ID NO: 207 - DNA, 35 bp, Artificial Sequence
  Synthetic oligonucleotide
gctgttctcagactggtagccctggcctgggctgg

SEQ ID NO: 208 - DNA, 35 bp, Artificial Sequence
  Synthetic oligonucleotide
gcccaggccagggcacctactctgagaacagctgc

SEQ ID NO: 209 - DNA, 35 bp, Artificial Sequence
  Synthetic oligonucleotide
gcagctgttctcagagtaggtgccctggcctgggc

SEQ ID NO: 210 - DNA, 34 bp, Artificial Sequence
  Synthetic oligonucleotide
ggccagggcacccagtacgagaacagctgcaccc

SEQ ID NO: 211 - DNA, 34 bp, Artificial Sequence
  Synthetic oligonucleotide
gggtgcagctgttctcgtactgggtgccctggcc

SEQ ID NO: 212 - DNA, 35 bp, Artificial Sequence
  Synthetic oligonucleotide
gccagggcacccagtcttacaacagctgcacccac

SEQ ID NO: 213 - DNA, 35 bp, Artificial Sequence
  Synthetic oligonucleotide
gtgggtgcagctgttgtaagactgggtgccctggc

SEQ ID NO: 214 - DNA, 35 bp, Artificial Sequence
  Synthetic oligonucleotide
gggcacccagtctgagtacagctgcacccacttcc

SEQ ID NO: 215 - DNA, 35 bp, Artificial Sequence
  Synthetic oligonucleotide
ggaagtgggtgcagctgtactcagactgggtgccc

SEQ ID NO: 216 - DNA, 35 bp, Artificial Sequence
  Synthetic oligonucleotide
ggcacccagtctgagaactactgcacccacttccc

SEQ ID NO: 217 - DNA, 35 bp, Artificial Sequence
  Synthetic oligonucleotide
gggaagtgggtgcagtagttctcagactgggtgcc

SEQ ID NO: 218 - DNA, 35 bp, Artificial Sequence
  Synthetic oligonucleotide
gtctgagaacagctgctaccacttcccaggcaacc

SEQ ID NO: 219 - DNA, 35 bp, Artificial Sequence
  Synthetic oligonucleotide
ggttgcctgggaagtggtagcagctgttctcagac

SEQ ID NO: 220 - DNA, 35 bp, Artificial Sequence
  Synthetic oligonucleotide
ctgagaacagctgcacctacttcccaggcaacctg

SEQ ID NO: 221 - DNA, 35 bp, Artificial Sequence
  Synthetic oligonucleotide
caggttgcctgggaagtaggtgcagctgttctcag

SEQ ID NO: 222 - DNA, 35 bp, Artificial Sequence
  Synthetic oligonucleotide
ctgcacccacttcccatacaacctgcctaacatgc

SEQ ID NO: 223 - DNA, 35 bp, Artificial Sequence
  Synthetic oligonucleotide
gcatgttaggcaggttgtatgggaagtgggtgcag

SEQ ID NO: 224 - DNA, 35 bp, Artificial Sequence
  Synthetic oligonucleotide
cacccacttcccaggctacctgcctaacatgcttc

SEQ ID NO: 225 - DNA, 35 bp, Artificial Sequence
  Synthetic oligonucleotide
gaagcatgttaggcaggtagcctgggaagtgggtg

SEQ ID NO: 226 - DNA, 34 bp, Artificial Sequence
  Synthetic oligonucleotide
gcctaacatgcttcgatacctccgagatgccttc

SEQ ID NO: 227 - DNA, 34 bp, Artificial Sequence
  Synthetic oligonucleotide
gaaggcatctcggaggtatcgaagcatgttaggc

SEQ ID NO: 228 - DNA, 36 bp, Artificial Sequence
  Synthetic oligonucleotide
gatctccgagatgccttctacagagtgaagactttc

SEQ ID NO: 229 - DNA, 36 bp, Artificial Sequence
  Synthetic oligonucleotide
gaaagtcttcactctgtagaaggcatctcggagatc

SEQ ID NO: 230 - DNA, 35 bp, Artificial Sequence
  Synthetic oligonucleotide
ccgagatgccttcagctacgtgaagactttctttc

SEQ ID NO: 231 - DNA, 35 bp, Artificial Sequence
  Synthetic oligonucleotide
gaaagaaagtcttcacgtagctgaaggcatctcgg

SEQ ID NO: 232 - DNA, 34 bp, Artificial Sequence
  Synthetic oligonucleotide
gactttctttcaaatgtacgatcagctggacaac

SEQ ID NO: 233 - DNA, 34 bp, Artificial Sequence
  Synthetic oligonucleotide
gttgtccagctgatcgtacatttgaaagaaagtc

SEQ ID NO: 234 - DNA, 35 bp, Artificial Sequence
  Synthetic oligonucleotide
ggacaacttgttgttatacgagtccttgctggagg

SEQ ID NO: 235 - DNA, 35 bp, Artificial Sequence
  Synthetic oligonucleotide
cctccagcaaggactcgtataacaacaagttgtcc

SEQ ID NO: 236 - DNA, 34 bp, Artificial Sequence
  Synthetic oligonucleotide
caacttgttgttaaagtactccttgctggaggac

SEQ ID NO: 237 - DNA, 34 bp, Artificial Sequence
  Synthetic oligonucleotide
gtcctccagcaaggagtactttaacaacaagttg

SEQ ID NO: 238 - DNA, 38 bp, Artificial Sequence
  Synthetic oligonucleotide
cttgttgttaaaggagtacttgctggaggactttaagg

SEQ ID NO: 239 - DNA, 38 bp, Artificial Sequence
  Synthetic oligonucleotide
ccttaaagtcctccagcaagtactcctttaacaacaag

SEQ ID NO: 240 - DNA, 35 bp, Artificial Sequence
  Synthetic oligonucleotide
aaaggagtccttgctgtacgactttaagggttacc

SEQ ID NO: 241 - DNA, 35 bp, Artificial Sequence
  Synthetic oligonucleotide
ggtaacccttaaagtcgtacagcaaggactccttt

SEQ ID NO: 242 - DNA, 35 bp, Artificial Sequence
  Synthetic oligonucleotide
ggagtccttgctggagtactttaagggttacctgg

SEQ ID NO: 243 - DNA, 35 bp, Artificial Sequence
  Synthetic oligonucleotide
ccaggtaacccttaaagtactccagcaaggactcc

SEQ ID NO: 244 - DNA, 35 bp, Artificial Sequence
  Synthetic oligonucleotide
cttgctggaggacttttacggttacctgggttgcc

SEQ ID NO: 245 - DNA, 35 bp, Artificial Sequence
  Synthetic oligonucleotide
ggcaacccaggtaaccgtaaaagtcctccagcaag

SEQ ID NO: 246 - DNA, 35 bp, Artificial Sequence
  Synthetic oligonucleotide
gctggaggactttaagtactacctgggttgccaag

SEQ ID NO: 247 - DNA, 35 bp, Artificial Sequence
  Synthetic oligonucleotide
cttggcaacccaggtagtacttaaagtcctccagc

SEQ ID NO: 248 - DNA, 34 bp, Artificial Sequence
  Synthetic oligonucleotide
ggactttaagggttactacggttgccaagccttg

SEQ ID NO: 249 - DNA, 34 bp, Artificial Sequence
  Synthetic oligonucleotide
caaggcttggcaaccgtagtaacccttaaagtcc

SEQ ID NO: 250 - DNA, 35 bp, Artificial Sequence
  Synthetic oligonucleotide
ctttaagggttacctgtactgccaagccttgtctg

SEQ ID NO: 251 - DNA, 35 bp, Artificial Sequence
  Synthetic oligonucleotide
cagacaaggcttggcagtacaggtaacccttaaag

SEQ ID NO: 252 - DNA, 34 bp, Artificial Sequence
  Synthetic oligonucleotide
gggttacctgggttgctacgccttgtctgagatg

SEQ ID NO: 253 - DNA, 34 bp, Artificial Sequence
  Synthetic oligonucleotide
catctcagacaaggcgtagcaacccaggtaaccc

SEQ ID NO: 254 - DNA, 35 bp, Artificial Sequence
  Synthetic oligonucleotide
gttgccaagccttgtacgagatgatccagttttac

SEQ ID NO: 255 - DNA, 35 bp, Artificial Sequence
  Synthetic oligonucleotide
gtaaaactggatcatctcgtacaaggcttggcaac

SEQ ID NO: 256 - DNA, 35 bp, Artificial Sequence
  Synthetic oligonucleotide
gttgccaagccttgtcttacatgatccagttttac

SEQ ID NO: 257 - DNA, 35 bp, Artificial Sequence
  Synthetic oligonucleotide
gtaaaactggatcatgtaagacaaggcttggcaac

SEQ ID NO: 258 - DNA, 35 bp, Artificial Sequence
  Synthetic oligonucleotide
cttgtctgagatgatctacttttacctggaggagg

SEQ ID NO: 259 - DNA, 35 bp, Artificial Sequence
  Synthetic oligonucleotide
cctcctccaggtaaaagtagatcatctcagacaag

SEQ ID NO: 260 - DNA, 35 bp, Artificial Sequence
  Synthetic oligonucleotide
gatccagttttacctgtacgaggtgatgccccaag

SEQ ID NO: 261 - DNA, 35 bp, Artificial Sequence
  Synthetic oligonucleotide
cttggggcatcacctcgtacaggtaaaactggatc

SEQ ID NO: 262 - DNA, 35 bp, Artificial Sequence
  Synthetic oligonucleotide
ccagttttacctggagtacgtgatgccccaagctg

SEQ ID NO: 263 - DNA, 35 bp, Artificial Sequence
  Synthetic oligonucleotide
cagcttggggcatcacgtactccaggtaaaactgg

SEQ ID NO: 264 - DNA, 35 bp, Artificial Sequence
  Synthetic oligonucleotide
cctggaggaggtgatgtaccaagctgagaaccaag

SEQ ID NO: 265 - DNA, 35 bp, Artificial Sequence
  Synthetic oligonucleotide
cttggttctcagcttggtacatcacctcctccagg

SEQ ID NO: 266 - DNA, 35 bp, Artificial Sequence
  Synthetic oligonucleotide
ggaggaggtgatgccctacgctgagaaccaagacc

SEQ ID NO: 267 - DNA, 35 bp, Artificial Sequence
  Synthetic oligonucleotide
ggtcttggttctcagcgtagggcatcacctcctcc

SEQ ID NO: 268 - DNA, 34 bp, Artificial Sequence
  Synthetic oligonucleotide
ggtgatgccccaagcttacaaccaagacccagac

SEQ ID NO: 269 - DNA, 34 bp, Artificial Sequence
  Synthetic oligonucleotide
gtctgggtcttggttgtaagcttggggcatcacc

SEQ ID NO: 270 - DNA, 34 bp, Artificial Sequence
  Synthetic oligonucleotide
gatgccccaagctgagtaccaagacccagacatc

SEQ ID NO: 271 - DNA, 34 bp, Artificial Sequence
  Synthetic oligonucleotide
gatgtctgggtcttggtactcagcttggggcatc

SEQ ID NO: 272 - DNA, 35 bp, Artificial Sequence
  Synthetic oligonucleotide
gccccaagctgagaactacgacccagacatcaagg

SEQ ID NO: 273 - DNA, 35 bp, Artificial Sequence
  Synthetic oligonucleotide
ccttgatgtctgggtcgtagttctcagcttggggc

SEQ ID NO: 274 - DNA, 35 bp, Artificial Sequence
  Synthetic oligonucleotide
ccaagctgagaaccaatacccagacatcaaggcgc

SEQ ID NO: 275 - DNA, 35 bp, Artificial Sequence
  Synthetic oligonucleotide
gcgccttgatgtctgggtattggttctcagcttgg

SEQ ID NO: 276 - DNA, 34 bp, Artificial Sequence
  Synthetic oligonucleotide
gctgagaaccaagactacgacatcaaggcgcatg

SEQ ID NO: 277 - DNA, 34 bp, Artificial Sequence
  Synthetic oligonucleotide
catgcgccttgatgtcgtagtcttggttctcagc

SEQ ID NO: 278 - DNA, 35 bp, Artificial Sequence
  Synthetic oligonucleotide
ctgagaaccaagacccatacatcaaggcgcatgtg

SEQ ID NO: 279 - DNA, 35 bp, Artificial Sequence
  Synthetic oligonucleotide
cacatgcgccttgatgtatgggtcttggttctcag

SEQ ID NO: 280 - DNA, 35 bp, Artificial Sequence
  Synthetic oligonucleotide
ccaagacccagacatctacgcgcatgtgaactccc

SEQ ID NO: 281 - DNA, 35 bp, Artificial Sequence
  Synthetic oligonucleotide
gggagttcacatgcgcgtagatgtctgggtcttgg

SEQ ID NO: 282 - DNA, 35 bp, Artificial Sequence
  Synthetic oligonucleotide
gacccagacatcaagtaccatgtgaactccctggg

SEQ ID NO: 283 - DNA, 35 bp, Artificial Sequence
  Synthetic oligonucleotide
cccagggagttcacatggtacttgatgtctgggtc

SEQ ID NO: 284 - DNA, 35 bp, Artificial Sequence
  Synthetic oligonucleotide
cccagacatcaaggcgtacgtgaactccctggggg

SEQ ID NO: 285 - DNA, 35 bp, Artificial Sequence
  Synthetic oligonucleotide
cccccagggagttcacgtacgccttgatgtctggg

SEQ ID NO: 286 - DNA, 23 bp, Artificial Sequence
  Synthetic oligonucleotide
ggcgcatgtgtactccctggggg

SEQ ID NO: 287 - DNA, 23 bp, Artificial Sequence
  Synthetic oligonucleotide
cccccagggagtacacatgcgcc

SEQ ID NO: 288 - DNA, 28 bp, Artificial Sequence
  Synthetic oligonucleotide
ggcgcatgtgaactacctgggggagaac

SEQ ID NO: 289 - DNA, 28 bp, Artificial Sequence
  Synthetic oligonucleotide
gttctcccccaggtagttcacatgcgcc

SEQ ID NO: 290 - DNA, 35 bp, Artificial Sequence
  Synthetic oligonucleotide
gcatgtgaactccctgtacgagaacctgaagaccc

SEQ ID NO: 291 - DNA, 35 bp, Artificial Sequence
  Synthetic oligonucleotide
gggtcttcaggttctcgtacagggagttcacatgc

SEQ ID NO: 292 - DNA, 35 bp, Artificial Sequence
  Synthetic oligonucleotide
gtgaactccctggggtacaacctgaagaccctcag

SEQ ID NO: 293 - DNA, 35 bp, Artificial Sequence
  Synthetic oligonucleotide
ctgagggtcttcaggttgtaccccagggagttcac

SEQ ID NO: 294 - DNA, 35 bp, Artificial Sequence
  Synthetic oligonucleotide
gaactccctgggggagtacctgaagaccctcaggc

SEQ ID NO: 295 - DNA, 35 bp, Artificial Sequence
  Synthetic oligonucleotide
gcctgagggtcttcaggtactcccccagggagttc

SEQ ID NO: 296 - DNA, 35 bp, Artificial Sequence
  Synthetic oligonucleotide
cctgggggagaacctgtacaccctcaggctgaggc

SEQ ID NO: 297 - DNA, 35 bp, Artificial Sequence
  Synthetic oligonucleotide
gcctcagcctgagggtgtacaggttctcccccagg

SEQ ID NO: 298 - DNA, 28 bp, Artificial Sequence
  Synthetic oligonucleotide
ggagaacctgaagtacctcaggctgagg

SEQ ID NO: 299 - DNA, 28 bp, Artificial Sequence
  Synthetic oligonucleotide
cctcagcctgaggtacttcaggttctcc

SEQ ID NO: 300 - DNA, 34 bp, Artificial Sequence
  Synthetic oligonucleotide
gaacctgaagaccctctacctgaggctacggcgc

SEQ ID NO: 301 - DNA, 34 bp, Artificial Sequence
  Synthetic oligonucleotide
gcgccgtagcctcaggtagagggtcttcaggttc

SEQ ID NO: 302 - DNA, 35 bp, Artificial Sequence
  Synthetic oligonucleotide
cctgaagaccctcaggtacaggctacggcgctgtc

SEQ ID NO: 303 - DNA, 35 bp, Artificial Sequence
  Synthetic oligonucleotide
gacagcgccgtagcctgtacctgagggtcttcagg

SEQ ID NO: 304 - DNA, 35 bp, Artificial Sequence
  Synthetic oligonucleotide
gaagaccctcaggctgtacctacggcgctgtcatc

SEQ ID NO: 305 - DNA, 35 bp, Artificial Sequence
  Synthetic oligonucleotide
gatgacagcgccgtaggtacagcctgagggtcttc

SEQ ID NO: 306 - DNA, 35 bp, Artificial Sequence
  Synthetic oligonucleotide
cctcaggctgaggctataccgctgtcatcgatttc

SEQ ID NO: 307 - DNA, 35 bp, Artificial Sequence
  Synthetic oligonucleotide
gaaatcgatgacagcggtatagcctcagcctgagg

SEQ ID NO: 308 - DNA, 35 bp, Artificial Sequence
  Synthetic oligonucleotide
caggctgaggctacggtactgtcatcgatttcttc

SEQ ID NO: 309 - DNA, 35 bp, Artificial Sequence
  Synthetic oligonucleotide
gaagaaatcgatgacagtaccgtagcctcagcctg

SEQ ID NO: 310 - DNA, 35 bp, Artificial Sequence
  Synthetic oligonucleotide
gaggctacggcgctgttaccgatttcttccctgtg

SEQ ID NO: 311 - DNA, 35 bp, Artificial Sequence
  Synthetic oligonucleotide
cacagggaagaaatcggtaacagcgccgtagcctc

SEQ ID NO: 312 - DNA, 37 bp, Artificial Sequence
  Synthetic oligonucleotide
gctacggcgctgtcattactttcttccctgtgaaaac

SEQ ID NO: 313 - DNA, 37 bp, Artificial Sequence
  Synthetic oligonucleotide
gttttcacagggaagaaagtaatgacagcgccgtagc

SEQ ID NO: 314 - DNA, 35 bp, Artificial Sequence
  Synthetic oligonucleotide
gctgtcatcgatttctttactgtgaaaacaagagc

SEQ ID NO: 315 - DNA, 35 bp, Artificial Sequence
  Synthetic oligonucleotide
gctcttgttttcacagtaaagaaatcgatgacagc

SEQ ID NO: 316 - DNA, 34 bp, Artificial Sequence
  Synthetic oligonucleotide
cgatttcttccctgttacaacaagagcaaggccg

SEQ ID NO: 317 - DNA, 34 bp, Artificial Sequence
  Synthetic oligonucleotide
cggccttgctcttgttgtaacagggaagaaatcg

SEQ ID NO: 318 - DNA, 35 bp, Artificial Sequence
  Synthetic oligonucleotide
gatttcttccctgtgaatacaagagcaaggccgtg

SEQ ID NO: 319 - DNA, 35 bp, Artificial Sequence
  Synthetic oligonucleotide
cacggccttgctcttgtattcacagggaagaaatc

SEQ ID NO: 320 - DNA, 34 bp, Artificial Sequence
  Synthetic oligonucleotide
cttccctgtgaaaactacagcaaggccgtggagc

SEQ ID NO: 321 - DNA, 34 bp, Artificial Sequence
  Synthetic oligonucleotide
gctccacggccttgctgtagttttcacagggaag

SEQ ID NO: 322 - DNA, 34 bp, Artificial Sequence
  Synthetic oligonucleotide
ccctgtgaaaacaagtacaaggccgtggagcagg

SEQ ID NO: 323 - DNA, 34 bp, Artificial Sequence
  Synthetic oligonucleotide
cctgctccacggccttgtacttgttttcacaggg

SEQ ID NO: 324 - DNA, 34 bp, Artificial Sequence
  Synthetic oligonucleotide
ctgtgaaaacaagagctacgccgtggagcaggtg

SEQ ID NO: 325 - DNA, 34 bp, Artificial Sequence
  Synthetic oligonucleotide
cacctgctccacggcgtagctcttgttttcacag

SEQ ID NO: 326 - DNA, 34 bp, Artificial Sequence
  Synthetic oligonucleotide
caagagcaaggccgtgtaccaggtgaagaatgcc

SEQ ID NO: 327 - DNA, 34 bp, Artificial Sequence
  Synthetic oligonucleotide
ggcattcttcacctggtacacggccttgctcttg

SEQ ID NO: 328 - DNA, 35 bp, Artificial Sequence
  Synthetic oligonucleotide
gagcaaggccgtggagtacgtgaagaatgccttta

SEQ ID NO: 329 - DNA, 35 bp, Artificial Sequence
  Synthetic oligonucleotide
taaaggcattcttcacgtactccacggccttgctc

SEQ ID NO: 330 - DNA, 41 bp, Artificial Sequence
  Synthetic oligonucleotide
caaggccgtggagcaggtgtacaatgcctttaataagctcc

SEQ ID NO: 331 - DNA, 41 bp, Artificial Sequence
  Synthetic oligonucleotide
ggagcttattaaaggcattgtacacctgctccacggccttg

SEQ ID NO: 332 - DNA, 35 bp, Artificial Sequence
  Synthetic oligonucleotide
cgtggagcaggtgaagtacgcctttaataagctcc

SEQ ID NO: 333 - DNA, 35 bp, Artificial Sequence
  Synthetic oligonucleotide
ggagcttattaaaggcgtacttcacctgctccacg

SEQ ID NO: 334 - DNA, 28 bp, Artificial Sequence
  Synthetic oligonucleotide
gaagaatgccttttacaagctccaagag

SEQ ID NO: 335 - DNA, 28 bp, Artificial Sequence
  Synthetic oligonucleotide
ctcttggagcttgtaaaaggcattcttc

SEQ ID NO: 336 - DNA, 34 bp, Artificial Sequence
  Synthetic oligonucleotide
gaagaatgcctttaattacctccaagagaaaggc

SEQ ID NO: 337 - DNA, 34 bp, Artificial Sequence
  Synthetic oligonucleotide
gcctttctcttggaggtaattaaaggcattcttc

SEQ ID NO: 338 - DNA, 33 bp, Artificial Sequence
  Synthetic oligonucleotide
gcctttaataagctctacgagaaaggcatctac

SEQ ID NO: 339 - DNA, 33 bp, Artificial Sequence
  Synthetic oligonucleotide
gtagatgcctttctcgtagagcttattaaaggc

SEQ ID NO: 340 - DNA, 35 bp, Artificial Sequence
  Synthetic oligonucleotide
ctttaataagctccaatacaaaggcatctacaaag

SEQ ID NO: 341 - DNA, 35 bp, Artificial Sequence
  Synthetic oligonucleotide
ctttgtagatgcctttgtattggagcttattaaag

SEQ ID NO: 342 - DNA, 37 bp, Artificial Sequence
  Synthetic oligonucleotide
ctttaataagctccaagagtacggcatctacaaagcc

SEQ ID NO: 343 - DNA, 37 bp, Artificial Sequence
  Synthetic oligonucleotide
ggctttgtagatgccgtactcttggagcttattaaag

SEQ ID NO: 344 - DNA, 35 bp, Artificial Sequence
  Synthetic oligonucleotide
gctccaagagaaatacatctacaaagccatgagtg

SEQ ID NO: 345 - DNA, 35 bp, Artificial Sequence
  Synthetic oligonucleotide
cactcatggctttgtagatgtatttctcttggagc

SEQ ID NO: 346 - DNA, 35 bp, Artificial Sequence
  Synthetic oligonucleotide
gcctacatgacaatgtacatacgaaactgagggcc

SEQ ID NO: 347 - DNA, 35 bp, Artificial Sequence
  Synthetic oligonucleotide
ggccctcagtttcgtatgtacattgtcatgtaggc

SEQ ID NO: 348 - DNA, 35 bp, Artificial Sequence
  Synthetic oligonucleotide
catgacaatgaagatacgatactgagggcccgaac

SEQ ID NO: 349 - DNA, 35 bp, Artificial Sequence
  Synthetic oligonucleotide
gttcgggccctcagtatcgtatcttcattgtcatg

SEQ ID NO: 350 - DNA, 35 bp, Artificial Sequence
  Synthetic oligonucleotide
gactggggtgagggccaacccaggccagggcaccc

SEQ ID NO: 351 - DNA, 35 bp, Artificial Sequence
  Synthetic oligonucleotide
gggtgccctggcctgggttggccctcaccccagtc

SEQ ID NO: 352 - DNA, 34 bp, Artificial Sequence
  Synthetic oligonucleotide
gggtgagggccaacccaagccagggcacccagtc

SEQ ID NO: 353 - DNA, 34 bp, Artificial Sequence
  Synthetic oligonucleotide
gactgggtgccctggcttgggttggccctcaccc

SEQ ID NO: 354 - DNA, 33 bp, Artificial Sequence
  Synthetic oligonucleotide
ggggtgagggccaacggtagccagggcacccag

SEQ ID NO: 355 - DNA, 33 bp, Artificial Sequence
  Synthetic oligonucleotide
ctgggtgccctggctaccgttggccctcacccc

SEQ ID NO: 356 - DNA, 35 bp, Artificial Sequence
  Synthetic oligonucleotide
ggtgagggccaacccaacccagggcacccagtctg

SEQ ID NO: 357 - DNA, 35 bp, Artificial Sequence
  Synthetic oligonucleotide
cagactgggtgccctgggttgggttggccctcacc

SEQ ID NO: 358 - DNA, 29 bp, Artificial Sequence
  Synthetic oligonucleotide
ggtgagggccaacggtacccagggcaccc

SEQ ID NO: 359 - DNA, 29 bp, Artificial Sequence
  Synthetic oligonucleotide
gggtgccctgggtaccgttggccctcacc

SEQ ID NO: 360 - DNA, 35 bp, Artificial Sequence
  Synthetic oligonucleotide
ggggtgagggccagcaacggccagggcacccagtc

SEQ ID NO: 361 - DNA, 35 bp, Artificial Sequence
  Synthetic oligonucleotide
gactgggtgccctggccgttgctggccctcacccc

SEQ ID NO: 362 - DNA, 35 bp, Artificial Sequence
  Synthetic oligonucleotide
gggccagcaacggcagcggcacccagtctgagaac

SEQ ID NO: 363 - DNA, 35 bp, Artificial Sequence
  Synthetic oligonucleotide
gttctcagactgggtgccgctgccgttgctggccc

SEQ ID NO: 364 - DNA, 34 bp, Artificial Sequence
  Synthetic oligonucleotide
gagggccagcaacggcaccggcacccagtctgag

SEQ ID NO: 365 - DNA, 34 bp, Artificial Sequence
  Synthetic oligonucleotide
ctcagactgggtgccggtgccgttgctggccctc

SEQ ID NO: 366 - DNA, 35 bp, Artificial Sequence
  Synthetic oligonucleotide
ggtgagggccagcccaaaccagggcacccagtctg

SEQ ID NO: 367 - DNA, 35 bp, Artificial Sequence
  Synthetic oligonucleotide
cagactgggtgccctggtttgggctggccctcacc

SEQ ID NO: 368 - DNA, 35 bp, Artificial Sequence
  Synthetic oligonucleotide
gggccagcccaaaccagagcacccagtctgagaac

SEQ ID NO: 369 - DNA, 35 bp, Artificial Sequence
  Synthetic oligonucleotide
gttctcagactgggtgctctggtttgggctggccc

SEQ ID NO: 370 - DNA, 35 bp, Artificial Sequence
  Synthetic oligonucleotide
gggccagcccaaaccagaccacccagtctgagaac

SEQ ID NO: 371 - DNA, 35 bp, Artificial Sequence
  Synthetic oligonucleotide
gttctcagactgggtggtctggtttgggctggccc

SEQ ID NO: 372 - DNA, 34 bp, Artificial Sequence
  Synthetic oligonucleotide
gagggccagcccaggcaacggcacccagtctgag

SEQ ID NO: 373 - DNA, 34 bp, Artificial Sequence
  Synthetic oligonucleotide
ctcagactgggtgccgttgcctgggctggccctc

SEQ ID NO: 374 - DNA, 34 bp, Artificial Sequence
  Synthetic oligonucleotide
cagcccaggcaacggcagccagtctgagaacagc

SEQ ID NO: 375 - DNA, 34 bp, Artificial Sequence
  Synthetic oligonucleotide
gctgttctcagactggctgccgttgcctgggctg

SEQ ID NO: 376 - DNA, 35 bp, Artificial Sequence
  Synthetic oligonucleotide
gggccagcccaggccagaacacccagtctgagaac

SEQ ID NO: 377 - DNA, 35 bp, Artificial Sequence
  Synthetic oligonucleotide
gttctcagactgggtgttctggcctgggctggccc

SEQ ID NO: 378 - DNA, 35 bp, Artificial Sequence
  Synthetic oligonucleotide
gcccaggccagaacaccagctctgagaacagctgc

SEQ ID NO: 379 - DNA, 35 bp, Artificial Sequence
  Synthetic oligonucleotide
gcagctgttctcagagctggtgttctggcctgggc

SEQ ID NO: 380 - DNA, 36 bp, Artificial Sequence
  Synthetic oligonucleotide
cccaggccagaacaccacctctgagaacagctgcac

SEQ ID NO: 381 - DNA, 36 bp, Artificial Sequence
  Synthetic oligonucleotide
gtgcagctgttctcagaggtggtgttctggcctggg

SEQ ID NO: 382 - DNA, 35 bp, Artificial Sequence
  Synthetic oligonucleotide
ccagcccaggccagggcaaccagtctgagaacagc

SEQ ID NO: 383 - DNA, 35 bp, Artificial Sequence
  Synthetic oligonucleotide
gctgttctcagactggttgccctggcctgggctgg

SEQ ID NO: 384 - DNA, 35 bp, Artificial Sequence
  Synthetic oligonucleotide
caggccagggcaaccagaccgagaacagctgcacc

SEQ ID NO: 385 - DNA, 35 bp, Artificial Sequence
  Synthetic oligonucleotide
ggtgcagctgttctcggtctggttgccctggcctg

SEQ ID NO: 386 - DNA, 35 bp, Artificial Sequence
  Synthetic oligonucleotide
ccaggccagggcaccaactctgagaacagctgcac

SEQ ID NO: 387 - DNA, 35 bp, Artificial Sequence
  Synthetic oligonucleotide
gtgcagctgttctcagagttggtgccctggcctgg

SEQ ID NO: 388 - DNA, 35 bp, Artificial Sequence
  Synthetic oligonucleotide
gccagggcaccaactctagcaacagctgcacccac

SEQ ID NO: 389 - DNA, 35 bp, Artificial Sequence
  Synthetic oligonucleotide
gtgggtgcagctgttgctagagttggtgccctggc

SEQ ID NO: 390 - DNA, 35 bp, Artificial Sequence
  Synthetic oligonucleotide
gccagggcaccaactctaccaacagctgcacccac

SEQ ID NO: 391 - DNA, 35 bp, Artificial Sequence
  Synthetic oligonucleotide
gtgggtgcagctgttggtagagttggtgccctggc

SEQ ID NO: 392 - DNA, 34 bp, Artificial Sequence
  Synthetic oligonucleotide
ggccagggcacccagaacgagaacagctgcaccc

SEQ ID NO: 393 - DNA, 34 bp, Artificial Sequence
  Synthetic oligonucleotide
gggtgcagctgttctcgttctgggtgccctggcc

SEQ ID NO: 394 - DNA, 35 bp, Artificial Sequence
  Synthetic oligonucleotide
gggcacccagaacgagagcagctgcacccacttcc

SEQ ID NO: 395 - DNA, 35 bp, Artificial Sequence
  Synthetic oligonucleotide
ggaagtgggtgcagctgctctcgttctgggtgccc

SEQ ID NO: 396 - DNA, 35 bp, Artificial Sequence
  Synthetic oligonucleotide
gggcacccagaacgagaccagctgcacccacttcc

SEQ ID NO: 397 - DNA, 35 bp, Artificial Sequence
  Synthetic oligonucleotide
ggaagtgggtgcagctggtctcgttctgggtgccc

SEQ ID NO: 398 - DNA, 34 bp, Artificial Sequence
  Synthetic oligonucleotide
ccagggcacccagtctaacaacagctgcacccac

SEQ ID NO: 399 - DNA, 34 bp, Artificial Sequence
  Synthetic oligonucleotide
gtgggtgcagctgttgttagactgggtgccctgg

SEQ ID NO: 400 - DNA, 35 bp, Artificial Sequence
  Synthetic oligonucleotide
cacccagtctaacaacacctgcacccacttcccag

SEQ ID NO: 401 - DNA, 35 bp, Artificial Sequence
  Synthetic oligonucleotide
ctgggaagtgggtgcaggtgttgttagactgggtg

SEQ ID NO: 402 - DNA, 35 bp, Artificial Sequence
  Synthetic oligonucleotide
cacccagtctgagaacaactgcacccacttcccag

SEQ ID NO: 403 - DNA, 35 bp, Artificial Sequence
  Synthetic oligonucleotide
ctgggaagtgggtgcagttgttctcagactgggtg

SEQ ID NO: 404 - DNA, 35 bp, Artificial Sequence
  Synthetic oligonucleotide
gtctgagaacaactgcagccacttcccaggcaacc

SEQ ID NO: 405 - DNA, 35 bp, Artificial Sequence
  Synthetic oligonucleotide
ggttgcctgggaagtggctgcagttgttctcagac

SEQ ID NO: 406 - DNA, 35 bp, Artificial Sequence
  Synthetic oligonucleotide
gtctgagaacagctgcaaccacttcccaggcaacc

SEQ ID NO: 407 - DNA, 35 bp, Artificial Sequence
  Synthetic oligonucleotide
ggttgcctgggaagtggttgcagctgttctcagac

SEQ ID NO: 408 - DNA, 33 bp, Artificial Sequence
  Synthetic oligonucleotide
gaacagctgcaaccacagcccaggcaacctgcc

SEQ ID NO: 409 - DNA, 33 bp, Artificial Sequence
  Synthetic oligonucleotide
ggcaggttgcctgggctgtggttgcagctgttc

SEQ ID NO: 410 - DNA, 35 bp, Artificial Sequence
  Synthetic oligonucleotide
gagaacagctgcaaccacaccccaggcaacctgcc

SEQ ID NO: 411 - DNA, 35 bp, Artificial Sequence
  Synthetic oligonucleotide
ggcaggttgcctggggtgtggttgcagctgttctc

SEQ ID NO: 412 - DNA, 35 bp, Artificial Sequence
  Synthetic oligonucleotide
ctgagaacagctgcaccaacttcccaggcaacctg

SEQ ID NO: 413 - DNA, 35 bp, Artificial Sequence
  Synthetic oligonucleotide
caggttgcctgggaagttggtgcagctgttctcag

SEQ ID NO: 414 - DNA, 35 bp, Artificial Sequence
  Synthetic oligonucleotide
gctgcaccaacttcagcggcaacctgcctaacatg

SEQ ID NO: 415 - DNA, 35 bp, Artificial Sequence
  Synthetic oligonucleotide
catgttaggcaggttgccgctgaagttggtgcagc

SEQ ID NO: 416 - DNA, 34 bp, Artificial Sequence
  Synthetic oligonucleotide
cagctgcaccaacttcaccggcaacctgcctaac

SEQ ID NO: 417 - DNA, 34 bp, Artificial Sequence
  Synthetic oligonucleotide
gttaggcaggttgccggtgaagttggtgcagctg

SEQ ID NO: 418 - DNA, 35 bp, Artificial Sequence
  Synthetic oligonucleotide
ctgcacccacttcccaaacaacctgcctaacatgc

SEQ ID NO: 419 - DNA, 35 bp, Artificial Sequence
  Synthetic oligonucleotide
gcatgttaggcaggttgtttgggaagtgggtgcag

SEQ ID NO: 420 - DNA, 35 bp, Artificial Sequence
  Synthetic oligonucleotide
ccacttcccaaacaacagccctaacatgcttcgag

SEQ ID NO: 421 - DNA, 35 bp, Artificial Sequence
  Synthetic oligonucleotide
ctcgaagcatgttagggctgttgtttgggaagtgg

SEQ ID NO: 422 - DNA, 35 bp, Artificial Sequence
  Synthetic oligonucleotide
ccacttcccaaacaacacccctaacatgcttcgag

SEQ ID NO: 423 - DNA, 35 bp, Artificial Sequence
  Synthetic oligonucleotide
ctcgaagcatgttaggggtgttgtttgggaagtgg

SEQ ID NO: 424 - DNA, 35 bp, Artificial Sequence
  Synthetic oligonucleotide
cttcccaggcaacctgagcaacatgcttcgagatc

SEQ ID NO: 425 - DNA, 35 bp, Artificial Sequence
  Synthetic oligonucleotide
gatctcgaagcatgttgctcaggttgcctgggaag

SEQ ID NO: 426 - DNA, 35 bp, Artificial Sequence
  Synthetic oligonucleotide
cttcccaggcaacctgaccaacatgcttcgagatc

SEQ ID NO: 427 - DNA, 35 bp, Artificial Sequence
  Synthetic oligonucleotide
gatctcgaagcatgttggtcaggttgcctgggaag

SEQ ID NO: 428 - DNA, 35 bp, Artificial Sequence
  Synthetic oligonucleotide
cctaacatgcttcgaaacctccgagatgccttcag

SEQ ID NO: 429 - DNA, 35 bp, Artificial Sequence
  Synthetic oligonucleotide
ctgaaggcatctcggaggtttcgaagcatgttagg

SEQ ID NO: 430 - DNA, 35 bp, Artificial Sequence
  Synthetic oligonucleotide
catgcttcgaaacctcagcgatgccttcagcagag

SEQ ID NO: 431 - DNA, 35 bp, Artificial Sequence
  Synthetic oligonucleotide
ctctgctgaaggcatcgctgaggtttcgaagcatg

SEQ ID NO: 432 - DNA, 35 bp, Artificial Sequence
  Synthetic oligonucleotide
catgcttcgaaacctcaccgatgccttcagcagag

SEQ ID NO: 433 - DNA, 35 bp, Artificial Sequence
  Synthetic oligonucleotide
ctctgctgaaggcatcggtgaggtttcgaagcatg

SEQ ID NO: 434 - DNA, 33 bp, Artificial Sequence
  Synthetic oligonucleotide
ctccgagatgccttcaacagagtgaagactttc

SEQ ID NO: 435 - DNA, 33 bp, Artificial Sequence
  Synthetic oligonucleotide
gaaagtcttcactctgttgaaggcatctcggag

SEQ ID NO: 436 - DNA, 35 bp, Artificial Sequence
  Synthetic oligonucleotide
gatgccttcaacagaagcaagactttctttcaaat

SEQ ID NO: 437 - DNA, 35 bp, Artificial Sequence
  Synthetic oligonucleotide
atttgaaagaaagtcttgcttctgttgaaggcatc

SEQ ID NO: 438 - DNA, 36 bp, Artificial Sequence
  Synthetic oligonucleotide
gatgccttcaacagaaccaagactttctttcaaatg

SEQ ID NO: 439 - DNA, 36 bp, Artificial Sequence
  Synthetic oligonucleotide
catttgaaagaaagtcttggttctgttgaaggcatc

SEQ ID NO: 440 - DNA, 35 bp, Artificial Sequence
  Synthetic oligonucleotide
ccgagatgccttcagcaacgtgaagactttctttc

SEQ ID NO: 441 - DNA, 35 bp, Artificial Sequence
  Synthetic oligonucleotide
gaaagaaagtcttcacgttgctgaaggcatctcgg

SEQ ID NO: 442 - DNA, 35 bp, Artificial Sequence
  Synthetic oligonucleotide
ccttcagcaacgtgagcactttctttcaaatgaag

SEQ ID NO: 443 - DNA, 35 bp, Artificial Sequence
  Synthetic oligonucleotide
cttcatttgaaagaaagtgctcacgttgctgaagg

SEQ ID NO: 444 - DNA, 36 bp, Artificial Sequence
  Synthetic oligonucleotide
gccttcagcaacgtgaccactttctttcaaatgaag

SEQ ID NO: 445 - DNA, 36 bp, Artificial Sequence
  Synthetic oligonucleotide
cttcatttgaaagaaagtggtcacgttgctgaaggc

SEQ ID NO: 446 - DNA, 35 bp, Artificial Sequence
  Synthetic oligonucleotide
ctttctttcaaatgaacgatcagctggacaacttg

SEQ ID NO: 447 - DNA, 35 bp, Artificial Sequence
  Synthetic oligonucleotide
caagttgtccagctgatcgttcatttgaaagaaag

SEQ ID NO: 448 - DNA, 34 bp, Artificial Sequence
  Synthetic oligonucleotide
ctttcaaatgaacgatagcctggacaacttgttg

SEQ ID NO: 449 - DNA, 34 bp, Artificial Sequence
  Synthetic oligonucleotide
caacaagttgtccaggctatcgttcatttgaaag

SEQ ID NO: 450 - DNA, 34 bp, Artificial Sequence
  Synthetic oligonucleotide
ctttcaaatgaacgataccctggacaacttgttg

SEQ ID NO: 451 - DNA, 34 bp, Artificial Sequence
  Synthetic oligonucleotide
caacaagttgtccagggtatcgttcatttgaaag

SEQ ID NO: 452 - DNA, 26 bp, Artificial Sequence
  Synthetic oligonucleotide
gttgttaaatgagtccttgctggagg

SEQ ID NO: 453 - DNA, 26 bp, Artificial Sequence
  Synthetic oligonucleotide
cctccagcaaggactcatttaacaac

SEQ ID NO: 454 - DNA, 35 bp, Artificial Sequence
  Synthetic oligonucleotide
tgttgttaaatgagagcttgctggaggactttaag

SEQ ID NO: 455 - DNA, 35 bp, Artificial Sequence
  Synthetic oligonucleotide
cttaaagtcctccagcaagctctcatttaacaaca

SEQ ID NO: 456 - DNA, 34 bp, Artificial Sequence
  Synthetic oligonucleotide
caacttgttgttaaagaactccttgctggaggac

SEQ ID NO: 457 - DNA, 34 bp, Artificial Sequence
  Synthetic oligonucleotide
gtcctccagcaaggagttctttaacaacaagttg

SEQ ID NO: 458 - DNA, 35 bp, Artificial Sequence
  Synthetic oligonucleotide
gttgttaaagaactccagcctggaggactttaagg

SEQ ID NO: 459 - DNA, 35 bp, Artificial Sequence
  Synthetic oligonucleotide
ccttaaagtcctccaggctggagttctttaacaac

SEQ ID NO: 460 - DNA, 35 bp, Artificial Sequence
  Synthetic oligonucleotide
gttgttaaagaactccaccctggaggactttaagg

SEQ ID NO: 461 - DNA, 35 bp, Artificial Sequence
  Synthetic oligonucleotide
ccttaaagtcctccagggtggagttctttaacaac

SEQ ID NO: 462 - DNA, 35 bp, Artificial Sequence
  Synthetic oligonucleotide
tgttgttaaaggagaacttgctggaggactttaag

SEQ ID NO: 463 - DNA, 35 bp, Artificial Sequence
  Synthetic oligonucleotide
cttaaagtcctccagcaagttctcctttaacaaca

SEQ ID NO: 464 - DNA, 35 bp, Artificial Sequence
  Synthetic oligonucleotide
gttaaaggagaacttgagcgaggactttaagggtt

SEQ ID NO: 465 - DNA, 35 bp, Artificial Sequence
  Synthetic oligonucleotide
aacccttaaagtcctcgctcaagttctcctttaac

SEQ ID NO: 466 - DNA, 37 bp, Artificial Sequence
  Synthetic oligonucleotide
gttaaaggagaacttgaccgaggactttaagggttac

SEQ ID NO: 467 - DNA, 37 bp, Artificial Sequence
  Synthetic oligonucleotide
gtaacccttaaagtcctcggtcaagttctcctttaac

SEQ ID NO: 468 - DNA, 35 bp, Artificial Sequence
  Synthetic oligonucleotide
ggagtccttgctgaacgactttaagggttacctgg

SEQ ID NO: 469 - DNA, 35 bp, Artificial Sequence
  Synthetic oligonucleotide
ccaggtaacccttaaagtcgttcagcaaggactcc

SEQ ID NO: 470 - DNA, 33 bp, Artificial Sequence
  Synthetic oligonucleotide
gtccttgctgaacgacagcaagggttacctggg

SEQ ID NO: 471 - DNA, 33 bp, Artificial Sequence
  Synthetic oligonucleotide
cccaggtaacccttgctgtcgttcagcaaggac

SEQ ID NO: 472 - DNA, 36 bp, Artificial Sequence
  Synthetic oligonucleotide
gtccttgctgaacgacaccaagggttacctgggttg

SEQ ID NO: 473 - DNA, 36 bp, Artificial Sequence
  Synthetic oligonucleotide
caacccaggtaacccttggtgtcgttcagcaaggac

SEQ ID NO: 474 - DNA, 35 bp, Artificial Sequence
  Synthetic oligonucleotide
ggagtccttgctggagaactttaagggttacctgg

SEQ ID NO: 475 - DNA, 35 bp, Artificial Sequence
  Synthetic oligonucleotide
ccaggtaacccttaaagttctccagcaaggactcc

SEQ ID NO: 476 - DNA, 35 bp, Artificial Sequence
  Synthetic oligonucleotide
cttgctggagaactttagcggttacctgggttgcc

SEQ ID NO: 477 - DNA, 35 bp, Artificial Sequence
  Synthetic oligonucleotide
ggcaacccaggtaaccgctaaagttctccagcaag

SEQ ID NO: 478 - DNA, 35 bp, Artificial Sequence
  Synthetic oligonucleotide
cttgctggagaactttaccggttacctgggttgcc

SEQ ID NO: 479 - DNA, 35 bp, Artificial Sequence
  Synthetic oligonucleotide
ggcaacccaggtaaccggtaaagttctccagcaag

SEQ ID NO: 480 - DNA, 35 bp, Artificial Sequence
  Synthetic oligonucleotide
cttgctggaggactttaacggttacctgggttgcc

SEQ ID NO: 481 - DNA, 35 bp, Artificial Sequence
  Synthetic oligonucleotide
ggcaacccaggtaaccgttaaagtcctccagcaag

SEQ ID NO: 482 - DNA, 34 bp, Artificial Sequence
  Synthetic oligonucleotide
ggaggactttaacggtagcctgggttgccaagcc

SEQ ID NO: 483 - DNA, 34 bp, Artificial Sequence
  Synthetic oligonucleotide
ggcttggcaacccaggctaccgttaaagtcctcc

SEQ ID NO: 484 - DNA, 34 bp, Artificial Sequence
  Synthetic oligonucleotide
ggaggactttaacggtaccctgggttgccaagcc

SEQ ID NO: 485 - DNA, 34 bp, Artificial Sequence
  Synthetic oligonucleotide
ggcttggcaacccagggtaccgttaaagtcctcc

SEQ ID NO: 486 - DNA, 35 bp, Artificial Sequence
  Synthetic oligonucleotide
gctggaggactttaagaactacctgggttgccaag

SEQ ID NO: 487 - DNA, 35 bp, Artificial Sequence
  Synthetic oligonucleotide
cttggcaacccaggtagttcttaaagtcctccagc

SEQ ID NO: 488 - DNA, 34 bp, Artificial Sequence
  Synthetic oligonucleotide
ggactttaagaactacagcggttgccaagccttg

SEQ ID NO: 489 - DNA, 34 bp, Artificial Sequence
  Synthetic oligonucleotide
caaggcttggcaaccgctgtagttcttaaagtcc

SEQ ID NO: 490 - DNA, 34 bp, Artificial Sequence
  Synthetic oligonucleotide
ggactttaagaactacaccggttgccaagccttg

SEQ ID NO: 491 - DNA, 34 bp, Artificial Sequence
  Synthetic oligonucleotide
caaggcttggcaaccggtgtagttcttaaagtcc

SEQ ID NO: 492 - DNA, 35 bp, Artificial Sequence
  Synthetic oligonucleotide
ctggaggactttaagggtaacctgggttgccaagc

SEQ ID NO: 493 - DNA, 35 bp, Artificial Sequence
  Synthetic oligonucleotide
gcttggcaacccaggttacccttaaagtcctccag

SEQ ID NO: 494 - DNA, 31 bp, Artificial Sequence
  Synthetic oligonucleotide
ttaagggtaacctgagctgccaagccttgtc

SEQ ID NO: 495 - DNA, 31 bp, Artificial Sequence
  Synthetic oligonucleotide
gacaaggcttggcagctcaggttacccttaa

SEQ ID NO: 496 - DNA, 35 bp, Artificial Sequence
  Synthetic oligonucleotide
ctttaagggttacctgaactgccaagccttgtctg

SEQ ID NO: 497 - DNA, 35 bp, Artificial Sequence
  Synthetic oligonucleotide
cagacaaggcttggcagttcaggtaacccttaaag

SEQ ID NO: 498 - DNA, 34 bp, Artificial Sequence
  Synthetic oligonucleotide
gggttacctgaactgcagcgccttgtctgagatg

SEQ ID NO: 499 - DNA, 34 bp, Artificial Sequence
  Synthetic oligonucleotide
catctcagacaaggcgctgcagttcaggtaaccc

SEQ ID NO: 500 - DNA, 34 bp, Artificial Sequence
  Synthetic oligonucleotide
gggttacctgaactgcaccgccttgtctgagatg

SEQ ID NO: 501 - DNA, 34 bp, Artificial Sequence
  Synthetic oligonucleotide
catctcagacaaggcggtgcagttcaggtaaccc

SEQ ID NO: 502 - DNA, 34 bp, Artificial Sequence
  Synthetic oligonucleotide
gggttacctgggttgcaacgccttgtctgagatg

SEQ ID NO: 503 - DNA, 34 bp, Artificial Sequence
  Synthetic oligonucleotide
catctcagacaaggcgttgcaacccaggtaaccc

SEQ ID NO: 504 - DNA, 34 bp, Artificial Sequence
  Synthetic oligonucleotide
cctgggttgcaacgccagctctgagatgatccag

SEQ ID NO: 505 - DNA, 34 bp, Artificial Sequence
  Synthetic oligonucleotide
ctggatcatctcagagctggcgttgcaacccagg

SEQ ID NO: 506 - DNA, 34 bp, Artificial Sequence
  Synthetic oligonucleotide
cctgggttgcaacgccacctctgagatgatccag

SEQ ID NO: 507 - DNA, 34 bp, Artificial Sequence
  Synthetic oligonucleotide
ctggatcatctcagaggtggcgttgcaacccagg

SEQ ID NO: 508 - DNA, 35 bp, Artificial Sequence
  Synthetic oligonucleotide
gttgccaagccttgaacgagatgatccagttttac

SEQ ID NO: 509 - DNA, 35 bp, Artificial Sequence
  Synthetic oligonucleotide
gtaaaactggatcatctcgttcaaggcttggcaac

SEQ ID NO: 510 - DNA, 35 bp, Artificial Sequence
  Synthetic oligonucleotide
ccaagccttgaacgagagcatccagttttacctgg

SEQ ID NO: 511 - DNA, 35 bp, Artificial Sequence
  Synthetic oligonucleotide
ccaggtaaaactggatgctctcgttcaaggcttgg

SEQ ID NO: 512 - DNA, 35 bp, Artificial Sequence
  Synthetic oligonucleotide
ccaagccttgaacgagaccatccagttttacctgg

SEQ ID NO: 513 - DNA, 35 bp, Artificial Sequence
  Synthetic oligonucleotide
ccaggtaaaactggatggtctcgttcaaggcttgg

SEQ ID NO: 514 - DNA, 35 bp, Artificial Sequence
  Synthetic oligonucleotide
gttgccaagccttgtctaacatgatccagttttac

SEQ ID NO: 515 - DNA, 35 bp, Artificial Sequence
  Synthetic oligonucleotide
gtaaaactggatcatgttagacaaggcttggcaac

SEQ ID NO: 516 - DNA, 34 bp, Artificial Sequence
  Synthetic oligonucleotide
gccttgtctaacatgagccagttttacctggagg

SEQ ID NO: 517 - DNA, 34 bp, Artificial Sequence
  Synthetic oligonucleotide
cctccaggtaaaactggctcatgttagacaaggc

SEQ ID NO: 518 - DNA, 34 bp, Artificial Sequence
  Synthetic oligonucleotide
gccttgtctaacatgacccagttttacctggagg

SEQ ID NO: 519 - DNA, 34 bp, Artificial Sequence
  Synthetic oligonucleotide
cctccaggtaaaactgggtcatgttagacaaggc

SEQ ID NO: 520 - DNA, 35 bp, Artificial Sequence
  Synthetic oligonucleotide
cttgtctgagatgatcaacttttacctggaggagg

SEQ ID NO: 521 - DNA, 35 bp, Artificial Sequence
  Synthetic oligonucleotide
cctcctccaggtaaaagttgatcatctcagacaag

SEQ ID NO: 522 - DNA, 35 bp, Artificial Sequence
  Synthetic oligonucleotide
ctgagatgatcaactttagcctggaggaggtgatg

SEQ ID NO: 523 - DNA, 35 bp, Artificial Sequence
  Synthetic oligonucleotide
catcacctcctccaggctaaagttgatcatctcag

SEQ ID NO: 524 - DNA, 35 bp, Artificial Sequence
  Synthetic oligonucleotide
ctgagatgatcaactttaccctggaggaggtgatg

SEQ ID NO: 525 - DNA, 35 bp, Artificial Sequence
  Synthetic oligonucleotide
catcacctcctccagggtaaagttgatcatctcag

SEQ ID NO: 526 - DNA, 35 bp, Artificial Sequence
  Synthetic oligonucleotide
gatccagttttacctgaacgaggtgatgccccaag

SEQ ID NO: 527 - DNA, 35 bp, Artificial Sequence
  Synthetic oligonucleotide
cttggggcatcacctcgttcaggtaaaactggatc

SEQ ID NO: 528 - DNA, 34 bp, Artificial Sequence
  Synthetic oligonucleotide
gttttacctgaacgagagcatgccccaagctgag

SEQ ID NO: 529 - DNA, 34 bp, Artificial Sequence
  Synthetic oligonucleotide
ctcagcttggggcatgctctcgttcaggtaaaac

SEQ ID NO: 530 - DNA, 34 bp, Artificial Sequence
  Synthetic oligonucleotide
gttttacctgaacgagaccatgccccaagctgag

SEQ ID NO: 531 - DNA, 34 bp, Artificial Sequence
  Synthetic oligonucleotide
ctcagcttggggcatggtctcgttcaggtaaaac

SEQ ID NO: 532 - DNA, 35 bp, Artificial Sequence
  Synthetic oligonucleotide
ccagttttacctggagaacgtgatgccccaagctg

SEQ ID NO: 533 - DNA, 35 bp, Artificial Sequence
  Synthetic oligonucleotide
cagcttggggcatcacgttctccaggtaaaactgg

SEQ ID NO: 534 - DNA, 35 bp, Artificial Sequence
  Synthetic oligonucleotide
cctggagaacgtgagcccccaagctgagaaccaag

SEQ ID NO: 535 - DNA, 35 bp, Artificial Sequence
  Synthetic oligonucleotide
cttggttctcagcttgggggctcacgttctccagg

SEQ ID NO: 536 - DNA, 35 bp, Artificial Sequence
  Synthetic oligonucleotide
cctggagaacgtgaccccccaagctgagaaccaag

SEQ ID NO: 537 - DNA, 35 bp, Artificial Sequence
  Synthetic oligonucleotide
cttggttctcagcttggggggtcacgttctccagg

SEQ ID NO: 538 - DNA, 35 bp, Artificial Sequence
  Synthetic oligonucleotide
cctggaggaggtgatgaaccaagctgagaaccaag

SEQ ID NO: 539 - DNA, 35 bp, Artificial Sequence
  Synthetic oligonucleotide
cttggttctcagcttggttcatcacctcctccagg

SEQ ID NO: 540 - DNA, 35 bp, Artificial Sequence
  Synthetic oligonucleotide
ggaggtgatgaaccaaagcgagaaccaagacccag

SEQ ID NO: 541 - DNA, 35 bp, Artificial Sequence
  Synthetic oligonucleotide
ctgggtcttggttctcgctttggttcatcacctcc

SEQ ID NO: 542 - DNA, 35 bp, Artificial Sequence
  Synthetic oligonucleotide
ggaggtgatgaaccaaaccgagaaccaagacccag

SEQ ID NO: 543 - DNA, 35 bp, Artificial Sequence
  Synthetic oligonucleotide
ctgggtcttggttctcggtttggttcatcacctcc

SEQ ID NO: 544 - DNA, 35 bp, Artificial Sequence
  Synthetic oligonucleotide
ggaggaggtgatgcccaacgctgagaaccaagacc

SEQ ID NO: 545 - DNA, 35 bp, Artificial Sequence
  Synthetic oligonucleotide
ggtcttggttctcagcgttgggcatcacctcctcc

SEQ ID NO: 546 - DNA, 34 bp, Artificial Sequence
  Synthetic oligonucleotide
ggtgatgcccaacgctagcaaccaagacccagac

SEQ ID NO: 547 - DNA, 34 bp, Artificial Sequence
  Synthetic oligonucleotide
gtctgggtcttggttgctagcgttgggcatcacc

SEQ ID NO: 548 - DNA, 34 bp, Artificial Sequence
  Synthetic oligonucleotide
ggtgatgcccaacgctaccaaccaagacccagac

SEQ ID NO: 549 - DNA, 34 bp, Artificial Sequence
  Synthetic oligonucleotide
gtctgggtcttggttggtagcgttgggcatcacc

SEQ ID NO: 550 - DNA, 34 bp, Artificial Sequence
  Synthetic oligonucleotide
ggtgatgccccaagctaacaaccaagacccagac

SEQ ID NO: 551 - DNA, 34 bp, Artificial Sequence
  Synthetic oligonucleotide
gtctgggtcttggttgttagcttggggcatcacc

SEQ ID NO: 552 - DNA, 35 bp, Artificial Sequence
  Synthetic oligonucleotide
gccccaagctaacaacagcgacccagacatcaagg

SEQ ID NO: 553 - DNA, 35 bp, Artificial Sequence
  Synthetic oligonucleotide
ccttgatgtctgggtcgctgttgttagcttggggc

SEQ ID NO: 554 - DNA, 35 bp, Artificial Sequence
  Synthetic oligonucleotide
gccccaagctaacaacaccgacccagacatcaagg

SEQ ID NO: 555 - DNA, 35 bp, Artificial Sequence
  Synthetic oligonucleotide
ccttgatgtctgggtcggtgttgttagcttggggc

SEQ ID NO: 556 - DNA, 35 bp, Artificial Sequence
  Synthetic oligonucleotide
gccccaagctgagaacaacgacccagacatcaagg

SEQ ID NO: 557 - DNA, 35 bp, Artificial Sequence
  Synthetic oligonucleotide
ccttgatgtctgggtcgttgttctcagcttggggc

SEQ ID NO: 558 - DNA, 34 bp, Artificial Sequence
  Synthetic oligonucleotide
gctgagaacaacgacagcgacatcaaggcgcatg

SEQ ID NO: 559 - DNA, 34 bp, Artificial Sequence
  Synthetic oligonucleotide
catgcgccttgatgtcgctgtcgttgttctcagc

SEQ ID NO: 560 - DNA, 34 bp, Artificial Sequence
  Synthetic oligonucleotide
gctgagaacaacgacaccgacatcaaggcgcatg

SEQ ID NO: 561 - DNA, 34 bp, Artificial Sequence
  Synthetic oligonucleotide
catgcgccttgatgtcggtgtcgttgttctcagc

SEQ ID NO: 562 - DNA, 37 bp, Artificial Sequence
  Synthetic oligonucleotide
gccccaagctgagaaccaaagcccagacatcaaggcg

SEQ ID NO: 563 - DNA, 37 bp, Artificial Sequence
  Synthetic oligonucleotide
cgccttgatgtctgggctttggttctcagcttggggc

SEQ ID NO: 564 - DNA, 35 bp, Artificial Sequence
  Synthetic oligonucleotide
ccaagctgagaaccaaaccccagacatcaaggcgc

SEQ ID NO: 565 - DNA, 35 bp, Artificial Sequence
  Synthetic oligonucleotide
gcgccttgatgtctggggtttggttctcagcttgg

SEQ ID NO: 566 - DNA, 35 bp, Artificial Sequence
  Synthetic oligonucleotide
ccaagctgagaaccaaaacccagacatcaaggcgc

SEQ ID NO: 567 - DNA, 35 bp, Artificial Sequence
  Synthetic oligonucleotide
gcgccttgatgtctgggttttggttctcagcttgg

SEQ ID NO: 568 - DNA, 35 bp, Artificial Sequence
  Synthetic oligonucleotide
ctgagaaccaaaacccaagcatcaaggcgcatgtg

SEQ ID NO: 569 - DNA, 35 bp, Artificial Sequence
  Synthetic oligonucleotide
cacatgcgccttgatgcttgggttttggttctcag

SEQ ID NO: 570 - DNA, 35 bp, Artificial Sequence
  Synthetic oligonucleotide
ctgagaaccaaaacccaaccatcaaggcgcatgtg

SEQ ID NO: 571 - DNA, 35 bp, Artificial Sequence
  Synthetic oligonucleotide
cacatgcgccttgatggttgggttttggttctcag

SEQ ID NO: 572 - DNA, 34 bp, Artificial Sequence
  Synthetic oligonucleotide
gctgagaaccaagacaacgacatcaaggcgcatg

SEQ ID NO: 573 - DNA, 34 bp, Artificial Sequence
  Synthetic oligonucleotide
catgcgccttgatgtcgttgtcttggttctcagc

SEQ ID NO: 574 - DNA, 34 bp, Artificial Sequence
  Synthetic oligonucleotide
gaaccaagacaacgacagcaaggcgcatgtgaac

SEQ ID NO: 575 - DNA, 34 bp, Artificial Sequence
  Synthetic oligonucleotide
gttcacatgcgccttgctgtcgttgtcttggttc

SEQ ID NO: 576 - DNA, 34 bp, Artificial Sequence
  Synthetic oligonucleotide
gaaccaagacaacgacaccaaggcgcatgtgaac

SEQ ID NO: 577 - DNA, 34 bp, Artificial Sequence
  Synthetic oligonucleotide
gttcacatgcgccttggtgtcgttgtcttggttc

SEQ ID NO: 578 - DNA, 35 bp, Artificial Sequence
  Synthetic oligonucleotide
ctgagaaccaagacccaaacatcaaggcgcatgtg

SEQ ID NO: 579 - DNA, 35 bp, Artificial Sequence
  Synthetic oligonucleotide
cacatgcgccttgatgtttgggtcttggttctcag

SEQ ID NO: 580 - DNA, 35 bp, Artificial Sequence
  Synthetic oligonucleotide
ccaagacccaaacatcagcgcgcatgtgaactccc

SEQ ID NO: 581 - DNA, 35 bp, Artificial Sequence
  Synthetic oligonucleotide
gggagttcacatgcgcgctgatgtttgggtcttgg

SEQ ID NO: 582 - DNA, 35 bp, Artificial Sequence
  Synthetic oligonucleotide
ccaagacccaaacatcaccgcgcatgtgaactccc

SEQ ID NO: 583 - DNA, 35 bp, Artificial Sequence
  Synthetic oligonucleotide
gggagttcacatgcgcggtgatgtttgggtcttgg

SEQ ID NO: 584 - DNA, 35 bp, Artificial Sequence
  Synthetic oligonucleotide
ccaagacccagacatcaacgcgcatgtgaactccc

SEQ ID NO: 585 - DNA, 35 bp, Artificial Sequence
  Synthetic oligonucleotide
gggagttcacatgcgcgttgatgtctgggtcttgg

SEQ ID NO: 586 - DNA, 35 bp, Artificial Sequence
  Synthetic oligonucleotide
cccagacatcaacgcgagcgtgaactccctggggg

SEQ ID NO: 587 - DNA, 35 bp, Artificial Sequence
  Synthetic oligonucleotide
cccccagggagttcacgctcgcgttgatgtctggg

SEQ ID NO: 588 - DNA, 35 bp, Artificial Sequence
  Synthetic oligonucleotide
cccagacatcaacgcgaccgtgaactccctggggg

SEQ ID NO: 589 - DNA, 35 bp, Artificial Sequence
  Synthetic oligonucleotide
cccccagggagttcacggtcgcgttgatgtctggg

SEQ ID NO: 590 - DNA, 35 bp, Artificial Sequence
  Synthetic oligonucleotide
gacccagacatcaagaaccatgtgaactccctggg

SEQ ID NO: 591 - DNA, 35 bp, Artificial Sequence
  Synthetic oligonucleotide
cccagggagttcacatggttcttgatgtctgggtc

SEQ ID NO: 592 - DNA, 35 bp, Artificial Sequence
  Synthetic oligonucleotide
cagacatcaagaaccatagcaactccctgggggag

SEQ ID NO: 593 - DNA, 35 bp, Artificial Sequence
  Synthetic oligonucleotide
ctcccccagggagttgctatggttcttgatgtctg

SEQ ID NO: 594 - DNA, 33 bp, Artificial Sequence
  Synthetic oligonucleotide
gacatcaagaaccataccaactccctgggggag

SEQ ID NO: 595 - DNA, 33 bp, Artificial Sequence
  Synthetic oligonucleotide
ctcccccagggagttggtatggttcttgatgtc

SEQ ID NO: 596 - DNA, 35 bp, Artificial Sequence
  Synthetic oligonucleotide
cccagacatcaaggcgaacgtgaactccctggggg

SEQ ID NO: 597 - DNA, 35 bp, Artificial Sequence
  Synthetic oligonucleotide
cccccagggagttcacgttcgccttgatgtctggg

SEQ ID NO: 598 - DNA, 35 bp, Artificial Sequence
  Synthetic oligonucleotide
catcaaggcgaacgtgacctccctgggggagaacc

SEQ ID NO: 599 - DNA, 35 bp, Artificial Sequence
  Synthetic oligonucleotide
ggttctcccccagggaggtcacgttcgccttgatg

SEQ ID NO: 600 - DNA, 34 bp, Artificial Sequence
  Synthetic oligonucleotide
caaggcgcatgtgaacaacctgggggagaacctg

SEQ ID NO: 601 - DNA, 34 bp, Artificial Sequence
  Synthetic oligonucleotide
caggttctcccccaggttgttcacatgcgccttg

SEQ ID NO: 602 - DNA, 35 bp, Artificial Sequence
  Synthetic oligonucleotide
gcatgtgaacaacctgagcgagaacctgaagaccc

SEQ ID NO: 603 - DNA, 35 bp, Artificial Sequence
  Synthetic oligonucleotide
gggtcttcaggttctcgctcaggttgttcacatgc

SEQ ID NO: 604 - DNA, 35 bp, Artificial Sequence
  Synthetic oligonucleotide
gcatgtgaacaacctgaccgagaacctgaagaccc

SEQ ID NO: 605 - DNA, 35 bp, Artificial Sequence
  Synthetic oligonucleotide
gggtcttcaggttctcggtcaggttgttcacatgc

SEQ ID NO: 606 - DNA, 29 bp, Artificial Sequence
  Synthetic oligonucleotide
cgcatgtgaactcctcgggggagaacctg

SEQ ID NO: 607 - DNA, 29 bp, Artificial Sequence
  Synthetic oligonucleotide
caggttctcccccgaggagttcacatgcg

SEQ ID NO: 608 - DNA, 34 bp, Artificial Sequence
  Synthetic oligonucleotide
ggcgcatgtgaactccaccggggagaacctgaag

SEQ ID NO: 609 - DNA, 34 bp, Artificial Sequence
  Synthetic oligonucleotide
cttcaggttctccccggtggagttcacatgcgcc

SEQ ID NO: 610 - DNA, 35 bp, Artificial Sequence
  Synthetic oligonucleotide
gcatgtgaactccctgaacgagaacctgaagaccc

SEQ ID NO: 611 - DNA, 35 bp, Artificial Sequence
  Synthetic oligonucleotide
gggtcttcaggttctcgttcagggagttcacatgc

SEQ ID NO: 612 - DNA, 35 bp, Artificial Sequence
  Synthetic oligonucleotide
gaactccctgaacgagagcctgaagaccctcaggc

SEQ ID NO: 613 - DNA, 35 bp, Artificial Sequence
  Synthetic oligonucleotide
gcctgagggtcttcaggctctcgttcagggagttc

SEQ ID NO: 614 - DNA, 35 bp, Artificial Sequence
  Synthetic oligonucleotide
gaactccctgaacgagaccctgaagaccctcaggc

SEQ ID NO: 615 - DNA, 35 bp, Artificial Sequence
  Synthetic oligonucleotide
gcctgagggtcttcagggtctcgttcagggagttc

SEQ ID NO: 616 - DNA, 35 bp, Artificial Sequence
  Synthetic oligonucleotide
gtgaactccctggggaacaacctgaagaccctcag

SEQ ID NO: 617 - DNA, 35 bp, Artificial Sequence
  Synthetic oligonucleotide
ctgagggtcttcaggttgttccccagggagttcac

SEQ ID NO: 618 - DNA, 34 bp, Artificial Sequence
  Synthetic oligonucleotide
ctccctggggaacaacagcaagaccctcaggctg

SEQ ID NO: 619 - DNA, 34 bp, Artificial Sequence
  Synthetic oligonucleotide
cagcctgagggtcttgctgttgttccccagggag

SEQ ID NO: 620 - DNA, 34 bp, Artificial Sequence
  Synthetic oligonucleotide
ctccctggggaacaacaccaagaccctcaggctg

SEQ ID NO: 621 - DNA, 34 bp, Artificial Sequence
  Synthetic oligonucleotide
cagcctgagggtcttggtgttgttccccagggag

SEQ ID NO: 622 - DNA, 35 bp, Artificial Sequence
  Synthetic oligonucleotide
cctgggggagaacctgagcaccctcaggctgaggc

SEQ ID NO: 623 - DNA, 35 bp, Artificial Sequence
  Synthetic oligonucleotide
gcctcagcctgagggtgctcaggttctcccccagg

SEQ ID NO: 624 - DNA, 35 bp, Artificial Sequence
  Synthetic oligonucleotide
cctgggggagaacctgaccaccctcaggctgaggc

SEQ ID NO: 625 - DNA, 35 bp, Artificial Sequence
  Synthetic oligonucleotide
gcctcagcctgagggtggtcaggttctcccccagg

SEQ ID NO: 626 - DNA, 35 bp, Artificial Sequence
  Synthetic oligonucleotide
cctgggggagaacctgaacaccctcaggctgaggc

SEQ ID NO: 627 - DNA, 35 bp, Artificial Sequence
  Synthetic oligonucleotide
gcctcagcctgagggtgttcaggttctcccccagg

SEQ ID NO: 628 - DNA, 35 bp, Artificial Sequence
  Synthetic oligonucleotide
ggagaacctgaacaccagcaggctgaggctacggc

SEQ ID NO: 629 - DNA, 35 bp, Artificial Sequence
  Synthetic oligonucleotide
gccgtagcctcagcctgctggtgttcaggttctcc

SEQ ID NO: 630 - DNA, 35 bp, Artificial Sequence
  Synthetic oligonucleotide
ggagaacctgaacaccaccaggctgaggctacggc

SEQ ID NO: 631 - DNA, 35 bp, Artificial Sequence
  Synthetic oligonucleotide
gccgtagcctcagcctggtggtgttcaggttctcc

SEQ ID NO: 632 - DNA, 29 bp, Artificial Sequence
  Synthetic oligonucleotide
ggagaacctgaagaacctcaggctgaggc

SEQ ID NO: 633 - DNA, 29 bp, Artificial Sequence
  Synthetic oligonucleotide
gcctcagcctgaggttcttcaggttctcc

SEQ ID NO: 634 - DNA, 35 bp, Artificial Sequence
  Synthetic oligonucleotide
cctgaagaacctcagcctgaggctacggcgctgtc

SEQ ID NO: 635 - DNA, 35 bp, Artificial Sequence
  Synthetic oligonucleotide
gacagcgccgtagcctcaggctgaggttcttcagg

SEQ ID NO: 636 - DNA, 34 bp, Artificial Sequence
  Synthetic oligonucleotide
gaacctgaagaacctcaccctgaggctacggcgc

SEQ ID NO: 637 - DNA, 34 bp, Artificial Sequence
  Synthetic oligonucleotide
gcgccgtagcctcagggtgaggttcttcaggttc

SEQ ID NO: 638 - DNA, 34 bp, Artificial Sequence
  Synthetic oligonucleotide
gaacctgaagaccctcaacctgaggctacggcgc

SEQ ID NO: 639 - DNA, 34 bp, Artificial Sequence
  Synthetic oligonucleotide
gcgccgtagcctcaggttgagggtcttcaggttc

SEQ ID NO: 640 - DNA, 33 bp, Artificial Sequence
  Synthetic oligonucleotide
gaccctcaacctgagcctacggcgctgtcatcg

SEQ ID NO: 641 - DNA, 33 bp, Artificial Sequence
  Synthetic oligonucleotide
cgatgacagcgccgtaggctcaggttgagggtc

SEQ ID NO: 642 - DNA, 36 bp, Artificial Sequence
  Synthetic oligonucleotide
gaagaccctcaacctgaccctacggcgctgtcatcg

SEQ ID NO: 643 - DNA, 36 bp, Artificial Sequence
  Synthetic oligonucleotide
cgatgacagcgccgtagggtcaggttgagggtcttc

SEQ ID NO: 644 - DNA, 35 bp, Artificial Sequence
  Synthetic oligonucleotide
cctgaagaccctcaggaacaggctacggcgctgtc

SEQ ID NO: 645 - DNA, 35 bp, Artificial Sequence
  Synthetic oligonucleotide
gacagcgccgtagcctgttcctgagggtcttcagg

SEQ ID NO: 646 - DNA, 35 bp, Artificial Sequence
  Synthetic oligonucleotide
gaccctcaggaacaggagccggcgctgtcatcgat

SEQ ID NO: 647 - DNA, 35 bp, Artificial Sequence
  Synthetic oligonucleotide
atcgatgacagcgccggctcctgttcctgagggtc

SEQ ID NO: 648 - DNA, 33 bp, Artificial Sequence
  Synthetic oligonucleotide
gaccctcaggaacaggacccggcgctgtcatcg

SEQ ID NO: 649 - DNA, 33 bp, Artificial Sequence
  Synthetic oligonucleotide
cgatgacagcgccgggtcctgttcctgagggtc

SEQ ID NO: 650 - DNA, 35 bp, Artificial Sequence
  Synthetic oligonucleotide
gaagaccctcaggctgaacctacggcgctgtcatc

SEQ ID NO: 651 - DNA, 35 bp, Artificial Sequence
  Synthetic oligonucleotide
gatgacagcgccgtaggttcagcctgagggtcttc

SEQ ID NO: 652 - DNA, 35 bp, Artificial Sequence
  Synthetic oligonucleotide
cctcaggctgaacctaagccgctgtcatcgatttc

SEQ ID NO: 653 - DNA, 35 bp, Artificial Sequence
  Synthetic oligonucleotide
gaaatcgatgacagcggcttaggttcagcctgagg

SEQ ID NO: 654 - DNA, 35 bp, Artificial Sequence
  Synthetic oligonucleotide
cctcaggctgaacctaacccgctgtcatcgatttc

SEQ ID NO: 655 - DNA, 35 bp, Artificial Sequence
  Synthetic oligonucleotide
gaaatcgatgacagcgggttaggttcagcctgagg

SEQ ID NO: 656 - DNA, 35 bp, Artificial Sequence
  Synthetic oligonucleotide
caggctgaggctacggaactgtcatcgatttcttc

SEQ ID NO: 657 - DNA, 35 bp, Artificial Sequence
  Synthetic oligonucleotide
gaagaaatcgatgacagttccgtagcctcagcctg

SEQ ID NO: 658 - DNA, 35 bp, Artificial Sequence
  Synthetic oligonucleotide
gaggctacggaactgtagccgatttcttccctgtg

SEQ ID NO: 659 - DNA, 35 bp, Artificial Sequence
  Synthetic oligonucleotide
cacagggaagaaatcggctacagttccgtagcctc

SEQ ID NO: 660 - DNA, 35 bp, Artificial Sequence
  Synthetic oligonucleotide
gaggctacggaactgtacccgatttcttccctgtg

SEQ ID NO: 661 - DNA, 35 bp, Artificial Sequence
  Synthetic oligonucleotide
cacagggaagaaatcgggtacagttccgtagcctc

SEQ ID NO: 662 - DNA, 35 bp, Artificial Sequence
  Synthetic oligonucleotide
gaggctacggcgctgtaaccgatttcttccctgtg

SEQ ID NO: 663 - DNA, 35 bp, Artificial Sequence
  Synthetic oligonucleotide
cacagggaagaaatcggttacagcgccgtagcctc

SEQ ID NO: 664 - DNA, 35 bp, Artificial Sequence
  Synthetic oligonucleotide
ggcgctgtaaccgaagccttccctgtgaaaacaag

SEQ ID NO: 665 - DNA, 35 bp, Artificial Sequence
  Synthetic oligonucleotide
cttgttttcacagggaaggcttcggttacagcgcc

SEQ ID NO: 666 - DNA, 36 bp, Artificial Sequence
  Synthetic oligonucleotide
cggcgctgtaaccgaacccttccctgtgaaaacaag

SEQ ID NO: 667 - DNA, 36 bp, Artificial Sequence
  Synthetic oligonucleotide
cttgttttcacagggaagggttcggttacagcgccg

SEQ ID NO: 668 - DNA, 34 bp, Artificial Sequence
  Synthetic oligonucleotide
gctacggcgctgtcataactttcttccctgtgaa

SEQ ID NO: 669 - DNA, 34 bp, Artificial Sequence
  Synthetic oligonucleotide
ttcacagggaagaaagttatgacagcgccgtagc

SEQ ID NO: 670 - DNA, 35 bp, Artificial Sequence
  Synthetic oligonucleotide
cgctgtcataactttagcccctgtgaaaacaagag

SEQ ID NO: 671 - DNA, 35 bp, Artificial Sequence
  Synthetic oligonucleotide
ctcttgttttcacaggggctaaagttatgacagcg

SEQ ID NO: 672 - DNA, 35 bp, Artificial Sequence
  Synthetic oligonucleotide
ggcgctgtcataactttaccccctgtgaaaacaag

SEQ ID NO: 673 - DNA, 35 bp, Artificial Sequence
  Synthetic oligonucleotide
cttgttttcacagggggtaaagttatgacagcgcc

SEQ ID NO: 674 - DNA, 35 bp, Artificial Sequence
  Synthetic oligonucleotide
gctgtcatcgatttcttaactgtgaaaacaagagc

SEQ ID NO: 675 - DNA, 35 bp, Artificial Sequence
  Synthetic oligonucleotide
gctcttgttttcacagttaagaaatcgatgacagc

SEQ ID NO: 676 - DNA, 34 bp, Artificial Sequence
  Synthetic oligonucleotide
cgatttcttaactgtagcaacaagagcaaggccg

SEQ ID NO: 677 - DNA, 34 bp, Artificial Sequence
  Synthetic oligonucleotide
cggccttgctcttgttgctacagttaagaaatcg

SEQ ID NO: 678 - DNA, 34 bp, Artificial Sequence
  Synthetic oligonucleotide
cgatttcttaactgtaccaacaagagcaaggccg

SEQ ID NO: 679 - DNA, 34 bp, Artificial Sequence
  Synthetic oligonucleotide
cggccttgctcttgttggtacagttaagaaatcg

SEQ ID NO: 680 - DNA, 34 bp, Artificial Sequence
  Synthetic oligonucleotide
cgatttcttccctgtaacaacaagagcaaggccg

SEQ ID NO: 681 - DNA, 34 bp, Artificial Sequence
  Synthetic oligonucleotide
cggccttgctcttgttgttacagggaagaaatcg

SEQ ID NO: 682 - DNA, 34 bp, Artificial Sequence
  Synthetic oligonucleotide
cttccctgtaacaacagcagcaaggccgtggagc

SEQ ID NO: 683 - DNA, 34 bp, Artificial Sequence
  Synthetic oligonucleotide
gctccacggccttgctgctgttgttacagggaag

SEQ ID NO: 684 - DNA, 34 bp, Artificial Sequence
  Synthetic oligonucleotide
cttccctgtaacaacaccagcaaggccgtggagc

SEQ ID NO: 685 - DNA, 34 bp, Artificial Sequence
  Synthetic oligonucleotide
gctccacggccttgctggtgttgttacagggaag

SEQ ID NO: 686 - DNA, 34 bp, Artificial Sequence
  Synthetic oligonucleotide
ccctgtgaaaacaagaccaaggccgtggagcagg

SEQ ID NO: 687 - DNA, 34 bp, Artificial Sequence
  Synthetic oligonucleotide
cctgctccacggccttggtcttgttttcacaggg

SEQ ID NO: 688 - DNA, 34 bp, Artificial Sequence
  Synthetic oligonucleotide
cttccctgtgaaaacaacagcaaggccgtggagc

SEQ ID NO: 689 - DNA, 34 bp, Artificial Sequence
  Synthetic oligonucleotide
gctccacggccttgctgttgttttcacagggaag

SEQ ID NO: 690 - DNA, 35 bp, Artificial Sequence
  Synthetic oligonucleotide
gtgaaaacaacagcagcgccgtggagcaggtgaag

SEQ ID NO: 691 - DNA, 35 bp, Artificial Sequence
  Synthetic oligonucleotide
cttcacctgctccacggcgctgctgttgttttcac

SEQ ID NO: 692 - DNA, 35 bp, Artificial Sequence
  Synthetic oligonucleotide
gtgaaaacaacagcaccgccgtggagcaggtgaag

SEQ ID NO: 693 - DNA, 35 bp, Artificial Sequence
  Synthetic oligonucleotide
cttcacctgctccacggcggtgctgttgttttcac

SEQ ID NO: 694 - DNA, 34 bp, Artificial Sequence
  Synthetic oligonucleotide
ccctgtgaaaacaagaacaaggccgtggagcagg

SEQ ID NO: 695 - DNA, 34 bp, Artificial Sequence
  Synthetic oligonucleotide
cctgctccacggccttgttcttgttttcacaggg

SEQ ID NO: 696 - DNA, 35 bp, Artificial Sequence
  Synthetic oligonucleotide
gtgaaaacaagaacaagagcgtggagcaggtgaag

SEQ ID NO: 697 - DNA, 35 bp, Artificial Sequence
  Synthetic oligonucleotide
cttcacctgctccacgctcttgttcttgttttcac

SEQ ID NO: 698 - DNA, 35 bp, Artificial Sequence
  Synthetic oligonucleotide
gtgaaaacaagaacaagaccgtggagcaggtgaag

SEQ ID NO: 699 - DNA, 35 bp, Artificial Sequence
  Synthetic oligonucleotide
cttcacctgctccacggtcttgttcttgttttcac

SEQ ID NO: 700 - DNA, 35 bp, Artificial Sequence
  Synthetic oligonucleotide
gtgaaaacaagagcaacgccgtggagcaggtgaag

SEQ ID NO: 701 - DNA, 35 bp, Artificial Sequence
  Synthetic oligonucleotide
cttcacctgctccacggcgttgctcttgttttcac

SEQ ID NO: 702 - DNA, 35 bp, Artificial Sequence
  Synthetic oligonucleotide
aaacaagagcaacgccagcgagcaggtgaagaatg

SEQ ID NO: 703 - DNA, 35 bp, Artificial Sequence
  Synthetic oligonucleotide
cattcttcacctgctcgctggcgttgctcttgttt

SEQ ID NO: 704 - DNA, 37 bp, Artificial Sequence
  Synthetic oligonucleotide
gaaaacaagagcaacgccaccgagcaggtgaagaatg

SEQ ID NO: 705 - DNA, 37 bp, Artificial Sequence
  Synthetic oligonucleotide
cattcttcacctgctcggtggcgttgctcttgttttc

SEQ ID NO: 706 - DNA, 34 bp, Artificial Sequence
  Synthetic oligonucleotide
caagagcaaggccgtgaaccaggtgaagaatgcc

SEQ ID NO: 707 - DNA, 34 bp, Artificial Sequence
  Synthetic oligonucleotide
ggcattcttcacctggttcacggccttgctcttg

SEQ ID NO: 708 - DNA, 35 bp, Artificial Sequence
  Synthetic oligonucleotide
ggccgtgaaccagagcaagaatgcctttaataagc

SEQ ID NO: 709 - DNA, 35 bp, Artificial Sequence
  Synthetic oligonucleotide
gcttattaaaggcattcttgctctggttcacggcc

SEQ ID NO: 710 - DNA, 35 bp, Artificial Sequence
  Synthetic oligonucleotide
gagcaaggccgtggagaacgtgaagaatgccttta

SEQ ID NO: 711 - DNA, 35 bp, Artificial Sequence
  Synthetic oligonucleotide
taaaggcattcttcacgttctccacggccttgctc

SEQ ID NO: 712 - DNA, 35 bp, Artificial Sequence
  Synthetic oligonucleotide
ggccgtggagaacgtgagcaatgcctttaataagc

SEQ ID NO: 713 - DNA, 35 bp, Artificial Sequence
  Synthetic oligonucleotide
gcttattaaaggcattgctcacgttctccacggcc

SEQ ID NO: 714 - DNA, 30 bp, Artificial Sequence
  Synthetic oligonucleotide
gtggagcaggtgaacaatgcctttaataag

SEQ ID NO: 715 - DNA, 30 bp, Artificial Sequence
  Synthetic oligonucleotide
cttattaaaggcattgttcacctgctccac

SEQ ID NO: 716 - DNA, 35 bp, Artificial Sequence
  Synthetic oligonucleotide
ggagcaggtgaacaatagctttaataagctccaag

SEQ ID NO: 717 - DNA, 35 bp, Artificial Sequence
  Synthetic oligonucleotide
cttggagcttattaaagctattgttcacctgctcc

SEQ ID NO: 718 - DNA, 35 bp, Artificial Sequence
  Synthetic oligonucleotide
ggagcaggtgaacaatacctttaataagctccaag

SEQ ID NO: 719 - DNA, 35 bp, Artificial Sequence
  Synthetic oligonucleotide
cttggagcttattaaaggtattgttcacctgctcc

SEQ ID NO: 720 - DNA, 39 bp, Artificial Sequence
  Synthetic oligonucleotide
caggtgaagaatgcctctaataagctccaagagaaaggc

SEQ ID NO: 721 - DNA, 39 bp, Artificial Sequence
  Synthetic oligonucleotide
gcctttctcttggagcttattagaggcattcttcacctg

SEQ ID NO: 722 - DNA, 34 bp, Artificial Sequence
  Synthetic oligonucleotide
gcaggtgaagaatgccaccaataagctccaagag

SEQ ID NO: 723 - DNA, 34 bp, Artificial Sequence
  Synthetic oligonucleotide
ctcttggagcttattggtggcattcttcacctgc

SEQ ID NO: 724 - DNA, 34 bp, Artificial Sequence
  Synthetic oligonucleotide
gaatgcctttaataacctccaagagaaaggcatc

SEQ ID NO: 725 - DNA, 34 bp, Artificial Sequence
  Synthetic oligonucleotide
gatgcctttctcttggaggttattaaaggcattc

SEQ ID NO: 726 - DNA, 33 bp, Artificial Sequence
  Synthetic oligonucleotide
gcctttaataacctcagcgagaaaggcatctac

SEQ ID NO: 727 - DNA, 33 bp, Artificial Sequence
  Synthetic oligonucleotide
gtagatgcctttctcgctgaggttattaaaggc

SEQ ID NO: 728 - DNA, 33 bp, Artificial Sequence
  Synthetic oligonucleotide
gcctttaataacctcaccgagaaaggcatctac

SEQ ID NO: 729 - DNA, 33 bp, Artificial Sequence
  Synthetic oligonucleotide
gtagatgcctttctcggtgaggttattaaaggc

SEQ ID NO: 730 - DNA, 39 bp, Artificial Sequence
  Synthetic oligonucleotide
caggtgaagaatgcctttaataagagccaagagaaaggc

SEQ ID NO: 731 - DNA, 39 bp, Artificial Sequence
  Synthetic oligonucleotide
gcctttctcttggctcttattaaaggcattcttcacctg

SEQ ID NO: 732 - DNA, 34 bp, Artificial Sequence
  Synthetic oligonucleotide
gaatgcctttaataagacccaagagaaaggcatc

SEQ ID NO: 733 - DNA, 34 bp, Artificial Sequence
  Synthetic oligonucleotide
gatgcctttctcttgggtcttattaaaggcattc

SEQ ID NO: 734 - DNA, 35 bp, Artificial Sequence
  Synthetic oligonucleotide
gcctttaataagctcaacgagaaaggcatctacaa

SEQ ID NO: 735 - DNA, 35 bp, Artificial Sequence
  Synthetic oligonucleotide
ttgtagatgcctttctcgttgagcttattaaaggc

SEQ ID NO: 736 - DNA, 35 bp, Artificial Sequence
  Synthetic oligonucleotide
ttaataagctcaacgagagcggcatctacaaagcc

SEQ ID NO: 737 - DNA, 35 bp, Artificial Sequence
  Synthetic oligonucleotide
ggctttgtagatgccgctctcgttgagcttattaa

SEQ ID NO: 738 - DNA, 35 bp, Artificial Sequence
  Synthetic oligonucleotide
ttaataagctcaacgagaccggcatctacaaagcc

SEQ ID NO: 739 - DNA, 35 bp, Artificial Sequence
  Synthetic oligonucleotide
ggctttgtagatgccggtctcgttgagcttattaa

SEQ ID NO: 740 - DNA, 35 bp, Artificial Sequence
  Synthetic oligonucleotide
ctttaataagctccaaaacaaaggcatctacaaag

SEQ ID NO: 741 - DNA, 35 bp, Artificial Sequence
  Synthetic oligonucleotide
ctttgtagatgcctttgttttggagcttattaaag

SEQ ID NO: 742 - DNA, 35 bp, Artificial Sequence
  Synthetic oligonucleotide
ataagctccaaaacaaaagcatctacaaagccatg

SEQ ID NO: 743 - DNA, 35 bp, Artificial Sequence
  Synthetic oligonucleotide
catggctttgtagatgcttttgttttggagcttat

SEQ ID NO: 744 - DNA, 35 bp, Artificial Sequence
  Synthetic oligonucleotide
ataagctccaaaacaaaaccatctacaaagccatg

SEQ ID NO: 745 - DNA, 35 bp, Artificial Sequence
  Synthetic oligonucleotide
catggctttgtagatggttttgttttggagcttat

SEQ ID NO: 746 - DNA, 35 bp, Artificial Sequence
  Synthetic oligonucleotide
ataagctccaagagaacggcatctacaaagccatg

SEQ ID NO: 747 - DNA, 35 bp, Artificial Sequence
  Synthetic oligonucleotide
catggctttgtagatgccgttctcttggagcttat

SEQ ID NO: 748 - DNA, 35 bp, Artificial Sequence
  Synthetic oligonucleotide
gctccaagagaacggcagctacaaagccatgagtg

SEQ ID NO: 749 - DNA, 35 bp, Artificial Sequence
  Synthetic oligonucleotide
cactcatggctttgtagctgccgttctcttggagc

SEQ ID NO: 750 - DNA, 35 bp, Artificial Sequence
  Synthetic oligonucleotide
gctccaagagaacggcacctacaaagccatgagtg

SEQ ID NO: 751 - DNA, 35 bp, Artificial Sequence
  Synthetic oligonucleotide
cactcatggctttgtaggtgccgttctcttggagc

SEQ ID NO: 752 - DNA, 35 bp, Artificial Sequence
  Synthetic oligonucleotide
ataagctccaagagaaaaacatctacaaagccatg

SEQ ID NO: 753 - DNA, 35 bp, Artificial Sequence
  Synthetic oligonucleotide
catggctttgtagatgtttttctcttggagcttat

SEQ ID NO: 754 - DNA, 34 bp, Artificial Sequence
  Synthetic oligonucleotide
ccaagagaaaaacatcagcaaagccatgagtgag

SEQ ID NO: 755 - DNA, 34 bp, Artificial Sequence
  Synthetic oligonucleotide
ctcactcatggctttgctgatgtttttctcttgg

SEQ ID NO: 756 - DNA, 34 bp, Artificial Sequence
  Synthetic oligonucleotide
ccaagagaaaaacatcaccaaagccatgagtgag

SEQ ID NO: 757 - DNA, 34 bp, Artificial Sequence
  Synthetic oligonucleotide
ctcactcatggctttggtgatgtttttctcttgg

SEQ ID NO: 758 - DNA, 35 bp, Artificial Sequence
  Synthetic oligonucleotide
gcctacatgacaatgaacatacgaaactgagggcc

SEQ ID NO: 759 - DNA, 35 bp, Artificial Sequence
  Synthetic oligonucleotide
ggccctcagtttcgtatgttcattgtcatgtaggc

SEQ ID NO: 760 - DNA, 35 bp, Artificial Sequence
  Synthetic oligonucleotide
catgacaatgaacataagcaactgagggcccgaac

SEQ ID NO: 761 - DNA, 35 bp, Artificial Sequence
  Synthetic oligonucleotide
gttcgggccctcagttgcttatgttcattgtcatg

SEQ ID NO: 762 - DNA, 35 bp, Artificial Sequence
  Synthetic oligonucleotide
catgacaatgaacataaccaactgagggcccgaac

SEQ ID NO: 763 - DNA, 35 bp, Artificial Sequence
  Synthetic oligonucleotide
gttcgggccctcagttggttatgttcattgtcatg